SEQUENCE LISTING (454 sequences)

SEQ ID NO: 1 - RNA, 14 nt, Artificial Sequence
  source: /note=“Description of Artificial Sequence Synthetic oligonucleotide”
ucuacuguuguaga

SEQ ID NO: 2 - RNA, 15 nt, Artificial Sequence
  source: /note=“Description of Artificial Sequence Synthetic oligonucleotide”
ucuacuguuguagau

SEQ ID NO: 3 - RNA, 14 nt, Artificial Sequence
  source: /note=“Description of Artificial Sequence Synthetic oligonucleotide”
ucugcuguugcaga

SEQ ID NO: 4 - RNA, 15 nt, Artificial Sequence
  source: /note=“Description of Artificial Sequence Synthetic oligonucleotide”
ucugcuguugcagau

SEQ ID NO: 5 - RNA, 14 nt, Artificial Sequence
  source: /note=“Description of Artificial Sequence Synthetic oligonucleotide”
uccacuguugugga

SEQ ID NO: 6 - RNA, 15 nt, Artificial Sequence
  source: /note=“Description of Artificial Sequence Synthetic oligonucleotide”
uccacuguuguggau

SEQ ID NO: 7 - RNA, 14 nt, Artificial Sequence
  source: /note=“Description of Artificial Sequence Synthetic oligonucleotide”
ccuacuguuguagg

SEQ ID NO: 8 - RNA, 15 nt, Artificial Sequence
  source: /note=“Description of Artificial Sequence Synthetic oligonucleotide”
ccuacuguuguaggu

SEQ ID NO: 9 - RNA, 14 nt, Artificial Sequence
  source: /note=“Description of Artificial Sequence Synthetic oligonucleotide”
ucuacuauuguaga

SEQ ID NO: 10 - RNA, 15 nt, Artificial Sequence
  source: /note=“Description of Artificial Sequence Synthetic oligonucleotide”
ucuacuauuguagau

SEQ ID NO: 11 - RNA, 15 nt, Artificial Sequence
  source: /note=“Description of Artificial Sequence Synthetic oligonucleotide”
ucuacugcuguagau

SEQ ID NO: 12 - RNA, 16 nt, Artificial Sequence
  source: /note=“Description of Artificial Sequence Synthetic oligonucleotide”
ucuacugcuguagauu

SEQ ID NO: 13 - RNA, 14 nt, Artificial Sequence
  source: /note=“Description of Artificial Sequence Synthetic oligonucleotide”
ucuacuuucuagau

SEQ ID NO: 14 - RNA, 15 nt, Artificial Sequence
  source: /note=“Description of Artificial Sequence Synthetic oligonucleotide”
ucuacuuucuagauu

SEQ ID NO: 15 - RNA, 13 nt, Artificial Sequence
  source: /note=“Description of Artificial Sequence Synthetic oligonucleotide”
ucuacuuuguaga

SEQ ID NO: 16 - RNA, 14 nt, Artificial Sequence
  source: /note=“Description of Artificial Sequence Synthetic oligonucleotide”
ucuacuuuguagau

SEQ ID NO: 17 - RNA, 12 nt, Artificial Sequence
  source: /note=“Description of Artificial Sequence Synthetic oligonucleotide”
ucuacuuguaga

SEQ ID NO: 18 - RNA, 13 nt, Artificial Sequence
  source: /note=“Description of Artificial Sequence Synthetic oligonucleotide”
ucuacuuguagau

SEQ ID NO: 19 - RNA, 20 nt, Artificial Sequence
  source: /note=“Description of Artificial Sequence Synthetic oligonucleotide”
uaauuucuacuguuguagau

SEQ ID NO: 20 - RNA, 21 nt, Artificial Sequence
  source: /note=“Description of Artificial Sequence Synthetic oligonucleotide”
agaaaugcaugguucucaugc

SEQ ID NO: 21 - RNA, 21 nt, Artificial Sequence
  source: /note=“Description of Artificial Sequence Synthetic oligonucleotide”
aaaauuaccuaguaauuaggu

SEQ ID NO: 22 - RNA, 20 nt, Artificial Sequence
  source: /note=“Description of Artificial Sequence Synthetic oligonucleotide”
ggauuucuacuuuuguagau

SEQ ID NO: 23 - RNA, 20 nt, Artificial Sequence
  source: /note=“Description of Artificial Sequence Synthetic oligonucleotide”
aaauuucuacuuuuguagau

SEQ ID NO: 24 - RNA, 21 nt, Artificial Sequence
  source: /note=“Description of Artificial Sequence Synthetic oligonucleotide”
cgcgcccacgcggggcgcgac

SEQ ID NO: 25 - RNA, 20 nt, Artificial Sequence
  source: /note=“Description of Artificial Sequence Synthetic oligonucleotide”
uaauuucuacucuuguagau

SEQ ID NO: 26 - RNA, 20 nt, Artificial Sequence
  source: /note=“Description of Artificial Sequence Synthetic oligonucleotide”
gaauuucuacuauuguagau

SEQ ID NO: 27 - RNA, 21 nt, Artificial Sequence
  source: /note=“Description of Artificial Sequence Synthetic oligonucleotide”
gaaucucuacucuuuguagau

SEQ ID NO: 28 - RNA, 19 nt, Artificial Sequence
  source: /note=“Description of Artificial Sequence Synthetic oligonucleotide”
uaauuucuacuuuguagau

SEQ ID NO: 29 - RNA, 21 nt, Artificial Sequence
  source: /note=“Description of Artificial Sequence Synthetic oligonucleotide”
aaauuucuacuguuuguagau

SEQ ID NO: 30 - RNA, 20 nt, Artificial Sequence
  source: /note=“Description of Artificial Sequence Synthetic oligonucleotide”
gaauuucuacuuuuguagau

SEQ ID NO: 31 - RNA, 21 nt, Artificial Sequence
  source: /note=“Description of Artificial Sequence Synthetic oligonucleotide”
uaauuucuacuaaguguagau

SEQ ID NO: 32 - RNA, 20 nt, Artificial Sequence
  source: /note=“Description of Artificial Sequence Synthetic oligonucleotide”
uaauuucuacuauuguagau

SEQ ID NO: 33 - RNA, 20 nt, Artificial Sequence
  source: /note=“Description of Artificial Sequence Synthetic oligonucleotide”
uaauuucuacuucgguagau

SEQ ID NO: 34 - RNA, 18 nt, Artificial Sequence
  source: /note=“Description of Artificial Sequence Synthetic oligonucleotide”
auuucuacuaguguagau

SEQ ID NO: 35 - RNA, 18 nt, Artificial Sequence
  source: /note=“Description of Artificial Sequence Synthetic oligonucleotide”
auuucuacuguguguaga

SEQ ID NO: 36 - RNA, 18 nt, Artificial Sequence
  source: /note=“Description of Artificial Sequence Synthetic oligonucleotide”
auuucuacuauuguagau

SEQ ID NO: 37 - RNA, 18 nt, Artificial Sequence
  source: /note=“Description of Artificial Sequence Synthetic oligonucleotide”
auuucuacuuugguagau

SEQ ID NO: 38 - DNA, 23 bp, Artificial Sequence
  source: /note=“Description of Artificial Sequence Synthetic oligonucleotide”
catagaaaacactgcaaataaca

SEQ ID NO: 39 - DNA, 23 bp, Artificial Sequence
  source: /note=“Description of Artificial Sequence Synthetic oligonucleotide”
agggtgtcttactcaccatttta

SEQ ID NO: 40 - DNA, 23 bp, Artificial Sequence
  source: /note=“Description of Artificial Sequence Synthetic oligonucleotide”
tacttgagatgtaagtaaggtta

SEQ ID NO: 41 - DNA, 23 bp, Artificial Sequence
  source: /note=“Description of Artificial Sequence Synthetic oligonucleotide”
atagtaaccttacttacatctca

SEQ ID NO: 42 - DNA, 23 bp, Artificial Sequence
  source: /note=“Description of Artificial Sequence Synthetic oligonucleotide”
aaattccatcttaccaattctaa

SEQ ID NO: 43 - DNA, 23 bp, Artificial Sequence
  source: /note=“Description of Artificial Sequence Synthetic oligonucleotide”
aacgttaaaattccatcttacca

SEQ ID NO: 44 - DNA, 23 bp, Artificial Sequence
  source: /note=“Description of Artificial Sequence Synthetic oligonucleotide”
tgacctttggtaagtcatctaat

SEQ ID NO: 45 - DNA, 23 bp, Artificial Sequence
  source: /note=“Description of Artificial Sequence Synthetic oligonucleotide”
cctttgtgacctttggtaagtca

SEQ ID NO: 46 - DNA, 23 bp, Artificial Sequence
  source: /note=“Description of Artificial Sequence Synthetic oligonucleotide”
ttgatcacataacaaggtcagtt

SEQ ID NO: 47 - DNA, 23 bp, Artificial Sequence
  source: /note=“Description of Artificial Sequence Synthetic oligonucleotide”
atcacataacaaggtcagtttat

SEQ ID NO: 48 - DNA, 23 bp, Artificial Sequence
  source: /note=“Description of Artificial Sequence Synthetic oligonucleotide”
agttatgataaactgaccttgtt

SEQ ID NO: 49 - DNA, 23 bp, Artificial Sequence
  source: /note=“Description of Artificial Sequence Synthetic oligonucleotide”
tgataaactgaccttgttatgtg

SEQ ID NO: 50 - DNA, 23 bp, Artificial Sequence
  source: /note=“Description of Artificial Sequence Synthetic oligonucleotide”
tcttccttggttttgcagcttct

SEQ ID NO: 51 - DNA, 23 bp, Artificial Sequence
  source: /note=“Description of Artificial Sequence Synthetic oligonucleotide”
ttggttttgcagcttctcgagtt

SEQ ID NO: 52 - DNA, 23 bp, Artificial Sequence
  source: /note=“Description of Artificial Sequence Synthetic oligonucleotide”
ctctttctcttccttggttttgc

SEQ ID NO: 53 - DNA, 23 bp, Artificial Sequence
  source: /note=“Description of Artificial Sequence Synthetic oligonucleotide”
cttgtttctctacataggttgaa

SEQ ID NO: 54 - DNA, 23 bp, Artificial Sequence
  source: /note=“Description of Artificial Sequence Synthetic oligonucleotide”
tcctctctatccacctcccccag

SEQ ID NO: 55 - DNA, 23 bp, Artificial Sequence
  source: /note=“Description of Artificial Sequence Synthetic oligonucleotide”
cctcccccagacccttctctgca

SEQ ID NO: 56 - DNA, 23 bp, Artificial Sequence
  source: /note=“Description of Artificial Sequence Synthetic oligonucleotide”
cccctcctctctatccactcccc

SEQ ID NO: 57 - DNA, 23 bp, Artificial Sequence
  source: /note=“Description of Artificial Sequence Synthetic oligonucleotide”
cctcctctctatccacctccccc

SEQ ID NO: 58 - DNA, 23 bp, Artificial Sequence
  source: /note=“Description of Artificial Sequence Synthetic oligonucleotide”
caaaaacccaaaatattttagct

SEQ ID NO: 59 - DNA, 23 bp, Artificial Sequence
  source: /note=“Description of Artificial Sequence Synthetic oligonucleotide”
ctttttgcaaaaacccaaaatat

SEQ ID NO: 60 - DNA, 23 bp, Artificial Sequence
  source: /note=“Description of Artificial Sequence Synthetic oligonucleotide”
tttttgcaaaaacccaaaatatt

SEQ ID NO: 61 - DNA, 23 bp, Artificial Sequence
  source: /note=“Description of Artificial Sequence Synthetic oligonucleotide”
tgtcaccagagtaacagtctgag

SEQ ID NO: 62 - DNA, 23 bp, Artificial Sequence
  source: /note=“Description of Artificial Sequence Synthetic oligonucleotide”
gctcctactcagactgttactct

SEQ ID NO: 63 - DNA, 23 bp, Artificial Sequence
  source: /note=“Description of Artificial Sequence Synthetic oligonucleotide”
tgggttaaggtaatagcaatatc

SEQ ID NO: 64 - DNA, 23 bp, Artificial Sequence
  source: /note=“Description of Artificial Sequence Synthetic oligonucleotide”
tatgcagagatattgctattacc

SEQ ID NO: 65 - DNA, 23 bp, Artificial Sequence
  source: /note=“Description of Artificial Sequence Synthetic oligonucleotide”
ctattaccttaacccagaaatta

SEQ ID NO: 66 - DNA, 23 bp, Artificial Sequence
  source: /note=“Description of Artificial Sequence Synthetic oligonucleotide”
cagagatattgctattaccttaa

SEQ ID NO: 67 - DNA, 23 bp, Artificial Sequence
  source: /note=“Description of Artificial Sequence Synthetic oligonucleotide”
tgcatataaattgtaactgaggt

SEQ ID NO: 68 - DNA, 23 bp, Artificial Sequence
  source: /note=“Description of Artificial Sequence Synthetic oligonucleotide”
aattgtaactgaggtaagaggtt

SEQ ID NO: 69 - DNA, 23 bp, Artificial Sequence
  source: /note=“Description of Artificial Sequence Synthetic oligonucleotide”
aaacctcttacctcagttacaat

SEQ ID NO: 70 - DNA, 23 bp, Artificial Sequence
  source: /note=“Description of Artificial Sequence Synthetic oligonucleotide”
gcaatatgaaacctcttacctca

SEQ ID NO: 71 - DNA, 23 bp, Artificial Sequence
  source: /note=“Description of Artificial Sequence Synthetic oligonucleotide”
ctaatagcagctacaatccaggt

SEQ ID NO: 72 - DNA, 23 bp, Artificial Sequence
  source: /note=“Description of Artificial Sequence Synthetic oligonucleotide”
ttttgcatacctgttcgttacct

SEQ ID NO: 73 - DNA, 23 bp, Artificial Sequence
  source: /note=“Description of Artificial Sequence Synthetic oligonucleotide”
aaatagaatgattttattttgca

SEQ ID NO: 74 - DNA, 23 bp, Artificial Sequence
  source: /note=“Description of Artificial Sequence Synthetic oligonucleotide”
tggtaagttacactaaccttagt

SEQ ID NO: 75 - DNA, 23 bp, Artificial Sequence
  source: /note=“Description of Artificial Sequence Synthetic oligonucleotide”
tcatctgtaaaataagagtaaaa

SEQ ID NO: 76 - DNA, 23 bp, Artificial Sequence
  source: /note=“Description of Artificial Sequence Synthetic oligonucleotide”
ccatgtctccccactaaagtgta

SEQ ID NO: 77 - DNA, 23 bp, Artificial Sequence
  source: /note=“Description of Artificial Sequence Synthetic oligonucleotide”
aggtgtggcttaggtacgagatg

SEQ ID NO: 78 - DNA, 23 bp, Artificial Sequence
  source: /note=“Description of Artificial Sequence Synthetic oligonucleotide”
taaaattcttacatacctttgaa

SEQ ID NO: 79 - DNA, 23 bp, Artificial Sequence
  source: /note=“Description of Artificial Sequence Synthetic oligonucleotide”
aaaaatcttactcagattatgac

SEQ ID NO: 80 - DNA, 23 bp, Artificial Sequence
  source: /note=“Description of Artificial Sequence Synthetic oligonucleotide”
tttaaaaaatcttactcagatta

SEQ ID NO: 81 - DNA, 23 bp, Artificial Sequence
  source: /note=“Description of Artificial Sequence Synthetic oligonucleotide”
agttgtaattgtgagtatctcat

SEQ ID NO: 82 - DNA, 22 bp, Artificial Sequence
  source: /note=“Description of Artificial Sequence Synthetic oligonucleotide”
tccatccacaccgcagggagag

SEQ ID NO: 83 - DNA, 23 bp, Artificial Sequence
  source: /note=“Description of Artificial Sequence Synthetic oligonucleotide”
tggatttattttagtttacagaa

SEQ ID NO: 84 - DNA, 23 bp, Artificial Sequence
  source: /note=“Description of Artificial Sequence Synthetic oligonucleotide”
ttttagtttacagaacctggacc

SEQ ID NO: 85 - DNA, 23 bp, Artificial Sequence
  source: /note=“Description of Artificial Sequence Synthetic oligonucleotide”
caagaggtctgactttctggatt

SEQ ID NO: 86 - DNA, 23 bp, Artificial Sequence
  source: /note=“Description of Artificial Sequence Synthetic oligonucleotide”
agaggtctgactttctggattta

SEQ ID NO: 87 - DNA, 23 bp, Artificial Sequence
  source: /note=“Description of Artificial Sequence Synthetic oligonucleotide”
ggttctgtaaactaaaataaatc

SEQ ID NO: 88 - DNA, 23 bp, Artificial Sequence
  source: /note=“Description of Artificial Sequence Synthetic oligonucleotide”
tatgtctgtacacataccttgtt

SEQ ID NO: 89 - DNA, 23 bp, Artificial Sequence
  source: /note=“Description of Artificial Sequence Synthetic oligonucleotide”
atatgtctgtacacataccttgt

SEQ ID NO: 90 - DNA, 23 bp, Artificial Sequence
  source: /note=“Description of Artificial Sequence Synthetic oligonucleotide”
ttaaagatgatctcttaccttgg

SEQ ID NO: 91 - DNA, 23 bp, Artificial Sequence
  source: /note=“Description of Artificial Sequence Synthetic oligonucleotide”
ccaaggtaagagatcatctttaa

SEQ ID NO: 92 - DNA, 23 bp, Artificial Sequence
  source: /note=“Description of Artificial Sequence Synthetic oligonucleotide”
aaattgaacacctctcctttccc

SEQ ID NO: 93 - DNA, 23 bp, Artificial Sequence
  source: /note=“Description of Artificial Sequence Synthetic oligonucleotide”
aagatgatctcttaccttgggaa

SEQ ID NO: 94 - DNA, 24 bp, Artificial Sequence
  source: /note=“Description of Artificial Sequence Synthetic oligonucleotide”
agctgctttcagcttcctctccag

SEQ ID NO: 95 - DNA, 23 bp, Artificial Sequence
  source: /note=“Description of Artificial Sequence Synthetic oligonucleotide”
tggagaggaagctgaaagcagct

SEQ ID NO: 96 - DNA, 23 bp, Artificial Sequence
  source: /note=“Description of Artificial Sequence Synthetic oligonucleotide”
tgtgattctggagaggaagctga

SEQ ID NO: 97 - DNA, 23 bp, Artificial Sequence
  source: /note=“Description of Artificial Sequence Synthetic oligonucleotide”
acttgtgtgattctggagaggaa

SEQ ID NO: 98 - DNA, 23 bp, Artificial Sequence
  source: /note=“Description of Artificial Sequence Synthetic oligonucleotide”
tgctgagcccgcttgcttctccc

SEQ ID NO: 99 - DNA, 23 bp, Artificial Sequence
  source: /note=“Description of Artificial Sequence Synthetic oligonucleotide”
gcctccctgctgagcccgcttgc

SEQ ID NO: 100 - DNA, 23 bp, Artificial Sequence
  source: /note=“Description of Artificial Sequence Synthetic oligonucleotide”
tcccgcctccctgctgagcccgc

SEQ ID NO: 101 - DNA, 23 bp, Artificial Sequence
  source: /note=“Description of Artificial Sequence Synthetic oligonucleotide”
tcctccctccctcaggaagtcgg

SEQ ID NO: 102 - DNA, 23 bp, Artificial Sequence
  source: /note=“Description of Artificial Sequence Synthetic oligonucleotide”
aaggctccctcctccctccctca

SEQ ID NO: 103 - DNA, 23 bp, Artificial Sequence
  source: /note=“Description of Artificial Sequence Synthetic oligonucleotide”
tccctcaggaagtcggcgttggc

SEQ ID NO: 104 - DNA, 23 bp, Artificial Sequence
  source: /note=“Description of Artificial Sequence Synthetic oligonucleotide”
tgatatgattattctaatttagt

SEQ ID NO: 105 - DNA, 23 bp, Artificial Sequence
  source: /note=“Description of Artificial Sequence Synthetic oligonucleotide”
gtctttttcaggtacaagatatt

SEQ ID NO: 106 - DNA, 23 bp, Artificial Sequence
  source: /note=“Description of Artificial Sequence Synthetic oligonucleotide”
ataatatcttgtacctgaaaaag

SEQ ID NO: 107 - DNA, 23 bp, Artificial Sequence
  source: /note=“Description of Artificial Sequence Synthetic oligonucleotide”
tgttatttgcagtgttttctatg

SEQ ID NO: 108 - DNA, 23 bp, Artificial Sequence
  source: /note=“Description of Artificial Sequence Synthetic oligonucleotide”
cagtgttttctatggaaatattt

SEQ ID NO: 109 - DNA, 23 bp, Artificial Sequence
  source: /note=“Description of Artificial Sequence Synthetic oligonucleotide”
catagaaaacattgcaaataaca

SEQ ID NO: 110 - DNA, 33 bp, Artificial Sequence
  source: /note=“Description of Artificial Sequence Synthetic oligonucleotide”
acatttgtgatatgattattctaatttagtctt

SEQ ID NO: 111 - DNA, 33 bp, Artificial Sequence
  source: /note=“Description of Artificial Sequence Synthetic oligonucleotide”
taatttagtctttttcaggtacaagatattatg

SEQ ID NO: 112 - DNA, 33 bp, Artificial Sequence
  source: /note=“Description of Artificial Sequence Synthetic oligonucleotide”
taatttcataatatcttgtacctgaaaaagact

SEQ ID NO: 113 - DNA, 33 bp, Artificial Sequence
  source: /note=“Description of Artificial Sequence Synthetic oligonucleotide”
gtgtttatgttatttgcagtgttttctatggaa

SEQ ID NO: 114 - DNA, 33 bp, Artificial Sequence
  source: /note=“Description of Artificial Sequence Synthetic oligonucleotide”
ttatttgcagtgttttctatggaaatatttcac

SEQ ID NO: 115 - DNA, 33 bp, Artificial Sequence
  source: /note=“Description of Artificial Sequence Synthetic oligonucleotide”
atatttccatagaaaacactgcaaataacataa

SEQ ID NO: 116 - DNA, 33 bp, Artificial Sequence
  source: /note=“Description of Artificial Sequence Synthetic oligonucleotide”
atatttccatagaaaacattgcaaataacataa

SEQ ID NO: 117 - DNA, 50 bp, Artificial Sequence
  source: /note=“Description of Artificial Sequence Synthetic primer”
atggtaccggtgaccttctgcctcttaccatatttgacttcatccagttg

SEQ ID NO: 118 - DNA, 51 bp, Artificial Sequence
  source: /note=“Description of Artificial Sequence Synthetic primer”
ttaccatatttgacttcatccagttgttattaattgtgattggagctatag

SEQ ID NO: 119 - DNA, 54 bp, Artificial Sequence
  source: /note=“Description of Artificial Sequence Synthetic primer”
tgtagaattcttaggatccctcgcctgttgttaaaatggaaatgaaggtaacag

SEQ ID NO: 120 - DNA, 33 bp, Artificial Sequence
  source: /note=“Description of Artificial Sequence Synthetic primer”
atggtctcagtgttttctatggaaatatttcac

SEQ ID NO: 121 - DNA, 37 bp, Artificial Sequence
  source: /note=“Description of Artificial Sequence Synthetic primer”
atggtctcaacactgcaaataacataaacacaaaatg

SEQ ID NO: 122 - DNA, 18 bp, Artificial Sequence
  source: /note=“Description of Artificial Sequence Synthetic primer”
tagaaggcacagtcgagg

SEQ ID NO: 123 - DNA, 61 bp, Artificial Sequence
  source: /note=“Description of Artificial Sequence Synthetic primer”
tcgtcggcagcgtcagatgtgtataagagacaggcttgtaacaagatgagtgaaaattgga

SEQ ID NO: 124 - DNA, 62 bp, Artificial Sequence
  source: /note=“Description of Artificial Sequence Synthetic primer”
gtctcgtgggctcggagatgtgtataagagacagatatctattcaaagaatggcaccagtgt

SEQ ID NO: 125 - DNA, 33 bp, Artificial Sequence
  source: /note=“Description of Artificial Sequence Synthetic oligonucleotide”
tcctttcagggtgtcttactcaccattttaata

SEQ ID NO: 126 - DNA, 33 bp, Artificial Sequence
  source: /note=“Description of Artificial Sequence Synthetic oligonucleotide”
tcctttcagggtgtcttactcgccattttaata

SEQ ID NO: 127 - DNA, 36 bp, Artificial Sequence
  source: /note=“Description of Artificial Sequence Synthetic primer”
atatctcgagatgcgatctgtgagccgagtctttaa

SEQ ID NO: 128 - DNA, 40 bp, Artificial Sequence
  source: /note=“Description of Artificial Sequence Synthetic primer”
tacgtctcatatattcagtgggtataagcagcatattctc

SEQ ID NO: 129 - DNA, 48 bp, Artificial Sequence
  source: /note=“Description of Artificial Sequence Synthetic primer”
tatggatccagatcgtctcgaaaggtcagtgataaaggaagtctgcat

SEQ ID NO: 130 - DNA, 52 bp, Artificial Sequence
  source: /note=“Description of Artificial Sequence Synthetic primer”
tatggatccagatcgtctcgatataggttcaggactctgcaaattaaatttc

SEQ ID NO: 131 - DNA, 40 bp, Artificial Sequence
  source: /note=“Description of Artificial Sequence Synthetic primer”
atcgtctctctttaggcttctcagtgatctgttgaataag

SEQ ID NO: 132 - DNA, 38 bp, Artificial Sequence
  source: /note=“Description of Artificial Sequence Synthetic primer”
atagtgcggccgcctgtggtatcactccaaaggctttc

SEQ ID NO: 133 - DNA, 45 bp, Artificial Sequence
  source: /note=“Description of Artificial Sequence Synthetic primer”
ccatctgttgcagtattaaaatggtgagtaagacaccctgaaagg

SEQ ID NO: 134 - DNA, 45 bp, Artificial Sequence
  source: /note=“Description of Artificial Sequence Synthetic primer”
cctttcagggtgtcttactcaccattttaatactgcaacagatgg

SEQ ID NO: 135 - DNA, 32 bp, Artificial Sequence
  source: /note=“Description of Artificial Sequence Synthetic primer”
tacttaatacgactcactataggctagcctcg

SEQ ID NO: 136 - DNA, 26 bp, Artificial Sequence
  source: /note=“Description of Artificial Sequence Synthetic primer”
ctgctttctccatttgtagtctcttg

SEQ ID NO: 137 - DNA, 26 bp, Artificial Sequence
  source: /note=“Description of Artificial Sequence Synthetic primer”
tgctggtaatgcatgatatctgacac

SEQ ID NO: 138 - DNA, 20 bp, Artificial Sequence
  source: /note=“Description of Artificial Sequence Synthetic oligonucleotide”
aatcatatcacaaatgtcat

SEQ ID NO: 139 - DNA, 20 bp, Artificial Sequence
  source: /note=“Description of Artificial Sequence Synthetic oligonucleotide”
gtacctgaaaaagactaaat

SEQ ID NO: 140 - DNA, 20 bp, Artificial Sequence
  source: /note=“Description of Artificial Sequence Synthetic oligonucleotide”
ataatatcttgtacctgaaa

SEQ ID NO: 141 - DNA, 20 bp, Artificial Sequence
  source: /note=“Description of Artificial Sequence Synthetic oligonucleotide”
attctaatttagtctttttc

SEQ ID NO: 142 - DNA, 20 bp, Artificial Sequence
  source: /note=“Description of Artificial Sequence Synthetic oligonucleotide”
ttttgtgtttatgttatttg

SEQ ID NO: 143 - DNA, 20 bp, Artificial Sequence
  source: /note=“Description of Artificial Sequence Synthetic oligonucleotide”
ctgcctgtgaaatatttcca

SEQ ID NO: 144 - DNA, 20 bp, Artificial Sequence
  source: /note=“Description of Artificial Sequence Synthetic oligonucleotide”
gttatttgcagtgttttcta

SEQ ID NO: 145 - DNA, 20 bp, Artificial Sequence
  source: /note=“Description of Artificial Sequence Synthetic oligonucleotide”
ttttctatggaaatatttca

SEQ ID NO: 146 - DNA, 29 bp, Artificial Sequence
  source: /note=“Description of Artificial Sequence Synthetic oligonucleotide”
aataatcatatcacaaatgtcattggtta

SEQ ID NO: 147 - DNA, 29 bp, Artificial Sequence
  source: /note=“Description of Artificial Sequence Synthetic oligonucleotide”
cttgtacctgaaaaagactaaattagaat

SEQ ID NO: 148 - DNA, 29 bp, Artificial Sequence
  source: /note=“Description of Artificial Sequence Synthetic oligonucleotide”
ttcataatatcttgtacctgaaaaagact

SEQ ID NO: 149 - DNA, 29 bp, Artificial Sequence
  source: /note=“Description of Artificial Sequence Synthetic oligonucleotide”
attattctaatttagtctttttcaggtac

SEQ ID NO: 150 - DNA, 29 bp, Artificial Sequence
  source: /note=“Description of Artificial Sequence Synthetic oligonucleotide”
acattttgtgtttatgttatttgcagtgt

SEQ ID NO: 151 - DNA, 29 bp, Artificial Sequence
  source: /note=“Description of Artificial Sequence Synthetic oligonucleotide”
ctcctgcctgtgaaatatttccatagaaa

SEQ ID NO: 152 - DNA, 29 bp, Artificial Sequence
  source: /note=“Description of Artificial Sequence Synthetic oligonucleotide”
tatgttatttgcagtgttttctatggaaa

SEQ ID NO: 153 - DNA, 29 bp, Artificial Sequence
  source: /note=“Description of Artificial Sequence Synthetic oligonucleotide”
gtgttttctatggaaatatttcacaggca

SEQ ID NO: 154 - DNA, 20 bp, Artificial Sequence
  source: /note=“Description of Artificial Sequence Synthetic oligonucleotide”
attcaattataatcaccttg

SEQ ID NO: 155 - DNA, 20 bp, Artificial Sequence
  source: /note=“Description of Artificial Sequence Synthetic oligonucleotide”
aactgaaatttagatccaca

SEQ ID NO: 156 - DNA, 20 bp, Artificial Sequence
  source: /note=“Description of Artificial Sequence Synthetic oligonucleotide”
cttgatttctggagaccaca

SEQ ID NO: 157 - DNA, 20 bp, Artificial Sequence
  source: /note=“Description of Artificial Sequence Synthetic oligonucleotide”
gaaaggaaatgttctattca

SEQ ID NO: 158 - DNA, 20 bp, Artificial Sequence
  source: /note=“Description of Artificial Sequence Synthetic oligonucleotide”
cacctcctccctgagaatgt

SEQ ID NO: 159 - DNA, 20 bp, Artificial Sequence
  source: /note=“Description of Artificial Sequence Synthetic oligonucleotide”
ttgatccaacattctcaggg

SEQ ID NO: 160 - DNA, 29 bp, Artificial Sequence
  source: /note=“Description of Artificial Sequence Synthetic oligonucleotide”
aagattcaattataatcaccttgtggatc

SEQ ID NO: 161 - DNA, 29 bp, Artificial Sequence
  source: /note=“Description of Artificial Sequence Synthetic oligonucleotide”
gtcaactgaaatttagatccacaaggtga

SEQ ID NO: 162 - DNA, 29 bp, Artificial Sequence
  source: /note=“Description of Artificial Sequence Synthetic oligonucleotide”
catcttgatttctggagaccacaaggtaa

SEQ ID NO: 163 - DNA, 29 bp, Artificial Sequence
  source: /note=“Description of Artificial Sequence Synthetic oligonucleotide”
cttgaaaggaaatgttctattcatggtac

SEQ ID NO: 164 - DNA, 29 bp, Artificial Sequence
  source: /note=“Description of Artificial Sequence Synthetic oligonucleotide”
atgcacctcctccctgagaatgttggatc

SEQ ID NO: 165 - DNA, 29 bp, Artificial Sequence
  source: /note=“Description of Artificial Sequence Synthetic oligonucleotide”
atcttgatccaacattctcagggaggagg

SEQ ID NO: 166 - DNA, 65 bp, Artificial Sequence
  source: /note=“Description of Artificial Sequence Synthetic oligonucleotide”
gagccaccgcacctggccccagttgtaattgtgartatctcatacctatccctattggcagtgtc

SEQ ID NO: 167 - DNA, 27 bp, Artificial Sequence
  source: /note=“Description of Artificial Sequence Synthetic oligonucleotide”
ccccagttgtaattgtgagtatctcat

SEQ ID NO: 168 - DNA, 54 bp, Artificial Sequence
  source: /note=“Description of Artificial Sequence Synthetic primer”
ggggacaagtttgtacaaaaaagcaggcttcggccgctctttctcaaaagtggc

SEQ ID NO: 169 - DNA, 51 bp, Artificial Sequence
  source: /note=“Description of Artificial Sequence Synthetic primer”
ggggaccactttgtacaagaaagctgggtgcttggtggggttaagtacagg

SEQ ID NO: 170 - DNA, 43 bp, Artificial Sequence
  source: /note=“Description of Artificial Sequence Synthetic primer”
cacctggccccagttgtaattgtgagtatctcatacctatccc

SEQ ID NO: 171 - DNA, 43 bp, Artificial Sequence
  source: /note=“Description of Artificial Sequence Synthetic primer”
gggataggtatgagatactcacaattacaactggggccaggtg

SEQ ID NO: 172 - DNA, 23 bp, Artificial Sequence
  source: /note=“Description of Artificial Sequence Synthetic primer”
tgctaagtacagggacatcttgc

SEQ ID NO: 173 - DNA, 25 bp, Artificial Sequence
  source: /note=“Description of Artificial Sequence Synthetic primer”
agactccacttgttcttttaaggag

SEQ ID NO: 174 - DNA, 101 bp, Artificial Sequence
  source: /note=“Description of Artificial Sequence Synthetic polynucleotide”
ggcttttaagggggaaacaaatcatgaaattgaaattgaacacctctcctttcccaagrgtaagagatcatctttaagaaaaggctgtgtattgtgggggt

SEQ ID NO: 175 - DNA, 88 bp, Artificial Sequence
  source: /note=“Description of Artificial Sequence Synthetic oligonucleotide”
ttttaacacttccctagccaaaggagctaattaagctgctttcagcttcctctccagaatcacacaagttaaaggacccttctgcaac

SEQ ID NO: 176 - DNA, 27 bp, Artificial Sequence
  source: /note=“Description of Artificial Sequence Synthetic oligonucleotide”
tttcttaaagatgatctcttaccttgg

SEQ ID NO: 177 - DNA, 27 bp, Artificial Sequence
  source: /note=“Description of Artificial Sequence Synthetic oligonucleotide”
tttcccaaggtaagagatcatctttaa

SEQ ID NO: 178 - DNA, 27 bp, Artificial Sequence
  source: /note=“Description of Artificial Sequence Synthetic oligonucleotide”
attgaaattgaacacctctcctttccc

SEQ ID NO: 179 - DNA, 27 bp, Artificial Sequence
  source: /note=“Description of Artificial Sequence Synthetic oligonucleotide”
cttaaagatgatctcttaccttgggaa

SEQ ID NO: 180 - DNA, 28 bp, Artificial Sequence
  source: /note=“Description of Artificial Sequence Synthetic oligonucleotide”
attaagctgctttcagcttcctctccag

SEQ ID NO: 181 - DNA, 27 bp, Artificial Sequence
  source: /note=“Description of Artificial Sequence Synthetic oligonucleotide”
attctggagaggaagctgaaagcagct

SEQ ID NO: 182 - DNA, 27 bp, Artificial Sequence
  source: /note=“Description of Artificial Sequence Synthetic oligonucleotide”
cttgtgtgattctggagaggaagctga

SEQ ID NO: 183 - DNA, 27 bp, Artificial Sequence
  source: /note=“Description of Artificial Sequence Synthetic oligonucleotide”
tttaacttgtgtgattctggagaggaa

SEQ ID NO: 184 - DNA, 20 bp, Artificial Sequence
  source: /note=“Description of Artificial Sequence Synthetic primer”
cggaggtcaacaacgagtct

SEQ ID NO: 185 - DNA, 20 bp, Artificial Sequence
  source: /note=“Description of Artificial Sequence Synthetic primer”
aggtgtaggggatgggagac

SEQ ID NO: 186 - DNA, 21 bp, Artificial Sequence
  source: /note=“Description of Artificial Sequence Synthetic primer”
gctctcccagataccaactcc

SEQ ID NO: 187 - DNA, 20 bp, Artificial Sequence
  source: /note=“Description of Artificial Sequence Synthetic primer”
gattcacatgcctgaccctc

SEQ ID NO: 188 - DNA, 101 bp, Artificial Sequence
  source: /note=“Description of Artificial Sequence Synthetic polynucleotide”
tttttctttttcttcttttttcctttttgcaaaaacccaaaatattttagctcctactcagactgttactctggtgacacaacctgtggttactaaggaaa

SEQ ID NO: 189 - DNA, 27 bp, Artificial Sequence
  source: /note=“Description of Artificial Sequence Synthetic oligonucleotide”
tttgcaaaaacccaaaatattttagct

SEQ ID NO: 190 - DNA, 27 bp, Artificial Sequence
  source: /note=“Description of Artificial Sequence Synthetic oligonucleotide”
tttcctttttgcaaaaacccaaaatat

SEQ ID NO: 191 - DNA, 27 bp, Artificial Sequence
  source: /note=“Description of Artificial Sequence Synthetic oligonucleotide”
ttcctttttgcaaaaacccaaaatatt

SEQ ID NO: 192 - DNA, 27 bp, Artificial Sequence
  source: /note=“Description of Artificial Sequence Synthetic oligonucleotide”
gttgtgtcaccagagtaacagtctgag

SEQ ID NO: 193 - DNA, 27 bp, Artificial Sequence
  source: /note=“Description of Artificial Sequence Synthetic oligonucleotide”
tttagctcctactcagactgttactct

SEQ ID NO: 194 - DNA, 32 bp, Artificial Sequence
  source: /note=“Description of Artificial Sequence Synthetic primer”
gaaactgaaatagcagttcaagctaaacaacc

SEQ ID NO: 195 - DNA, 35 bp, Artificial Sequence
  source: /note=“Description of Artificial Sequence Synthetic primer”
gccttaagatcacaatatataaataggatatgctg

SEQ ID NO: 196 - DNA, 31 bp, Artificial Sequence
  source: /note=“Description of Artificial Sequence Synthetic primer”
tgaatcttttcattttctaccatgtattgct

SEQ ID NO: 197 - DNA, 33 bp, Artificial Sequence
  source: /note=“Description of Artificial Sequence Synthetic primer”
ctttttaatgtatggctacttttgttatttgca

SEQ ID NO: 198 - DNA, 41 bp, Artificial Sequence
  source: /note=“Description of Artificial Sequence Synthetic primer”
tgaaatatttttgatatctaagaatgaaacatatttcctgt

SEQ ID NO: 199 - DNA, 29 bp, Artificial Sequence
  source: /note=“Description of Artificial Sequence Synthetic primer”
ttcgatccgtaatgattgttctagcctct

SEQ ID NO: 200 - DNA, 55 bp, Artificial Sequence
  source: /note=“Description of Artificial Sequence Synthetic oligonucleotide”
ccaccagttacctttgtgacctttggyaagtcatctaatttttctatttccattt

SEQ ID NO: 201 - DNA, 65 bp, Artificial Sequence
  source: /note=“Description of Artificial Sequence Synthetic oligonucleotide”
gtcctgatagtcgattattgatcacataacaaggtcartttatcataactgaagtgcgatcgatt

SEQ ID NO: 202 - DNA, 70 bp, Artificial Sequence
  source: /note=“Description of Artificial Sequence Synthetic oligonucleotide”
tattataaaattactctttctcttccttggttttgcrgcttctcgagttcataggagactttcagtttcc

SEQ ID NO: 203 - DNA, 73 bp, Artificial Sequence
  source: /note=“Description of Artificial Sequence Synthetic oligonucleotide”
acctagtttgtaataagccatatttccttgtttctctacatwggttgaatctgttcctgcagcaactagtaac

SEQ ID NO: 204 - DNA, 70 bp, Artificial Sequence
  source: /note=“Description of Artificial Sequence Synthetic oligonucleotide”
tttccccctcctctctatccactcccccaracccttctctgcagatcacggtcagtctagcacagggata

SEQ ID NO: 205 - DNA, 64 bp, Artificial Sequence
  source: /note=“Description of Artificial Sequence Synthetic oligonucleotide”
aattactgtggcctaccaggtaacgaacaggtatgcaaaataaaatcattctatttgaaatggg

SEQ ID NO: 206 - DNA, 94 bp, Artificial Sequence
  source: /note=“Description of Artificial Sequence Synthetic oligonucleotide”
ctttttttccaaagcaattaaaaaaactgccaaagtaagagtgactgcggaactaaggttastgtaacttaccatggaggattaagggtagcgt

SEQ ID NO: 207 - DNA, 133 bp, Artificial Sequence
  source: /note=“Description of Artificial Sequence Synthetic polynucleotide”
ttctttcagggcaataatgatacaatgtatcatgcctctttgcaccattctaaagaataacagtgataatttctgggttaaggtaatagcaatatctctgcatataaatatttctgcatataaattgtaactg

SEQ ID NO: 208 - DNA, 184 bp, Artificial Sequence
  source: /note=“Description of Artificial Sequence Synthetic polynucleotide”
ttccctaatctctttctttcagggcaataatgatacaatgtatcatgcctctttgcaccattctaaagaataacagtgataatttctgggttaaggcaatagcaatatctctgcatataaatatttctgcatataaattgtaactgakgtaagaggtttcatattgctaatagcagctacaatc

SEQ ID NO: 209 - DNA, 204 bp, Artificial Sequence
  source: /note=“Description of Artificial Sequence Synthetic polynucleotide”
tcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagstaccattctgcttttattttatggttgggataag

SEQ ID NO: 210 - DNA, 94 bp, Artificial Sequence
  source: /note=“Description of Artificial Sequence Synthetic oligonucleotide”
acagaagtaccaacaattacatgtataaacagagaatcctatgtacttgagatrtaagtaaggttactatcaatcacacctgaaaaatttaaat

SEQ ID NO: 211 - DNA, 85 bp, Artificial Sequence
  source: /note=“Description of Artificial Sequence Synthetic oligonucleotide”
gcttgatcaatggcatgggaaaacaggcaatacagttagaattggtaagatggaattttaacgttcaattaaggatctatctcta

SEQ ID NO: 212 - DNA, 65 bp, Artificial Sequence
  source: /note=“Description of Artificial Sequence Synthetic oligonucleotide”
gttttgtcatctgtaaaataagrtaaaatagtgtctcctttatatatatggtggttgtaccttgt

SEQ ID NO: 213 - DNA, 95 bp, Artificial Sequence
  source: /note=“Description of Artificial Sequence Synthetic oligonucleotide”
ttctcagagctccacactatttggaagtatttgttgacttgttaccatgtctccccactaragtgtaagtttcatgagggcagggaccttgtctg

SEQ ID NO: 214 - DNA, 115 bp, Artificial Sequence
  source: /note=“Description of Artificial Sequence Synthetic polynucleotide”
gggcaaccggaagaccatcttggaggatgaaaagaggctggcccaccccttctccttggccgtctttgaggtgtggcttasgtacgagatgcaagcacttaggtggcggatagac

SEQ ID NO: 215 - DNA, 75 bp, Artificial Sequence
  source: /note=“Description of Artificial Sequence Synthetic oligonucleotide”
gtagatgaaggctgagactcaggtttcaaaggwatgtaagaattttataacttgttgctaatactttaaaaactt

SEQ ID NO: 216 - DNA, 65 bp, Artificial Sequence
  source: /note=“Description of Artificial Sequence Synthetic oligonucleotide”
aagtcctgtgaaccagcaggtcataatctgartaagattttttaaagaaaatctgtatctgaaac

SEQ ID NO: 217 - DNA, 51 bp, Artificial Sequence
  source: /note=“Description of Artificial Sequence Synthetic oligonucleotide”
tttctccatccacaccgcrgggagagggagtctgatcctgatttgtgccgc

SEQ ID NO: 218 - DNA, 95 bp, Artificial Sequence
  source: /note=“Description of Artificial Sequence Synthetic oligonucleotide”
gaggtgggacatttccaagaggtctgactttctggatttattttastttacagaacctggacctgtagttcctccgattcttctggatgtgaagt

SEQ ID NO: 219 - DNA, 65 bp, Artificial Sequence
  source: /note=“Description of Artificial Sequence Synthetic oligonucleotide”
ggcagaaggatgaagaaactaacaaggyatgtgtacagacatatgaactcatggtatagcctact

SEQ ID NO: 220 - DNA, 72 bp, Artificial Sequence
  source: /note=“Description of Artificial Sequence Synthetic oligonucleotide”
agtgccgcccctcccgcctccctgctgagcccgcttkcttctcccgcaggcctgtaggagctgtccaggcca

SEQ ID NO: 221 - DNA, 69 bp, Artificial Sequence
  source: /note=“Description of Artificial Sequence Synthetic oligonucleotide”
gcccccgccccaaggctccctcctccctccctcakgaagtcggcgttggcctgcaggatacccgttcat

SEQ ID NO: 222 - DNA, 27 bp, Artificial Sequence
  source: /note=“Description of Artificial Sequence Synthetic oligonucleotide”
tttgtgacctttggtaagtcatctaat

SEQ ID NO: 223 - DNA, 27 bp, Artificial Sequence
  source: /note=“Description of Artificial Sequence Synthetic oligonucleotide”
gttacctttgtgacctttggtaagtca

SEQ ID NO: 224 - DNA, 27 bp, Artificial Sequence
  source: /note=“Description of Artificial Sequence Synthetic oligonucleotide”
attattgatcacataacaaggtcagtt

SEQ ID NO: 225 - DNA, 27 bp, Artificial Sequence
  source: /note=“Description of Artificial Sequence Synthetic oligonucleotide”
attgatcacataacaaggtcagtttat

SEQ ID NO: 226 - DNA, 27 bp, Artificial Sequence
  source: /note=“Description of Artificial Sequence Synthetic oligonucleotide”
cttcagttatgataaactgaccttgtt

SEQ ID NO: 227 - DNA, 27 bp, Artificial Sequence
  source: /note=“Description of Artificial Sequence Synthetic oligonucleotide”
gttatgataaactgaccttgttatgtg

SEQ ID NO: 228 - DNA, 27 bp, Artificial Sequence
  source: /note=“Description of Artificial Sequence Synthetic oligonucleotide”
tttctcttccttggttttgcagcttct

SEQ ID NO: 229 - DNA, 27 bp, Artificial Sequence
  source: /note=“Description of Artificial Sequence Synthetic oligonucleotide”
ttccttggttttgcagcttctcgagtt

SEQ ID NO: 230 - DNA, 27 bp, Artificial Sequence
  source: /note=“Description of Artificial Sequence Synthetic oligonucleotide”
attactctttctcttccttggttttgc

SEQ ID NO: 231 - DNA, 27 bp, Artificial Sequence
  source: /note=“Description of Artificial Sequence Synthetic oligonucleotide”
tttccttgtttctctacataggttgaa

SEQ ID NO: 232 - DNA, 27 bp, Artificial Sequence
  source: /note=“Description of Artificial Sequence Synthetic oligonucleotide”
cccctcctctctatccacctcccccag

SEQ ID NO: 233 - DNA, 27 bp, Artificial Sequence
  source: /note=“Description of Artificial Sequence Synthetic oligonucleotide”
tttccccctcctctctatccactcccc

SEQ ID NO: 234 - DNA, 27 bp, Artificial Sequence
  source: /note=“Description of Artificial Sequence Synthetic oligonucleotide”
tccacctcccccagacccttctctgca

SEQ ID NO: 235 - DNA, 27 bp, Artificial Sequence
  source: /note=“Description of Artificial Sequence Synthetic oligonucleotide”
tccccctcctctctatccacctccccc

SEQ ID NO: 236 - DNA, 27 bp, Artificial Sequence
  source: /note=“Description of Artificial Sequence Synthetic oligonucleotide”
tttattttgcatacctgttcgttacct

SEQ ID NO: 237 - DNA, 27 bp, Artificial Sequence
  source: /note=“Description of Artificial Sequence Synthetic oligonucleotide”
tttcaaatagaatgattttattttgca

SEQ ID NO: 238 - DNA, 27 bp, Artificial Sequence
  source: /note=“Description of Artificial Sequence Synthetic oligonucleotide”
tccatggtaagttacactaaccttagt

SEQ ID NO: 239 - DNA, 27 bp, Artificial Sequence
  source: /note=“Description of Artificial Sequence Synthetic oligonucleotide”
tttctgggttaaggtaatagcaatatc

SEQ ID NO: 240 - DNA, 27 bp, Artificial Sequence
  source: /note=“Description of Artificial Sequence Synthetic oligonucleotide”
tttatatgcagagatattgctattacc

SEQ ID NO: 241 - DNA, 27 bp, Artificial Sequence
  source: /note=“Description of Artificial Sequence Synthetic oligonucleotide”
attgctattaccttaacccagaaatta

SEQ ID NO: 242 - DNA, 27 bp, Artificial Sequence
  source: /note=“Description of Artificial Sequence Synthetic oligonucleotide”
tatgcagagatattgctattaccttaa

SEQ ID NO: 243 - DNA, 27 bp, Artificial Sequence
  source: /note=“Description of Artificial Sequence Synthetic oligonucleotide”
tttctgcatataaattgtaactgaggt

SEQ ID NO: 244 - DNA, 27 bp, Artificial Sequence
  source: /note=“Description of Artificial Sequence Synthetic oligonucleotide”
tataaattgtaactgaggtaagaggtt

SEQ ID NO: 245 - DNA, 27 bp, Artificial Sequence
  source: /note=“Description of Artificial Sequence Synthetic oligonucleotide”
tatgaaacctcttacctcagttacaat

SEQ ID NO: 246 - DNA, 27 bp, Artificial Sequence
  source: /note=“Description of Artificial Sequence Synthetic oligonucleotide”
attagcaatatgaaacctcttacctca

SEQ ID NO: 247 - DNA, 27 bp, Artificial Sequence
  source: /note=“Description of Artificial Sequence Synthetic oligonucleotide”
attgctaatagcagctacaatccaggt

SEQ ID NO: 248 - DNA, 27 bp, Artificial Sequence
  source: /note=“Description of Artificial Sequence Synthetic oligonucleotide”
tatgtacttgagatgtaagtaaggtta

SEQ ID NO: 249 - DNA, 27 bp, Artificial Sequence
  source: /note=“Description of Artificial Sequence Synthetic oligonucleotide”
attgatagtaaccttacttacatctca

SEQ ID NO: 250 - DNA, 27 bp, Artificial Sequence
  source: /note=“Description of Artificial Sequence Synthetic oligonucleotide”
gttaaaattccatcttaccaattctaa

SEQ ID NO: 251 - DNA, 27 bp, Artificial Sequence
  source: /note=“Description of Artificial Sequence Synthetic oligonucleotide”
attgaacgttaaaattccatcttacca

SEQ ID NO: 252 - DNA, 27 bp, Artificial Sequence
  source: /note=“Description of Artificial Sequence Synthetic oligonucleotide”
tttgtcatctgtaaaataagagtaaaa

SEQ ID NO: 253 - DNA, 27 bp, Artificial Sequence
  source: /note=“Description of Artificial Sequence Synthetic oligonucleotide”
gttaccatgtctccccactaaagtgta

SEQ ID NO: 254 - DNA, 27 bp, Artificial Sequence
  source: /note=“Description of Artificial Sequence Synthetic oligonucleotide”
tttgaggtgtggcttaggtacgagatg

SEQ ID NO: 255 - DNA, 27 bp, Artificial Sequence
  source: /note=“Description of Artificial Sequence Synthetic oligonucleotide”
gttataaaattcttacatacctttgaa

SEQ ID NO: 256 - DNA, 27 bp, Artificial Sequence
  source: /note=“Description of Artificial Sequence Synthetic oligonucleotide”
tttaaaaaatcttactcagattatgac

SEQ ID NO: 257 - DNA, 27 bp, Artificial Sequence
  source: /note=“Description of Artificial Sequence Synthetic oligonucleotide”
tttctttaaaaaatcttactcagatta

SEQ ID NO: 258 - DNA, 26 bp, Artificial Sequence
  source: /note=“Description of Artificial Sequence Synthetic oligonucleotide”
tttctccatccacaccgcagggagag

SEQ ID NO: 259 - DNA, 27 bp, Artificial Sequence
  source: /note=“Description of Artificial Sequence Synthetic oligonucleotide”
tttctggatttattttagtttacagaa

SEQ ID NO: 260 - DNA, 27 bp, Artificial Sequence
  source: /note=“Description of Artificial Sequence Synthetic oligonucleotide”
tttccaagaggtctgactttctggatt

SEQ ID NO: 261 - DNA, 27 bp, Artificial Sequence
  source: /note=“Description of Artificial Sequence Synthetic oligonucleotide”
tccaagaggtctgactttctggattta

SEQ ID NO: 262 - DNA, 27 bp, Artificial Sequence
  source: /note=“Description of Artificial Sequence Synthetic oligonucleotide”
tccaggttctgtaaactaaaataaatc

SEQ ID NO: 263 - DNA, 27 bp, Artificial Sequence
  source: /note=“Description of Artificial Sequence Synthetic oligonucleotide”
tttattttagtttacagaacctggacc

SEQ ID NO: 264 - DNA, 27 bp, Artificial Sequence
  source: /note=“Description of Artificial Sequence Synthetic oligonucleotide”
ttcatatgtctgtacacataccttgtt

SEQ ID NO: 265 - DNA, 27 bp, Artificial Sequence
  source: /note=“Description of Artificial Sequence Synthetic oligonucleotide”
gttcatatgtctgtacacataccttgt

SEQ ID NO: 266 - DNA, 27 bp, Artificial Sequence
  source: /note=“Description of Artificial Sequence Synthetic oligonucleotide”
tccctgctgagcccgcttgcttctccc

SEQ ID NO: 267 - DNA, 27 bp, Artificial Sequence
  source: /note=“Description of Artificial Sequence Synthetic oligonucleotide”
tcccgcctccctgctgagcccgcttgc

SEQ ID NO: 268 - DNA, 27 bp, Artificial Sequence
  source: /note=“Description of Artificial Sequence Synthetic oligonucleotide”
cccctcccgcctccctgctgagcccgc

SEQ ID NO: 269 - DNA, 27 bp, Artificial Sequence
  source: /note=“Description of Artificial Sequence Synthetic oligonucleotide”
tccctcctccctccctcaggaagtcgg

SEQ ID NO: 270 - DNA, 27 bp, Artificial Sequence
  source: /note=“Description of Artificial Sequence Synthetic oligonucleotide”
ccccaaggctccctcctccctccctca

SEQ ID NO: 271 - DNA, 27 bp, Artificial Sequence
  source: /note=“Description of Artificial Sequence Synthetic oligonucleotide”
tccctccctcaggaagtcggcgttggc

SEQ ID NO: 272 - DNA, 27 bp, Artificial Sequence
  source: /note=“Description of Artificial Sequence Synthetic oligonucleotide”
agatttaaagatgatctcttaccttgg

SEQ ID NO: 273 - DNA, 27 bp, Artificial Sequence
  source: /note=“Description of Artificial Sequence Synthetic oligonucleotide”
agatccaaggtaagagatcatctttaa

SEQ ID NO: 274 - DNA, 27 bp, Artificial Sequence
  source: /note=“Description of Artificial Sequence Synthetic oligonucleotide”
agatacttgtgtgattctggagaggaa

SEQ ID NO: 275 - DNA, 27 bp, Artificial Sequence
  source: /note=“Description of Artificial Sequence Synthetic oligonucleotide”
aaaaccaaggtaagagatcatctttaa

SEQ ID NO: 276 - DNA, 27 bp, Artificial Sequence
  source: /note=“Description of Artificial Sequence Synthetic oligonucleotide”
aaaattaaagatgatctcttaccttgg

SEQ ID NO: 277 - DNA, 27 bp, Artificial Sequence
  source: /note=“Description of Artificial Sequence Synthetic oligonucleotide”
aaaattcctctccagaatcacacaagt

SEQ ID NO: 278 - DNA, 35 bp, Artificial Sequence
  source: /note=“Description of Artificial Sequence Synthetic primer”
ttaggtaccgagttattttccaatccttctgcatc

SEQ ID NO: 279 - DNA, 34 bp, Artificial Sequence
  source: /note=“Description of Artificial Sequence Synthetic primer”
ttccgtctcataatgcctaaccctccaaccctcc

SEQ ID NO: 280 - DNA, 33 bp, Artificial Sequence
  source: /note=“Description of Artificial Sequence Synthetic primer”
gaacgtctctattactctattttaggctggggc

SEQ ID NO: 281 - DNA, 38 bp, Artificial Sequence
  source: /note=“Description of Artificial Sequence Synthetic primer”
tctcgtctcatcaatgtatcctatttgaagagaaatcc

SEQ ID NO: 282 - DNA, 34 bp, Artificial Sequence
  source: /note=“Description of Artificial Sequence Synthetic primer”
agacgtctcattgataaagctgtcttagagagga

SEQ ID NO: 283 - DNA, 29 bp, Artificial Sequence
  source: /note=“Description of Artificial Sequence Synthetic primer”
taatagatctctggactgcatcgggttcc

SEQ ID NO: 284 - DNA, 34 bp, Artificial Sequence
  source: /note=“Description of Artificial Sequence Synthetic primer”
ctctcctttcccaaggtaagagatcatctttaag

SEQ ID NO: 285 - DNA, 34 bp, Artificial Sequence
  source: /note=“Description of Artificial Sequence Synthetic primer”
cttaaagatgatctcttaccttgggaaaggagag

SEQ ID NO: 286 - DNA, 20 bp, Artificial Sequence
  source: /note=“Description of Artificial Sequence Synthetic primer”
ggcaaaccaatcccaaaccc

SEQ ID NO: 287 - DNA, 20 bp, Artificial Sequence
  source: /note=“Description of Artificial Sequence Synthetic primer”
attggaggcaaccaaccgaa

SEQ ID NO: 288 - DNA, 20 bp, Artificial Sequence
  source: /note=“Description of Artificial Sequence Synthetic primer”
cacagtccagtcctgggaac

SEQ ID NO: 289 - DNA, 20 bp, Artificial Sequence
  source: /note=“Description of Artificial Sequence Synthetic primer”
tagtagccgggccctacttt

SEQ ID NO: 290 - DNA, 21 bp, Artificial Sequence
  source: /note=“Description of Artificial Sequence Synthetic primer”
caaaccaatcccaaacccact

SEQ ID NO: 291 - DNA, 22 bp, Artificial Sequence
  source: /note=“Description of Artificial Sequence Synthetic primer”
cgccctggaagtataaattctc

SEQ ID NO: 292 - DNA, 20 bp, Artificial Sequence
  source: /note=“Description of Artificial Sequence Synthetic primer”
agttgcaggccagttgattt

SEQ ID NO: 293 - DNA, 23 bp, Artificial Sequence
  source: /note=“Description of Artificial Sequence Synthetic oligonucleotide”
ggtaatagcaatatttctgcata

SEQ ID NO: 294 - DNA, 39 bp, Artificial Sequence
  source: /note=“Description of Artificial Sequence Synthetic oligonucleotide”
  modified_base (7)..(7): a, c, t, g, unknown or other
  modified_base (13)..(35): a, c, t, g, unknown or other
  misc_feature (13)..(34): /note=“This region may encompass 0-22 nucleotides”
  modified_base (39)..(39): a, c, t, g, unknown or other
yyyyyynyaggtnnnnnnnnnnnnnnnnnnnnnnnaaan

SEQ ID NO: 295 - DNA, 10 bp, Artificial Sequence
  source: /note=“Description of Artificial Sequence Synthetic oligonucleotide”
  modified_base (5)..(10): a, c, t, g, unknown or other
aggtnnnnnn

SEQ ID NO: 296 - DNA, 35 bp, Artificial Sequence
  source: /note=“Description of Artificial Sequence Synthetic oligonucleotide”
  modified_base (8)..(30): a, c, t, g, unknown or other
  misc_feature (9)..(30): /note=“This region may encompass 0-22 nucleotides”
yyyytttnnnnnnnnnnnnnnnnnnnnnnnyaggt

SEQ ID NO: 297 - DNA, 38 bp, Artificial Sequence
  source: /note=“Description of Artificial Sequence Synthetic oligonucleotide”
  modified_base (7)..(7): a, c, t, g, unknown or other
  modified_base (13)..(35): a, c, t, g, unknown or other
  misc_feature (13)..(35): /note=“This region may encompass 0-22 nucleotides”
yyyyyynyaggtnnnnnnnnnnnnnnnnnnnnnnnaaa

SEQ ID NO: 298 - DNA, 35 bp, Artificial Sequence
  source: /note=“Description of Artificial Sequence Synthetic oligonucleotide”
  modified_base (7)..(7): a, c, t, g, unknown or other
  modified_base (9)..(30): a, c, t, g, unknown or other
  misc_feature (9)..(30): /note=“This region may encompass 0-22 nucleotides”
yyytttnynnnnnnnnnnnnnnnnnnnnnnyaggt

SEQ ID NO: 299 - DNA, 37 bp, Artificial Sequence
  source: /note=“Description of Artificial Sequence Synthetic oligonucleotide”
  modified_base (6)..(6): a, c, t, g, unknown or other
  modified_base (12)..(34): a, c, t, g, unknown or other
  misc_feature (12)..(33): /note=“This region may encompass 0-22 nucleotides”
yyyyynyaggtnnnnnnnnnnnnnnnnnnnnnnnaaa

SEQ ID NO: 300 - DNA, 34 bp, Artificial Sequence
  source: /note=“Description of Artificial Sequence Synthetic oligonucleotide”
  modified_base (7)..(29): a, c, t, g, unknown or other
  misc_feature (8)..(29): /note=“This region may encompass 0-22 nucleotides”
yyytttnnnnnnnnnnnnnnnnnnnnnnnyaggt

SEQ ID NO: 301 - DNA, 31 bp, Artificial Sequence
  source: /note=“Description of Artificial Sequence Synthetic oligonucleotide”
  modified_base (1)..(3): a, c, t, g, unknown or other
  modified_base (8)..(28): a, c, t, g, unknown or other
  misc_feature (8)..(27): /note=“This region may encompass 6-20 nucleotides”
nnnaggtnnnnnnnnnnnnnnnnnnnnnaaa

SEQ ID NO: 302 - DNA, 36 bp, Artificial Sequence
  source: /note=“Description of Artificial Sequence Synthetic oligonucleotide”
  modified_base (4)..(24): a, c, t, g, unknown or other
  misc_feature (5)..(24): /note=“This region may encompass 6-20 nucleotides”
  modified_base (29)..(36): a, c, t, g, unknown or other
tttnnnnnnnnnnnnnnnnnnnnnaggtnnnnnnnn

SEQ ID NO: 303 - DNA, 30 bp, Artificial Sequence
  source: /note=“Description of Artificial Sequence Synthetic oligonucleotide”
  modified_base (5)..(27): a, c, t, g, unknown or other
  misc_feature (5)..(26): /note=“This region may encompass 0-22 nucleotides”
aggtnnnnnnnnnnnnnnnnnnnnnnnaaa

SEQ ID NO: 304 - DNA, 38 bp, Artificial Sequence
  source: /note=“Description of Artificial Sequence Synthetic oligonucleotide”
  modified_base (4)..(26): a, c, t, g, unknown or other
  misc_feature (5)..(26): /note=“This region may encompass 0-22 nucleotides”
  modified_base (31)..(38): a, c, t, g, unknown or other
tttnnnnnnnnnnnnnnnnnnnnnnnaggtnnnnnnnn

SEQ ID NO: 305 - DNA, 33 bp, Artificial Sequence
  source: /note=“Description of Artificial Sequence Synthetic oligonucleotide”
  modified_base (1)..(3): a, c, t, g, unknown or other
  modified_base (8)..(30): a, c, t, g, unknown or other
  misc_feature (8)..(29): /note=“This region may encompass 0-22 nucleotides”
nnnaggtnnnnnnnnnnnnnnnnnnnnnnnaaa

SEQ ID NO: 306 - DNA, 89 bp, Artificial Sequence
  source: /note=“Description of Artificial Sequence Synthetic oligonucleotide”
ggtacaagatattatgaaattacattttgtgtttatgttatttgcagtgttttctatggaaatatttcacaggcaggagtccaattttc

SEQ ID NO: 307 - DNA, 89 bp, Artificial Sequence
  source: /note=“Description of Artificial Sequence Synthetic oligonucleotide”
gaaaattggactcctgcctgtgaaatatttccatagaaaacactgcaaataacataaacacaaaatgtaatttcataatatcttgtacc

SEQ ID NO: 308 - DNA, 68 bp, Artificial Sequence
  source: /note=“Description of Artificial Sequence Synthetic oligonucleotide”
acagcaactcaaacaactggaatctgaaggcaggagtccaattttcactcatcttgttacaagcttaa

SEQ ID NO: 309 - DNA, 68 bp, Artificial Sequence
  source: /note=“Description of Artificial Sequence Synthetic oligonucleotide”
tcaaacaactggaatctgaagtgttttctatggaaatatttcacaggcaggagtccaattttcactca

SEQ ID NO: 310 - DNA, 27 bp, Artificial Sequence
  source: /note=“Description of Artificial Sequence Synthetic oligonucleotide”
tgttatttgcagtgttttctatggaaa

SEQ ID NO: 311 - DNA, 55 bp, Artificial Sequence
  source: /note=“Description of Artificial Sequence Synthetic oligonucleotide”
gatattatgaaattacattttgtgtttatgttatttgcaatgttttctatggaaa

SEQ ID NO: 312 - DNA, 55 bp, Artificial Sequence
  source: /note=“Description of Artificial Sequence Synthetic oligonucleotide”
gatattatgaaattacattttgtgtttatgttatttgcagtgttttctatggaaa

SEQ ID NO: 313 - DNA, 37 bp, Artificial Sequence
  source: /note=“Description of Artificial Sequence Synthetic oligonucleotide”
gatattatgaaattacattttgtgttttctatggaaa

SEQ ID NO: 314 - DNA, 56 bp, Artificial Sequence
  source: /note=“Description of Artificial Sequence Synthetic oligonucleotide”
gatattatgaaattacattttgtgtttatgttattttgcagtgttttctatggaaa

SEQ ID NO: 315 - DNA, 54 bp, Artificial Sequence
  source: /note=“Description of Artificial Sequence Synthetic oligonucleotide”
gatattatgaaattacattttgtgtttatgttatagcagtgttttctatggaaa

SEQ ID NO: 316 - DNA, 52 bp, Artificial Sequence
  source: /note=“Description of Artificial Sequence Synthetic oligonucleotide”
gatattatgaaattacattttgtgtttatgtttgcagtgttttctatggaaa

SEQ ID NO: 317 - DNA, 51 bp, Artificial Sequence
  source: /note=“Description of Artificial Sequence Synthetic oligonucleotide”
gatattatgaaattacattttgttatgttattgcagtgttttctatggaaa

SEQ ID NO: 318 - DNA, 51 bp, Artificial Sequence
  source: /note=“Description of Artificial Sequence Synthetic oligonucleotide”
gatattatgaaattacattttgtgtttatgttatagtgttttctatggaaa

SEQ ID NO: 319 - DNA, 51 bp, Artificial Sequence
  source: /note=“Description of Artificial Sequence Synthetic oligonucleotide”
gatattatgaaattacattttgtgtttatgttgcagtgttttctatggaaa

SEQ ID NO: 320 - DNA, 49 bp, Artificial Sequence
  source: /note=“Description of Artificial Sequence Synthetic oligonucleotide”
gatattatgaaattacattttgtgtttatgttagtgttttctatggaaa

SEQ ID NO: 321 - DNA, 49 bp, Artificial Sequence
  source: /note=“Description of Artificial Sequence Synthetic oligonucleotide”
gatattatgaaattacattttgtgtttattgcagtgttttctatggaaa

SEQ ID NO: 322 - DNA, 48 bp, Artificial Sequence
  source: /note=“Description of Artificial Sequence Synthetic oligonucleotide”
gatattatgaaattacattttgtgtttatgcagtgttttctatggaaa

SEQ ID NO: 323 - DNA, 48 bp, Artificial Sequence
  source: /note=“Description of Artificial Sequence Synthetic oligonucleotide”
gatattatgaaattacattttgtgtttttgcagtgttttctatggaaa

SEQ ID NO: 324 - DNA, 48 bp, Artificial Sequence
  source: /note=“Description of Artificial Sequence Synthetic oligonucleotide”
gatattatgaaattacattttgtgtttatgttgtgttttctatggaaa

SEQ ID NO: 325 - DNA, 47 bp, Artificial Sequence
  source: /note=“Description of Artificial Sequence Synthetic oligonucleotide”
gatattatgaaattacattttgtgttttgcagtgttttctatggaaa

SEQ ID NO: 326 - DNA, 47 bp, Artificial Sequence
  source: /note=“Description of Artificial Sequence Synthetic oligonucleotide”
gatattatgaaattacattttgtgtttattagtgttttctatggaaa

SEQ ID NO: 327 - DNA, 46 bp, Artificial Sequence
  source: /note=“Description of Artificial Sequence Synthetic oligonucleotide”
gatattatgaaattacattttgtgtttgcagtgttttctatggaaa

SEQ ID NO: 328 - DNA, 45 bp, Artificial Sequence
  source: /note=“Description of Artificial Sequence Synthetic oligonucleotide”
gatattatgaaattaaattttgtgtttatgtgttttctatggaaa

SEQ ID NO: 329 - DNA, 45 bp, Artificial Sequence
  source: /note=“Description of Artificial Sequence Synthetic oligonucleotide”
gatattatgaaattacattttgttttgcagtgttttctatggaaa

SEQ ID NO: 330 - DNA, 44 bp, Artificial Sequence
  source: /note=“Description of Artificial Sequence Synthetic oligonucleotide”
gatattatgaaattacattttgtgtttagtgttttctatggaaa

SEQ ID NO: 331 - DNA, 42 bp, Artificial Sequence
  source: /note=“Description of Artificial Sequence Synthetic oligonucleotide”
gatattatgaaattacattttttgcagtgttttctatggaaa

SEQ ID NO: 332 - DNA, 42 bp, Artificial Sequence
  source: /note=“Description of Artificial Sequence Synthetic oligonucleotide”
gatattatgaaattacattttgtgcagtgttttctatggaaa

SEQ ID NO: 333 - DNA, 42 bp, Artificial Sequence
  source: /note=“Description of Artificial Sequence Synthetic oligonucleotide”
gatattatgaaattacattttgtgttgtgttttctatggaaa

SEQ ID NO: 334 - DNA, 41 bp, Artificial Sequence
  source: /note=“Description of Artificial Sequence Synthetic oligonucleotide”
gatattatgaaattacattttgtgagtgttttctatggaaa

SEQ ID NO: 335 - DNA, 38 bp, Artificial Sequence
  source: /note=“Description of Artificial Sequence Synthetic oligonucleotide”
gatattatgaaattactttgcagtgttttctatggaaa

SEQ ID NO: 336 - DNA, 32 bp, Artificial Sequence
  source: /note=“Description of Artificial Sequence Synthetic oligonucleotide”
gatgttatgaaattaagtgttttctatggaaa

SEQ ID NO: 337 - DNA, 31 bp, Artificial Sequence
  source: /note=“Description of Artificial Sequence Synthetic oligonucleotide”
gatattatgaaattagtgttttctatggaaa

SEQ ID NO: 338 - DNA, 29 bp, Artificial Sequence
  source: /note=“Description of Artificial Sequence Synthetic oligonucleotide”
gatattatgaaattacattttgtgggaaa

SEQ ID NO: 339 - DNA, 27 bp, Artificial Sequence
  source: /note=“Description of Artificial Sequence Synthetic oligonucleotide”
  modified_base (4)..(4): a, c, t, g, unknown or other
tttncatagaaaacactgcaaataaca

SEQ ID NO: 340 - DNA, 27 bp, Artificial Sequence
  source: /note=“Description of Artificial Sequence Synthetic oligonucleotide”
tttccatagaaaacactgcaaataaca

SEQ ID NO: 341 - DNA, 48 bp, Artificial Sequence
  source: /note=“Description of Artificial Sequence Synthetic oligonucleotide”
tgaaattacattttgtgtttatgttatttgcaatgttttctatggaaa

SEQ ID NO: 342 - DNA, 48 bp, Artificial Sequence
  source: /note=“Description of Artificial Sequence Synthetic oligonucleotide”
tgaaattacattttgtgtttatgttatttgcagtgttttctatggaaa

SEQ ID NO: 343 - DNA, 30 bp, Artificial Sequence
  source: /note=“Description of Artificial Sequence Synthetic oligonucleotide”
tgaaattacattttgtgttttctatggaaa

SEQ ID NO: 344 - DNA, 41 bp, Artificial Sequence
  source: /note=“Description of Artificial Sequence Synthetic oligonucleotide”
tgaaattacattttgtgtttatgcagtgttttctatggaaa

SEQ ID NO: 345 - DNA, 42 bp, Artificial Sequence
  source: /note=“Description of Artificial Sequence Synthetic oligonucleotide”
tgaaattacattttgtgtttatgttagtgttttctatggaaa

SEQ ID NO: 346 - DNA, 44 bp, Artificial Sequence
  source: /note=“Description of Artificial Sequence Synthetic oligonucleotide”
tgaaattacattttgtgtttatgttgcagtgttttctatggaaa

SEQ ID NO: 347 - DNA, 45 bp, Artificial Sequence
  source: /note=“Description of Artificial Sequence Synthetic oligonucleotide”
tgaaattacattttgtgtttatgtttgcagtgttttctatggaaa

SEQ ID NO: 348 - DNA, 41 bp, Artificial Sequence
  source: /note=“Description of Artificial Sequence Synthetic oligonucleotide”
tgaaattacattttgtgtttttgcagtgttttctatggaaa

SEQ ID NO: 349 - DNA, 37 bp, Artificial Sequence
  source: /note=“Description of Artificial Sequence Synthetic oligonucleotide”
tgaaattacattttgtgtttagtgttttctatggaaa

SEQ ID NO: 350 - DNA, 39 bp, Artificial Sequence
  source: /note=“Description of Artificial Sequence Synthetic oligonucleotide”
tgaaattacattttgtgtttgcagtgttttctatggaaa

SEQ ID NO: 351 - DNA, 41 bp, Artificial Sequence
  source: /note=“Description of Artificial Sequence Synthetic oligonucleotide”
tgaaattacattttgtgtttatgttgtgttttctatggaaa

SEQ ID NO: 352 - DNA, 40 bp, Artificial Sequence
  source: /note=“Description of Artificial Sequence Synthetic oligonucleotide”
tgaaattacattttgtgttttgcagtgttttctatggaaa

SEQ ID NO: 353 - DNA, 42 bp, Artificial Sequence
  source: /note=“Description of Artificial Sequence Synthetic oligonucleotide”
tgaaattacattttgtgtttattgcagtgttttctatggaaa

SEQ ID NO: 354 - DNA, 47 bp, Artificial Sequence
  source: /note=“Description of Artificial Sequence Synthetic oligonucleotide”
tgaaattacattttgtgtttatgttatagcagtgttttctatggaaa

SEQ ID NO: 355 - DNA, 68 bp, Artificial Sequence
  source: /note=“Description of Artificial Sequence Synthetic oligonucleotide”
tcacagcaactcaaacaactggaatctgaaggcatgagtccaattttcactcatcttgttacaagctt

SEQ ID NO: 356 - DNA, 67 bp, Artificial Sequence
  source: /note=“Description of Artificial Sequence Synthetic oligonucleotide”
tcacagcaactcaaacaactggaatctgaaggcaggagtccaattttcactcatcttgttacaagct

SEQ ID NO: 357 - DNA, 81 bp, Artificial Sequence
  source: /note=“Description of Artificial Sequence Synthetic oligonucleotide”
ggtaatgaaaaataattacaagagtcttccatctgttgcagtattaaaatggtgagtaagacaccctgaaaggaaatgttc

SEQ ID NO: 358 - DNA, 81 bp, Artificial Sequence
  source: /note=“Description of Artificial Sequence Synthetic oligonucleotide”
gaacatttcctttcagggtgtcttactcaccattttaatactgcaacagatggaagactcttgtaattatttttcattacc

SEQ ID NO: 359 - DNA, 52 bp, Artificial Sequence
  source: /note=“Description of Artificial Sequence Synthetic oligonucleotide”
cttctcaataagtcctggccagagggtgggcctcttgggaagaactggatca

SEQ ID NO: 360 - DNA, 105 bp, Artificial Sequence
  source: /note=“Description of Artificial Sequence Synthetic polynucleotide”
  modified_base (23)..(83): a, c, t, g, unknown or other
tggccagaggttgacttgtcatnnnnnnnnnnnnnnnnnnnnnnnnnnnnnnnnnnnnnnnnnnnnnnnnnnnnnnnnnnnnngtattaaaatggtgggcctctt

SEQ ID NO: 361 - DNA, 20 bp, Artificial Sequence
  source: /note=“Description of Artificial Sequence Synthetic oligonucleotide”
taaaatggtgcaccctgaaa

SEQ ID NO: 362 - DNA, 53 bp, Artificial Sequence
  source: /note=“Description of Artificial Sequence Synthetic oligonucleotide”
caagagtcttccatctgttgcagtattaaaatggcgagtaagacaccctgaaa

SEQ ID NO: 363 - DNA, 53 bp, Artificial Sequence
  source: /note=“Description of Artificial Sequence Synthetic oligonucleotide”
caagagtcttccatctgttgcagtattaaaatggtgagtaagacaccctgaaa

SEQ ID NO: 364 - DNA, 47 bp, Artificial Sequence
  source: /note=“Description of Artificial Sequence Synthetic oligonucleotide”
caagagtcttccatctgttgcagtattatgagtaagacaccctgaaa

SEQ ID NO: 365 - DNA, 44 bp, Artificial Sequence
  source: /note=“Description of Artificial Sequence Synthetic oligonucleotide”
caagagtcttccatctgttgcagtatgagtaagacaccctgaaa

SEQ ID NO: 366 - DNA, 31 bp, Artificial Sequence
  source: /note=“Description of Artificial Sequence Synthetic oligonucleotide”
caagagtcttccatagtaagacaccctgaaa

SEQ ID NO: 367 - DNA, 45 bp, Artificial Sequence
  source: /note=“Description of Artificial Sequence Synthetic oligonucleotide”
caagagtcttccatctgttgcagtattgagtaagacaccctgaaa

SEQ ID NO: 368 - DNA, 48 bp, Artificial Sequence
  source: /note=“Description of Artificial Sequence Synthetic oligonucleotide”
caagagtcttccatctgttgcagtattagtgagtaagacaccctgaaa

SEQ ID NO: 369 - DNA, 50 bp, Artificial Sequence
  source: /note=“Description of Artificial Sequence Synthetic oligonucleotide”
caagagtcttccatctgttgcagtataatggtgagtaagacaccctgaaa

SEQ ID NO: 370 - DNA, 46 bp, Artificial Sequence
  source: /note=“Description of Artificial Sequence Synthetic oligonucleotide”
caagagtcttccatctgttgcagtattagagtaagacaccctgaaa

SEQ ID NO: 371 - DNA, 43 bp, Artificial Sequence
  source: /note=“Description of Artificial Sequence Synthetic oligonucleotide”
caagagtcttccatctgttgcagtattgtaagacaccctgaaa

SEQ ID NO: 372 - DNA, 41 bp, Artificial Sequence
  source: /note=“Description of Artificial Sequence Synthetic oligonucleotide”
caagagtcttccatctgttgcagtagtaagacaccctgaaa

SEQ ID NO: 373 - DNA, 43 bp, Artificial Sequence
  source: /note=“Description of Artificial Sequence Synthetic oligonucleotide”
caagagtcttccatctgttgcagtatagtaagacaccctgaaa

SEQ ID NO: 374 - DNA, 30 bp, Artificial Sequence
  source: /note=“Description of Artificial Sequence Synthetic oligonucleotide”
caagagtcttccaagtaagacaccctgaaa

SEQ ID NO: 375 - DNA, 51 bp, Artificial Sequence
  source: /note=“Description of Artificial Sequence Synthetic oligonucleotide”
caagagtcttccatctgttgcagtattaatggtgagtaagacaccctgaaa

SEQ ID NO: 376 - DNA, 31 bp, Artificial Sequence
  source: /note=“Description of Artificial Sequence Synthetic oligonucleotide”
caagagtcttccatctgaagacaccctgaaa

SEQ ID NO: 377 - DNA, 47 bp, Artificial Sequence
  source: /note=“Description of Artificial Sequence Synthetic oligonucleotide”
caagagtcttccatctgttgcaaaatggtgagtaagacaccctgaaa

SEQ ID NO: 378 - DNA, 24 bp, Artificial Sequence
  source: /note=“Description of Artificial Sequence Synthetic oligonucleotide”
caagagtcttccatcaccctgaaa

SEQ ID NO: 379 - DNA, 38 bp, Artificial Sequence
  source: /note=“Description of Artificial Sequence Synthetic oligonucleotide”
caagagtcttccatctgttgcagtaagacaccctgaaa

SEQ ID NO: 380 - DNA, 49 bp, Artificial Sequence
  source: /note=“Description of Artificial Sequence Synthetic oligonucleotide”
caagagtcttccatctgttgcagtattaggtgagtaagacaccctgaaa

SEQ ID NO: 381 - DNA, 42 bp, Artificial Sequence
  source: /note=“Description of Artificial Sequence Synthetic oligonucleotide”
caagagtcttccatctgttgcagtgagtaagacaccctgaaa

SEQ ID NO: 382 - DNA, 27 bp, Artificial Sequence
  source: /note=“Description of Artificial Sequence Synthetic oligonucleotide”
caagagtcttccatctgcaccctgaaa

SEQ ID NO: 383 - DNA, 33 bp, Artificial Sequence
  source: /note=“Description of Artificial Sequence Synthetic oligonucleotide”
caagagtcttccatctagtaagacaccctgaaa

SEQ ID NO: 384 - DNA, 29 bp, Artificial Sequence
  source: /note=“Description of Artificial Sequence Synthetic oligonucleotide”
caagagtcttccagtaagacaccctgaaa

SEQ ID NO: 385 - DNA, 45 bp, Artificial Sequence
  source: /note=“Description of Artificial Sequence Synthetic oligonucleotide”
caagagtcttccatctgttgcagtagtgagtaagacaccctgaaa

SEQ ID NO: 386 - DNA, 52 bp, Artificial Sequence
  source: /note=“Description of Artificial Sequence Synthetic oligonucleotide”
caagagtcttccatctgttgcagtattaaatggtgagtaagacaccctgaaa

SEQ ID NO: 387 - DNA, 43 bp, Artificial Sequence
  source: /note=“Description of Artificial Sequence Synthetic oligonucleotide”
caagagtcttccatctgttgcagtattataagacaccctgaaa

SEQ ID NO: 388 - DNA, 39 bp, Artificial Sequence
  source: /note=“Description of Artificial Sequence Synthetic oligonucleotide”
caagagtcttccatctgttgcaagtaagacaccctgaaa

SEQ ID NO: 389 - DNA, 35 bp, Artificial Sequence
  source: /note=“Description of Artificial Sequence Synthetic oligonucleotide”
caagagtcttccatctgtagtaagacaccctgaaa

SEQ ID NO: 390 - DNA, 39 bp, Artificial Sequence
  source: /note=“Description of Artificial Sequence Synthetic oligonucleotide”
caagagtcttccatctgttgcaggtaagacaccctgaaa

SEQ ID NO: 391 - DNA, 23 bp, Artificial Sequence
  source: /note=“Description of Artificial Sequence Synthetic oligonucleotide”
  modified_base (21)..(21): a, c, t, g, unknown or other
attcaattataatcaccttgngg

SEQ ID NO: 392 - DNA, 23 bp, Artificial Sequence
  source: /note=“Description of Artificial Sequence Synthetic oligonucleotide”
attcaattataatcaccttgtgg

SEQ ID NO: 393 - DNA, 23 bp, Artificial Sequence
  source: /note=“Description of Artificial Sequence Synthetic oligonucleotide”
  modified_base (21)..(21): a, c, t, g, unknown or other
cacctcctccctgagaatgtngg

SEQ ID NO: 394 - DNA, 23 bp, Artificial Sequence
  source: /note=“Description of Artificial Sequence Synthetic oligonucleotide”
cacctcctccctgagaatgttgg

SEQ ID NO: 395 - DNA, 23 bp, Artificial Sequence
  source: /note=“Description of Artificial Sequence Synthetic oligonucleotide”
  modified_base (21)..(21): a, c, t, g, unknown or other
gacttcctccccgagaatgtngg

SEQ ID NO: 396 - DNA, 23 bp, Artificial Sequence
  source: /note=“Description of Artificial Sequence Synthetic oligonucleotide”
  modified_base (21)..(21): a, c, t, g, unknown or other
cccatccccccagagaatgtngg

SEQ ID NO: 397 - DNA, 23 bp, Artificial Sequence
  source: /note=“Description of Artificial Sequence Synthetic oligonucleotide”
  modified_base (21)..(21): a, c, t, g, unknown or other
ttcctcctcccagagaatatngg

SEQ ID NO: 398 - DNA, 23 bp, Artificial Sequence
  source: /note=“Description of Artificial Sequence Synthetic oligonucleotide”
  modified_base (21)..(21): a, c, t, g, unknown or other
tgtatcctccctgagaatgtnag

SEQ ID NO: 399 - DNA, 23 bp, Artificial Sequence
  source: /note=“Description of Artificial Sequence Synthetic oligonucleotide”
  modified_base (21)..(21): a, c, t, g, unknown or other
tgtctcccccatgagaatgtngg

SEQ ID NO: 400 - DNA, 23 bp, Artificial Sequence
  source: /note=“Description of Artificial Sequence Synthetic oligonucleotide”
  modified_base (21)..(21): a, c, t, g, unknown or other
agcctccccctcgagaatgtngg

SEQ ID NO: 401 - DNA, 23 bp, Artificial Sequence
  source: /note=“Description of Artificial Sequence Synthetic oligonucleotide”
  modified_base (21)..(21): a, c, t, g, unknown or other
cctctcctccctgagaatctngg

SEQ ID NO: 402 - DNA, 23 bp, Artificial Sequence
  source: /note=“Description of Artificial Sequence Synthetic oligonucleotide”
  modified_base (21)..(21): a, c, t, g, unknown or other
ctagtcctccctgagaatgtngg

SEQ ID NO: 403 - DNA, 23 bp, Artificial Sequence
  source: /note=“Description of Artificial Sequence Synthetic oligonucleotide”
  modified_base (21)..(21): a, c, t, g, unknown or other
cacatcctctctaagaatgtngg

SEQ ID NO: 404 - DNA, 23 bp, Artificial Sequence
  source: /note=“Description of Artificial Sequence Synthetic oligonucleotide”
  modified_base (21)..(21): a, c, t, g, unknown or other
ccactccacccagagaatgtngg

SEQ ID NO: 405 - DNA, 68 bp, Artificial Sequence
  source: /note=“Description of Artificial Sequence Synthetic oligonucleotide”
ctcacagcaactcaaacaactggaatctgaaggcaggagtccaattttcactcatcttgttacaagct

SEQ ID NO: 406 - DNA, 1482 bp, Artificial Sequence
  source: /note=“Description of Artificial Sequence Synthetic polynucleotide”
aagcttgtaccatgggcaaaccaatcccaaacccactgctgggtctggatggtaccggtgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggtatgacagtgaatgtgcgatactcatcttgtaaaaaagctataagagctatttgagattctttattgttaatctacttaaaaaaaattctgcttttaaacttttacatcatataacaataatttttttctacatgcatgtgtatataaaaggaaactatattacaaagtacacatggattttttttcttaattaatgaccatgtgacttcattttggttttaaaataggtatatagaatcttaccacagttggtgtacaggacattcatttataataaacttatatcagtcaaattaaacaaggatagtgctgctattactaaaggtttctctgggttcccaaatgatacttgaccaaatttgtccctttggcttgttgtcttcagacaccctttcttcatgtgttggagctgccatttcgtgtgcccccaaactctacttgagctgttagggaatcacattttgcagtgacagccttagtgtgggtgcattttcaggcaatactttttcagtatatttctgctttgtagattattagctaaatcaagtcacataaacttccttaatttagatacttgaaaaaattgtcttaaaagaaaatttttttagtaagaattaatttagaattagccagaaaactcccagtggtagccaagaaagaggaataaatattggtggtaattttttaagttcccatctctggtagccaagtaaaaaaagagggtaactcattaataaaataacaaatcatatctattcaaagaatggcaccagtgtgaaaaaaagctttttaaccaatgacatttgtgatatgattattctaatttagtctttttcaggtacaagatattatgaaattacattttgtgtttatgttatttgcaatgttttctatggaaatatttcacaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggcgagggatccggagagaatttatacttccagggcgggaattcgggctccatttcttctagtatctttaaaaatgaaggttaagcggccgctctaga

SEQ ID NO: 407 - DNA, 1482 bp, Artificial Sequence
  source: /note=“Description of Artificial Sequence Synthetic polynucleotide”
aagcttgtaccatgggcaaaccaatcccaaacccactgctgggtctggatggtaccggtgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggtatgacagtgaatgtgcgatactcatcttgtaaaaaagctataagagctatttgagattctttattgttaatctacttaaaaaaaattctgcttttaaacttttacatcatataacaataatttttttctacatgcatgtgtatataaaaggaaactatattacaaagtacacatggattttttttcttaattaatgaccatgtgacttcattttggttttaaaataggtatatagaatcttaccacagttggtgtacaggacattcatttataataaacttatatcagtcaaattaaacaaggatagtgctgctattactaaaggtttctctgggttcccaaatgatacttgaccaaatttgtccctttggcttgttgtcttcagacaccctttcttcatgtgttggagctgccatttcgtgtgcccccaaactctacttgagctgttagggaatcacattttgcagtgacagccttagtgtgggtgcattttcaggcaatactttttcagtatatttctgctttgtagattattagctaaatcaagtcacataaacttccttaatttagatacttgaaaaaattgtcttaaaagaaaatttttttagtaagaattaatttagaattagccagaaaactcccagtggtagccaagaaagaggaataaatattggtggtaattttttaagttcccatctctggtagccaagtaaaaaaagagggtaactcattaataaaataacaaatcatatctattcaaagaatggcaccagtgtgaaaaaaagctttttaaccaatgacatttgtgatatgattattctaatttagtctttttcaggtacaagatattatgaaattacattttgtgtttatgttatttgcagtgttttctatggaaatatttcacaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggcgagggatccggagagaatttatacttccagggcgggaattcgggctccatttcttctagtatctttaaaaatgaaggttaagcggccgctctaga

SEQ ID NO: 408 - DNA, 1343 bp, Artificial Sequence
  source: /note=“Description of Artificial Sequence Synthetic polynucleotide”
tacttaatacgactcactataggctagcctcgagatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgagatttgaacactgcttgctttgttagactgtgttcagtaagtgaatcccagtagcctgaagcaatgtgttagcagaatctatttgtaacattattattgtacagtagaatcaatattaaacacacatgttttattatatggagtcattatttttaatatgaaatttaatttgcagagtcctgaacctatatattcagtgggtataagcagcatattctcaatactatgtttcattaataattaatagagatatatgaacacataaaagattcaattataatcaccttgtggatctaaatttcagttgacttgtcatcttgatttctggagaccacaaggtaatgaaaaataattacaagagtcttccatctgttgcagtattaaaatggcgagtaagacaccctgaaaggaaatgttctattcatggtacaatgcaattacagctagcaccaaattcaacactgtttaactttcaacatattattttgatttatcttgatccaacattctcagggaggaggtgcattgaagttattagaaaacactgacttagatttagggtatgtcttaaaagcttatttgcgggaagtactctagccttattcaacagatcactgagaagcctaaaggtcagtgataaaggaagtctgcatcaggggtccaattccttatggccagtttctctattctgttccaaggttgtttgtctccatatatcaacattggtcaggattgaaagtgtgcaacaaggtttgaatgaataagtgaaaatcttccactggtgacaggataaaatattccaatggtttttattgaagtacaatactgaattatgtttatggcatggtacctatatgtcacagaagtgatcccatcacttttaccttataggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacaggcggccgct

SEQ ID NO: 409 - DNA, 1342 bp, Artificial Sequence
  source: /note=“Description of Artificial Sequence Synthetic polynucleotide”
tacttaatacgactcactataggctagcctcgagatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgagatttgaacactgcttgctttgttagactgtgttcagtaagtgaatcccagtagcctgaagcaatgtgttagcagaatctatttgtaacattattattgtacagtagaatcaatattaaacacacatgttttattatatggagtcattatttttaatatgaaatttaatttgcagagtcctgaacctatatattcagtgggtataagcagcatattctcaatactatgtttcattaataattaatagagatatatgaacacataaaagattcaattataatcaccttgtggatctaaatttcagttgacttgtcatcttgatttctggagaccacaaggtaatgaaaaataattacaagagtcttccatctgttgcagtattaaaatggtgagtaagacaccctgaaaggaaatgttctattcatggtacaatgcaattacagctagcaccaaattcaacactgtttaactttcaacatattattttgatttatcttgatccaacattctcagggaggaggtgcattgaagttattagaaaacactgacttagatttagggtatgtcttaaaagcttatttgcgggaagtactctagccttattcaacagatcactgagaagcctaaaggtcagtgataaaggaagtctgcatcaggggtccaattccttatggccagtttctctattctgttccaaggttgtttgtctccatatatcaacattggtcaggattgaaagtgtgcaacaaggtttgaatgaataagtgaaaatcttccactggtgacaggataaaatattccaatggtttttattgaagtacaatactgaattatgtttatggcatggtacctatatgtcacagaagtgatcccatcacttttaccttataggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacaggcggccgc

SEQ ID NO: 410 - DNA, 400 bp, Artificial Sequence
  source: /note=“Description of Artificial Sequence Synthetic polynucleotide”
tgatttgtatatagaattagatgattcggcttatcattttaaagcactaaattgaaagagtgccaggagtcaggttttaacacttccctagccaaaggagctaattaagctgctttcagcttcctctccagaatcacacaagttaaaggacccttctgcaacaagagcagcgaatctactcagccagagcaggaagctaataaaatgtatgctggcttttaagggggaaacaaatcatgaaattgaaattgaacacctctcctttcccaaggtaagagatcatctttaagaaaaggctgtgtattgtgggggtttgaagtgcaagttcatctcattatcatggatgtttcacccataatactatcatcatatgcaggagaaataaaagccttatccccca

SEQ ID NO: 411 - DNA, 400 bp, Artificial Sequence
  source: /note=“Description of Artificial Sequence Synthetic polynucleotide”
tgggggataaggcttttatttctcctgcatatgatgatagtattatgggtgaaacatccatgataatgagatgaacttgcacttcaaacccccacaatacacagccttttcttaaagatgatctcttaccttgggaaaggagaggtgttcaatttcaatttcatgatttgtttcccccttaaaagccagcatacattttattagcttcctgctctggctgagtagattcgctgctcttgttgcagaagggtcctttaacttgtgtgattctggagaggaagctgaaagcagcttaattagctcctttggctagggaagtgttaaaacctgactcctggcactctttcaatttagtgctttaaaatgataagccgaatcatctaattctatatacaaatca

SEQ ID NO: 412 - DNA, 69 bp, Artificial Sequence
  source: /note=“Description of Artificial Sequence Synthetic oligonucleotide”
gaaattgaacacctctcctttcccaagagatcatctttaagaaaaggctgtgtattgtggggtttgaag

SEQ ID NO: 413 - DNA, 70 bp, Artificial Sequence
  source: /note=“Description of Artificial Sequence Synthetic oligonucleotide”
gaaattgaacacctctcctttcccaaagagatcatctttaagaaaaggctgtgtattgtggggtttgaag

SEQ ID NO: 414 - DNA, 71 bp, Artificial Sequence
  source: /note=“Description of Artificial Sequence Synthetic oligonucleotide”
gaaattgaacacctctcctttcccataagagatcatctttaagaaaaggctgtgtattgtggggtttgaag

SEQ ID NO: 415 - DNA, 69 bp, Artificial Sequence
  source: /note=“Description of Artificial Sequence Synthetic oligonucleotide”
gaaattgaacacctctcctttcctaagagatcatctttaagaaaaggctgtgtattgtggggtttgaag

SEQ ID NO: 416 - DNA, 74 bp, Artificial Sequence
  source: /note=“Description of Artificial Sequence Synthetic oligonucleotide”
gaaattgaacacctctcctttcccaaggtaagagatcatctttaagaaaaggctgtgtattgtggggtttgaag

SEQ ID NO: 417 - DNA, 66 bp, Artificial Sequence
  source: /note=“Description of Artificial Sequence Synthetic oligonucleotide”
gaaattgaacacctctcctttcagagatcatctttaagaaaaggctgtgtattgtggggtttgaag

SEQ ID NO: 418 - DNA, 68 bp, Artificial Sequence
  source: /note=“Description of Artificial Sequence Synthetic oligonucleotide”
gaaattgaacacctctcctttccaagagatcatctttaagaaaaggctgtgtattgtggggtttgaag

SEQ ID NO: 419 - DNA, 72 bp, Artificial Sequence
  source: /note=“Description of Artificial Sequence Synthetic oligonucleotide”
gaaattgaacacctctcctttcccagtaagagatcatctttaagaaaaggctgtgtattgtggggtttgaag

SEQ ID NO: 420 - DNA, 73 bp, Artificial Sequence
  source: /note=“Description of Artificial Sequence Synthetic oligonucleotide”
gaaattgaacacctctcctttcccaggtaagagatcatctttaagaaaaggctgtgtattgtggggtttgaag

SEQ ID NO: 421 - DNA, 59 bp, Artificial Sequence
  source: /note=“Description of Artificial Sequence Synthetic oligonucleotide”
gaaattgaacacctctcctttcatctttaagaaaaggctgtgtattgtggggtttgaag

SEQ ID NO: 422 - DNA, 67 bp, Artificial Sequence
  source: /note=“Description of Artificial Sequence Synthetic oligonucleotide”
gaaattgaacacctctcctttcaagagatcatctttaagaaaaggctgtgtattgtggggtttgaag

SEQ ID NO: 423 - DNA, 62 bp, Artificial Sequence
  source: /note=“Description of Artificial Sequence Synthetic oligonucleotide”
gaaattgaacacctctcctttcatcatctttaagaaaaggctgtgtattgtggggtttgaag

SEQ ID NO: 424 - DNA, 56 bp, Artificial Sequence
  source: /note=“Description of Artificial Sequence Synthetic oligonucleotide”
gaaattgaacacctctcctttctttaagaaaaggctgtgtattgtggggtttgaag

SEQ ID NO: 425 - DNA, 64 bp, Artificial Sequence
  source: /note=“Description of Artificial Sequence Synthetic oligonucleotide”
gaaattgaacacctctcctttcagatcatctttaagaaaaggctgtgtattgtggggtttgaag

SEQ ID NO: 426 - DNA, 69 bp, Artificial Sequence
  source: /note=“Description of Artificial Sequence Synthetic oligonucleotide”
gaaattgaacacctctcctttcgtaagagatcatctttaagaaaaggctgtgtattgtggggtttgaag

SEQ ID NO: 427 - DNA, 60 bp, Artificial Sequence
  source: /note=“Description of Artificial Sequence Synthetic oligonucleotide”
gaaattgaacacctctcctttcccactttaagaaaaggctgtgtattgtggggtttgaag

SEQ ID NO: 428 - DNA, 67 bp, Artificial Sequence
  source: /note=“Description of Artificial Sequence Synthetic oligonucleotide”
gaaattgaacacctctcctttcccaagatcatctttaagaaaaggctgtgtattgtggggtttgaag

SEQ ID NO: 429 - DNA, 52 bp, Artificial Sequence
  source: /note=“Description of Artificial Sequence Synthetic oligonucleotide”
gaaattgaacacctctcctttaagaaaaggctgtgtattgtggggtttgaag

SEQ ID NO: 430 - DNA, 59 bp, Artificial Sequence
  source: /note=“Description of Artificial Sequence Synthetic oligonucleotide”
gaaattgaacacctctcctttcccctttaagaaaaggctgtgtattgtggggtttgaag

SEQ ID NO: 431 - DNA, 66 bp, Artificial Sequence
  source: /note=“Description of Artificial Sequence Synthetic oligonucleotide”
gaaattgaacacctctcctttcccagatcatctttaagaaaaggctgtgtattgtggggtttgaag

SEQ ID NO: 432 - DNA, 60 bp, Artificial Sequence
  source: /note=“Description of Artificial Sequence Synthetic oligonucleotide”
gaaattgaacacctctcctttccatctttaagaaaaggctgtgtattgtggggtttgaag

SEQ ID NO: 433 - DNA, 71 bp, Artificial Sequence
  source: /note=“Description of Artificial Sequence Synthetic oligonucleotide”
gaaattgaacacctctcctttcccgtaagagatcatctttaagaaaaggctgtgtattgtggggtttgaag

SEQ ID NO: 434 - DNA, 58 bp, Artificial Sequence
  source: /note=“Description of Artificial Sequence Synthetic oligonucleotide”
gaaattgaacacctctcctttccctttaagaaaaggctgtgtattgtggggtttgaag

SEQ ID NO: 435 - DNA, 70 bp, Artificial Sequence
  source: /note=“Description of Artificial Sequence Synthetic oligonucleotide”
agctaattaagctgctttcagcttccagaatcacacaagttaaaggacccttctgcaacaagagcagcga

SEQ ID NO: 436 - DNA, 75 bp, Artificial Sequence
  source: /note=“Description of Artificial Sequence Synthetic oligonucleotide”
agctaattaagctgctttcagcttcctctccagaatcacacaagttaaaggacccttctgcaacaagagcagcga

SEQ ID NO: 437 - DNA, 57 bp, Artificial Sequence
  source: /note=“Description of Artificial Sequence Synthetic oligonucleotide”
agctaattaagctgctttcagccaagttaaaggacccttctgcaacaagagcagcga

SEQ ID NO: 438 - DNA, 66 bp, Artificial Sequence
  source: /note=“Description of Artificial Sequence Synthetic oligonucleotide”
agctaattaagctgctttcagcttcatcacacaagttaaaggacccttctgcaacaagagcagcga

SEQ ID NO: 439 - DNA, 69 bp, Artificial Sequence
  source: /note=“Description of Artificial Sequence Synthetic oligonucleotide”
agctaattaagctgctttcagcttcagaatcacacaagttaaaggacccttctgcaacaagagcagcga

SEQ ID NO: 440 - DNA, 63 bp, Artificial Sequence
  source: /note=“Description of Artificial Sequence Synthetic oligonucleotide”
agctaattaagctgctttcagcttcacacaagttaaaggacccttctgcaacaagagcagcga

SEQ ID NO: 441 - DNA, 72 bp, Artificial Sequence
  source: /note=“Description of Artificial Sequence Synthetic oligonucleotide”
agctaattaagctgctttcagcttctccagaatcacacaagttaaaggacccttctgcaacaagagcagcga

SEQ ID NO: 442 - DNA, 75 bp, Artificial Sequence
  source: /note=“Description of Artificial Sequence Synthetic oligonucleotide”
  modified_base (26)..(26): a, c, t, g, unknown or other
agctaattaagctgctttcagcttcnctctccagaatcacacaagttaaaggacccttctgcaacaagagcagcg

SEQ ID NO: 443 - DNA, 57 bp, Artificial Sequence
  source: /note=“Description of Artificial Sequence Synthetic oligonucleotide”
agctaattaagctgctttcacacaagttaaaggacccttctgcaacaagagcagcga

SEQ ID NO: 444 - DNA, 56 bp, Artificial Sequence
  source: /note=“Description of Artificial Sequence Synthetic oligonucleotide”
agctaattaagctgctttcagcaagttaaaggacccttctgcaacaagagcagcga

SEQ ID NO: 445 - DNA, 67 bp, Artificial Sequence
  source: /note=“Description of Artificial Sequence Synthetic oligonucleotide”
agctaattaagctgctttcagcttgaatcacacaagttaaaggacccttctgcaacaagagcagcga

SEQ ID NO: 446 - DNA, 68 bp, Artificial Sequence
  source: /note=“Description of Artificial Sequence Synthetic oligonucleotide”
agctaattaagctgctttcagcttagaatcacacaagttaaaggacccttctgcaacaagagcagcga

SEQ ID NO: 447 - DNA, 71 bp, Artificial Sequence
  source: /note=“Description of Artificial Sequence Synthetic oligonucleotide”
agctaattaagctgctttcagctttccagaatcacacaagttaaaggacccttctgcaacaagagcagcga

SEQ ID NO: 448 - DNA, 73 bp, Artificial Sequence
  source: /note=“Description of Artificial Sequence Synthetic oligonucleotide”
agctaattaagctgctttcagctttctccagaatcacacaagttaaaggacccttctgcaacaagagcagcga

SEQ ID NO: 449 - DNA, 62 bp, Artificial Sequence
  source: /note=“Description of Artificial Sequence Synthetic oligonucleotide”
agctaattaagctgctttcagcttccacaagttaaaggacccttctgcaacaagagcagcga

SEQ ID NO: 450 - DNA, 71 bp, Artificial Sequence
  source: /note=“Description of Artificial Sequence Synthetic oligonucleotide”
agctaattaagctgctttcagcttcccagaatcacacaagttaaaggacccttctgcaacaagagcagcga

SEQ ID NO: 451 - DNA, 73 bp, Artificial Sequence
  source: /note=“Description of Artificial Sequence Synthetic oligonucleotide”
agctaattaagctgctttcagcttcctccagaatcacacaagttaaaggacccttctgcaacaagagcagcga

SEQ ID NO: 452 - DNA, 75 bp, Artificial Sequence
  source: /note=“Description of Artificial Sequence Synthetic oligonucleotide”
  modified_base (26)..(40): a, c, t, g, unknown or other
agctaattaagctgctttcagcttcnnnnnnnnnnnnnnnctctccagaatcacacaagttaaaggacccttctg

SEQ ID NO: 453 - DNA, 55 bp, Artificial Sequence
  source: /note=“Description of Artificial Sequence Synthetic oligonucleotide”
agctaattaagctgctttcacaagttaaaggacccttctgcaacaagagcagcga

SEQ ID NO: 454 - DNA, 23 bp, Artificial Sequence
  source: /note=“Description of Artificial Sequence Synthetic oligonucleotide”
agggtgtcttactcgccatttta

(19) United States
(12) Patent Application Publication
(10) Pub. No.: US 2022/0145305 A1
(43) Pub. Date: May 12, 2022

(54) CAS12a GUIDE RNA MOLECULES AND USES THEREOF

(71) Applicants: UNIVERSITA' DEGLI STUDI DI TRENTO, Trento (IT); KATHOLIEKE UNIVERSITEIT LEUVEN, Leuven (BE)

(72) Inventors: Gianluca Petris, Sauris (IT); Giulia Maule, Volano (IT); Marianne Carlon, Holsbeek (BE); Antonio Casini, Trento (IT); Anna Cereseto, Trento (IT)

(73) Assignees: UNIVERSITA' DEGLI STUDI DI TRENTO, Trento (IT); KATHOLIEKE UNIVERSITEIT LEUVEN, Leuven (BE)

(21) Appl. No.: 17/430,092

(22) PCT Filed: Feb. 11, 2020
(86) PCT No.: PCT/IB2020/051089

§ 371 Date: Aug. 11, 2021

Related U.S. Application Data
(60) Provisional application No. 62/804,591, filed on Feb. 12, 2019.

(51) Int. Cl. C12N 15/113; C12N 9/22; C12N 15/90; C12N 15/11

(52) U.S. Cl. CPC C12N 15/1138; C12N 9/22; C12N 15/90; C12N 2320/34; C12N 2310/20; C12N 2320/11; C12N 2320/33; C12N 15/111

(57) ABSTRACT

Engineered Cas12a guide RNA (gRNA) molecules useful, for example, for correcting aberrant RNA splicing resulting from mutations in a genomic DNA sequence and for preventing exon inclusion in mature mRNA.

### 1. CROSS-REFERENCE TO RELATED APPLICATIONS

This application claims the priority benefit of U.S. provisional application No. 62/804,591, filed Feb. 12, 2019 the contents of which are incorporated herein in their entireties by reference thereto.

### 2. SEQUENCE LISTING

The instant application contains a Sequence Listing which has been submitted electronically in ASCII format and is hereby incorporated by reference in its entirety. Said ASCII copy, created on Feb. 10, 2020, is named ALA-002WO_SL.txt and is 135,245 bytes in size.

### 3. BACKGROUND

Genetic mutations are responsible for a plethora of defects, disorders, and disease conditions. Over 16,000 mutations, ranging from single base pair changes to large-scale chromosomal defects, are known to contribute to at least 6,000 different conditions. Duchenne muscular dystrophy, beta-thalassemia, hemophilia, sickle-cell disease, amyotrophic lateral sclerosis, familial hypercholesterolemia, cystic fibrosis, Usher syndrome, type II are a few of the more well known disease conditions caused by genetic mutations.

Cystic fibrosis (CF) is a lethal autosomal recessive disorder inherited in approximately 1 in 2,500 births. CF is the result of mutations in the Cystic Fibrosis Transmembrane conductance Regulator (CFTR) gene, a gene that is expressed in the apical membrane of all epithelial cells, thus affecting multiple organs. The primary cause of mortality in CF patients is bacterial infection of the airways, provoking chronic lung disease and, ultimately, respiratory failure.

Current CF treatments are not curative, being limited only to the reduction of clinical symptoms such as attacking chronic bacterial infections or alleviating airway blockages. The deleterious effects of a limited number of CFTR genetic mutations can be lessened by the use of recently developed small molecules such as CFTR correctors and potentiators. However, the success of potentiator treatment is strongly dependent upon residual CFTR protein, which is often very low and highly variable among patients. Moreover, the easing of symptoms through the use of current treatments brings only temporary alleviation to those suffering from CF; patients must undergo repeated cycles of discomfort and treatment followed by short-lived periods of relief. In addition, current treatments are associated with side effects that can be exacerbated by repeated administration.

Despite recent advances in gene therapy, little progress has been made towards a curative solution for CF and other genetically based disease conditions. In the case of CF, current gene therapies are based upon the delivery, typically via the lungs, of a functional copy of the CFTR gene to a patient in an attempt to compensate for the faulty CFTR gene. Such therapies are inefficient at best, hampered by poor lung transduction, transient and low levels of gene expression, the rapid turnover of pulmonary epithelial cells, and the disease symptoms which remain when the administered CFTR gene expression drops below a therapeutically effective level.

Mutations in the USH2A gene can cause Usher syndrome, type II. Usher syndrome, type II is characterized by hearing and vision loss. Treatment options for Usher syndrome, type II. Current treatments for Usher syndrome, type II are not curative. Instead, current treatments involve managing hearing and vision loss. Thus, there remains a significant need for new treatments and cures for cystic fibrosis, Usher syndrome, type II and other genetic diseases such as Duchenne muscular dystrophy, hemophilia, and amyotrophic lateral sclerosis.

### 4. SUMMARY

The disclosure provides Cas12a guide RNA (gRNA) molecules engineered to contain a targeting sequence and a loop domain. The Cas12a gRNA molecules of the disclosure, in combination with Cas12a proteins, can be used, for example, to correct or modify aberrant splicing of a pre-mRNA molecule by editing a genomic DNA sequence encoding the pre-mRNA. The present disclosure is based, in part, on the discovery that allele specific repair of splicing mutations in the CFTR gene could be accomplished through the use of single Cas12a gRNAs targeting the vicinity of the splicing mutations. Unexpectedly, it was discovered that efficient correction of splicing errors resulting from splicing mutations in the CFTR gene does not require deletion or correction of the mutation itself when using Cas12a gRNAs as described herein. Instead, and without being bound by theory, it is believed that splicing corrections can be obtained from the deletion of nucleotides in or near the splicing regulatory elements close to the mutation rather than correction of the mutation. The deletion of nucleotides can result in removal or inactivation of splicing regulatory elements near the mutation, although in some instances the mutation itself can be deleted. Moreover, the strategy of using a single Cas12a gRNA to repair splicing mutations has been found surprisingly superior to the conventional approach of using Cas9 in combination with sgRNAs to induce genetic deletion. The genome editing approach exemplified with respect to the CFTR gene can be applied to correct splicing defects in various other genes associated with genetic diseases as well as applied to restore expression of functional protein, such as through exon skipping of exons having deleterious mutations such as premature stop codons.

Accordingly, the present disclosure provides Cas12a gRNA molecules that target genomic sequences that encode mutant splice sites. As illustrated in FIG. 1 and FIG. 2, the Cas12a gRNA molecules of the disclosure each comprise (a) a protospacer domain containing a targeting sequence and (b) a loop domain. As further illustrated in FIG. 1 and FIG. 2, the targeting sequence corresponds to a target domain in a genomic DNA sequence, and the target domain is adjacent to a protospacer-adjacent motif (PAM) recognized by a Cas12a protein. The target domain can be, for example, in a eukaryotic, e.g., mammalian, genomic DNA sequence. Preferably, the target domain is in a human genomic sequence. The human genomic sequence can be a within a gene associated with a genetic disease, for example, a Cystic Fibrosis Transmembrane conductance Regulator (CFTR) gene.

In certain aspects, the Cas12a gRNAs have a targeting sequence corresponding to a target domain that includes a splice site (e.g., as shown schematically in FIGS. 1A and 2A) or that is close to a splice site (e.g., as shown schematically in FIGS. 1B and 2B).

The splice site can be, for example, a cryptic splice site activated by or introduced by a mutation in the genomic DNA. The mutation in the genomic DNA can be within the target domain (e.g., as shown schematically in FIGS. 1A and 1B) or near the target domain (e.g., as shown schematically in FIGS. 2A and 2B).

Splicing of pre-mRNA molecules at cryptic splice sites can result in a disease phenotype, and reducing the activity of a cryptic splice site by editing the genomic DNA with a Cas12a gRNA in combination with a Cas12a protein can restore normal splicing. For example, CFTR mutations 3272-26A>G, 3849+10kbC>T, IVS11+194A>G, and IVS19+11505C>G result in cystic fibrosis, and Cas12a gRNAs of the disclosure can be used to restore normal CFTR splicing.

Including the mutation in the targeting sequence can allow for allele specific cleavage of the genomic DNA. The protospacer domain of most Cas12a proteins is typically 23 nucleotides in length, and as such, specific cleavage of the chromosome containing the mutation (as opposed to the wild-type allele) can be achieved by selecting a target domain that is 1 to 23 nucleotides away from a Cas12a PAM sequence.

The splice site can alternatively be a canonical splice site. Reducing the activity of a canonical splice site by editing the genomic DNA with a Cas12a gRNA in combination with a Cas12a protein can be used, for example, to cause exon skipping in a gene having a deleterious mutation (e.g., a mutation, for example in an exon, that results in a truncated protein). Generally, the mutation will be outside of the target domain. By skipping an exon, production of an altered, yet possibly still functional, protein can be achieved. For example, mutations in exon 50 of the DMD gene can cause premature truncation of the dystrophin protein encoded by the gene, but exon skipping of exon 51 can restore the reading frame and restore expression of functional dystrophin protein (see, Amoasii et al., 2017, Science Translational Medicine, 9(418):eaan8081). Cas12a gRNAs of the disclosure can be used, for example, to edit a DMD gene having mutations in exon 50 so that exon 51 is skipped, thereby restoring expression of functional dystrophin protein.

The activity of a splice site can be reduced by using a Cas12a gRNA designed so that upon introduction of the gRNA and the Cas12a protein into a cell containing the genomic sequence, the Cas12a protein cleaves the genomic DNA close to the splice site (e.g., up to 15 nucleotides from the splice site). Indels introduced during repair of the cleaved genomic DNA can reduce activity of the splice site (partially or completely). With knowledge of the PAM sequence recognized by a particular Cas12a protein (e.g., TTTV for AsCas12a), knowledge of where the Cas12a protein cuts (e.g. after the 19th base following the PAM sequence on the strand having the target domain sequence and after the 23^{rd} base following the PAM sequence on the complementary strand for AsCas12a), and knowledge of the position of the splice site relative to the PAM sequence in the genomic DNA, a targeting sequence can be selected such that upon introduction of the gRNA and the Cas12a protein into a cell containing the genomic sequence, the Cas12a will cleave the genomic DNA up to 15 nucleotides from the splice site.

In some embodiments, the Cas12a gRNAs have a targeting sequence corresponding to a target domain adjacent to a Cas12a PAM sequence that is within 40 nucleotides (e.g., 4 to 38 nucleotides) of a splice site encoded by the genomic DNA sequence.

Exemplary features of genomic DNA that can be targeted and exemplary features of gRNA molecules of the disclosure are described in Sections 6.2 and 6.3 and numbered embodiments 1 to 283, infra. Exemplary Cas12a proteins which can be used in conjunction with gRNAs of the disclosure are described in Section 6.4, infra.

The disclosure further provides nucleic acids encoding gRNAs of the disclosure and cells containing the nucleic acids. Features of exemplary nucleic acids encoding gRNAs and exemplary cells are described in Section 6.5 and numbered embodiments 284 to 287 and 302 to 305, infra.

The disclosure further provides systems and particles containing Cas12a gRNAs of the disclosure. Exemplary systems and particles are described in Section 6.6 and numbered embodiments 296 to 301, infra.

The disclosure further provides methods of using the gRNAs, systems, and particles of the disclosure for altering cells. Methods of the disclosure can be used, for example, to treat subjects having a genetic disease, for example cystic fibrosis or muscular dystrophy. Exemplary methods of altering cells are described in Section 6.7 and numbered embodiments 306 to 376, infra.

### 5. BRIEF DESCRIPTION OF THE FIGURES

It should be understood that the figures are exemplary and do not limit the scope of this disclosure. FIGS. 1-6 are illustrations not necessarily drawn to scale.

FIGS. 1A-1B illustrate Cas12a gRNAs having targeting sequences corresponding to target domains in genomic DNA sequences having mutations in the target domains, where the genomic DNA encodes a splice site within the target domain (FIG. 1A) or outside the target domain (FIG. 1B). In the genomic DNA, PAM sequences are shown by gridded sections; target domains are shown by dotted sections; mutations are shown by an asterisk (*); splice sites are shown by bidirectional arrows. In the gRNAs, loop domains are shown by dotted lines; protospacer domains comprising targeting sequences are shown by dashed lines.

FIGS. 2A-2B illustrate Cas12a gRNAs having targeting sequences corresponding to target domains in genomic DNA sequences having mutations outside of the target domains, where the genomic DNA encodes a splice site within the target domain (FIG. 2A) or outside the target domain (FIG. 2B). In the genomic DNA, PAM sequences are shown by gridded sections; target domains are shown by dotted sections; mutations are shown by an asterisk (*); splice sites are shown by bidirectional arrows. In the gRNAs, loop domains are shown by dotted lines; protospacer domains comprising targeting sequences are shown by dashed lines.

FIGS. 3A-3B illustrate Cas12a gRNAs targeting cryptic 3′ splice sites. FIG. 3A illustrates Cas12a gRNAs targeting a cryptic 3′ splice site which is upstream of a canonical 3′ splice site. Splicing at the cryptic 3′ splice rather than the canonical 3′ splice site results in a longer than normal exon sequence in the mature mRNA. The additional nucleotides of the longer than normal exon sequence are represented in the figure by light shading and the nucleotides of the normal exon sequence are represented in the figure by dark shading. The PAM sequence (or its complement) is boxed. Mutations are shown with bold, underlined text. Figure discloses SEQ ID NOS 294-296 and 295, respectively, in order of appearance. FIG. 3B illustrates Cas12a gRNAs targeting a cryptic 3′ splice site which is upstream of a cryptic 5′ splice site. Splicing at the cryptic 3′ splice and the cryptic 5′ splice site results in inclusion of a pseudo-exon sequence in the mature mRNA. The nucleotides of the pseudo-exon are represented in the figure by light shading. The PAM sequence (or its complement) is boxed. Mutations are shown with bold, underlined text. As shown schematically in the upper and lower portions of the FIGS. 1A-1B, gRNAs can be designed to target either strand of the genomic DNA. Figure discloses SEQ ID NOS 297, 295, 298, and 295, respectively, in order of appearance.

FIG. 4 illustrates Cas12a gRNAs targeting a canonical 3′ splice site. Exon nucleotides represented in the figure by light shading. The PAM sequence (or its complement) is boxed. Reducing the activity of a canonical 3′ splice site by editing the genomic DNA with Cas12a and a Cas12a gRNA targeting the canonical 3′ splice site can be used to prevent inclusion of the exon in the mature mRNA. As shown schematically in the upper and lower portions of the figure, gRNAs can be designed to target either strand of the genomic DNA. Figure discloses SEQ ID NOS 299, 295, 300, and 295, respectively, in order of appearance.

FIGS. 5A-5B illustrate Cas12a gRNAs targeting cryptic 5′ splice sites. FIG. 5A illustrates Cas12a gRNAs targeting a cryptic 5′ splice site which is downstream of a cryptic 3′ splice site. Splicing at the cryptic 3′ splice and the cryptic 5′ splice site results in inclusion of a pseudo-exon in the mature mRNA. The nucleotides of the pseudo-exon are represented in the figure by light shading. The PAM sequence (or its complement) is boxed. Mutations are shown with bold, underlined text. Figure discloses SEQ ID NOS 301 and 302, respectively, in order of appearance. FIG. 5B illustrates Cas12a gRNAs targeting a cryptic 5′ splice site which is downstream of a canonical 5′ splice site. Splicing at the cryptic 5′ splice rather than the canonical 5′ splice site results in a longer than normal exon. The additional nucleotides of the longer than normal exon are represented in the figure by light shading, and the nucleotides of the normal exon represented in the figure by dark shading. The PAM sequence (or its complement) is boxed. Mutations are shown with bold, underlined text. As shown schematically in the upper and lower portions of FIGS. 5A-5B, gRNAs can be designed to target either strand of the genomic DNA. Figure discloses SEQ ID NOS 303 and 304, respectively, in order of appearance.

FIG. 6 illustrates Cas12a gRNAs targeting a canonical 5′ splice site. Exon nucleotides are represented in the figure by light shading. The PAM sequence (or its complement) is boxed. Reducing the activity of a canonical 5′ splice site by editing the genomic DNA with Cas12a and a gRNA targeting the canonical 5′ splice site can prevent exon inclusion in the mature mRNA. As shown schematically in the upper and lower portions of the figure, gRNAs can be designed to target either strand of the genomic DNA. Figure discloses SEQ ID NOS 305 and 304, respectively, in order of appearance.

FIG. 7 illustrates a scheme of CFTR minigenes containing an approximately 1.3 Kb sequence corresponding to the CFTR region extending from exon 19 to 20 either wild-type (pMG3272-26WT) or 3272-26A>G mutated (pMG3272-26A>G). Exons are shown as boxes, introns as lines; the expected spliced transcripts are represented on the right according to the presence or absence of the 3272-26 A>G mutation. The lower panel shows the nucleotide sequence and intron-exon boundaries near the 3272-26A>G mutation (labelled in bold) and the target crRNA positions (underlined, with the PAM depicted by thicker underline). Figure discloses SEQ ID NOS 306 and 307, respectively, in order of appearance.

FIGS. 8A-8B illustrate the validation of intron 19 splicing in pMG3272-26WT and pMG3272-26A>G CFTR minigene models. FIG. 8A: Splicing pattern of CFTR wild-type (pMG3272-26WT) and mutated (pMG3272-26A>G) minigene models, transfected in HEK293T cells, by agarose gel electrophoresis analysis of RT-PCR products. Black-solid arrow indicates aberrant splicing; white-empty arrow indicates correct splicing. FIG. 8B: Sanger sequencing chromatogram of minigene splicing products from FIG. 8A. Vertical lines represent the boundary between exons. Figure discloses SEQ ID NOS 308 and 309, respectively, in order of appearance.

FIGS. 9A-9D illustrate the correction of altered intron 19 splicing in CFTR 3272-26A>G minigene model by AsCas12a DNA editing. FIG. 9A: Splicing pattern analyzed by RT-PCR in HEK293/pMG3272-26A>G cells following treatments with AsCas12a-crRNA control (Ctr) or specific for the 3272-26A>G mutation (+11 and −2). Black-solid arrow indicates aberrant splicing; white-empty arrow indicates correct splicing. Representative data of n=2 independent runs. FIG. 9B: Percentages of correct splicing measured by densitometry as in FIG. 9A. FIG. 9C: editing efficiency analyzed by TIDE in cells treated as in FIG. 9A. Data are means±SEM from n=2 independent runs. FIG. 9D: Indels triggered by AsCas12a-crRNA+11. The 3272-26A>G locus from cells edited using crRNA+11 were amplified, cloned in the minigene backbone, and Sanger sequenced (34 different clones, left panel), or analyzed as in FIG. 9A to visualize the splicing pattern. pMG3272-26WT and pMG3272-26A>G were used as references. Figure discloses SEQ ID NOS 310-338, respectively, in order of appearance.

FIGS. 10A-10C illustrate the target specificity of AsCas12a-crRNA+11 editing. Editing efficiency by TIDE analysis in HEK293/pMG3272-26WT or HEK293/pMG3272-26A>G cells (FIG. 10A) and in Caco-2 cells (FIG. 10B) following lentiviral transduction of Cas12a-crRNA+11 or +11/wt as indicated. Data are means±SEM from n=2 independent runs. FIG. 10C: GUIDE-seq analysis of crRNA+11. Figure discloses SEQ ID NOS 339 and 340, respectively, in order of appearance.

FIGS. 11A-D illustrate the repair pattern after AsCas12a-crRNA+11 cleavage. FIG. 11A-C: Indels spectrum by TIDE analysis from HEK293/pMG3272-26A>G cells after AsCas12a-crRNA+11 editing from n=3 independent runs. FIG. 11D: Agarose gel electrophoresis of RT-PCR products showing splicing pattern of edited sites cloned into the minigene plasmid and transfected in HEK293T cells.

FIGS. 12A-B illustrate the unchanged WT CFTR splicing after AsCas12a-crRNA+11 or crRNA+11/wt DNA editing. RT-PCR product analysis after AsCas12a-crRNA+11 or +11/wt editing in HEK293/pMG3272-26WT or A>G minigene (FIG. 12A) and in Caco-2 cells (FIG. 12B) having the WT CFTR sequence. Cells were transduced with lentiviral vectors carrying AcCas12a-crRNA+11 or +11/wt and selected with puromycin for 10 days. Images are representative of two independent runs.

FIGS. 13A-H illustrate AsCas12a-crRNA+11 genome editing analysis in 3272-26A>G mutated CF patient organoids. FIG. 13A: Splicing pattern analysis by RT-PCR in 3272-26A>G organoids following lentiviral transduction (14 days) of AsCas12a-crRNA control (Ctr) or specific for the 3272-26A>G mutation (+11) or with CFTR cDNA. Black-solid arrow indicates aberrant splicing; white-empty arrow indicates correct splicing. The percentages of aberrant splicing (% of 25 nucleotide (nt) insertion into mRNA) was measured by chromatogram decomposition analysis. FIG. 13B: Editing efficiency in 3272-26A>G organoids measured by T7E1 assay following lentiviral transduction as in FIG. 13A. FIG. 13C: Deep sequencing analysis of the CFTR on-target locus after AsCas12a-crRNA+11 transduction of the 3272-26A>G organoids (average from n=2 independent runs). Figure discloses SEQ ID NOS 310 and 341-354, respectively, in order of appearance. FIG. 13D: Percentage of deep sequencing reads of the edited and non-edited 3272-26A>G or WT alleles from FIG. 13C. FIG. 13E: Schematic representation of CFTR dependent swelling in organoids models. FIG. 13F: Representative confocal images of calcein labelled 3272-26A>G organoids before (T=0 min) and after (T=60 min) Forskolin Induced Swelling (FIS) assay. Scale bar=200 μm. FIG. 13G: Quantification of organoids area following lentiviral transduction of AsCas12a-crRNA Ctr, AsCas12a-crRNA+11 or with CFTR cDNA as indicated. Each dot represents the average organoid areas analyzed in each well (number of organoids per well: 25-300) from 4 independent runs. FIG. 13H: Fold change of organoids area before (T=0 min) and after (T=60 min) FIS assay, each dot represents the average increase organoid areas analyzed in each well (number of organoids per well: 25-300) from n=4 independent runs. Data are means±SD. **P<0.01, ****P<0.0001, n.s. non-significant.

FIGS. 14A-140 illustrate CFTR splicing and functional characterization of 3272-26A>G mutated CF patient's organoids after genome editing with AsCas12a-crRNA+11. FIG. 14A: Chromatogram of RT-PCR products from FIG. 3A. Upper panel represents the mixed population of mRNA transcripts of 3272-26A>G/4218insT organoids, the lower panel shows transcripts after AsCas12a-crRNA+11 editing in these organoids. Sequence to the right of the vertical line indicates chromatogram area after the exon19-exon 20 junction. Figure discloses SEQ ID NOS 355 and 356, respectively, in order of appearance. FIG. 14B-14C: Chromatogram deconvolution analysis was used to evaluate the amount of mutated splicing (inclusion of +25 nt from intron 19) before (FIG. 14B) and after (FIG. 14C) AsCas12a-crRNA+11 cleavage. FIG. 14D: FIS assay of n=4 independent runs; each line represents one well (n=25-300). Data are means±SD.

FIG. 15 illustrates a scheme of CFTR wild-type (pMG3849+10kbWT) and 3849+10KbC>T (pMG3849+10kbC>T) minigenes carrying exon 22, portions of intron 22 encompassing the 3849+10KbC>T mutation, and exon 23 of the CFTR gene. Exons are shown as boxes and introns as lines; the expected spliced transcripts are represented on the right according to the presence or absence of the 3849+10kbC>T mutation. The lower panel shows the nucleotide sequence near the 3849+10kbC>T mutation (labelled in bold) and the AsCas12a-crRNA+14 target position (underlined, with the PAM (CTTT) in darker underline). Figure discloses SEQ ID NOS 357 and 358, respectively, in order of appearance.

FIGS. 16A-16B illustrates the validation of intron 22 splicing in pMG3849+10kbWT and pMG3849+10kbC>T CFTR minigene models. FIG. 16A: Splicing pattern of CFTR wild-type (pMG3849+10kbWT) and mutated (pMG3849+10kbC>T) minigene models, transfected in HEK293T cells, by agarose gel electrophoresis analysis of RT-PCR products. Black-solid arrow indicates aberrant splicing; white-empty arrow indicates correct splicing; Δ indicates alternative splicing product. FIG. 16B: Sanger sequencing chromatogram of minigene splicing products from FIG. 16A. Vertical lines represent the boundary between exons. Figure discloses SEQ ID NOS 359 and 360, respectively, in order of appearance.

FIGS. 17A-17C illustrate the correction of the 3849+10kbC>T splicing defect by AsCas12a-crRNA+14 editing in a minigene model and human intestinal patient-derived organoids. FIG. 17A: Splicing pattern analyzed by RT-PCR in HEK293/pMG3849+10kbC>T cells following treatments with AsCas12a-crRNA control (Ctr) or specific for the 3272-26A>G mutation (+14). Black-solid arrow indicates aberrant splicing; white-empty arrow indicates correct splicing; Δ indicates a minigene splicing artifact. FIG. 17B: Caco-2 cells lentivirally transduced with AsCas12a-crRNA+14 or +14/wt were analyzed for editing in CFTR intron 22 by SYNTHEGO ICE editing analysis. Data are means±SEM from n=2 independent runs. FIG. 17C: GUIDE-seq analysis of crRNA+14.

FIGS. 18A-18C illustrate the correction of the 3849+10kbC>T splicing defect by AsCas12a-crRNA+14 editing in a minigene model and human intestinal patient's derived organoids. FIG. 18A: 3849+10Kb C>T patient's derived intestinal organoids were lentivirally transduced with AsCas12a-crRNA control (Ctr) or crRNA+14 and analyzed for intron 22 editing by SYNTHEGO ICE. FIG. 18B: Confocal images of calcein labelled 3849+10KbC>T organoids transduced with AsCas12a-crRNA+14 or CFTR cDNA. Scale bar 200 μm. FIG. 18C: Quantification of organoids area as in FIG. 18B; each dot represents the average area of organoids analyzed in each well (number of organoids per well: 3-30). Data are means±SD. **P<0.01, n.s. non-significant.

FIG. 19 illustrates AsCas12a editing of CFTR 3849+10kbC>T organoids. SINTHEGO ICE analysis of AsCas12a-crRNA+14 editing in organoids samples. Predicted repair outcomes are represented with their abundance. Figure discloses SEQ ID NOS 361-376, 375, 377-384, 362-364, 370, 385, 380, 379, 368, 369, 386, 372, 387, 384, 373, 371, 388, 381, 389, and 390, respectively, in order of appearance.

FIGS. 20A-20H illustrates the SpCas9-sgRNA correction of the 3849+10 kb splicing defect in a minigene model and CF patient-derived organoids. FIG. 20A: Screening of SpCas9-sgRNA pairs in pMG3849+10kbC>T transfected in HEK293T cells. RT-PCR products were analyzed by agarose gel electrophoresis. Δ indicates alternative splicing products of pMG3849+10kbWT or C>T. FIG. 20B: Agarose gel electrophoretic analysis of targeted deletions in pMG3849+10kbC>T after cleavage of SpCas9-sgRNA pairs. FIG. 20C: RT-PCR products and FIG. 20D: targeted deletions in Caco-2 cells transduced with a SpCas9-sgRNA lentiviral vectors and after 10 days of puromycin selection. FIG. 20E: Editing in patient organoids analyzed by agarose gel electrophoresis. FIG. 20F: Confocal images of calcein labelled CF 3849+10kbC>T organoids at T=0 min transduced with 0.25, 0.5 or 1 RTU of SpCas9-sgRNAs-95/+119. Scale bar=200 μm. FIG. 20G: Quantification of steady-state organoid area; each dot represents the average area of organoids from one well (n=3-30). Data are means±SD. **P<0.01, ****P<0.0001. FIG. 20H: GUIDE-seq analysis of gRNA-95 and gRNA+119. Figure discloses SEQ ID NOS 391-404 and 396, respectively, in order of appearance.

FIGS. 21A-21G illustrate SpCas9 and AsCas12a gRNA functional screening for splicing correction of the 3272-26A>G minigene. FIGS. 21A-21B: SpCas9-sgRNA (FIG. 21A) and AsCas12a-crRNA (FIG. 21B) screening based on the ability to restore the correct splicing pattern of CFTR 3272-26A>G minigene. Nucleases and gRNAs single or in pair were transfected in HEK293T cells with pMG3272-26A>G. RT-PCR products were analyzed by agarose gel electrophoresis. pMG3272-26WT was used as a reference for correct intron 19 splicing. FIG. 21C and FIG. 21D: Agarose gel electrophoretic analysis of targeted deletions in 3272-26A>G minigene after cleavage with SpCas9-sgRNAs (FIG. 21C) and AsCas12a-crRNAs (FIG. 21D) measured by PCR. The larger band represents non-edited minigene sequences, the smaller band is the expected deletion product. FIG. 21E: Agarose gel electrophoresis of RT-PCR products. FIG. 21F: Agarose gel electrophoresis of PCR products of targeted deletion for SpCas9-sgRNA pairs selected from FIG. 21B in HEK293 cells having stable genomic integration of 3272-26A>G minigene (HEK293/pMG3272-26A>G cells). FIG. 21G: Sanger sequencing chromatogram of correct intron 19 splicing from 3272-26A>G integrated minigene after AsCas12a-crRNA+11 editing from FIG. 9A. Vertical line represents the boundary between exons 19-20. Figure discloses SEQ ID NO: 405.

FIGS. 22A-22D illustrate partial plasmid sequences representing the minigenes. FIG. 22A: pMG3272-26A>GWT (SEQ ID NO: 406). FIG. 22B: pMG3272-26A>G (SEQ ID NO: 407). FIG. 22C: pMG3849+10 kbWT (SEQ ID NO: 408). FIG. 22D: pMG3849+10kbC>T (SEQ ID NO: 409).

FIG. 23 is a schematic representation of the USH2A minigene models exploited to mimic USH2A splicing in Example 11. The minigenes include USH2A exon 40 and exon 41, as well as the portion of intron 40 giving rise to the pseudoexon 40 (PE40) in presence of the c.7595-2144A>G mutation. Protein tags were inserted at the 5′ and 3′-ends of the construct to aid expression, driven by a strong constitutive CMV promoter. The splicing products on the wild-type and mutated minigenes are shown at the bottom of the figure.

FIG. 24 is a representative agarose gel showing the splicing products for the wild-type and mutated USH2A minigenes detected by RT-PCR after transfection of HEK293 cells with the two minigenes generated in Example 11. The transcript produced by the mutated minigene is bigger due to the inclusion of PE40.

FIG. 25 schematically shows Cas12a guide RNA target domains for editing USH2A pseudoexon 40 (PE40) (Example 11). PE40 is highlighted in light grey. The position of the c.7595-2144A>G mutation is also indicated. Figure discloses SEQ ID NOS 410 and 411, respectively, in order of appearance.

FIGS. 26A-26D show correction of USH2A splicing by Cas12a in transiently transfected HEK293 cells (Example 11). FIG. 26A: Representative agarose gel showing the RT-PCR analysis of the splicing products obtained after transient transfection of HEK293 with AsCas12a in combination of the indicated gRNAs and wild-type or mutated USH2A minigenes, as indicated. Cells transfected with a vector encoding AsCas12a and a non-targeting scramble gRNA are shown as a control. The lower band corresponds to correctly spliced products, while the upper one includes the aberrant PE40. NTC: no template control. FIG. 26B: Percentage of correct splicing products generated at 6 days after co-transfection of HEK293 with wild-type and mutated minigenes together with AsCas12a and the indicated gRNAs obtained by densitometric analysis of data of FIG. 26A. Data are presented as mean±SEM for n=2 biologically independent studies. FIG. 26C: Representative agarose gel showing the RT-PCR analysis of the splicing products obtained after transient transfection of HEK293 with LbCas12a in combination of the indicated gRNAs and wild-type or mutated USH2A minigenes, as indicated. Cells transfected with a vector encoding LbCas12a and a non-targeting scramble gRNA are shown as a control. The lower band corresponds to correctly spliced products, while the upper one includes the aberrant PE40. NTC: no template control. FIG. 26D: Percentage of correct splicing products generated at 6 days after co-transfection of HEK293 with wild-type and mutated minigenes together with LbCas12a and the indicated gRNAs obtained by densitometric analysis of data of FIG. 26C. Data are presented as mean±SEM for n=2 biologically independent studies.

FIGS. 27A-27C show correction of USH2A splicing by LbCas12a in HEK293 clones stably expressing USH2A minigenes. FIG. 27A: Representative agarose gel showing the splicing patterns detected by RT-PCR of USH2A wild-type minigene in HEK293 stable clone 1 and USH2A mutated minigene in HEK293 stable clones 4 and 6 at 10 days post-transduction with a lentiviral vector expressing LbCas12a together either with guide 1 or guide 3, as indicated. FIG. 27B: Levels of correct splicing products measured by densitometry on data of FIG. 27A obtained at 10 days after transduction of HEK293 clones 4 and 6, stably expressing USH2A mutated minigene, with a lentiviral vector encoding LbCas12a together either with guide 1 or guide 3. FIG. 27C: Indel formation at 10 days post-transduction of HEK293 clones stably expressing either wild-type or mutated USH2A minigenes with a lentiviral vector encoding LbCas12a and the indicated gRNAs as measured by TIDE analysis. Data on the HEK293 clone bearing an integrated wild-type minigene (clone 1) are reported to evaluate the allele specificity of each gRNA in these study conditions. In FIG. 27B and FIG. 27C data are presented as mean SEM for n=2 biologically independent studies.

FIGS. 28A-28D shows indel profiles generated by LbCas12a on the c.7595-2144A>G USH2A minigene (Example 11). Indel profiles calculated from Sanger sequencing reads obtained from HEK293 c.7595-2144A>G USH2A clones 4 and 6 after transduction with lentiviral vectors encoding for LbCas12a and guide 1 (FIG. 28A-FIG. 28B) or guide 3 (FIG. 28C-FIG. 28D), as done in FIG. 27. Chromatogram analyses were performed using the Synthego ICE webtool and only report indels with a calculated frequency above or equal to 1%. Where present, the c.7595-2144A>G mutation is highlighted with a circle. FIG. 28A discloses SEQ ID NOS 412-428; FIG. 28B discloses SEQ ID NOS 412, 413, 416, 429, 414, 424, 418, 430, 431, 428, 420, 432, 433, 419, 421, 415, and 434; FIG. 28C discloses SEQ ID NOS 435-449; and FIG. 28D discloses SEQ ID NOS 435, 437, 436, 439, 440, 438, 441, 442, 444, and 450-453, all respectively, in order of appearance.

### 6. DETAILED DESCRIPTION

The disclosure provides Cas12a guide RNA (gRNA) molecules, which in combination with Cas12a proteins, can be used, for example, to correct aberrant RNA splicing resulting from mutations in a genomic DNA sequence or, as another example, to prevent inclusion of an exon in a mature mRNA (e.g., where exon skipping would be advantageous).

In one aspect, a gRNA of the disclosure is engineered to comprise a protospacer domain containing a targeting sequence and a loop domain. The targeting sequence corresponds to a target domain in a genomic DNA sequence, and the target domain is adjacent to a protospacer-adjacent motif (PAM) of a Cas12a protein.

Exemplary features of genomic DNA that can be targeted and exemplary features of gRNA molecules of the disclosure are described in Sections 6.2 and 6.3. Exemplary Cas12a proteins which can be used in conjunction with gRNAs of the disclosure are described in Section 6.4.

The disclosure further provides nucleic acids encoding gRNAs of the disclosure and host cells containing the nucleic acids. Features of exemplary nucleic acids encoding gRNAs and exemplary host cells are described in Section 6.5.

The disclosure further provides systems and particles containing Cas12a gRNAs of the disclosure. Exemplary systems and particles are described in Section 6.6.

The disclosure further provides methods of using the gRNAs, systems, and particles of the disclosure for altering cells. Methods of the disclosure can be useful, for example, for treating a genetic disease. Exemplary methods of altering cells are described in Section 6.7.

### 6.1. Definitions

Adjacent, when referring to two nucleotide sequences (e.g., a target domain and a PAM), means that the two nucleotide sequences are next to each other with no intervening nucleotides between the two sequences.

A Cas12a protein refers to a wild-type or engineered Cas12a protein. Cas12a proteins are also referred to in the art as Cpf1 proteins.

Corresponds to, when referring to a targeting sequence and a target domain, means that the targeting sequence is complementary to the complement of the target domain, with no more than 3 nucleotide mismatches. In some embodiments, the targeting sequence is complementary to the complement of the target domain, with no more than 2 nucleotide mismatches. In other embodiments, the targeting sequence is complementary to the complement of the target domain, with no more than 1 nucleotide mismatches. In other embodiments, the targeting sequence is complementary to the complement of the target domain, with no nucleotide mismatches.

Disrupted, in reference to a region of a genomic DNA sequence, means that the region has been altered by an indel.

Indels, in the context of this disclosure, refer to insertions and deletions in a genomic DNA sequence introduced during repair (e.g., by non-homologous end joining or homology-directed repair) of a genomic DNA sequence that has been cleaved by a Cas12a protein.

Loop domain is a component of a Cas12a gRNA of the disclosure comprising a stem-loop structure recognized by a Cas12a protein. Loop domains can comprise a nucleotide sequence of a naturally occurring stem-loop sequence recognized by a Cas12a protein or can comprise an engineered nucleotide sequence that forms a stem-loop structure recognized by a Cas12a protein. See, e.g., Zetsche et al., 2015, Cell 163:759-771.

Mutation, in the context of this disclosure, refers to an alteration of a wild-type genomic DNA sequence. A mutation can be an alteration at one or more nucleotides (e.g., a single nucleotide polymorphism (SNP)), a deletion, or an insertion relative to the wild-type genomic DNA sequence. A mutation which is a deletion or insertion can be, for example, a deletion or insertion from 1 to 10⁶ nucleotides (e.g., 1 to 10⁵ nucleotides, 1 to 10⁴ nucleotides, 1 to 10³ nucleotides, 1 to 100 nucleotides, or 1 to 10 nucleotides).

Protospacer domain refers to a region of a Cas12a gRNA molecule containing a targeting sequence. A protospacer domain is sometimes referred to as a crRNA.

Protospacer-adjacent motif (PAM), in the context of this disclosure, refers to a genomic DNA sequence, generally four nucleotides long, that is 5′ to a target domain in the genomic DNA sequence and which is required for cleavage of the genomic DNA by a Cas12a protein that recognizes the PAM. An exemplary PAM sequence is TTTV, which is the PAM sequence for wild-type AsCas12a and LbCas12a.

Splice site as used herein refers to an intron/exon junction in a precursor mRNA (pre-mRNA) molecule. A splice site can be a 5′ splice site (also referred to as a donor splice site), which is a splice site located at the 5′ end of an intron, or a 3′ splice site (also referred to as an acceptor splice site), which is a splice site located at the 3′ end of an intron. Splicing of pre-mRNA splicing at a canonical splice site is referred to herein as normal splicing. Pre-mRNA splicing that occurs at a cryptic splice site is referred to herein as aberrant splicing. Cryptic splice sites can be present in wild-type pre-mRNA molecules, but are generally dormant or used only at low levels unless activated by a mutation. Cryptic splice sites can also be created by a mutation.

Target Domain refers to a genomic DNA sequence targeted for cleavage by a Cas12a protein.

Targeting Sequence refers to a region of a Cas12a gRNA molecule corresponding to a target domain.

Wild-type, in reference to a genomic DNA sequence, refers to a genomic DNA sequence that predominates in a species, e.g., Homo sapiens.

### 6.2. Genomic DNA Sequences for Genome Editing

Cas12a gRNAs of the disclosure can be designed to target, in combination with a Cas12a protein, eukaryotic genomic sequences, such as mammalian genomic sequences. Preferably, the targeted genomic sequences are human genomic sequences. Genomic sequences of interest are typically genomic sequences encoding a mutated gene whose expression results in a disease phenotype. For example, the disease phenotype can be a disease phenotype resulting from a mutation which causes aberrant splicing of pre-mRNA, or disease phenotype resulting from a mutation in an exon (e.g., a mutation that introduces a stop codon into mRNA encoded by the genomic sequence).

Exemplary genomic DNA sequences that can be targeted include variant Cystic Fibrosis Transmembrane conductance Regulator (CFTR) genes (e.g., which are associated with cystic fibrosis), variant dystrophin (DMD) genes (e.g., which are associated with muscular dystrophies such as Duchenne muscular dystrophy or Becker muscular dystrophy), variant hemoglobin subunit beta (HBB) genes (e.g., which are associated with beta-thalassemia), variant fibrinogen beta chain (FGB) genes (e.g., which are associated with afibrinogenemia), variant superoxide dismutase 1 (SOD1) genes (e.g., which are associated with amyotrophic lateral sclerosis), variant quinoid dihydropteridine reductase (QDPR) genes (e.g., which are associated with dihydropteridine reductase deficiency), variant alpha-galactosidase (GLA) genes (e.g., which are associated with Fabry disease), variant low density lipoprotein receptor (LDLR) genes (e.g., which are associated with familial hypercholesterolemia), variant BRCA1-interacting protein 1 (BRIP1) genes (e.g., which are associated with Fanconi anemia), variant coagulation factor IX (F9) genes (e.g., which are associated with hemophilia B), variant centrosomal protein of 290 kDa (CEP290) genes (e.g., which are associated with Leber congenital amaurosis), variant collagen, type II, alpha 1 (COL2A1) genes (e.g., which are associated with Stickler syndrome), variant usherin (USH2A) genes (e.g., which are associated with Usher syndrome, type II), and variant acid alpha-glucosidase (AAG) genes (e.g., which are associated with glycogen storage disease, type II). Exemplary target domains in different variants of these genes (and which can be used to design a Cas12a gRNA as described herein) are described in Section 6.3.4.

### 6.2.1. Protospacer-Adjacent Motifs (PAMs)

One constraint on the use of CRISPR systems in general (e.g., both CRISPR-Cas9 and CRISPR-Cas12a) is the requirement for the target domain to be in close proximity to a PAM sequence (e.g., adjacent to a PAM sequence). Cas12a proteins generate staggered cuts when cleaving genomic DNA; in the case of AsCas12a and LbCas12a, DNA cleavage of a target genomic sequence occurs after the 19^{th} base following the PAM sequence on the strand having the target domain sequence and after the 23^{rd} base following the PAM sequence on the complementary strand. Thus, design of Cas12a gRNAs is constrained by the location and availability of PAM sequences in genomic DNA. However, Cas12a variants recognizing PAM sequences which are different from the PAM sequences recognized by wild-type Cas12a proteins have been designed (see Section 6.4), expanding the number of genomic DNA sequences that can potentially be targeted for editing with Cas12a.

The PAM recognized by AsCas12a and LbCas12a is TTTV, where V is A, C, or G, while the PAM of FnCas12 is NTTN, where N is any nucleotide. Engineered Cas12a proteins recognizing alternative PAM sequences have been designed, for example which recognize one or more of TYCV, where Y is C or T and V is A, C, or G; CCCC; ACCC; TATV, where V is A, C, or G; and RATR. Cas12a proteins which recognize these PAM sequences are described in Section 6.4.

### 6.2.2. Splice Sites

Cas12a gRNAs of the disclosure target genomic DNA sequences that are close to or include a splice site encoded by the genomic DNA. The splice site needs to be in close proximity to a Cas12a PAM sequence so that the genomic DNA can be cleaved by a Cas12a protein. For example, Cas12a gRNAs can be designed so that upon introduction of the gRNA and the Cas12a protein into a cell containing the genomic sequence, the Cas12a cleaves the genomic DNA up to 15 nucleotides (e.g., up to 10 nucleotides or 10-15 nucleotides) from a splice site encoded by the genomic DNA. Indels created during repair of the cleaved genomic DNA can cause a reduction (e.g., partial or complete) in the activity of the splice site, thereby altering the splicing of the pre-mRNA encoded by the genomic DNA. The splice site can be a cryptic splice site (e.g., one that results in a disease phenotype), or a canonical splice site (e.g., upstream of an exon containing a disease-causing mutation). The splice site (cryptic or canonical) can be a 5′ splice site or a 3′ splice site. Splice sites are described in greater detail in Section 6.3.2.

### 6.3. Cas12a Guide RNAs

In one aspect, the disclosure provides an engineered Cas12a guide RNA (gRNA) molecule comprising a protospacer domain containing a targeting sequence and a loop domain. The targeting sequence corresponds to a target domain in a genomic DNA sequence, and the target domain is adjacent to a protospacer-adjacent motif (PAM) of a Cas12a protein.

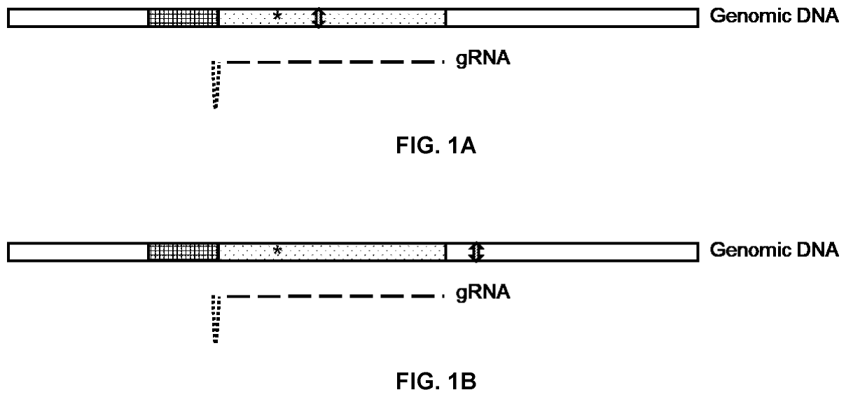

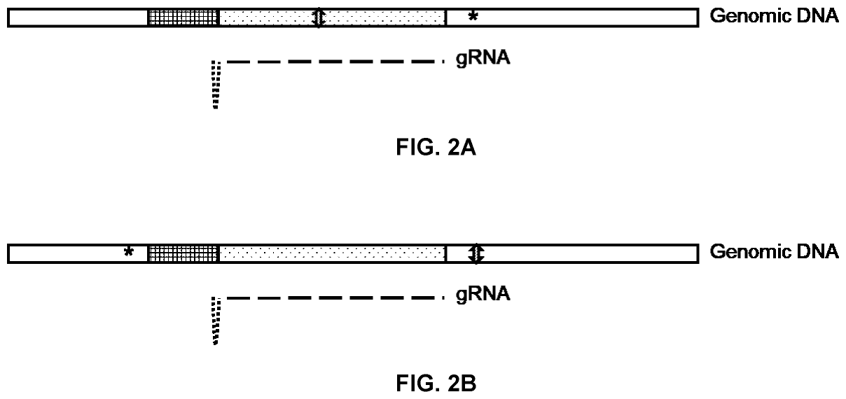

In certain aspects, the Cas12a gRNAs have a targeting sequence corresponding to a target domain that includes a splice site (shown schematically in FIG. 1) or that is close to a splice site (shown schematically in FIG. 2).

The splice site can be, for example, a cryptic splice site activated or introduced by a mutation in the genomic DNA. Splicing of pre-mRNA molecules at cryptic splice sites can result in a disease phenotype, and reducing the activity of a cryptic splice site by editing the genomic DNA with a Cas12a gRNA in combination with a Cas12a protein can restore normal splicing. Including the mutation in the targeting sequence (e.g., where the mutation is 1 to 23 nucleotides from a Cas12a PAM sequence) can allow for allele specific cleavage of the genomic DNA. In some embodiments, the gRNA has a targeting sequence corresponding to a target domain having a mutation that is 1 to 20 nucleotides, 1 to 15 nucleotides, 1 to 10 nucleotides, 1 to 5 nucleotides, 5 to 15 nucleotides, 10 to 20 nucleotides, or 15 to 23 nucleotides from the PAM sequence.

The splice site can alternatively be a canonical splice site. Reducing the activity of a canonical splice site by editing the genomic DNA with a Cas12a gRNA in combination with a Cas12a protein can be used, for example, to cause exon skipping of an exon in a gene having a deleterious mutation (e.g., a mutation that introduces a stop codon or otherwise affects the open reading frame). Through exon skipping, production of an altered, yet possibly still functional protein, can be achieved.

Genomic DNA can be edited close to the splice site (e.g., so that the activity of the splice site is reduced partially or completely) by using a Cas12a gRNA designed so that upon introduction of the gRNA and the Cas12a protein into a cell containing the genomic sequence, the Cas12a protein cleaves the genomic DNA up to 15 nucleotides from the splice site (e.g., up to 10 nucleotides or 10-15 nucleotides from the splice site).

When a Cas12a protein cleaves genomic DNA, it produces staggered cuts. For example, AsCas12a and LbCas12a proteins cleave genomic DNA after the 19^{th} base following the PAM sequence on the strand having the target domain sequence and after the 23^{rd} base following the PAM sequence on the complementary strand. It should be understood that in connection with the expression “the Cas12a protein cleaves the genomic DNA up to 15 nucleotides from a splice site encoded by the genomic DNA” and similar phrases (e.g., reciting a different number of nucleotides), that counting of the nucleotides should be performed from the overhang closest to the splice site. Moreover, it should be understood that the expression “the Cas12a protein cleaves the genomic DNA up to 15 nucleotides from a splice site encoded by the genomic DNA” and similar phrases encompasses embodiments in which the Cas12a protein cleaves the genomic DNA at the splice site. Thus, the expression “the Cas12a protein cleaves the genomic DNA up to 15 nucleotides from a splice site encoded by the genomic DNA” encompasses embodiments in which the Cas12a protein cleaves the genomic DNA at the splice site, 1 nucleotide from the splice site, 2 nucleotides from the splice site, 3 nucleotides from the splice site, 4 nucleotides from the splice site, 5 nucleotides from the splice site, 6 nucleotides from the splice site, 7 nucleotides from the splice site, 8 nucleotides from the splice site, 9 nucleotides from the splice site, 10 nucleotides from the splice site, 11 nucleotides from the splice site, 12 nucleotides from the splice site, 13 nucleotides from the splice site, 14 nucleotides from the splice site, or 15 nucleotides from the splice site.

With knowledge of the PAM sequence recognized by a particular Cas12a protein (e.g., TTTV for AsCas12a), knowledge of where the Cas12a protein cuts (e.g. 19 and 23 nucleotides after the PAM for AsCas12a), and knowledge of the position of a splice site relative to the PAM sequence in the genomic DNA, a targeting sequence can be selected such that upon introduction of the gRNA and the Cas12a protein into a cell containing the genomic sequence, the Cas12a protein will cleave the genomic DNA up to 15 nucleotides from the splice site. For example, when designing a gRNA for use with AsCas12a protein, the splice site can be after the 4th nucleotide following a TTTV sequence to after the 38th nucleotide following a TTTV sequence.

In some embodiments, the disclosure provides Cas12a gRNAs whose targeting sequence corresponds to a target domain adjacent to a PAM sequence that is within 40 nucleotides (e.g., 4 to 38 nucleotides, 5 to 35 nucleotides, 5 to 25 nucleotides, 5 to 15 nucleotides, 5 to 10 nucleotides, 10 to 35 nucleotides, 10 to 25 nucleotides, 10 to 20 nucleotides, 10 to 15 nucleotides, 15 to 35 nucleotides, 15 to 25 nucleotides, 20 to 35 nucleotides, 20 to 30 nucleotides, or 25 to 35 nucleotides) of a splice site.

Cas12a gRNAs of the disclosure are generally 40-44 nucleotides long (e.g., 40 nucleotides, 41 nucleotides, 42 nucleotides, or 43 nucleotides), but gRNAs of other lengths are also contemplated. For example, extending the 5′ end of a gRNA (e.g., as described in Park et al., 2018, Nature Communications, 9:3313) can be helpful for enhancing gene editing efficacy. Additionally, Cas12a gRNAs of the disclosure can optionally be chemically modified, which can be useful, for example, to enhance serum stability of a gRNA (see, e.g., Park et al., 2018, Nature Communications, 9:3313).

### 6.3.1. Protospacer Domains

The gRNAs of the disclosure comprise a protospacer domain containing a targeting sequence. In some embodiments, the sequence of the protospacer domain and the targeting sequence are the same. In other embodiments, the sequence of the protospacer domain and the targeting sequence are different (e.g., where the protospacer domain comprises one or more nucleotides 5′ and/or 3′ to the targeting sequence).

The protospacer domain can in some embodiments be 17 to 26 nucleotides in length (e.g., 17-20 nucleotides, 17-23 nucleotides, 20-26 nucleotides, or 20-24 nucleotides). In some embodiments, the protospacer domain is 17 nucleotides in length. In other embodiments, the protospacer domain is 18 nucleotides in length. In other embodiments, the protospacer domain is 19 nucleotides in length. In other embodiments, the protospacer domain is 20 nucleotides in length. In other embodiments, the protospacer domain is 21 nucleotides in length. In other embodiments, the protospacer domain is 22 nucleotides in length. In other embodiments, the protospacer domain is 23 nucleotides in length. In other embodiments, the protospacer domain is 24 nucleotides in length. In other embodiments, the protospacer domain is 25 nucleotides in length. In other embodiments, the protospacer domain is 26 nucleotides in length.

The targeting sequence corresponds to a target domain in a genomic DNA sequence. There are preferably no mismatches between the targeting sequence and the complement of the target domain, although embodiments with a small number of mismatches (e.g., 1 or 2) are envisioned. The targeting sequence can in some embodiments be 17 to 26 nucleotides in length (e.g., 20-24 nucleotides in length). In some embodiments, the targeting sequence is 17 nucleotides in length. In other embodiments, the targeting sequence is 18 nucleotides in length. In other embodiments, the targeting sequence is 19 nucleotides in length. In other embodiments, the targeting sequence is 20 nucleotides in length. In other embodiments, the targeting sequence is 21 nucleotides in length. In other embodiments, the targeting sequence is 22 nucleotides in length. In other embodiments, the targeting sequence is 23 nucleotides in length. In other embodiments, the targeting sequence is 24 nucleotides in length. In other embodiments, the targeting sequence is 25 nucleotides in length. In other embodiments, the targeting sequence is 26 nucleotides in length. In some embodiments, the sequence of the protospacer domain and the targeting sequence are the same.

The targeting sequence can, but does not necessarily, correspond to a target domain having a mutation (e.g., a single nucleotide polymorphism). In some embodiments, a Cas12a gRNA of the disclosure has a targeting sequence corresponding to a target domain having a mutation 1 to 23 nucleotides 3′ of a Cas12a PAM sequence (e.g., 1 to 20 nucleotides, 1 to 15 nucleotides, 1 to 10 nucleotides, 1 to 5 nucleotides, 5 to 15 nucleotides, 10 to 20 nucleotides, or 15 to 23 nucleotides from a Cas12a PAM sequence). Cas12a gRNAs having a targeting sequence corresponding to a target domain having a mutation can have allele specificity such that a Cas12a/Cas12a gRNA complex can preferentially cleave the mutant allele over the wild-type allele, thereby resulting in genome editing of only the mutant allele.

Without being bound by theory, it is believed that deletion, correction or other alteration of a mutation during repair of the genomic DNA following cleavage is not necessary to reduce the activity of a splice site. Thus, gRNAs of the disclosure can be effective to reduce the activity of a splice site even when introduction of the gRNA and a Cas12a protein into a cell containing the genomic sequence does not result in deletion, correction or other alteration of the mutation. Thus, in some embodiments, upon introduction of a gRNA of the disclosure and a Cas12a protein into a population cells containing the genomic sequence, cleavage of the genomic DNA by the Cas12a protein may not necessarily delete, correct, or otherwise alter the mutation in all of the resulting indels. For example, the mutation may be deleted, corrected or otherwise altered in 50% or fewer (e.g., 10% to 50%, 10% to 40%, 10% to 30%, or 10% to 20%) of the resulting indels.

### 6.3.2. Splice Sites

6.3.2.1. Cryptic Splice Sites

A cryptic splice site is a non-canonical splice site having the potential for interacting with the spliceosome. Mutations (e.g., splice site mutations) in the DNA encoding mRNA or errors during transcription can create or activate a cryptic splice site in part of the transcript that usually is not spliced. Creation or activation of a cryptic splice site can result in aberrant splicing and, in some cases, a disease phenotype. Thus, in some embodiments, Cas12a gRNAs of the disclosure target a cryptic splice site. In some embodiments, the target domain includes the cryptic splice site. In other embodiments, the target domain does not include the cryptic splice site. The cryptic splice site can be a 5′ cryptic splice site or a 3′ cryptic splice site.

In some embodiments, the cryptic splice is one that is created or activated by a mutation in a genomic DNA sequence. The mutation can be, for example, a single nucleotide polymorphism, an insertion (e.g., 1 to 10 nucleotides or 1 to 100 nucleotides), or a deletion (e.g., 1 to 10 nucleotides or 1 to 100 nucleotides). In some embodiments, the mutation is a single nucleotide polymorphism.

Upon introduction of a Cas12a gRNA and a Cas12a protein into a cell having the genomic DNA sequence encoding the cryptic splice site, the genomic DNA can be edited so that normal splicing is restored. For example, when the Cas12a gRNA is introduced with a Cas12a protein into a population of cells having the genomic DNA sequence (e.g., in vitro), normal splicing can be restored in a portion of the cells, e.g., at least 10% of the cells (e.g., 10% to 20% of the cells), at least 20% of the cells (e.g., 20% to 30% of the cells), at least 30% of the cells (e.g., 30% to 40% of the cells), at least 40% of the cells (e.g., 40% to 50% of the cells), at least 50% of the cells (e.g., 50% to 60% of the cells), at least 60% of the cells (e.g., 60% to 70% of the cells), or at least 70% of the cells (e.g., 70% to 80% of the cells or 70% to 90% of the cells). Without being bound by theory, it is believed that restoration of normal splicing in even a minority of cells can be advantageous for treating some genetic diseases, such as CF, familial hypercholesterolemia type 2, spinal muscular atrophy, hemophilia, and Duchenne muscular dystrophy. For example, it is believed that for a subject having CF, restoring normal splicing in as few as 10% of the subject's lung cells would be sufficient to alleviate the patient's symptoms.

6.3.2.1.1. Cryptic 3′ Splice Sites

A cryptic splice site targeted by a gRNA of the disclosure can be a cryptic 3′ splice site, for example, a splice site which is created by or activated by a mutation. Cryptic 3′ splice sites can be, for example, upstream of a 3′ canonical splice site or upstream of a 5′ cryptic splice site.

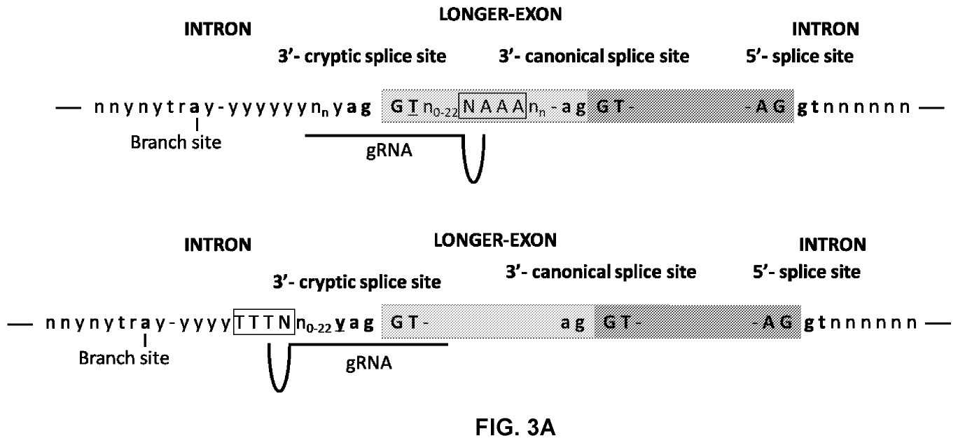

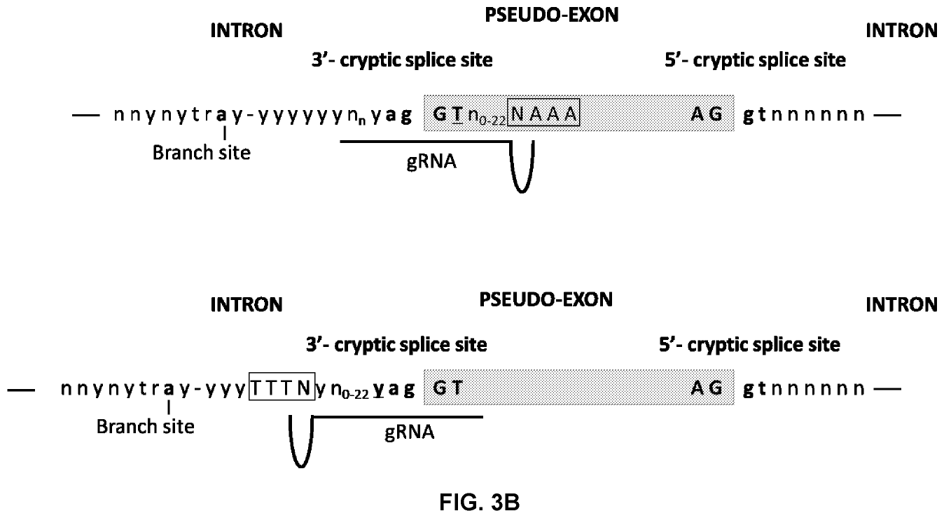

When the cryptic 3′ splice site is upstream of a 3′ canonical splice site, splicing at the cryptic 3′ splice site rather than the 3′ canonical splice site results in an elongated exon (shown schematically in FIG. 3A.) Normal splicing can be restored by reducing the activity of the cryptic 3′ splice site.

When the cryptic 3′ splice site is upstream of a 5′ cryptic splice site, splicing at the cryptic 3′ splice site and the cryptic 5′ splice site results in the inclusion of a pseudo-exon in the mature mRNA (shown schematically in FIG. 3B). Normal splicing can be restored by reducing the activity of the cryptic 3′ splice site so that the pseudo-exon is skipped during pre-mRNA splicing.

Reducing the activity of a cryptic 3′ splice site can be achieved, for example, by disrupting the splice site, disrupting the branch site upstream of the cryptic 3′ splice site (referred to herein as the “branch site of the cryptic 3′ splice site”), or disrupting the polypyrimidine tract upstream of the cryptic 3′ splice site (referred to herein as the “polypyrimidine tract of the cryptic 3′ splice site”). Thus, reducing the activity of a cryptic 3′ splice site can be achieved by using a Cas12a gRNA targeting, for example, the splice site, the branch site, or the polypyrimidine tract.

6.3.2.1.2. Cryptic 5′ Splice Sites

A cryptic splice site targeted by a gRNA of the disclosure can be a cryptic 5′ splice site, for example which has been created or activated by a mutation. Cryptic 5′ splice sites can be, for example, downstream of a cryptic 3′ splice site or downstream of a 5′ canonical splice site.

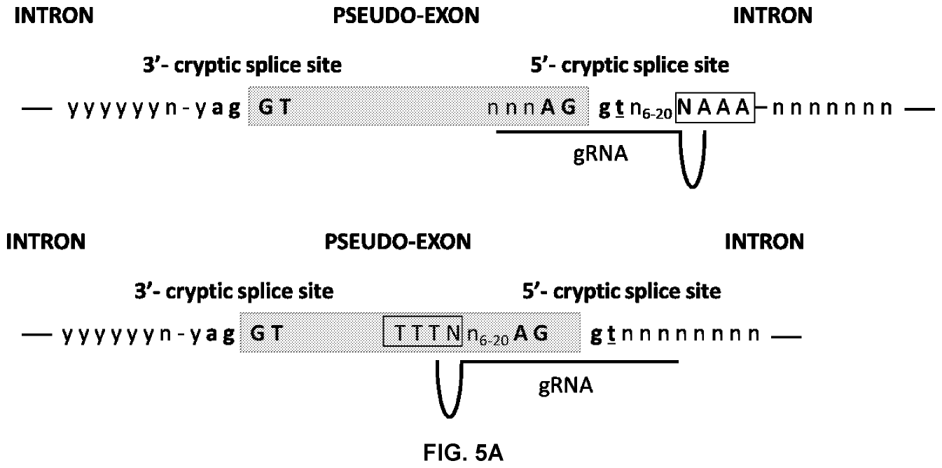

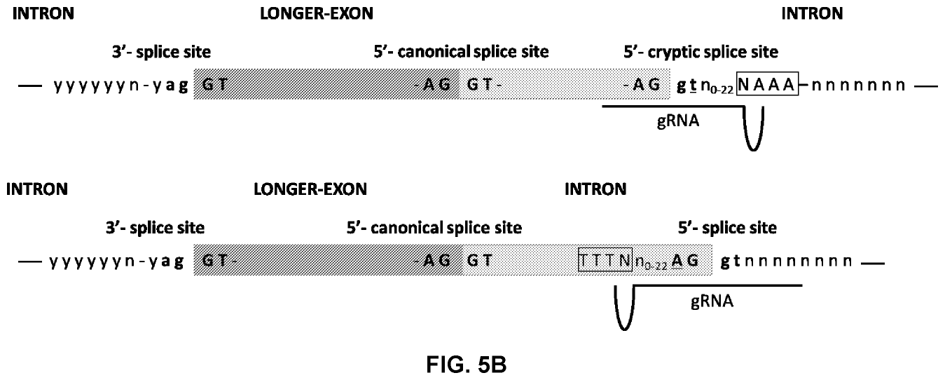

When the cryptic 5′ splice site is downstream of a cryptic 3′ splice site, splicing at the cryptic 3′ splice site and the cryptic 5′ splice site results in the inclusion of a pseudo-exon in the mature mRNA (shown schematically in FIG. 5A). When the cryptic 5′ splice site is downstream of a canonical 5′ splice site, splicing at the cryptic 5′ splice site rather than the canonical 5′ splice site results in a longer than normal exon in the mature mRNA (shown schematically in FIG. 5B). In both instances, normal splicing can be restored by reducing the activity of the cryptic 5′ splice site.

Reducing the activity of a cryptic 5′ splice site can be achieved, for example, by disrupting the cryptic 5′ splice site or surrounding sequence (e.g., from the three nucleotides 5′ of the cryptic splice site to the eight nucleotides 3′ of the cryptic 5′ splice site).

6.3.2.2. Canonical Splice Sites

A Cas12a gRNA of the disclosure can target a canonical splice site. A targeted canonical splice site can be a canonical 3′ splice site or a 5′ canonical splice site.

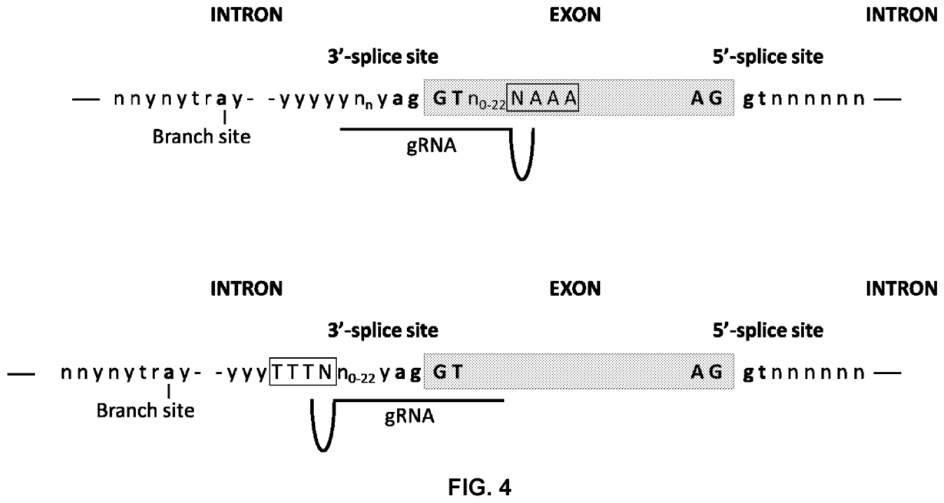

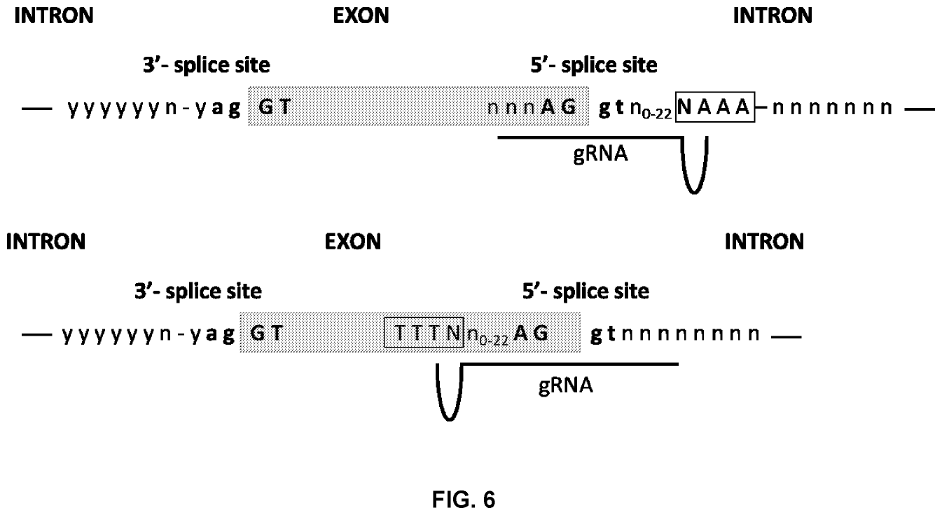

Reducing the activity of a canonical 3′ splice site or a 5′ canonical splice site can be used to cause exon skipping. Targeting of a canonical 3′ splice site is shown schematically in FIG. 4 and targeting of a canonical 5′ splice site is shown schematically in FIG. 6. Exon skipping can be useful, for example, to skip an exon having a deleterious mutation. Exon skipping can be used, for example, to restore the reading frame within a mRNA molecule, for example, a DMD pre-mRNA having a mutation in an exon that causes premature truncation of the dystrophin protein.

Reducing the activity of a canonical 3′ splice site can be achieved, for example, by disrupting the splice site, disrupting the branch site upstream of the canonical 3′ splice site (referred to herein as the “branch site of the canonical 3′ splice site”), or disrupting the polypyrimidine tract upstream of the canonical 3′ splice site (referred to herein as the “polypyrimidine tract of the canonical 3′ splice site”). Reducing the activity of a canonical 5′ splice site can be achieved, for example, by disrupting the canonical 5′ splice site or surrounding sequence (e.g., from the three nucleotides 5′ of the canonical splice site to the eight nucleotides 3′ of the canonical 5′ splice site).

### 6.3.3. Loop Domains

Cas12a is a single gRNA-guided endonuclease where the gRNA comprises a single loop domain having a direct repeat sequence, e.g., a loop domain 20 nucleotides in length. Cas12a proteins recognize the Cas12a gRNA via a combination of structural and sequence-specific features of the loop domain. Loop domains of gRNAs of the disclosure are typically at least 16 nucleotides in length, e.g., 16-20 nucleotides, 16-18 nucleotides, 18-20 nucleotides, 16 nucleotides, 17 nucleotides, 18 nucleotides, 19 nucleotides, or 20 nucleotides in length. In some embodiments, the loop domain is 20 nucleotides in length. Typically, the loop domain will be 5′ to the protospacer domain of a Cas12a gRNA.

Loop domains can comprise a stem-loop sequence that associates with a wild-type Cas12a protein or a variant thereof. See, e.g., Zetsche, et. al, 2015, Cell, 163:759-771, incorporated herein by reference in its entirety, which describes stem-loop sequences of various loop domains capable of associating with Cas12a proteins. Exemplary loop domains include loop domains comprising a nucleotide sequence selected from UCUACUGUUGUAGA (SEQ ID NO: 1), UCUACUGUUGUAGAU (SEQ ID NO: 2), UCUGCUGUUGCAGA (SEQ ID NO: 3), UCUGCUGUUGCAGAU (SEQ ID NO: 4), UCCACUGUUGUGGA (SEQ ID NO: 5), UCCACUGUUGUGGAU (SEQ ID NO: 6), CCUACUGUUGUAGG (SEQ ID NO: 7), CCUACUGUUGUAGGU (SEQ ID NO: 8), UCUACUAUUGUAGA (SEQ ID NO: 9), UCUACUAUUGUAGAU (SEQ ID NO: 10), UCUACUGCUGUAGAU (SEQ ID NO: 11), UCUACUGCUGUAGAUU (SEQ ID NO: 12), UCUACUUUCUAGAU (SEQ ID NO: 13), UCUACUUUCUAGAUU (SEQ ID NO: 14), UCUACUUUGUAGA (SEQ ID NO: 15), UCUACUUUGUAGAU (SEQ ID NO: 16), UCUACUUGUAGA (SEQ ID NO: 17), and UCUACUUGUAGAU (SEQ ID NO: 18).

In some embodiments, the loop domain comprises or consists of a nucleotide sequence selected from UAAUUUCUACUGUUGUAGAU (SEQ ID NO: 19), AGAAAUGCAUGGUUCUCAUGC (SEQ ID NO: 20), AAAAUUACCUAGUAAUUAGGU (SEQ ID NO: 21), GGAUUUCUACUUUUGUAGAU (SEQ ID NO: 22), AAAUUUCUACUUUUGUAGAU (SEQ ID NO: 23), CGCGCCCACGCGGGGCGCGAC (SEQ ID NO: 24), UAAUUUCUACUCUUGUAGAU (SEQ ID NO: 25), GAAUUUCUACUAUUGUAGAU (SEQ ID NO: 26), GAAUCUCUACUCUUUGUAGAU (SEQ ID NO: 27), UAAUUUCUACUUUGUAGAU (SEQ ID NO: 28), AAAUUUCUACUGUUUGUAGAU (SEQ ID NO: 29), GAAUUUCUACUUUUGUAGAU (SEQ ID NO: 30), UAAUUUCUACUAAGUGUAGAU (SEQ ID NO: 31), UAAUUUCUACUAUUGUAGAU (SEQ ID NO: 32), UAAUUUCUACUUCGGUAGAU (SEQ ID NO: 33), and UAAUUUCUACUAUUGUAGAU (SEQ ID NO: 32). In some embodiments, the loop domain comprises or consists of UAAUUUCUACUCUUGUAGAU (SEQ ID NO: 25), which is the loop domain sequence associated with AsCas12a. In some embodiments, the loop domain comprises or consists of UAAUUUCUACUAAGUGUAGAU (SEQ ID NO: 31), which is the loop domain sequence associated with LbCas12a.

Additional stem-loop sequences that associate with Cas12a proteins and which can be used in loop domains of the Cas12a gRNAs of the disclosure are described in Feng, et. al, 2019, Genome Biology, 20:15, incorporated herein by reference in its entirety. Exemplary nucleotide sequences described in Feng, et. al, 2019, Genome Biology, 20:15 and which can be included in loop domains of the Cas12a gRNAs of the disclosure include AUUUCUACUAGUGUAGAU (SEQ ID NO: 34), AUUUCUACUGUGUGUAGA (SEQ ID NO: 35), AUUUCUACUAUUGUAGAU (SEQ ID NO: 36), and AUUUCUACUUUGGUAGAU (SEQ ID NO: 37).

Loop domains having a nucleotide sequence varying from the nucleotide sequences described above can also be used. For example, mutations in a loop domain sequence that preserve the RNA duplex of the loop domain can be used. See, e.g., Zetsche, et. al, 2015, Cell, 163:759-771.

### 6.3.4. Exemplary Target Domains and Cas12a gRNAs

Cas12a gRNAs having targeting sequences corresponding to target domains in various genes can be designed as described herein. For example, a target domain can be in a variant CFTR gene, a variant DMD gene, a variant HBB gene, a variant FGB gene, a variant SOD1 gene, a variant QDPR gene, a variant GLA gene, a variant LDLR gene, a variant BRIP1 gene, a variant F9 gene, a variant CEP290 gene, a variant COL2A1 gene, a variant USH2A gene, or a variant GAA gene. The target domains described below can be used, for example, to design a Cas12a gRNA of the disclosure (e.g., a Cas12a gRNA comprising a targeting sequence corresponding to a target domain described below and a loop domain as described in Section 6.3.3). Such Cas12a gRNAs can be used, for example, together with an appropriate Cas12a protein to restore normal splicing of mRNA. Additional details regarding the specific mutations described in this section can be found in the DBASS database (www.dbass.org.uk).

In some embodiments, the target domain is in a CFTR gene, for example, a CFTR gene having a 3272-26A>G mutation, a 3849+10kbC>T mutation, a IVS11+194A>G mutation, or a IVS19+115050>G mutation. The 3272-26A>G mutation causes aberrant splicing at a cryptic 3′ splice site, whereas the 3849+10kbC>T mutation, IVS11+194A>G mutation, and IVS19+115050>G mutation each cause aberrant splicing at a cryptic 5′ splice site. Each of these mutations is associated with cystic fibrosis.

An exemplary Cas12a gRNA for editing a CFTR gene having a 3272-26A>G mutation can have a targeting sequence corresponding to a target domain comprising or consisting of the sequence CATAGAAAACACTGCAAATAACA (SEQ ID NO: 38).

An exemplary Cas12a gRNA for editing a CFTR gene having a 3849+10kbC>T mutation can have a targeting sequence corresponding to a target domain comprising or consisting of the sequence AGGGTGTCTTACTCACCATTTTA (SEQ ID NO: 39).

An exemplary Cas12a gRNA for editing a CFTR gene having a IVS11+194A>G mutation can have a targeting sequence corresponding to a target domain comprising or consisting of the sequence TACTTGAGATGTAAGTAAGGTTA (SEQ ID NO: 40). Another exemplary Cas12a gRNA for editing a CFTR gene having a IVS11+194A>G mutation can have a targeting sequence corresponding to a target domain comprising or consisting of the sequence ATAGTAACCTTACTTACATCTCA (SEQ ID NO: 41).

An exemplary Cas12a gRNA for editing a CFTR gene having a IVS19+115050>G mutation can have a targeting sequence corresponding to a target domain comprising or consisting of the sequence AAATTCCATCTTACCAATTCTAA (SEQ ID NO: 42). Another exemplary Cas12a gRNA for editing a CFTR gene having a IVS19+115050>G mutation can have a targeting sequence corresponding to a target domain comprising or consisting of the sequence AACGTTAAAATTCCATCTTACCA (SEQ ID NO: 43).

In other embodiments, the target domain is in a DMD gene, for example a DMD gene having a IVS9+468060>T mutation, a IVS62+62296A>G mutation, a IVS1+36947G>A mutation, a IVS1+36846G>A mutation, a IVS2+5591T>A mutation or a IVS8-15A>G mutation. The IVS1+36947G>A mutation, IVS1+36846G>A mutation, IVS2+5591T>A mutation and IVS8-15A>G mutation each cause aberrant splicing at a cryptic 3′ splice site, whereas the IVS9+468060>T mutation and IVS62+62296A>G mutation each cause aberrant splicing at a cryptic 5′ splice site. Each of these mutations is associated with muscular dystrophy.

An exemplary Cas12a gRNA for editing a DMD gene having a IVS9+468060>T mutation can have a targeting sequence corresponding to a target domain comprising or consisting of the sequence TGACCTTTGGTAAGTCATCTAAT (SEQ ID NO: 44). Another exemplary Cas12a gRNA for editing a DMD gene having a IVS9+46806C>T mutation can have a targeting sequence corresponding to a target domain comprising or consisting of the sequence CCTTTGTGACCTTTGGTAAGTCA (SEQ ID NO: 45).

An exemplary Cas12a gRNA for editing a DMD gene having a IVS62+62296A>G mutation can have a targeting sequence corresponding to a target domain comprising or consisting of the sequence TTGATCACATAACAAGGTCAGTT (SEQ ID NO: 46). Another exemplary Cas12a gRNA for editing a DMD gene having a IVS62+62296A>G mutation can have a targeting sequence corresponding to a target domain comprising or consisting of the sequence ATCACATAACAAGGTCAGTTTAT (SEQ ID NO: 47). Another exemplary Cas12a gRNA for editing a DMD gene having a IVS62+62296A>G mutation can have a targeting sequence corresponding to a target domain comprising or consisting of the sequence AGTTATGATAAACTGACCTTGTT (SEQ ID NO: 48). Another exemplary Cas12a gRNA for editing a DMD gene having a IVS62+62296A>G mutation can have a targeting sequence corresponding to a target domain comprising or consisting of the sequence TGATAAACTGACCTTGTTATGTG (SEQ ID NO: 49).

An exemplary Cas12a gRNA for editing a DMD gene having a IVS1+36947G>A mutation can have a targeting sequence corresponding to a target domain comprising or consisting of the sequence TCTTCCTTGGTTTTGCAGCTTCT (SEQ ID NO: 50). Another exemplary Cas12a gRNA for editing a DMD gene having a IVS1+36947G>A mutation can have a targeting sequence corresponding to a target domain comprising or consisting of the sequence TTGGTTTTGCAGCTTCTCGAGTT (SEQ ID NO: 51). Another exemplary Cas12a gRNA for editing a DMD gene having a IVS1+36947G>A mutation can have a targeting sequence corresponding to a target domain comprising or consisting of the sequence CTCTTTCTCTTCCTTGGTTTTGC (SEQ ID NO: 52).

An exemplary Cas12a gRNA for editing a DMD gene having a IVS2+5591T>A mutation can have a targeting sequence corresponding to a target domain comprising or consisting of the sequence CTTGTTTCTCTACATAGGTTGAA (SEQ ID NO: 53).

An exemplary Cas12a gRNA for editing a DMD gene having a IVS8-15A>G mutation can have a targeting sequence corresponding to a target domain comprising or consisting of the sequence TCCTCTCTATCCACCTCCCCCAG (SEQ ID NO: 54). Another exemplary Cas12a gRNA for editing a DMD gene having a IVS8-15A>G mutation can have a targeting sequence corresponding to a target domain comprising or consisting of the sequence CCTCCCCCAGACCCTTCTCTGCA (SEQ ID NO: 55). Another exemplary Cas12a gRNA for editing a DMD gene having a IVS8-15A>G mutation can have a targeting sequence corresponding to a target domain comprising or consisting of the sequence CCCCTCCTCTCTATCCACTCCCC (SEQ ID NO: 56). Another exemplary Cas12a gRNA for editing a DMD gene having a IVS8-15A>G mutation can have a targeting sequence corresponding to a target domain comprising or consisting of the sequence CCTCCTCTCTATCCACCTCCCCC (SEQ ID NO: 57).

An exemplary Cas12a gRNA for editing for causing exon skipping of exon 51 in a DMD gene having a mutation in exon 50 of DMD can have a targeting sequence corresponding to a target domain comprising or consisting of the sequence CAAAAACCCAAAATATTTTAGCT (SEQ ID NO: 58). Another exemplary Cas12a gRNA for causing exon skipping of exon 51 of a DMD gene having a mutation in exon 50 of DMD can have a targeting sequence corresponding to a target domain comprising or consisting of the sequence CTTTTTGCAAAAACCCAAAATAT (SEQ ID NO: 59). Another exemplary Cas12a gRNA for causing exon skipping of exon 51 of a DMD gene having a mutation in exon 50 of DMD can have a targeting sequence corresponding to a target domain comprising or consisting of the sequence TTTTTGCAAAAACCCAAAATATT (SEQ ID NO: 60). Another exemplary Cas12a gRNA for causing exon skipping of exon 51 of a DMD gene having a mutation in exon 50 of DMD can have a targeting sequence corresponding to a target domain comprising or consisting of the sequence TGTCACCAGAGTAACAGTCTGAG (SEQ ID NO: 61). Another exemplary Cas12a gRNA for causing exon skipping of exon 51 of a DMD gene having a mutation in exon 50 of DMD can have a targeting sequence corresponding to a target domain comprising or consisting of the sequence GCTCCTACTCAGACTGTTACTCT (SEQ ID NO: 62).

In other embodiments, the target domain is in a HBB gene, for example a HBB gene having a IVS2+645C>T mutation, a IVS2+705T>G mutation, or a IVS2+745C>G mutation. Each of these mutations causes aberrant splicing at a 5′ cryptic splice site and is associated with beta-thalassemia.

An exemplary Cas12a gRNA for editing a HBB gene having a IVS2+645C>T mutation can have a targeting sequence corresponding to a target domain comprising or consisting of the sequence TGGGTTAAGGTAATAGCAATATC (SEQ ID NO: 63). Another exemplary Cas12a gRNA for editing a HBB gene having a IVS2+645C>T mutation can have a targeting sequence corresponding to a target domain comprising or consisting of the sequence TATGCAGAGATATTGCTATTACC (SEQ ID NO: 64). Another exemplary Cas12a gRNA for editing a HBB gene having a IVS2+645C>T mutation can have a targeting sequence corresponding to a target domain comprising or consisting of the sequence CTATTACCTTAACCCAGAAATTA (SEQ ID NO: 65). Another exemplary Cas12a gRNA for editing a HBB gene having a IVS2+645C>T mutation can have a targeting sequence corresponding to a target domain comprising or consisting of the sequence CAGAGATATTGCTATTACCTTAA (SEQ ID NO: 66).

An exemplary Cas12a gRNA for editing a HBB gene having a IVS2+705T>G mutation can have a targeting sequence corresponding to a target domain comprising or consisting of the sequence TGCATATAAATTGTAACTGAGGT (SEQ ID NO: 67). Another exemplary Cas12a gRNA for editing a HBB gene having a IVS2+705T>G mutation can have a targeting sequence corresponding to a target domain comprising or consisting of the sequence AATTGTAACTGAGGTAAGAGGTT (SEQ ID NO: 68). Another exemplary Cas12a gRNA for editing a HBB gene having a IVS2+705T>G mutation can have a targeting sequence corresponding to a target domain comprising or consisting of the sequence AAACCTCTTACCTCAGTTACAAT (SEQ ID NO: 69). Another exemplary Cas12a gRNA for editing a HBB gene having a IVS2+705T>G mutation can have a targeting sequence corresponding to a target domain comprising or consisting of the sequence GCAATATGAAACCTCTTACCTCA (SEQ ID NO: 70).

An exemplary Cas12a gRNA for editing a HBB gene having a IVS2+745C>G mutation can have a targeting sequence corresponding to a target domain comprising or consisting of the sequence CTAATAGCAGCTACAATCCAGGT (SEQ ID NO: 71).

In other embodiments, the target domain is in a FGB gene, for example a FGB gene having a IVS6+13C>T mutation. This mutation causes aberrant splicing at cryptic 5′ splice site and is associated with afibrinogenemia. An exemplary Cas12a gRNA for editing a FGB gene having a IVS6+13C>T mutation can have a targeting sequence corresponding to a target domain comprising or consisting of the sequence TTTTGCATACCTGTTCGTTACCT (SEQ ID NO: 72). Another exemplary Cas12a gRNA for editing a FGB gene having a IVS6+13C>T mutation can have a targeting sequence corresponding to a target domain comprising or consisting of the sequence AAATAGAATGATTTTATTTTGCA (SEQ ID NO: 73).

In other embodiments, the target domain is in a SOD1 gene, for example a SOD1 gene having a IVS4+792C>G mutation. This mutation causes aberrant splicing at a cryptic 5′ splice site and is associated with amyotrophic lateral sclerosis. An exemplary Cas12a gRNA for editing a SOD1 gene having a IVS4+792C>G mutation can have a targeting sequence corresponding to a target domain comprising or consisting of the sequence TGGTAAGTTACACTAACCTTAGT (SEQ ID NO: 74).

In other embodiments, the target domain is in a QDPR gene, for example a QDPR gene having a IVS3+2552A>G mutation. This mutation causes aberrant splicing at a cryptic 5′ splice site and is associated with dihydropteridine reductase deficiency. An exemplary Cas12a gRNA for editing a QDPR gene having a QDPR a IVS3+2552A>G mutation can have a targeting sequence corresponding to a target domain comprising or consisting of the sequence TCATCTGTAAAATAAGAGTAAAA (SEQ ID NO: 75).

In other embodiments, the target domain is in a GLA gene, for example a GLA gene having a IVS4+919G>A mutation. This mutation causes aberrant splicing at a cryptic 5′ splice site and is associated with Fabry disease. An exemplary Cas12a gRNA for editing a GLA gene having a IVS4+919G>A mutation can have a targeting sequence corresponding to a target domain comprising or consisting of the sequence CCATGTCTCCCCACTAAAGTGTA (SEQ ID NO: 76).

In other embodiments, the target domain is in a LDLR gene, e.g., a LDLR gene having a IVS12+11C>G mutation. This mutation causes aberrant splicing at a cryptic 5′ splice site and is associated with familial hypercholesterolemia. An exemplary Cas12a gRNA for editing a LDLR gene having a IVS12+11C>G mutation can have a targeting sequence corresponding to a target domain comprising or consisting of the sequence AGGTGTGGCTTAGGTACGAGATG (SEQ ID NO: 77).

In other embodiments, the target domain is in a BRIP1 gene, for example a BRIP1 gene having a IVS11+2767A>T mutation. This mutation causes aberrant splicing at a cryptic 5′ splice site and is associated with Fanconi anemia. An exemplary Cas12a gRNA for editing a BRIP1 gene having a IVS11+2767A>T mutation can have a targeting sequence corresponding to a target domain comprising or consisting of the sequence TAAAATTCTTACATACCTTTGAA (SEQ ID NO: 78).

In other embodiments, the target domain is in a F9 gene, for example a F9 gene having a IVS5+13A>G mutation. This mutation causes aberrant splicing at a cryptic 5′ splice site and is associated with hemophilia B. An exemplary Cas12a gRNA for editing a F9 gene having a IVS5+13A>G mutation can have a targeting sequence corresponding to a target domain comprising or consisting of the sequence AAAAATCTTACTCAGATTATGAC (SEQ ID NO: 79). Another exemplary Cas12a gRNA for editing for a F9 gene having a IVS5+13A>G mutation can have a targeting sequence corresponding to a target domain comprising or consisting of the sequence TTTAAAAAATCTTACTCAGATTA (SEQ ID NO: 80).

In other embodiments, the target domain is in a CEP290 gene, for example a CEP290 gene having a IVS26+1655A>G mutation. This mutation causes aberrant splicing at a cryptic 5′ splice site and is associated with Leber congenital amaurosis. An exemplary Cas12a gRNA for editing a CEP290 gene having a VS26+1655A>G mutation can have a targeting sequence corresponding to a target domain comprising or consisting of the sequence AGTTGTAATTGTGAGTATCTCAT (SEQ ID NO: 81).

In other embodiments, the target domain is in a COL2A1 gene, for example a COL2A1 gene having a IVS23+135G>A mutation. This mutation causes aberrant splicing at a cryptic 3′ splice site and is associated with Stickler syndrome An exemplary Cas12a gRNA for editing a COL2A1 gene having a IVS23+135G>A mutation can have a targeting sequence corresponding to a target domain comprising or consisting of the sequence TCCATCCACACCGCAGGGAGAG (SEQ ID NO: 82).

In other embodiments, the target domain is in a USH2A gene, for example a USH2A gene having a IVS40-8C>G mutation, a IVS66+39C>T mutation, or a c.7595-2144A>G mutation. The IVS40-8C>G mutation causes aberrant splicing at a cryptic 3′ splice site and is associated with Usher syndrome, type II. The IVS66+39C>T mutation is associated with Usher syndrome and causes aberrant splicing at a cryptic 5′ splice site. The c.7595-2144A>G mutation is deep intronic mutation associated with Usher syndrome, type II and causes aberrant splicing at a cryptic 5′ splice site and a cryptic 3′ splice site.

An exemplary Cas12a gRNA for editing a USH2A gene having a IVS40-8C>G mutation can have a targeting sequence corresponding to a target domain comprising or consisting of the sequence TGGATTTATTTTAGTTTACAGAA (SEQ ID NO: 83). Another exemplary Cas12a gRNA for editing a USH2A gene having a IVS40-8C>G mutation can have a targeting sequence corresponding to a target domain comprising or consisting of the sequence TTTTAGTTTACAGAACCTGGACC (SEQ ID NO: 84). Another exemplary Cas12a gRNA for editing a USH2A gene having a IVS40-8C>G mutation can have a targeting sequence corresponding to a target domain comprising or consisting of the sequence CAAGAGGTCTGACTTTCTGGATT (SEQ ID NO: 85). Another exemplary Cas12a gRNA for editing a USH2A gene having a IVS40-8C>G mutation can have a targeting sequence corresponding to a target domain comprising or consisting of the sequence AGAGGTCTGACTTTCTGGATTTA (SEQ ID NO: 86). Another exemplary Cas12a gRNA for editing a USH2A gene having a IVS40-8C>G mutation can have a targeting sequence corresponding to a target domain comprising or consisting of the sequence GGTTCTGTAAACTAAAATAAATC (SEQ ID NO: 87).

An exemplary Cas12a gRNA for editing a USH2A gene having a IVS66+39C>T mutation can have a targeting sequence corresponding to a target domain comprising or consisting of the sequence TATGTCTGTACACATACCTTGTT (SEQ ID NO: 88). Another exemplary Cas12a gRNA for editing a USH2A gene having a IVS66+39C>T mutation can have a targeting sequence corresponding to a target domain comprising or consisting of the sequence ATATGTCTGTACACATACCTTGT (SEQ ID NO: 89).

An exemplary Cas12a gRNA for editing a USH2A gene having a c.7595-2144A>G mutation can have a targeting sequence corresponding to a target domain comprising or consisting of the sequence TTAAAGATGATCTCTTACCTTGG (SEQ ID NO: 90). Another exemplary Cas12a gRNA for editing a USH2A gene having a c.7595-2144A>G mutation can have a targeting sequence corresponding to a target domain comprising or consisting of the sequence CCAAGGTAAGAGATCATCTTTAA (SEQ ID NO: 91). Another exemplary Cas12a gRNA for editing a USH2A gene having a c.7595-2144A>G mutation can have a targeting sequence corresponding to a target domain comprising or consisting of the sequence AAATTGAACACCTCTCCTTTCCC (SEQ ID NO: 92). Another exemplary Cas12a gRNA for editing a USH2A gene having a c.7595-2144A>G mutation can have a targeting sequence corresponding to a target domain comprising or consisting of the sequence AAGATGATCTCTTACCTTGGGAA (SEQ ID NO: 93). The sequences identified in this paragraph can be used to edit the USH2A gene close to the cryptic 5′ splice site.

Another exemplary Cas12a gRNA for editing a USH2A gene having a c.7595-2144A>G mutation can have a targeting sequence corresponding to a target domain comprising or consisting of the sequence AGCTGCTTTCAGCTTCCTCTCCAG (SEQ ID NO: 94). Another exemplary Cas12a gRNA for editing a USH2A gene having a c.7595-2144A>G mutation can have a targeting sequence corresponding to a target domain comprising or consisting of the sequence TGGAGAGGAAGCTGAAAGCAGCT (SEQ ID NO: 95). Another exemplary Cas12a gRNA for editing a USH2A gene having a c.7595-2144A>G mutation can have a targeting sequence corresponding to a target domain comprising or consisting of the sequence TGTGATTCTGGAGAGGAAGCTGA (SEQ ID NO: 96). Another exemplary Cas12a gRNA for editing a USH2A gene having a c.7595-2144A>G mutation can have a targeting sequence corresponding to a target domain comprising or consisting of the sequence ACTTGTGTGATTCTGGAGAGGAA (SEQ ID NO: 97). The sequences identified in this paragraph can be used to edit the USH2A gene close to the cryptic 3′ splice site.

In other embodiments, the target domain is in a GAA gene, for example a GAA gene having a IVS1-13T>G mutation or a IVS6-22T>G mutation. Both of these mutations cause aberrant splicing at cryptic 3′ splice sites, and are associated with glycogen storage disease, type II.

An exemplary Cas12a gRNA for editing a GAA gene having a IVS1-13T>G mutation can have a targeting sequence corresponding to a target domain comprising or consisting of the sequence TGCTGAGCCCGCTTGCTTCTCCC (SEQ ID NO: 98). Another exemplary Cas12a gRNA for editing a GAA gene having a IVS1-13T>G mutation can have a targeting sequence corresponding to a target domain comprising or consisting of GCCTCCCTGCTGAGCCCGCTTGC (SEQ ID NO: 99). Another exemplary Cas12a gRNA for editing a GAA gene having a IVS1-13T>G mutation can have a targeting sequence corresponding to a target domain comprising or consisting of TCCCGCCTCCCTGCTGAGCCCGC (SEQ ID NO: 100).

An exemplary Cas12a gRNA for editing a GAA gene having a IVS6-22T>G mutation can have a targeting sequence corresponding to a target domain comprising or consisting of the sequence TCCTCCCTCCCTCAGGAAGTCGG (SEQ ID NO: 101). Another exemplary Cas12a gRNA for editing a GAA gene having a IVS6-22T>G mutation can have a targeting sequence corresponding to a target domain comprising or consisting of AAGGCTCCCTCCTCCCTCCCTCA (SEQ ID NO: 102). Another exemplary Cas12a gRNA for editing a GAA gene having a IVS6-22T>G mutation can have a targeting sequence corresponding to a target domain comprising or consisting of TCCCTCAGGAAGTCGGCGTTGGC (SEQ ID NO: 103).

### 6.4. Cas12a Proteins

Cas12a proteins have been isolated from a number of bacterial species, e.g., Alicyclobacillus acidoterrestris, Bacillus thermoamylovorans, Lachnospiraceae bacterium (e.g., LbCas12a, NCBI Reference Sequence WP_051666128.1), Acidaminococcus sp. BV3L6 (e.g., AsCas12a, NCBI Reference Sequence WP_021736722.1), Arcobacter butzleri L348 (e.g., AbCas12a, GeneBank ID: JAIQ01000039.1), Agathobacter rectalis strain 2789STDY5834884 (e.g., ArCas12a, GeneBank ID: CZAJ01000001.1), Bacteroidetes oraltaxon 274 str. F0058 (e.g., BoCas12a, GeneBank ID: NZ_GG774890.1), Butyrivibrio sp. NC3005 (e.g., BsCas12a, GeneBank ID: NZ_AUKC01000013.1), Candidate division WS6 bacterium GW2011_GWA2_37_6 US52_C0007 (e.g., C6Cas12a, GeneBank ID: LBTH01000007.1), Helcococcus kunzii ATCC 51366 (e.g., HkCas12a, GeneBank ID: JH601088.1/AGEI01000022.1), Lachnospira pectinoschiza strain 2789STDY5834836 (e.g., LpCas12a, GeneBank ID: CZAK01000004.), Oribacterium sp. NK2B42 (e.g., OsCas12a, GeneBank ID: NZ_KE384190.1), Pseudobutyrivibrio ruminis CF1b (e.g., PrCas12a, GeneBank ID: NZ_KE384121.1), Proteocatella sphenisci DSM 23131 (e.g., PsCas12a, GeneBank ID: NZ_KE384028.1), Pseudobutyrivibrio xylanivorans strain DSM 10317 (e.g., PxCas12a, GeneBank ID: FMWK01000002.1), Sneathia amnii strain SN35 (e.g., SaCas12a, GeneBank ID: CP011280.1), Francisella novicida, and Leptotrichia shahii. The Cas12a protein used in the systems, particles, and methods of the disclosure can be, for example, a wild-type Cas12a protein, for example AsCas12a, LbCas12a, or another wild-type Cas12a protein described herein. In some embodiments, the Cas12a protein is AsCas12a. In other embodiments, the Cas12a protein is LbCas12a.

The success of gene editing by CRSIPR-Cas systems relies, at least in part, upon the specificity of the Cas protein for the target sequence with the fewest off-target effects, e.g., editing of non-targeting DNA. Cas12a proteins can be engineered to exhibit increased specificity relative to wild-type proteins by, for example, the introduction of one or more mutations in amino acid residues involved with directing contact of the Cas12a protein with the DNA backbone of either the target or non-target DNA. Reducing the binding affinity of a Cas12a protein to DNA can improve Cas12a protein fidelity by increasing the ability of the Cas12a protein to discriminate against non-target DNA sequences. In some embodiments, the Cas12a protein used in the systems, particles, and methods of the disclosure can be, for example, an engineered Cas12a protein, e.g., an engineered LbCas12a or engineered AsCas12a having one or more amino acid substitutions compared to the wild-type protein.

Exemplary engineered LbCas12a proteins are described in US Patent Application Publication No. 2018/0030425, the contents of which are incorporated herein by reference in their entirety. Engineered LbCas12a proteins can include, but are not limited to, the amino acid sequence of SEQ ID NO:1 (corresponding to NCBI Reference Sequence WP_051666128.1) or SEQ ID NO:10 of US 2018/0030425, optionally comprising mutations, for example, replacement of a native amino acid with a different amino acid, e.g., alanine, glycine, or serine, at one or more positions in the sequence of SEQ ID NO:10 of US 2018/0030425, e.g., at position S186, e.g., at position N256, e.g., at position N260, e.g., at position K272, e.g., at position K349, e.g., at position K514, e.g., at position K591, e.g., at position K897, e.g., at position Q944, e.g., at position K945, e.g., at position K948, e.g., at position K984, or e.g., at position S985, or any combination thereof, or at positions analogous thereto in SEQ ID NO:1 of US 2018/0030425, e.g., at position S202, e.g., at position N274, e.g., at position N278, e.g., at position K290, e.g., at position K367, e.g., at position K532, e.g., at position K609, e.g., at position K915, e.g., at position Q962, e.g., at position K963, e.g., at position K966, e.g., at position K1002, or e.g., at position S1003 of SEQ ID NO:1 US 2018/0030425; or any combination thereof. In some embodiments, an engineered LbCas12a comprises mutations G532R/K595R and G532R/K538V/Y542R.

Exemplary engineered AsCas12a proteins are described in US Patent Application Publication No. 2018/0030425, the contents of which are incorporated herein by reference in their entirety. Engineered AsCas12a proteins include, but are not limited to, the amino acid sequence of SEQ ID NO:2 (corresponding to NCBI Reference Sequence WP_021736722.1) or SEQ ID NO:8 of US 2018/0030425, optionally comprising mutations, for example, replacement of a native amino acid with a different native amino acid, e.g., alanine, glycine, or serine, at one or more positions in the sequence of SEQ ID NO:2 of US 2018/0030425, e.g., at position N178, e.g., at position S186, e.g., at position N278, e.g., at position N282, e.g., at position R301, e.g., at position T315, e.g., at position S376, e.g., at position N515, e.g., at position K523, e.g., at position K524, e.g., at position K603, e.g., at position K965, e.g., at position Q1013, e.g., at position Q1014, or e.g., at position K1054 of SEQ ID NO:2, or a combination thereof.

Additional engineered LbCas12a and AsCas12a proteins are described in US Patent Application Publication No. 2019/0010481, the contents of which are incorporated herein by reference in their entirety. Such engineered Cas12a proteins can comprise, for example, an amino acid sequence that is at least 80% or at least 95% identical to the amino acid sequence of wild-type LbCas12a or wild-type AsCas12a. Engineered Cas12a proteins can include one or more of the mutations described in US Patent Application Publication No. 2019/0010481.

Engineered Cas12a proteins can be a fusion protein, for example, comprising a heterologous functional domain, e.g., a transcriptional activation domain, a transcriptional silencer or transcriptional repression domain, an enzyme that modifies the methylation state of DNA, an enzyme that modifies a histone subunit, a deaminase that modifies cytosine DNA bases, a deaminase that modifies adenosine DNA bases, an enzyme, domain, or peptide that inhibits or enhances endogenous DNA repair or base excision repair (BER) pathways, or a biological tether, as described in US Patent Application Publication No. 2019/0010481.

The success of gene editing by CRISPR-Cas systems also relies, at least in part, upon the specificity of the Cas protein for its PAM sequence(s). Wild-type LbCas12a and AsCas12a proteins recognize the PAM sequence TTTV, where V is A, C or G. Engineered AsCas12a proteins having S542R/K607R (RR Cas12a) and S542R/K548V/N552R (RVR Cas12a) mutations are described in Gao et. al, 2017, Nat Biotechnol., 35(8):789-792, and have altered PAM specificities compared to wild-type Cas12a. Table 1 shows PAM sequences recognized by various Cas12a proteins. See also Feng, et. al, 2019, Genome Biology, 20:15.

TABLE 1

| Cas12a Protein | PAM Sequence | |
|---|---|---|
| WT LbCas12a and | TTTV | V = A, C, or G |
| WT AsCas12a | | |
| WT FnCas12 | NTTN | N = A, G, C, or T |
| RR Cas12a | TYCV | Y = C or T; |
| | | V = A, C, or G |
| RR Cas12a | CCCC | |
| RR Cas12a | ACCC | |
| RVR Cas12a | TATV | V = A, C, or G |
| RVR Cas12a | RATR | R = G or A |
| HkCas12a | TCTN | N = A, G, C, or T |
| ArCas12a; | TTTN | N = A, G, C, or T |
| BsCas12a; | or | |
| HkCas12a; | TTN | |
| LpCas12a; | | |
| PrCas12a; | | |
| PxCas12a. | | |
| HkCas12a | YYN | Y = C or T; |
| | | N = A, G, C, or T |
| HkCas12a | YTN | Y = C or T; |
| | | N = A, G, C, or T |
| HkCas12a | TYYN | Y = C or T; |
| | | N = A, G, C, or T |

### 6.5. Nucleic Acids and Host Cells

The disclosure provides nucleic acids (e.g., DNA or RNA) encoding the Cas12a gRNAs of the disclosure. A nucleic acid encoding a Cas12a gRNA can be, for example, a plasmid or a virus genome (e.g., a lentivirus, retrovirus, adenovirus, or adeno-associated virus genome modified to encode the Cas12a gRNA). Plasmids can be, for example, plasmids for producing virus particles, e.g., lentivirus particles, or plasmids for propagating the Cas12a gRNA coding sequence in bacterial (e.g., E. coli) or eukaryotic (e.g., yeast) cells.

A nucleic acid encoding a gRNA can, in some embodiments, further encode a Cas12a protein, e.g., a Cas12a protein described in Section 6.4. An exemplary plasmid that can be used to encode a Cas12a gRNA of the disclosure and a Cas12a protein is pY108 lentiAsCas12a (Addgene Plasmid 84739), which encodes AsCas12a. Those of skill in the art will appreciate that plasmids encoding a Cas12a protein can be modified to encode a different Cas12a protein, e.g., a Cas12a variant as described in Section 6.4 or a Cas12a protein from a different species such as Lachnospiraceae bacterium or Francisella novicida.

Nucleic acids encoding a Cas12a protein can be codon optimized, e.g., where at least one non-common codon or less-common codon has been replaced by a codon that is common in a host cell. For example, a codon optimized nucleic acid can direct the synthesis of an optimized messenger mRNA, e.g., optimized for expression in a mammalian expression system.

Nucleic acids of the disclosure, e.g., plasmids, can comprise one or more regulatory elements such as promoters, enhancers, and other expression control elements (e.g., transcription termination signals, such as polyadenylation signals and poly-U sequences). Such regulatory elements are described, for example, in Goeddel, 1990, GENE EXPRESSION TECHNOLOGY: METHODS IN ENZYMOLOGY 185, Academic Press, San Diego, Calif. Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, pancreas), or in particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a nucleic acid of the disclosure comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof, e.g., to express a Cas12a gRNA and a Cas12a protein separately. Examples of pol III promoters include, but are not limited to, U6 and H1 promoters. Examples of pol II promoters include, but are not limited to, the retroviral Rous Sarcoma virus (RSV) LTR promoter (optionally with the RSV enhancer), the cytomegalovirus (CMV) promoter (optionally with the CMV enhancer) (see, e.g., Boshart et al, Cell, 1985, 41:521-530), the SV40 promoter, the dihydrofolate reductase promoter, the β-actin promoter, the phosphoglycerol kinase (PGK) promoter, and the EF1α promoter. Exemplary enhancer elements include WPRE; CMV enhancers; the R-U5′ segment in LTR of HTLV-I; SV40 enhancer; and the intron sequence between exons 2 and 3 of rabbit β-globin. It will be appreciated by those skilled in the art that the design of an expression vector can depend on such factors as the choice of the host cell, the level of expression desired, etc.

The disclosure also provides a host cell comprising a nucleic acid of the disclosure.

Such host cells can be used, for example, to produce virus particles encoding a Cas12a gRNA of the disclosure and, optionally, a Cas12a protein. Host cells can also be used to make vesicles containing a Cas12a gRNA and, optionally, a Cas12a protein (e.g., by adapting the methods described in Montagna et al., 2018, Molecular Therapy: Nucleic Acids, 12:453-462 to make vesicles comprising a Cas12a gRNA and a Cas12a protein rather than a Cas9 sgRNA and a Cas9 protein). Exemplary host cells include eukaryotic cells, e.g., mammalian cells. Exemplary mammalian host cells include human cell lines such as BHK-21, BSRT7/5, VERO, WI38, MRCS, A549, HEK293, HEK293T, Caco-2, B-50 or any other HeLa cells, HepG2, Saos-2, HuH7, and HT1080 cell lines. Host cells can be engineered host cells, for example, host cells engineered to express a DNA binding protein such a repressor (e.g., TetR), to regulate virus or vesicle production (see Petris et al., 2017, Nature Communications, 8:15334).

Host cells can also be used to propagate the Cas12a gRNA coding sequences of the disclosure. The host cell can be a eukaryote or prokaryote and includes, for example, yeast (such as Pichia pastoris or Saccharomyces cerevisiae), bacteria (such as E. coli or Bacillus subtilis), insect Sf9 cells (such as baculovirus-infected SF9 cells) or mammalian cells (such as Human Embryonic Kidney (HEK) cells, Chinese hamster ovary cells, HeLa cells, human 293 cells and monkey COS-7 cells).

### 6.6. Systems, Particles, and Cells Containing Cas12a gRNAs

The disclosure further provides systems comprising a Cas12a gRNA of the disclosure and a Cas12a protein. The systems can comprise a ribonucleoprotein particle (RNP) in which the Cas12a gRNA as described herein is complexed with a Cas12a protein. The Cas12a protein can be, for example, a Cas12a protein described in Section 6.4. Systems of the disclosure can further comprise genomic DNA complexed with the Cas12a gRNA and the Cas12a protein. Accordingly, the disclosure provides a system comprising a Cas12a gRNA of the disclosure comprising a targeting sequence, a genomic DNA comprising a corresponding target domain and a Cas12a PAM, and the Cas12a protein that recognizes PAM, all complexed with one another.

The systems of the disclosure can exist within a cell (whether the cell is in vivo, ex vivo, or in vitro) or outside a cell.

The disclosure further provides particles comprising a Cas12a gRNA of the disclosure. The particles can further comprise a Cas12a protein, e.g., a Cas12a protein described in Section 6.4. Exemplary particles include liposomes, vesicles, and gold nanoparticles. In some embodiments, a particle contains only a single species of gRNA.

The disclosure further provides cells and populations of cells (e.g., a population comprising 10 or more, 50 or more 100 or more, 1,000 or more, or 100,000 thousand or more cells) comprising a Cas12a gRNA of the disclosure. Such cells and populations can further comprise a Cas12a protein. In some embodiments, such cells and populations are isolated, e.g., isolated from cells not containing the Cas12a gRNA.

The cell populations of the disclosure can be cells in which gene editing by the systems of the disclosure has taken place, or cells in which the components of a system of the disclosure have been expressed but gene editing has not taken place, or a combination thereof. A cell population can comprise, for example, a population in which at least 20%, at least 30%, at least 40%, at least 50%, at least 60%, or at least 70% of the cells have undergone gene editing by a system of the disclosure.

In the systems, particles, cells and cell populations of the disclosure comprising a Cas12a protein, the Cas12a protein should be a Cas12a protein capable of recognizing a PAM adjacent to the target domain to which the targeting sequence of the Cas12a gRNA corresponds. For example, when the PAM sequence adjacent to the target domain is TTTV, the Cas12a protein can be, for example, a wild-type AsCas12a or a wild-type LbCas12a. As another example, when the PAM sequence is TYCV, CCCC, or ACCC, the Cas12a protein can be AsCas12a RR. As yet another example, when the PAM sequence is TATV or RATR, the Cas12a protein can be AsCas12a RVR.

### 6.7. Methods of Altering a Cell

The disclosure further provides methods of altering a cell comprising contacting the cell with a system or particle of the disclosure.

The cell can be contacted with a system or particle of the disclosure or encoding nucleic acid(s) in vitro, ex vivo, or in vivo.

Contacting a cell with a system or particle of the disclosure can result in editing of the genomic DNA of the cell so that the activity of a splice site encoded by the genomic DNA is reduced. Reducing the activity of a splice site can reduce aberrant splicing and restore normal splicing in the cell, for example, when the splice site is a cryptic splice site, or promote exon skipping, for example, when the splice site is a canonical splice site.

The term “contacting,” as used herein, refers to either contacting the cell directly with an assembled system or particle of the disclosure, by introducing into the cell one or more components of a system of the disclosure (or encoding nucleic acid that is expressed in the cell so that the system is assembled in situ), for example by introducing one or more encoding plasmids into the cell or contacting the cell with one or more viral particles capable of being taken up by the cell, or a combination thereof. When the components of the system are introduced as nucleic acids, preferably included are control elements that allow the nucleic acids to be expressed and assembled into a system of the disclosure in the cell.

Accordingly, contacting a cell with a system of the disclosure can comprise, for example, introducing the system to the cell by a physical delivery method, a vector delivery method (e.g., plasmid or virus), or a non-viral delivery method. Exemplary physical delivery methods include microinjection (e.g., by injecting a plasmid encoding a Cas12a gRNA and a Cas12a protein into the cell, injecting the Cas12a gRNA and mRNA encoding the Cas12a protein into the cell, or injecting a RNP comprising the Cas12a gRNA and Cas12a protein into the cell), electroporation (e.g., to introduce a plasmid encoding a Cas12a gRNA and a Cas12a protein into the cell or to introduce mRNA encoding a Cas12a protein and a Cas12a gRNA into the cell), and hydrodynamic delivery (e.g., using high pressure injection to introduce a plasmid encoding a Cas12a gRNA and a Cas12a protein into the cell or RNP comprising the Cas12a gRNA and Cas12a protein into the cell). Exemplary viral delivery methods include contacting the cell with a virus encoding the Cas12a gRNA and a Cas12a protein (e.g., an adeno-associated virus, an adenovirus, or a lentivirus). Exemplary non-viral delivery methods comprise contacting the cell with a particle containing the system, e.g., a particle as described in Section 6.6. Various methods for delivering a Cas12a gRNA and Cas12a protein to a cell or tissue of interest are described in U.S. Pat. No. 9,790,490, the contents of which are incorporated herein by reference in their entirety. See also, Lino et al., 2018, Drug Delivery, 25(1):1234-1257, which reviews several in vitro, ex vivo, and in vivo techniques for delivering CRISPR/Cas9 systems to cells in vitro, ex vivo, and in vivo. Such techniques can be adapted for delivering the Cas12a gRNAs and Cas12a proteins of the disclosure (e.g., by substituting a Cas12a system of the disclosure for the Cas9 gRNA and Cas9 protein).

Cells can come from a subject having a genetic disease (e.g., a stem cell) or derived from a subject having a genetic disease (e.g., an induced pluripotent stem (iPS) cell derived from a cell of the subject).

For example, the cell can be a human cell having a mutation in the CFTR gene, e.g., a 3272-26A>G mutation, a 3849+10kbC>T mutation, a IVS11+194A>G mutation, or a IVS19+11505C>G mutation. Exemplary gRNAs for incorporation into a system useful for correcting the foregoing mutations are described in Section 6.3.4.

As another example, the cell can be a human cell having a mutation in a DMD gene, e.g., a IVS9+46806C>T mutation, a IVS62+62296A>G mutation, a IVS1+36947G>A mutation, a IVS2+5591T>A mutation, or a IVS8-15A>G mutation, or a mutation in exon 50. Exemplary gRNAs for incorporation into a system useful for correcting the foregoing mutations are described in Section 6.3.4.

As another example, the cell can be a human cell having a mutation in a HBB gene, e.g., a IVS2+645C>T mutation, a IVS2+705T>G mutation, or a IVS2+745C>G mutation. Exemplary gRNAs for incorporation into a system useful for correcting the foregoing mutations are described in Section 6.3.4.

As another example, the cell can be a human cell having a mutation in a FGB gene, e.g., a IVS6+13C>T mutation, a IVS4+792C>G mutation, or a IVS3+2552A>G mutation. Exemplary gRNAs for incorporation into a system useful for correcting the foregoing mutations are described in Section 6.3.4.

As another example, the cell can be a human cell having a mutation in a GLA gene, e.g., a IVS4+919G>A mutation. Exemplary gRNAs for incorporation into a system useful for correcting the foregoing mutations are described in Section 6.3.4.

As another example, the cell can be a human cell having a mutation in a LDLR gene, e.g., a IVS12+11C>G mutation. Exemplary gRNAs for incorporation into a system useful for correcting the foregoing mutations are described in Section 6.3.4.

As another example, the cell can be a human cell having a mutation in a BRIP1 gene, e.g., a IVS11+2767A>T mutation. Exemplary gRNAs for incorporation into a system useful for correcting the foregoing mutations are described in Section 6.3.4.

As another example, the cell can be a human cell having a mutation in a F9 gene, e.g., a IVS5+13A>G mutation. Exemplary gRNAs for incorporation into a system useful for correcting the foregoing mutations are described in Section 6.3.4.

As another example, the cell can be a human cell having a mutation in a CEP290 gene, e.g., a IVS26+1655A>G mutation. Exemplary gRNAs for incorporation into a system useful for correcting the foregoing mutations are described in Section 6.3.4.

As another example, the cell can be a human cell having a mutation in a COL2A1 gene, e.g., a IVS23+135G>A mutation. Exemplary gRNAs for incorporation into a system useful for correcting the foregoing mutations are described in Section 6.3.4.

As another example, the cell can be a human cell having a mutation in a USH2A gene, e.g., a IVS40-8C>G mutation, a IVS66+39C>T mutation, or a c.7595-2144A>G mutation. Exemplary gRNAs for incorporation into a system useful for correcting the foregoing mutations are described in Section 6.3.4.

As another example, the cell can be a human cell having a mutation in a GAA gene, e.g., a IVS1-13T>G mutation or a IVS6-22T>G mutation. Exemplary gRNAs for incorporation into a system useful for correcting the foregoing mutations are described in Section 6.3.4.

Contacting of a cell with a system or particle of the disclosure can be performed in vitro, ex vivo or can be performed in vivo (e.g., to treat a subject having a genetic disease in need of treatment for such disease). When performed in vitro or ex vivo, the methods of the disclosure can further comprise a step of introducing the contacted cell to a subject, for example to treat a subject in need of treatment for a genetic disease.

A system can be delivered via any suitable delivery vehicle. Examples of delivery vehicles include viruses (lentivirus, adenovirus) and particles (nanospheres, liposomes, quantum dots, nanoparticles, microparticles, nanocapsules, vesicles, polyethylene glycol particles, hydrogels, and micelles).

Exemplary viral delivery vehicles can include adeno associated virus (AAV), lentivirus, retrovirus, adenovirus, herpes simplex virus I or II, parvovirus, reticuloendotheliosis virus, and or other viral vector types, for example, using formulations and doses from, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. The viruses can infect and transduce the cell in vivo, in vitro, or ex vivo.

Viral delivery vehicles can also be used in ex vivo and in vitro delivery methods, and the transduced cells can be administered to a subject in need of therapy. For ex vivo and in vitro applications, the transduced cells can be stem cells obtained or generated from (e.g., induced pluripotent stem cells generated from fibroblasts of) the subject in need of therapy.

The delivery vehicles can alternatively be particles. Particle delivery systems within the scope of the present disclosure may be provided in any form, including but not limited to solid, semi-solid, emulsion, or colloidal particles. It will be appreciated that reference made herein to particles or nanoparticles can be interchangeable, where appropriate. Cas12a protein mRNA and Cas12a gRNA may be delivered simultaneously using particles or lipid envelopes; for instance, a Cas12a gRNA and a Cas12a protein, e.g., as a complex, can be delivered via a particle as in Dahlman et al., WO2015089419 A2 and documents cited therein.

Delivery of a Cas12a gRNA and a Cas12a protein can be performed with liposomes. Liposomes are spherical vesicle structures composed of a uni- or multilamellar lipid bilayer surrounding internal aqueous compartments and a relatively impermeable outer lipophilic phospholipid bilayer. Liposomes have gained considerable attention as drug delivery carriers because they are biocompatible, nontoxic, can deliver both hydrophilic and lipophilic drug molecules, protect their cargo from degradation by plasma enzymes, and transport their load across biological membranes and the blood brain barrier (BBB). Liposomes can be made from several different types of lipids; however, phospholipids are most commonly used to generate liposomes as drug carriers. Although liposome formation is spontaneous when a lipid film is mixed with an aqueous solution, it can also be expedited by applying force in the form of shaking by using a homogenizer, sonicator, or an extrusion apparatus (see, e.g., Spuch and Navarro, 2011, Journal of Drug Delivery, vol. 2011, Article ID 469679, doi:10.1155/2011/469679 for review).

For administration to a subject, the systems, delivery vehicles and transduced cells can be administered by intravenously, parenterally, intraperitoneally, subcutaneously, intramuscular injection, transdermally, intranasally, mucosally, by direct injection, stereotaxic injection, by minipump infusion systems, by convection, catheters, or other delivery methods to a cell, tissue, or organ of a subject in need. Such delivery may be either via a single dose, or multiple doses.

Such a dosage may further contain, for example, a carrier (water, saline, ethanol, glycerol, lactose, sucrose, calcium phosphate, gelatin, dextran, agar, pectin, peanut oil, sesame oil, etc.), a diluent, a pharmaceutically-acceptable carrier (e.g., phosphate-buffered saline), a pharmaceutically-acceptable excipient, and/or other compounds known in the art. A thorough discussion of pharmaceutically acceptable excipients is available in REMINGTON'S PHARMACEUTICAL SCIENCES (Mack Pub. Co., N.J. 1991) which is incorporated by reference herein.

Frequency of administration is within the ambit of the medical or veterinary practitioner (e.g., physician, veterinarian), depending on usual factors including the age, sex, general health, other conditions of the patient or subject and the particular disease, condition or symptoms being addressed.

Specific cell types and delivery methods for use in the methods of the disclosure can be selected, for example, based upon the specific gene to be edited. For example, DMD is a genetic disorder characterized by progressive muscle degeneration and weakness and caused by splicing defects that inactivate the dystrophin protein. Recombinant AAV whose genome is engineered to encode gRNAs of the disclosure suitable for correcting splicing defects in the dystrophin gene (such as the gRNAs whose sequences are exemplified in Example 7) under the control of the muscle creatine kinase and desmin promoters, which can achieve high levels of expression in skeletal muscle (see, e.g., Naso et al., 2017, BioDrugs. 31(4): 317-334), can be delivered intramuscularly to subjects suffering from DMD. Below are illustrative embodiments for using the gRNA molecules of the disclosure to treat subjects suffering from cystic fibrosis.

### 6.7.1. Exemplary Methods of Treating Subjects Having Cystic Fibrosis

Cystic fibrosis affects epithelial cells, and in some embodiments, the cell being contacted in the method can be an epithelial cell from a subject having a CFTR mutation, e.g., a pulmonary epithelial cell, e.g., a bronchial epithelial cell or an alveolar epithelial cell. The contacting can be performed ex vivo and the contacted cell can be returned to the subject's body after the contacting step. In other embodiments, the contacting step can be performed in vivo.

Cells from a subject having cystic fibrosis can be harvested from, for example, the epidermis, pulmonary tree, hepatobiliary tree, gastrointestinal tract, reproductive tract, or other organ. In an embodiment, the cell is reprogrammed to an induced pluripotent stem (iPS) cell. In an embodiment, the iPS cell is differentiated into airway epithelium, pulmonary epithelium, submucosal glands, submucosal ducts, biliary epithelium, gastrointestinal epithelium, pancreatic duct cells, reproductive epithelium, epidydimal cells, and/or cells of the hepatobiliary tree, e.g., clara cells, e.g., ciliated cells, e.g., goblet cells, e.g., basal cells, e.g., acinus cells, e.g., bronchioalveolar stem cell e.g., lung epithelial cells, e.g., nasal epithelial cells, e.g., tracheal epithelial cells, e.g., bronchial epithelial cells, e.g., enteroendocrine cells, e.g., Brunner's gland cells, e.g., epididymal epithelium. In an embodiment, the CFTR gene in the cell is corrected with a method described herein. In an embodiment, the cell is re-introduced into an appropriate location in the subject, e.g., airway, pulmonary tree, bile duct system, gastrointestinal tract, pancreas, hepatobiliary tree, and/or reproductive tract.

In some embodiments, an autologous stem cell can be treated ex vivo, differentiated into airway epithelium, pulmonary epithelium, submucosal glands, submucosal ducts, biliary epithelium, gastrointestinal epithelium, pancreatic duct cells, reproductive epithelium, epidydimal cells, and/or cells of the hepatobiliary tree, e.g., clara cells, e.g., ciliated cells, e.g., goblet cells, e.g., basal cells, e.g., acinus cells, e.g., bronchioalveolar stem cell e.g., lung epithelial cells, e.g., nasal epithelial cells, e.g., tracheal epithelial cells, e.g., bronchial epithelial cells, e.g., enteroendocrine cells, e.g., Brunner's gland cells, e.g., epididymal epithelium, and transplanted into the subject. In other embodiments, a heterologous stem cell can be treated ex vivo and differentiated into airway epithelium, pulmonary epithelium, submucosal glands, submucosal ducts, biliary epithelium, gastrointestinal epithelium, pancreatic duct cells, reproductive epithelium, epidydimal cells, and/or cells of the hepatobiliary tree, e.g., clara cells, e.g., ciliated cells, e.g., goblet cells, e.g., basal cells, e.g., acinus cells, e.g., bronchioalveolar stem cell e.g., lung epithelial cells, e.g., nasal epithelial cells, e.g., tracheal epithelial cells, e.g., bronchial epithelial cells, e.g., enteroendocrine cells, e.g., Brunner's gland cells, e.g., epididymal epithelium, and transplanted into the subject.

In some embodiments, the method described herein comprises delivery of the Cas12a gRNA and a Cas12a protein (or one or more nucleic acid(s) encoding the Cas12a gRNA and Cas12a protein) to a subject having cystic fibrosis, by inhalation, e.g., via a nebulizer. In other embodiments, the method described herein comprises delivery of a Cas12a gRNA and a Cas12a protein (or one or more nucleic acid(s) encoding the Cas12a gRNA and Cas12a protein) by intravenous administration. In some embodiments, the method described herein comprises delivery of a Cas12a gRNA and a Cas12a protein (or one or more nucleic acid(s) encoding the Cas12a gRNA and Cas12a protein) by intraparenchymal injection into lung tissue. In other embodiments, the method described herein comprises delivery of a Cas12a gRNA and a Cas12a protein (or one or more nucleic acid(s) encoding the Cas12a gRNA and Cas12a protein) by intraparenchymal, intralveolar, intrabronchial, intratracheal injection into the trachea, bronchial tree and/or alveoli. In some embodiments, the method described herein comprises delivery of a Cas12a gRNA and a Cas12a protein (or one or more nucleic acid(s) encoding the Cas12a gRNA and Cas12a protein) by intravenous, intraparenchymal or other directed injection or administration to any of the following locations: the portal circulation, liver parenchyma, pancreas, pancreatic duct, bile duct, jejunum, ileum, duodenum, stomach, upper intestine, lower intestine, gastrointestinal tract, epididymis, or reproductive tract.

In some embodiments, a Cas12a gRNA and a Cas12a protein (or one or more nucleic acid(s) encoding the Cas12a gRNA and Cas12a protein) are delivered, e.g., to a subject having cystic fibrosis, by an AAV, e.g., via a nebulizer, or via nasal spray or inhaled, with or without accelerants to aid in absorption. In some embodiments, a Cas12a gRNA and a Cas12a protein (or one or more nucleic acid(s) encoding the Cas12a gRNA and Cas12a protein) are delivered, e.g., to a subject, by Sendai virus, adenovirus, lentivirus or other modified or unmodified viral delivery particle.

In some embodiments, a Cas12a gRNA and a Cas12a protein (or one or more nucleic acid(s) encoding the Cas12a gRNA and Cas12a protein) are delivered, e.g., to a subject, via a nebulizer or jet nebulizer, nasal spray, or inhalation. In some embodiments, a Cas12a gRNA and a Cas12a protein (or one or more nucleic acid(s) encoding the Cas12a gRNA and Cas12a protein), are formulated in an aerosolized cationic liposome, lipid nanoparticle, lipoplex, non-lipid polymer complex or dry powder, e.g., for delivery via nebulizer, with or without accelerants to aid in absorption.

In some embodiments, a Cas12a gRNA and a Cas12a protein (or one or more nucleic acid(s) encoding the Cas12a gRNA and Cas12a protein) are delivered, e.g., to a subject having cystic fibrosis, via liposome GL67A. GL67A is described, e.g., at www.cfgenetherapy.org.uk/clinical/article/GL67A_pGM169_Our_first_clinical_trial_product; Eastman et al., 1997, Hum Gene Ther. 8(6):765-73.

### 6.8. Examples

### 6.8.1. Example 1: CRISPR-Cas12a Correction of CFTR 3272-26A>G Splicing Mutation in Cells

The CFTR 3242-26A>G mutation is a point mutation that creates a new acceptor splice site causing the abnormal inclusion of 25 nucleotides within exon 20 of the CTFR gene. The resulting mRNA contains a frameshift in CFTR, producing a premature termination codon and consequent expression of a truncated, non-functional CFTR protein. A genome editing strategy using AsCas12a in combination with various Cas12a gRNAs to correct the splicing mutation was examined.

6.8.1.1. Materials and Methods

6.8.1.1.1. Oligonucleotides: Guide RNAs

AsCas12a gRNAs targeting a CFTR gene having a 3272-26A>G splicing mutation were designed with protospacer domains corresponding, with no mismatches, to the target domains set forth in Table 2. Each gRNA was designed to have a loop domain consisting of the sequence UAAUUUCUACUCUUGUAGAU (SEQ ID NO: 25). The gRNAs are referred to in this Example according to their protospacer domains, e.g., crRNA+11.

TABLE 2

| | | SEQ ID | Target domain and surrounding | SEQ |
|---|---|---|---|---|
| Name* | Target domain\hat{ } | NO: | genomic sequence^{#} | ID NO: |
| −77 | TGATATGATTATTCTAA | 104 | acaTTTGTGATATGATTATTCTAAT | 110 |
| | TTTAGT | | TTAGTctt | |
| −56 | GTCTTTTTCAGGTACA | 105 | taaTTTAGTCTTTTTCAGGTACAA | 111 |
| | AGATATT | | GATATTatg | |
| −27 | ATAATATCTTGTACCT | 106 | taaTTTCATAATATCTTGTACCTGA | 112 |
| | GAAAAAG | | AAAAGact | |
| −11 | TGTTATTTGCAGTGTT | 107 | gtgTTTATGTTATTTGCAGTGTTTT | 113 |
| | TTCTATG | | CTATGgaa | |
| −2 | CAGTGTTTTCTATGGA | 108 | ttaTTTGCAGTGTTTTCTATGGAAA | 114 |
| | AATATTT | | TATTTcac | |
| +11 | CATAGAAAACACTGCA | 38 | ataTTTCCATAGAAAACACTGCAA | 115 |
| | AATAACA | | ATAACAtaa | |
| +11/ | CATAGAAAACATTGCA | 109 | ataTTTCCATAGAAAACATTGCAA | 116 |
| wt | AATAACA | | ATAACAtaa | |

*value indicates the distance of the PAM sequence from the mutation;
+ or − indicates the position of the target domain before or after the mutation, respectively;
\hat{ }3272-26A>G mutation is highlighted in bolded font;
^{#}PAM sequence is underlined;
lower case font indicates nucleotides around the target domain

6.8.1.1.2. Other Oligonucleotides

Oligonucleotides for PCR, RT-PCR, cloning, site-directed mutagenesis, and sequencing were designed and prepared. These oligonucleotides are listed in Table 3.

TABLE 3
PCR and site-directed mutagenesis primers for CFTR 3272-26A>G

SEQ
ID NO:

Minigene cloning oligonucleotides*

| | | | |
|---|---|---|---|
| Primer | Kpnl-Agel exon | ATggtaccggtgaccttctgcctcTTACCATATTTGACT | 117 |
| 1f | 18-19 hCFTR for | TCATCCAGTTG | |
| Primer | TM exon 18 exon | ttaccatatttgacttcatccagTTGTTATTAATTGTGAT | 118 |
| 2f | 19 hCFTR for | TGGAGCTATAG | |
| Primer | exon 20 hCFTR | TGtAgaattcttaggatccctcgcCTGTTGTTAAAATG | 119 |
| 3r | rev | GAAATGAAGGTAACAG | |

Site directed mutagenesis oligonucleotides

| | | | |
|---|---|---|---|
| Primer | MUT 3272-26 A>G | ATGGTCTCAgTGTTTTCTATGGAAATATTTCA | 120 |
| 4mf | for | C | |
| Primer | MUT 3272-26 A>G | ATGGTCTCaacAcTGCAAATAACATAAACACA | 121 |
| 5mr | rev | AAATG | |

RT-PCR and PCR oligonucleotides

| | | | |
|---|---|---|---|
| Primer | oligo BGH rev | TAGAAGGCACAGTCGAGG | 122 |
| 6r | | | |
| Primer | TM exon 18 exon | ttaccatatttgacttcatccagTTGTTATTAATTGTGAT | 118 |
| 2f | 19 hCFTR for | TGGAGCTATAG | |
| Primer | exon 20 hCFTR | TGtAgaattcttaggatccctcgcCTGTTGTTAAAATG | 119 |
| 3r | rev | GAAATGAAGGTAACAG | |

Deep sequencing oligonucleotides

| | | | |
|---|---|---|---|
| Primer | DS 3272-26 A>G | tcgtcggcagcgtcagatgtgtataagagacagGCTTGTAA | 123 |
| 7f | for | CAAGATGAGTGAAAATTGGA | |
| Primer | DS 3272-26 A>G | gtctcgtgggctcggagatgtgtataagagacagATATCTAT | 124 |
| 8r | rev | TCAAAGAATGGCACCAGTGT | |

*for = forward; rev = reverse; exon sequences are represented by upper case letters; intron sequences are represented by lower case letters

6.8.1.1.3. Preparation of WT and Minigene Plasmids for CFTR 3272-26A>G Mutation

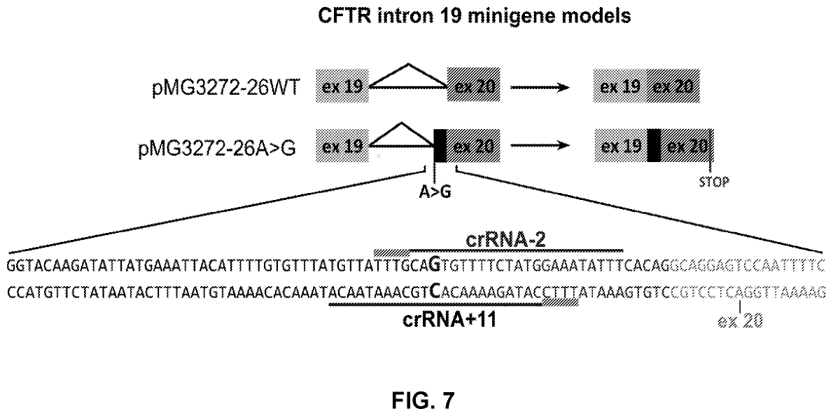

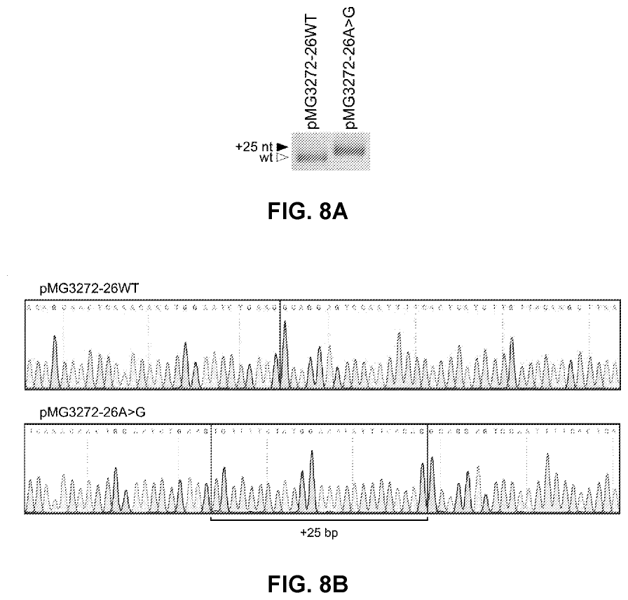

Minigene plasmid models were generated to mimic the splicing pattern of the CFTR gene corresponding to the region encompassing exons 19, 20 and intron 19. Plasmid pMG3272-26WT contained the wild-type allele; plasmid pMG3272-26A>G contained the mutated allele (see FIG. 7).

A wild-type minigene representing the CFTR 3272-26 locus was cloned into plasmid pcDNA3 (Invitrogen®). Primers 1f, 2f, and 3r were used to PCR amplify CFTR DNA of the wild-type sequence of exons 19, 20 and intron 19 from the genome of HEK293T cells. The amplified DNA was cloned into plasmid pcDNA3 (Invitrogen®) to generate plasmid pMG3272-26WT containing the wild-type allele of exons 19, 20 and intron 19. Primers 4mf and 5mr were used to carry out site-directed mutagenesis of the wild-type minigene housed in pMG3272-26WT to generate the 3272-26A>G mutation, creating plasmid pMG3272-26A>G.

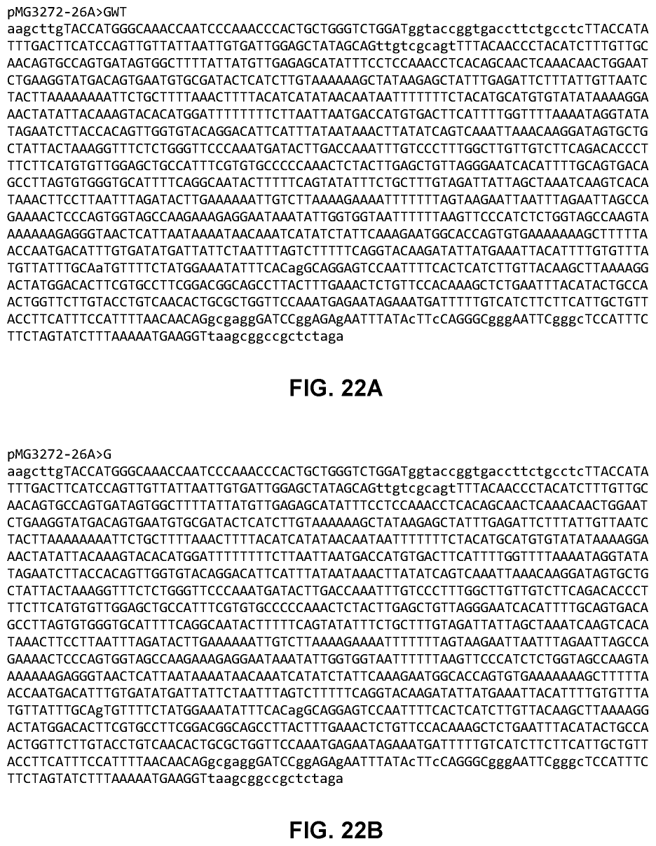

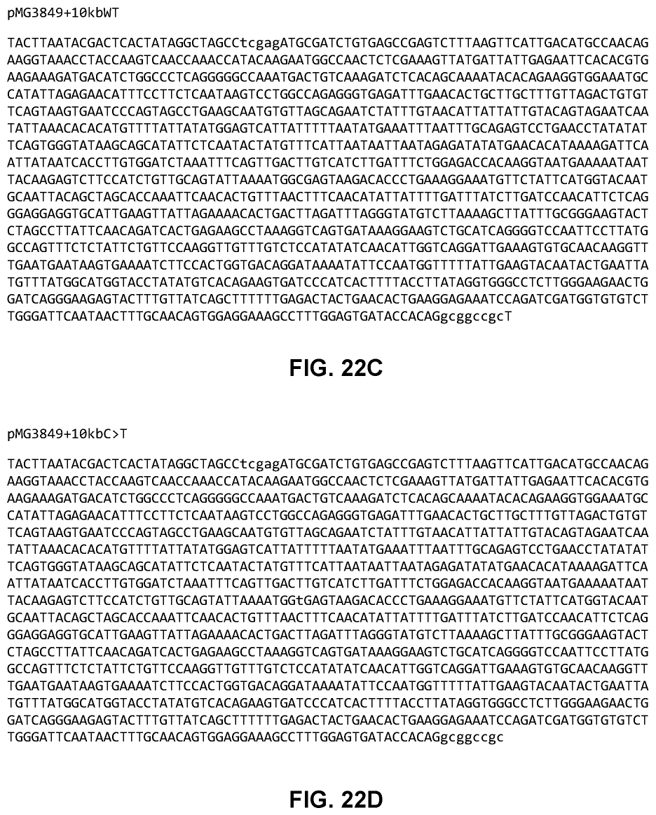

Sequences coding for guide RNAs were cloned into a commercially available plasmid, pY108 lentiAsCas12a (Addgene Plasmid 84739), using BsmBI restriction sites as previously described (Shalem, O., et al., 2014, Science, 343:84-87). The lenti virus-based plasmids allow for simultaneous delivery of the RNA-guided Cas12a protein and the gRNA to target cells in a single viral particle (see FIG. 22A and FIG. 22B).

6.8.1.1.4. Cell Lines

Human colorectal adenocarcinoma cells (Caco-2), human embryonic kidney cells HEK293T, and HEK293 cells were obtained from the American Type Culture Collection.

6.8.1.1.5. Transfection

Caco-2, HEK293T, and HEK293 cells stably expressing pMG3272-26WT (cell line HEK293/pMG3272-26WT) or 3272-26A>G (cell line HEK293/pMG3272-26A>G) were prepared. Cells were cultured in Dulbecco's modified Eagle's medium (DMEM; Life Technologies) supplemented with 10% fetal bovine serum (FBS; Life Technologies), 10 U/ml antibiotics (PenStrep, Life Technologies), and 2 mM L-glutamine at 37° C. in a 5% CO₂ humidified atmosphere.

Cells were seeded at 1.5×10⁵ cells/well in 24 well plates and transfected with 100 ng of Bgl-II linearized minigene plasmids pMG3272-26WT or pMG3272-26A>G complexed with polyethylenimine (PEI) and with 700 ng of plasmid pY108 lentiAsCas12a encoding both the AsCas12a protein and gRNA sequences. After 16 hours incubation, the cell medium was changed. Selection was carried out by exposing the transfected Caco-2 cells to 10 μg/ml puromycin; transfected HEK293T or HEK293 cells were selected by exposure to 2 μg/ml puromycin. Plasmid integration was selected for by the addition of 500 μg/ml of G418 added approximately 48 h after transfection. Single cell clones were isolated and characterized for the expression of the minigene constructs. Transfected cells were collected three days post-transfection.

6.8.1.1.6. Lentiviral Vector Production

Lentiviral particles were produced in HEK293T cells at 80% confluency in 10 cm plates. Ten μg of transfer vector pY108 lentiAsCas12a plasmid, 3.5 μg of VSV-G, and 6.5 μg of Δ8.91 packaging plasmid were transfected into the cells using PEI. After an overnight incubation, the medium was replaced with complete DMEM. The supernatant containing the viral particles was collected after 48 hours and filtered through a 0.45 μm PES filter. The lentiviral particles were concentrated and purified by ultracentrifugation for 2 hours at 4° C. and 150000×g with a 20% sucrose cushion. Pellets of lentivirus particles were resuspended in OptiMEM and aliquots stored at −80° C. Vector titres were measured as Reverse Transcriptase Units (RTU) using the SG-PERT method (see Casini, A., et al., 2015, J. Virol. 89:2966-2971).

6.8.1.1.7. Transduction

For transduction studies, HEK293/pMG3272-26WT, HEK293/pMG3272-26A>G and Caco-2 cells were seeded at a density of 3×10⁵ cells/well in 12 well plates. Following an overnight incubation, the cells were transduced with 3 RTU of lentiviral vectors. Forty-eight hours later, the cells were selected with puromycin (2 μg/ml for HEK293 or 10 μg/ml for Caco-2 cells) and collected 10 days from transduction.

6.8.1.1.8. Transcript Analysis

The splicing pattern produced by the mutated or wild-type minigenes in transfected HEK293T cells, either altered or correct respectively, was evaluated by RT-PCR and sequencing analyses (see Beck, S., et al., 1999, Hum. Mutat., 14:133-144). RNA was extracted from the collected cells using TRIzol™ Reagent (Invitrogen®) and resuspended in DEPC-ddH2O. cDNA was obtained from 500 ng of RNA using RevertAid Reverse Transcriptase (Thermo Scientific) according to the manufacturer's protocol. Target regions were amplified by PCR with Phusion High Fidelity DNA Polymerase (Thermo Fisher).

6.8.1.1.9. Detection of Nuclease Induced Genomic Mutations

Genomic DNA was extracted using QuickExtract DNA extraction solution (Epicentre) and the target locus amplified by PCR using Phusion High Fidelity DNA Polymerase (Thermo Fisher). In order to evaluate any indels resulting from the cleavage of a single gRNA, the purified PCR products were sequenced and analyzed using TIDE (see Table 3 primers 7f and 8r; Brinkman, E. K., et al., 2014, Nucleic Acids Res., 42: 1-8) or SYNTHEGO ICE software (see Hsiau, T., et al., 2018, bioRxiv, January 20, 1-14). In some studies, DNA editing was also measured using a T7 Endonuclease 1 (T7E1) assay (New England BioLabs) following manufacturer's instructions and as previously described (see Petris, G., et al., 2017, Nat. Commun. 8:1-9).

6.8.1.1.10. GUIDE-seq

Approximately 2×10⁵ HEK293T cells were transfected using Lipofectamine 3000 transfection reagent (Invitrogen) with 1 μg lenti Cas12a plasmid pY108 and 10 pmol of dsODNs designed according to the original GUIDE-seq protocol (see Tsai, S. Q., et al., 2015, Nat. Biotechnol., 33:187-198). One day post transfection, the cells were detached and selected with 2 μg/ml puromycin. Four days post transfection, the cells were collected and genomic DNA extracted using DNeasy Blood and Tissue kit (Qiagen) following manufacturer's instructions. The isolated genomic DNA was sonicated and sheared to an average length of 500 bp using a Bioruptor Pico sonication device (Diagenode). Library preparation, sequencing, and analysis was carried out using methods known to those of skill in the art (see, for example, Montagna, C., et al., 2018, Mol. Ther. Nucleic Acids, 12:453-462; Casini, A., et al., 2018, Nat. Biotechnol., 36:265-271).

6.8.1.1.11. Targeted Deep Sequencing

The locus of interest (3272-26A>G/4218insT) was amplified from genomic DNA extracted from the transfected cells 14 days after transduction with lentiAsCas12a-crRNA+11 or a control (CTR) using Phusion high-fidelity polymerase (Thermo Scientific) and primers 7f and 8r. Amplicons were indexed by PCR using Nextera indexes (Illumina), quantified with the Qubit dsDNA High Sensitivity Assay kit (Invitrogen), pooled in near-equimolar concentrations, and sequenced on an Illumina Miseq system using an Illumina Miseq Reagent kit V3-150 cycles (150 bp single read). Raw sequencing data (FASTQ files) were analyzed using CRISPResso online tool (see Pinello, L., et al., 2016, Nat. Biotechnol., 34:695-697; windows size=3, minimum average read quality (phred33 scale)=30, minimum single bp quality (phred33 scale)=10).

6.8.1.2. Results

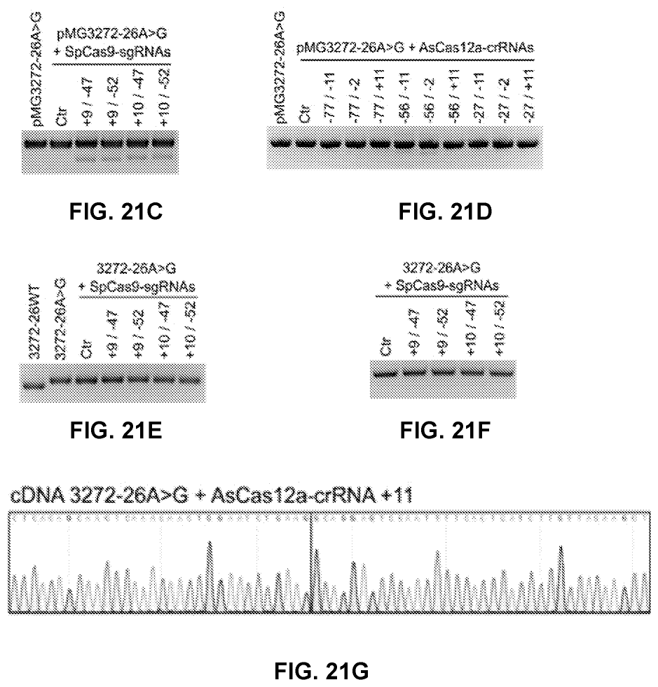

The splicing pattern of pMG3272-26A>G was evaluated after its co-transfection with the designed gRNAs. Increased levels of correctly spliced product resulted after editing by AsCas12a in combination with various gRNAs (FIG. 21B). Analysis of the deletions induced by gRNA pairs showed that no deletions were generated by AsCas12a (FIG. 21D).

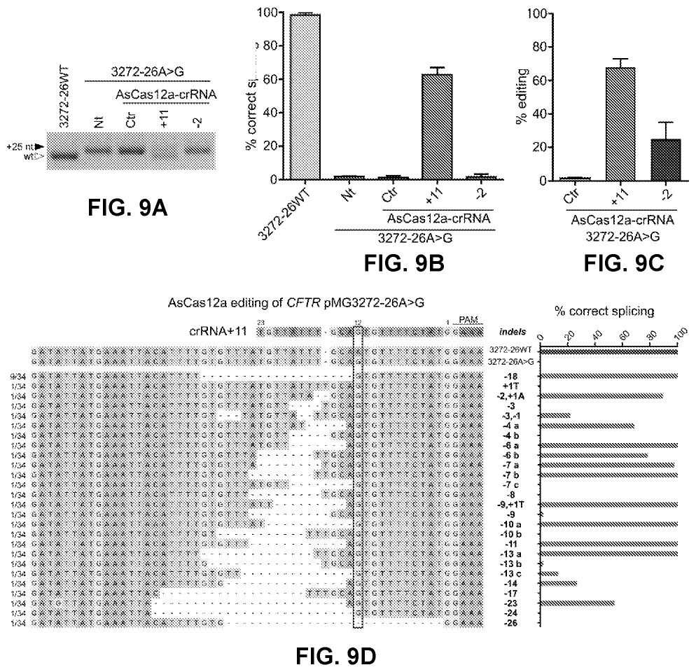

To further validate the activity of AsCas12a with selected gRNAs within a more physiological chromatin context, the splicing correction of CFTR intron 19 in HEK293 cells stably transfected with the pMG3272-26A>G minigene (HEK293/3272-26A>G) was tested. AsCas12a-crRNA+11 resulted in the formation of numerous correct transcripts, >60%, from the pMG3272-26A>G transgene (FIG. 9A-B and FIG. 21G) and efficient DNA editing (approximately 70%; FIG. 9C).

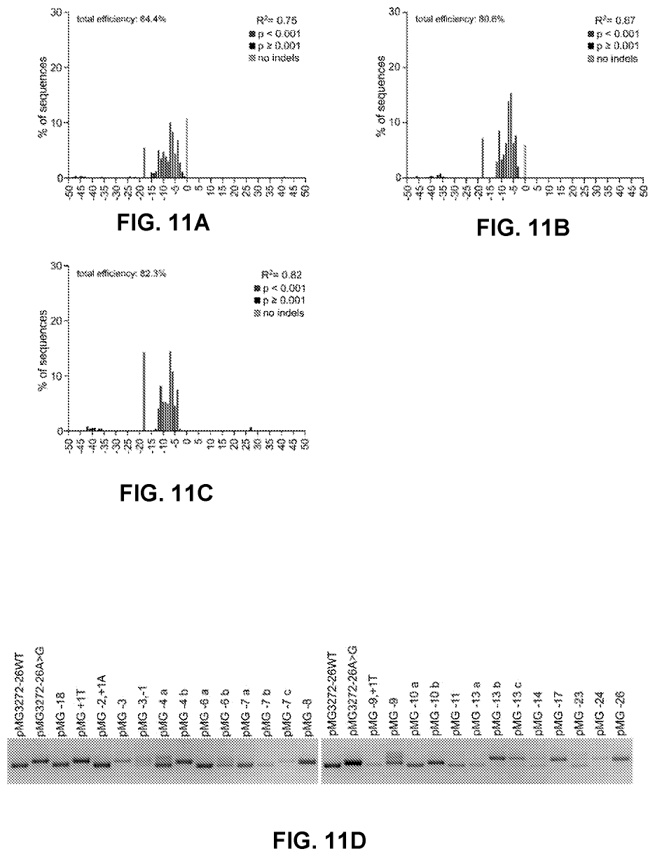

TIDE analysis of the integrated minigenes, following editing with AsCas12a-crRNA+11, revealed a heterogeneous pool of deletions (FIG. 11A-C). Edited variants were cloned into the pMG3272-26A>G minigene to analyze the derived splicing products. Sequence analysis of the edited sites showed a high frequency of an 18-nucleotide deletion which could also be observed in the chromatogram deconvolution (FIG. 11A-C) along with the persistence of the 3272-26A>G mutation (FIG. 9D). Notably, the splicing analysis revealed that the frequent 18 nucleotides deletion (9/34 clones) fully restored the correct splicing (FIG. 9D and FIG. 11D). Most of the remaining edited sites occurred at low frequency (1/34 clones) and generated correct splicing; in a few instances, an additional transcript product was observed (FIG. 11D). In summary, AsCas12a in combination with a single gRNA (having a crRNA+11 protospacer domain) generated small deletions upstream of the 3272-26A>G mutation in a minigene model and resulted in efficient recovery of the CF splicing defect. Nearly 70% of the analyzed editing events contributed to the effective restoration of normal splicing in cells.

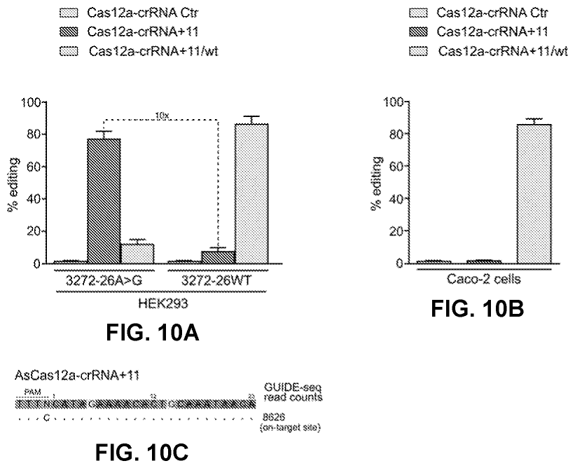

A large majority of CF patients are compound heterozygous for the 3272-26A>G mutation. As such, it was important to evaluate potential off-target effects of AsCas12a-crRNA+11, for example, potential modification within the wild-type allele. The cleavage properties of the AsCas12a-crRNA+11 were analyzed in stable cell lines expressing either pMG3272-26WT or pMG3272-26A>G (HEK293/3272-26WT and HEK293/3272-26A>G cells respectively). As shown in FIG. 10A, the cleavage efficiency of crRNA+11 dropped from nearly 80%, detected in HEK293/3272-26A>G, to less than 7.5% in HEK293/3272-26WT. Thus, the on-target effect of AsCas12a-crRNA+11, that is, the effect upon the 3272-26A>G mutation, exhibited an at least 10-fold differential cleavage compared to the wild-type, or off-target, allele. In reciprocal studies with crRNA+11/wt, targeting the CFTR 3272-26WT sequence, AsCas12a exhibited high cleavage efficiency (approximately 90%) in HEK293/3272-26WT cells and low indels formation (less than 15%) in HEK293/3272-26A>G cells (FIG. 10A). Taken together, these studies demonstrate the high allelic discrimination by AsCas12a with the selected gRNA having a crRNA+11 protospacer domain.

The specificity of the AsCas12a-crRNA+11 delivered by lentiviral vectors towards the wild-type intron was further confirmed in Caco-2 epithelial cells endogenously expressing the wild-type CFTR gene. Long term nuclease expression (10 days after transduction), which has been demonstrated to highly favor non-specific cleavages (Petris, G., et al., 2017, Nat. Commun. 8:1-9), did not generate any unspecific CFTR editing above TIDE background levels (about 1%; see Brinkman, E. K., et al., 2014, Nucleic Acids Res. 42:1-8); whereas AsCas12a-crRNA+11/wt efficiently edited the CFTR gene (more than 80%; FIG. 10B).

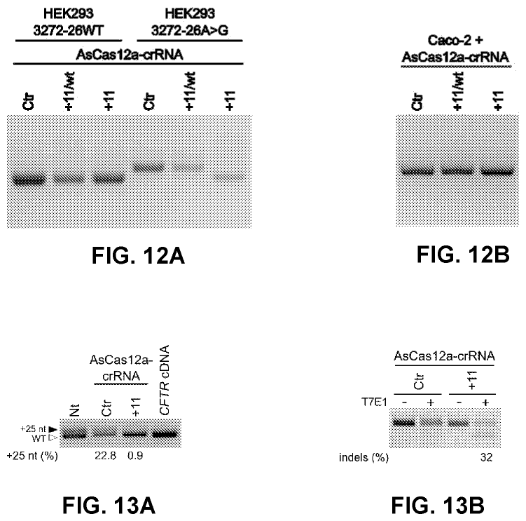

To exclude splicing alterations following potential wild-type intronic cleavages, the splicing pattern was evaluated in HEK293/3272-26WT and Caco-2 cells. No major alterations were observed following AsCas12a treatment in combination with either crRNA+11/wt or crRNA+11 (FIG. 12A-B).

The specificity of the AsCas12a-crRNA+11 editing was also tested in terms of off-target cleavages by a genome-wide survey, GUIDE-seq (Nissim-Rafinia, M. et al., 2000, Hum. Mol. Genet. 9:1771-1778, Kashima, T. et al., 2007, Hum. Mol. Genet. 16, 3149-3159). Off-target profiling of AsCas12a-crRNA+11 genome editing in HEK293/3272-26A>G cells (Tsai, S. Q., et al., 2015, Nat. Biotechnol. 33:187-198; Kleinstiver, B. P., et al., 2016, Nat. Biotechnol. 34:869-874) showed very high specificity, as demonstrated by exclusive editing of the 3272-26A>G CFTR locus, while no non-specific cleavages in the second allele, or any other genomic loci, could be detected (FIG. 10C).

6.8.2. Example 2: CRISPR-Cas12a Correction of 3272-26A>G Splicing Mutation in Organoids

Human organoids represent a near-physiological model for translational research (Fatehullah, A., et al., 2016, Nat. Cell Biol., 18:246-254). Intestinal organoids from CF patients are valuable tools to evaluate CFTR activity and functional recovery (Dekkers, J. F., et al., 2013, Nat. Med., 19:939-945; Dekkers, J. F., et al., 2016, Sci. Transl. Med. 8:344ra84; Sato, T., et al., 2011, Gastroenterology, 141:1762-1772).

The rescue potential of the CF phenotype by AsCas12a-crRNA+11 in human intestinal organoids compound heterozygous for the 3272-26A>G mutation (3272-26A>G/4218insT) was examined.

6.8.2.1. Materials and Methods

6.8.2.1.1. Human Intestinal Organoids Culture and Transduction

Human intestinal organoids of human cystic fibrosis subjects determined to be compound heterozygous for the 3272-26A>G splicing mutation (3272-26A>G/4218insT; n=1, CF-86) were cultured (see Dekkers, J. F., et al., 2013, Nat. Med., 19:939-945).

Cultured organioids were separated into single cells using trypsin 0.25% EDTA (Gibco). Approximately 3 to 4×10⁴ single cells were resuspended with 25 μl of lentiviral vector (0.25-1 RTU) and incubated for 10 min at 37° C. (see Vidovic, D., et al., 2016, Am. J. Respir. Crit. Care Med., 193:288-298). An equal volume of Matrigel (Corning) was added to the cell and vector solution and the mix plated in a 96-well plate. After polymerisation of the Matrigel drops at 37° C. for 7 minutes, the cells were covered with 100 μl of complete organoid medium (Dekkers, J. F., et al., 2013, Nat. Med., 19:939-945) containing 10 μM of Rock inhibitor (Y-27632 2HCI, Sigma Aldrich, Y0503) for three days to ensure optimal outgrowth of single stem cells (see Sato, T., et al., 2011, Gastroenterology, 141:1762-1772). The medium was replaced every 2-3 days until the day of organoid analysis.

6.8.2.1.2. Forskolin Induced Swelling (FIS) Assay and Analysis of CFTR Activity in Intestinal Organoids

Fourteen days after viral vector transduction, the organoids were incubated for 30 minutes with 0.5 μM calcein-green (Invitrogen, C3-100MP) and analysed by live cell confocal microscopy with a 5× objective (LSM800, Zeiss; Zen Blue software, version 2.3). The steady-state area of the organoids was determined by calculating the absolute area (xy plane, μm²) of each organoid using ImageJ software through the Analyse Particle algorithm. Organoid particles with an area less than 1500 μm were considered defective and were excluded from the analysis. Data were averaged for each different run and plotted in a box plot representing means±SD.

The FIS assay was performed by stimulation of the organoids with 5 μM of forskolin. The effect of the forskolin on the organoids was analysed by live cell confocal microscopy at 37° C. for 60 min, with one image taken every 10 min. The area of each organoid (xy plane) at each time point was calculated using ImageJ, as described above. Statistical analyses were performed by ordinary one-way analysis of variance (ANOVA) in GraphPad Prism version 6. Differences in the size of the organoids were considered statistically different at P<0.05.

6.8.2.2. Results

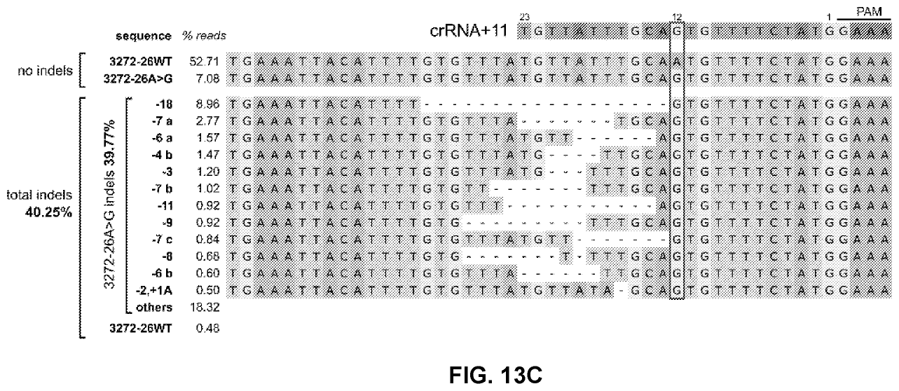

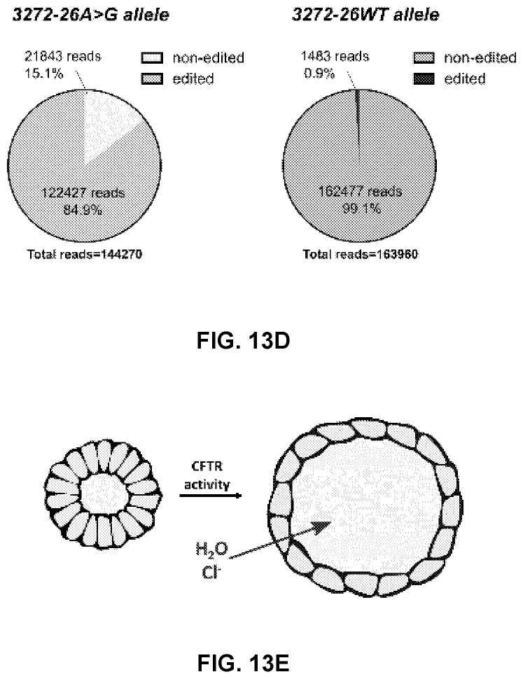

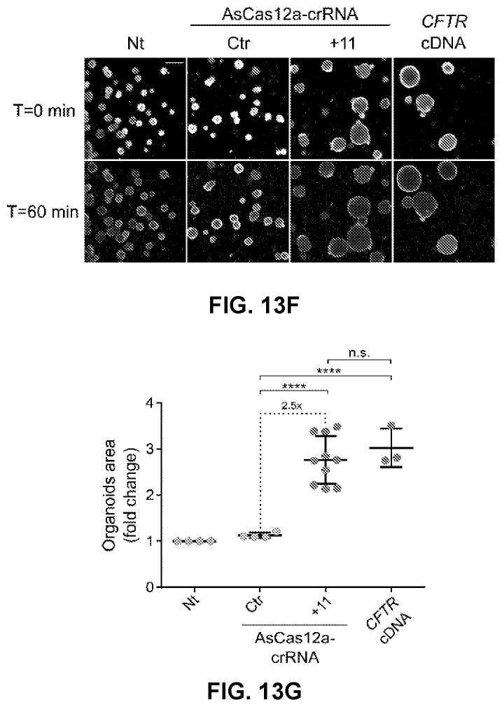

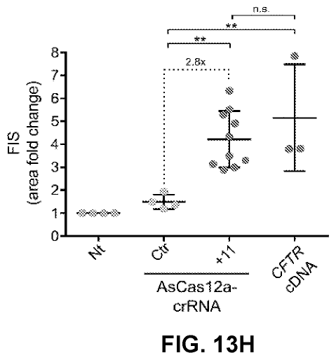

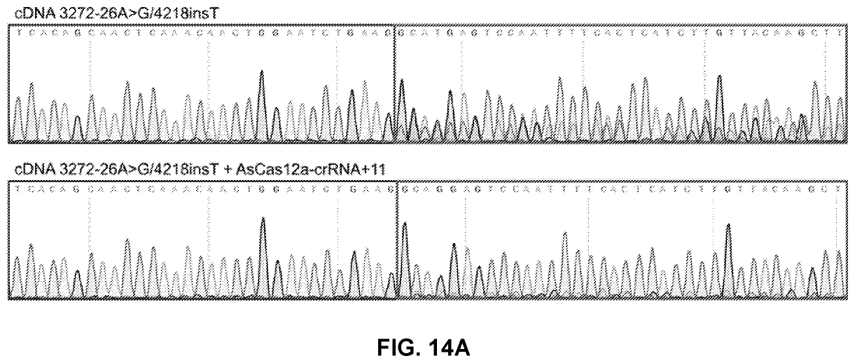

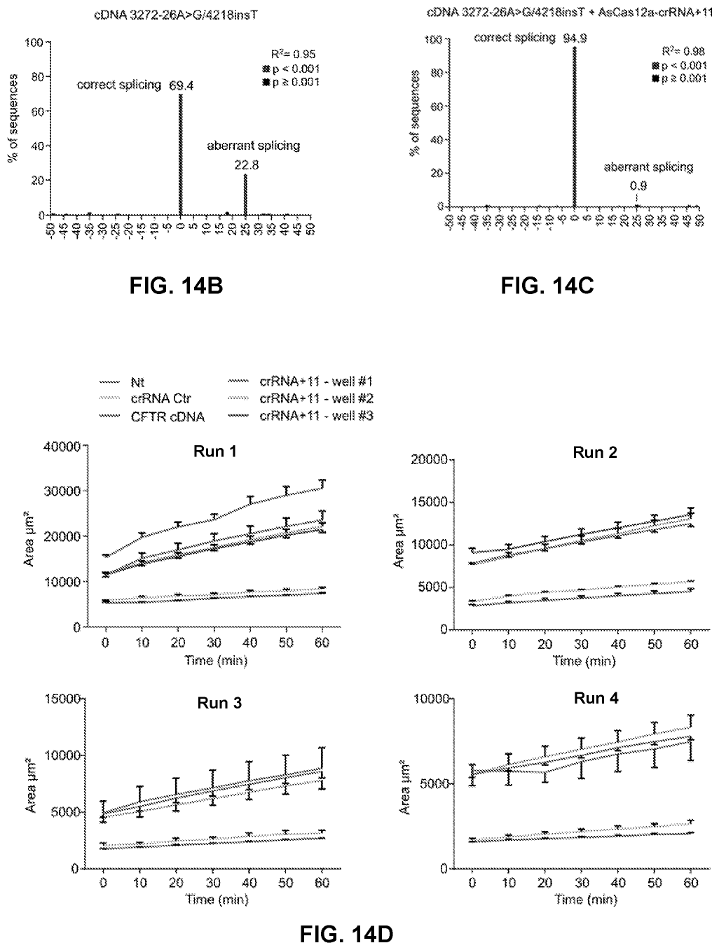

The splicing pattern of CFTR intron 19 in the crRNA control and untreated organoids showed two transcript variants (FIG. 13A); the difference in size and abundance of the variants is consistent with the heterozygosity for the 3272-26A>G mutation in the organoids and previous data (Beck, S., et al., 1999, Hum. Mutat. 14:133-144). Lentiviral delivery of AsCas12a-crRNA+11 showed nearly complete disappearance of the altered splicing product generated by the 3272-26A>G allele (+25nt) indicating efficient correction of the aberrant intron 19 splicing (FIG. 13A and FIG. 14A-B). The number of indels induced by AsCas12a-crRNA+11 evaluated by the T7 Endonuclease I assay showed approximately 30% editing of the CFTR locus (FIG. 13B), consistent with the restored splicing observed (FIG. 13A).

Deep sequencing analysis revealed 40.25% indels in the CFTR locus (39.77% within the 3272-26A>G allele and 0.48% within the other allele, FIG. 13C), thus confirming the high efficiency of AsCas12a-crRNA+11 editing observed with the T7 Endonuclease I assay (FIG. 13B). Further sequence analysis revealed that 84.9% of the sequencing reads including the 3272-26A>G mutation contained variable length deletions, while sequencing reads corresponding to the wild-type allele (3272-26WT) contained only 0.9% indels, thus indicating a 94-fold allelic discrimination (FIG. 13D).

In agreement with previous reports (van Overbeek, M., et al., 2016, Mol. Cell, 63, 63:633-646) and despite the heterogeneity of the editing observed, the repair events in patient's organoids were largely similar to those observed in the pMG3272-26A>G model, with the 18 nucleotide deletion being the most frequent repair observed (compare FIG. 13C with FIG. 9D). Notably, this 18-nucleotide deletion, as well as most of the other reported indels (with a frequency above 0.5% of total DNA repair events; (FIG. 13C)), generated splicing corrections when cloned in the pMG3272-26 model (FIG. 9D).

Lumen formation in intestinal organoids (swelling) depends on the activity of the CFTR anion channel (Dekkers, J. F., et al., 2013, Nat. Med. 19, 939-945; schematized in FIG. 13E) and thus can be used to measure the restoration of CFTR function after AsCas12a-crRNA+11 genome editing. Fourteen days post AsCas12a-crRNA+11 treatment, patient's organoids showed a 2.5-fold increased lumen area compared to the lumen of the control and untreated samples, indicating restored channel function following repair of the CFTR 3272-26A>G allele (FIG. 13F-G). Interestingly, there was no significant difference in organoids size between treatment with AsCas12a-crRNA+11 or transduction of WT CFTR cDNA (FIG. 13G), further demonstrating the remarkable efficiency of the AsCas12a-crRNA+11 system to edit the genotype and reverse the phenotype of the 3272-26A>G mutation.

Another assay used to evaluate CFTR activity is the Forskolin Induced Swelling (FIS) assay (Dekkers, J. F., et al., 2013, Nat. Med., 19, 939-945; FIG. 13F-H). Consistent with the organoid swelling studies (FIG. 13G), the FIS assay revealed an increase in AsCas12a-edited organoid area of 2.8-fold, similar to results obtained with lentiviral delivery of WT CFTR cDNA (FIG. 13H and FIG. 14C).

The AsCas12a-crRNA+11 modifications of the 3272-26A>G defect in CFTR organoids results in the efficient repair of the intron 19 splicing defect, leading to the full recovery of endogenous CFTR protein.

### 6.8.3. Example 3: CRISPR-Cas12a Correction of CFTR 3849+10KbC>T Splicing Mutation in Cells

The CFTR 3849+10kbC>T mutation creates a novel donor splice site inside intron 22 of the CFTR gene, leading to the insertion of the new cryptic exon of 84 nucleotides which results in an in-frame stop codon and consequent production of a truncated non-functional CFTR protein. A genome editing strategy using AsCas12a in combination with various Cas12a gRNAs to correct the splicing mutation was examined.

6.8.3.1. Materials and Methods

6.8.3.1.1. Oligonucleotides: Guide RNAs

An AsCas12a gRNA targeting a CTFR gene having a 3849+10KbC>T splicing mutation was designed with a protospacer domains corresponding, with no mismatches, to the target domain set forth in Table 4. An AsCas12a gRNA targeting the wild-type sequence was also designed. Each gRNA was designed to have a loop domain consisting of the sequence UAAUUUCUACUCUUGUAGAU (SEQ ID NO: 25). The gRNAs are referred to in this Example according to their protospacer domains, e.g., crRNA+14.

TABLE 4

| | | SEQ ID | Target domain and surrounding | SEQ ID |
|---|---|---|---|---|
| Name* | Target domain\hat{ } | NO: | genomic sequence^{#} | NO: |
| +14 | AGGGTGTCTTACTC | 39 | tccTTTCAGGGTGTCTTACTCAC | 125 |
| | ACCATTTTA | | CATTTTAata | |
| +14/ | AGGGTGTCTTACTC | 454 | tccTTTCAGGGTGTCTTACTCGC | 126 |
| wt | GCCATTTTA | | CATTTTAata | |

*value indicates the distance of the PAM from the mutation;
+indicates the position of the target domain before the mutation;
\hat{ }3849+10KbC>T mutation position is highlighted in larger bolded font;
^{#}PAM is underlined;
lower case font indicates nucleotides around the target site

6.8.3.1.2. Other Oligonucleotides

Oligonucleotides for PCR, RT-PCR, cloning, site-directed mutagenesis, and sequencing were designed and prepared. These oligonucleotides are listed in Table 5.

TABLE 5
PCR and site-directed mutagenesis primers for CFTR 3849+10KbC>T

SEQ ID
NO:

Minigene cloning oligonucleotides

| | | | |
|---|---|---|---|
| Primer | Xhol ex 22 | ATATctcgagATGCGATCTGTGAGCCGAGTCTTTAA | 127 |
| 9f | CFTR for1 | | |
| Primer | BsmBI_int22_new | tacgtctcATAtATTCAGTGGGTATAAGCAGCATATTCT | 128 |
| 10f | ex for2 | C | |
| Primer | BamHI- | tatggatccagatcgtctcgAAAGGTCAGTGATAAAGGAAG | 129 |
| 11f | BsmBI_ex 23 | TCTGCAT | |
| | for3 | | |
| Primer | int22 BsmBI- | tatggatccagatcgtctcgATATAGGTTCAGGACTCTGCA | 130 |
| 12r | BamHI rev1 | AATTAAATTTC | |
| Primer | new | atcgtctctCTTtAGGCTTCTCAGTGATCTGTTGAATAA | 131 |
| 13r | ex_BsmBI | G | |
| | rev2 | | |
| Primer | ex 23_Notl | atagtgcggccgcCTGTGGTATCACTCCAAAGGCTTTC | 132 |
| 14r | rev3 | | |

Site-directed mutagenesis oligonucleotides

| Primer | MUT 3849 | CCATCTGTTGCAGTATTAAAATGGtGAGTAAGACA | 133 |
|---|---|---|---|
| 15mf | 10kb C-T for | CCCTGAAAGG | |
| Primer | MUT 3849 | CCTTTCAGGGTGTCTTACTCaCCATTTTAATACTG | 134 |
| 16mr | 10kb C-T rev | CAACAGATGG | |
| RT-PCR oligonucleotide | | | |
| Primer | T7F2 (x pCI) | TACTTAATACGACTCACTATAGGCTAGCCTCG | 135 |
| 17 | | | |
| Primer | Xhol ex 22 | ATATctcgagATGCGATCTGTGAGCCGAGTUTTAA | 127 |
| 9f | CFTR for1 | | |
| Primer | BsmBI_int22_new | tacgtctcATAtATTCAGTGGGTATAAGCAGCATATTCT | 128 |
| 10f | ex for2 | C | |
| Primer | new | atcgtctctCTTtAGGCTTCTCAGTGATCTGTTGAATAA | 131 |
| 13r | ex_BsmBI | G | |
| | rev2 | | |
| Primer | ex 23_Notl | atagtgcggccgcCTGTGGTATCACTCCAAAGGCTTTC | 132 |
| 14r | rev3 | | |

PCR oligonucleotides for TIDE analysis

| | | | |
|---|---|---|---|
| Primer | CFTR 10kb int | CTGCTTTCTCCATTTGTAGTCTCTTG | 136 |
| 18f | 22 for | | |
| Primer | CFTR 10kb int | TGCTGGTAATGCATGATATCTGACAC | 137 |
| 19r | 22 rev | | |

*for = forward; rev =reverse; exon sequences are represented by upper case letters; intron sequences are represented by lower case letters

6.8.3.1.3. Preparation of WT and Minigene Plasmids for CFTR 3849+10KbC>T Mutation

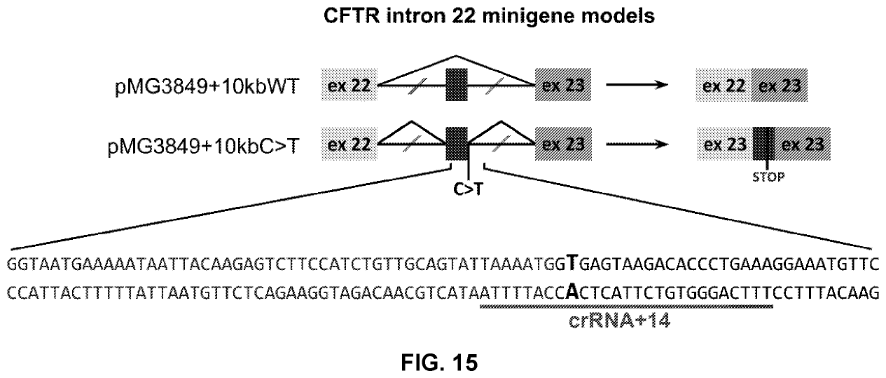

Minigene plasmid models were generated to mimic the splicing pattern of the CFTR gene corresponding to the region encompassing exons 22, 23 and part of intron 22. Plasmid pMG3849+10 kbWT contained the wild-type allele; plasmid pMG3849+10kbC>T contained the mutated allele (FIG. 15).

A wild-type minigene representing the CFTR 3849+10 kb locus was cloned into plasmid pcDNA3 (Invitrogen). Primers 9f, 10f, 11f, 12r, 13r and 14r were used to PCR amplify CFTR DNA of the wild-type sequence of exons 22, 23 and part of intron 22 from the genome of HEK293T cells. The amplified DNA was cloned into plasmid pcDNA3 to generate plasmid pMG3849+10 kbWT containing the wild-type allele of exons 22, 23 and part of intron 22. Primers 15mf and 16mr were used to carry out site-directed mutagenesis of the wild-type minigene housed in pMG3849+10 kbWT to generate the 3849+10kbC>T mutation, creating plasmid pMG3849+10kbC>T.

Sequences coding for guide RNAs (Table 4) were cloned into a commercially available plasmid to generate pY108 lentiAsCas12a (Addgene Plasmid 84739) using BsmBI restriction sites as described above (see FIG. 22C and FIG. 22D).

6.8.3.1.4. Cell Lines

Human colorectal adenocarcinoma cells (Caco-2), and human embryonic kidney cells HEK293T, and HEK293 cells were obtained from the American Type Culture Collection.

6.8.3.1.5. Transfection

Caco-2, HEK293T, and HEK293 cells stably expressing pMG3849+10 kbWT (cell line HEK293/pMG3849+10 kbWT) or 3849+10kbC>T (cell line HEK293/pMG3849+10kbC>T) were prepared and cultured as described in Example 1

Cells were seeded at 1.5×10⁵ cells/well in 24 well plates and transfected with 100 ng of Bgl-II linearized minigene plasmids pMG3849+10 kbWT or pMG3849+10kbC>T complexed with polyethylenimine (PEI) and with 700 ng of plasmid pY108 lentiAsCas12a encoding both the Cas nuclease and gRNA sequences. Cell culture, transfection, and selection for plasmid integration was carried out as described in Example 1. Single cell clones were isolated and characterized for the expression of the minigene construct. Transfected cells were collected three days post-transfection.

6.8.3.1.6. Lentiviral Vector Production

Lentiviral particles were produced in HEK293T cells as described in Example 1.

6.8.3.1.7. Transduction

For transduction studies, HEK293/pMG3849+10 kbWT, HEK293/pMG3849+10kbC>T and Caco-2 cells were seeded at a density of 3×10⁵ cells/well in 12 well plates and transduced as described in Example 1.

6.8.3.1.8. Transcript Analysis

The splicing pattern produced by the mutated or wild-type minigenes, either altered or correct respectively, was evaluated by RT-PCR and sequencing analyses in transfected HEK293T cells (see Beck, S., et al., 1999, Hum. Mutat., 14:133-144). RNA was extracted and target regions were amplified by RT-PCR as previously described. Oligonucleotides are listed in Table 5.

6.8.3.1.9. Detection of Nuclease Induced Genomic Mutations

Genomic DNA was extracted and the target locus amplified by PCR as described in Example 1. The purified PCR products were sequenced and analyzed using TIDE (see Table 4 primers 18f and 19r; Brinkman, E. K., et al., 2014, Nucleic Acids Res., 42: 1-8) or SYNTHEGO ICE software (see Hsiau, T., et al., 2018, bioRxiv, January 20, 1-14). In some studies, DNA editing was also measured using a T7 Endonuclease 1 (T7E1) assay (New England BioLabs) following manufacturer's instructions and as previously described (see Petris, G., et al., 2017, Nat. Commun. 8:1-9).

6.8.3.1.10. GUIDE-seq

Approximately 2×10⁵ HEK293T cells were transfected using Lipofectamine 3000 transfection reagent (Invitrogen) with 1 μg lenti Cas12a plasmid pY108 and 10 pmol of dsODNs designed according to the original GUIDE-seq protocol (see Tsai, S. Q., et al., 2015, Nat. Biotechnol., 33:187-198). Cell culture, genomic DNA extractions and shearing, library construction, sequencing, and analysis was carried out using methods known to those of skill in the art (see Example 1; also Montagna, C., et al., 2018, Mol. Ther. Nucleic Acids, 12:453-462; Casini, A., et al., 2018, Nat. Biotechnol., 36:265-271).

6.8.3.1.11. Targeted Deep Sequencing

The locus of interest, 3849+10Kb C>T/F508, was amplified from genomic DNA extracted from human intestinal organoids 14 days after transduction with lentiAsCas12a-crRNA+14 or a control (CTR) using Phusion high-fidelity polymerase (Thermo Scientific) and primers 18f and 19r. Amplicons were indexed by PCR, quantified, pooled, sequenced on an Illumina Miseq system, and raw sequencing data (FASTQ files) were analysed as described in Example 1.

6.8.3.2. Results

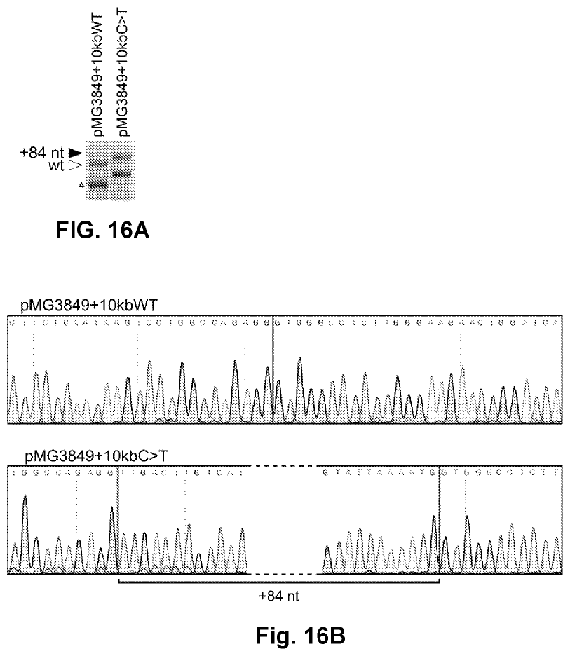

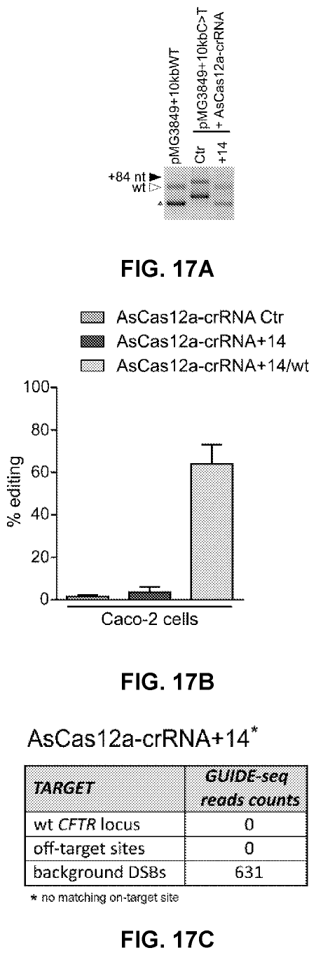

The minigene model containing exon 22, part of intron 22 and exon 23 (pMG3849+10kbWT and pMG3849+10kbC>T; see FIG. 15) successfully mimicked the CFTR splicing defect (FIG. 16A-B). Editing with AsCas12a-crRNA+14 corrected the 3849+10kbC>T splicing impairment in the minigene model (FIG. 17A). Lentiviral transduction of AsCas12a-crRNA+14 in Caco-2 cells generated indels near background levels (3.5%) in the wt CFTR gene. In contrast, AsCas12a-crRNA+14/wt, targeting the wild-type sequence in the same region, produced nearly 70% CFTR editing. These data demonstrate the specificity of the AsCas12a-crRNA+14 towards the mutant allele (FIG. 17B).

To further verify AsCas12a-crRNA+14 specificity, and to examine genome-wide off-target activity, GUIDE-seq analysis was performed in HEK293T cells. The studies revealed a complete absence of sequence reads in the CFTR locus or in any other off-target site; all 631 sequencing reads corresponding to spontaneous DNA breaks were indicative of the proper execution of the GUIDE-seq assay (FIG. 17C).

### 6.8.4. Example 4: CRISPR-Cas12a Correction of CFTR 3849+10KbC>T Splicing Mutation in Organoids

The rescue potential of the CF phenotype by AsCas12a-crRNA+14 in human intestinal organoids compound heterozygous for the 3849+10kbC>T mutation (3849+10kbC>T/ΔF508) was examined.

6.8.4.1. Materials and Methods

6.8.4.1.1. Human Intestinal Organoids Culture and Transduction

Human intestinal organoids of human cystic fibrosis subjects determined to be compound heterozygous for the 3849+10Kb C>T mutation (3849+10Kb C>T/F508, n=1, CF-110) were cultured (see Dekkers, J. F., et al., 2013, Nat. Med., 19:939-945). Cultured organoids were treated and transduced as previously described in Example 2.

6.8.4.1.2. Forskolin Induced Swelling (FIS) Assay and Analysis of CFTR Activity in Intestinal Organoids

Fourteen days after viral vector transduction, the organoids were incubated for 30 minutes with 0.5 μM calcein-green (Invitrogen, C3-100MP) and analyzed by live cell confocal microscopy with a 5× objective (LSM800, Zeiss; Zen Blue software, version 2.3). The steady-state area of the organoids was determined by calculating the absolute area (xy plane, μm²) of each organoid using ImageJ software through the Analyse Particle algorithm. Organoid particles with an area less than 3000 μm were considered defective and were excluded from the analysis. Data were averaged for each different run and plotted in a box plot representing means±SD. The FIS assay was performed by stimulation of the organoids and analysis carried out by live cell confocal microscopy and statistical analyses performed as described above.

6.8.4.2. Results

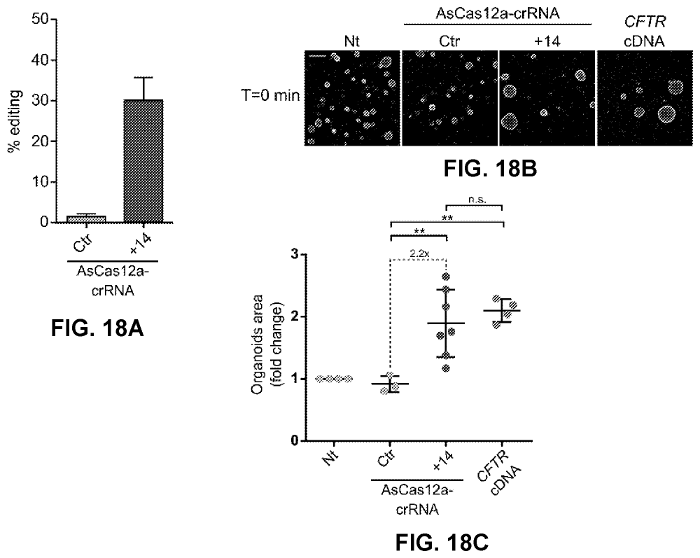

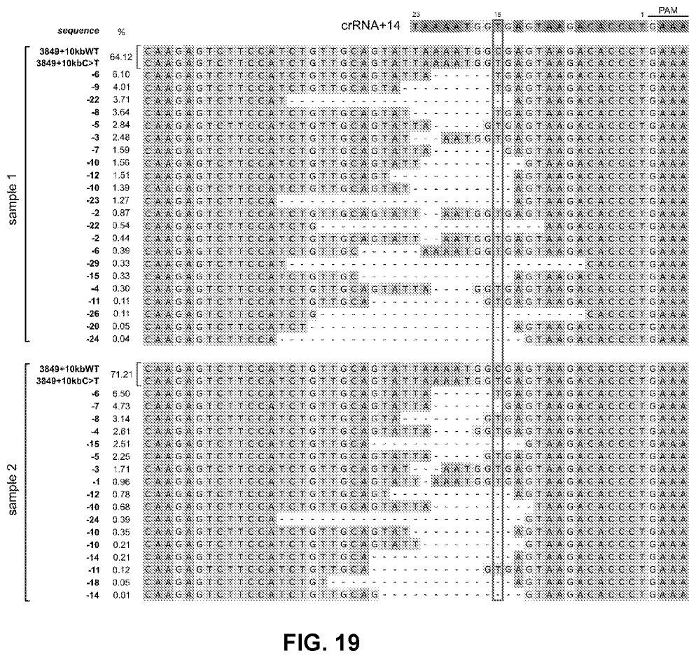

Efficient and precise correction of the CFTR 3849+10kbC>T splicing defect was obtained by using AsCas12a combined with a single allele specific crRNA in patient organoids. Lentiviral delivery of AsCas12a-crRNA+14 produced 31% indels in the CFTR locus (FIG. 18A and FIG. 19), resulting in the rescue of organoid swelling comparable to the rescue observed after wild-type CFTR cDNA gene addition (FIG. 18B-C).

### 6.8.5. Example 5: Comparison of CRISPR-Cas9 and CRISPR-Cas12a Editing of CFTR 3272-26A>G Splicing Mutation in Cells

CRISPR-Cas9 has been the traditional system of choice for gene editing and it was of interest to compare the ability of a SpCas9 system, utilizing multiple sgRNAs, with the AsCas12a system, utilizing single gRNAs, to edit the CFTR 3272-26A>G mutation.

6.8.5.1. Materials and Methods

SpCas9 sgRNAs targeting a CFTR gene having a 3272-26A>G splicing mutation were designed. Target domains are shown in Table 6.

TABLE 6

| | | SEQ | Target domain and surrounding | SEQ |
|---|---|---|---|---|
| Name* | Target domain\hat{ } | ID NO: | genomic sequence^{#} | ID NO: |
| −88 | AATCATATCACAAATG | 138 | aatAATCATATCACAAATGTCATT | 146 |
| | TCAT | | GGtta | |
| −62 | GTACCTGAAAAAGACT | 139 | cttGTACCTGAAAAAGACTAAATT | 147 |
| | AAAT | | AGaat | |
| −52 | ATAATATCTTGTACCT | 140 | ttcATAATATCTTGTACCTGAAAAA | 148 |
| | GAAA | | Gact | |
| −47 | ATTCTAATTTAGTCTTT | 141 | attATTCTAATTTAGTOTTTTTCAG | 149 |
| | TTC | | Gtac | |
| −0 | TTTTGTGTTTATGTTAT | 142 | acaTTTTGTGTTTATGTTATTTGC | 150 |
| | TTG | | AGtgt | |
| +9 | CTGCCTGTGAAATATT | 143 | ctcCTGCCTGTGAAATATTTCCAT | 151 |
| | TCCA | | AGaaa | |
| +10 | GTTATTTGCAGTGTTT | 144 | tatGTTATTTGCAGTGTTTTCTATG | 152 |
| | TCTA | | Gaaa | |
| +22 | TTTTCTATGGAAATATT | 145 | gtgTTTTCTATGGAAATATTTCAC | 153 |
| | TCA | | AGgca | |

*value indicates the distance of the PAM from the mutation;
+ or − indicates the position of the target domain before or after the mutation, respectively;
\hat{ }3272-26A>G mutation position is highlighted in bolded font;
^{#}PAM is underlined;
lower case font indicates nucleotides around the target site

Sequences encoding sgRNAs were cloned into lentiCRISPR v1 plasmid (Addgene Plasmid 49535), which expresses SpCas9, using BsmBI restriction sites. Lentiviral particle production, transduction, and CFTR gene editing analysis was performed as in Example 1.

6.8.5.2. Results

The splicing pattern of the pMG3272-26A>G was evaluated after its co-transfection with the designed sgRNAs in combination with SpCas9 (FIG. 21A). An increased level of correct splicing product using SpCas9 with at least 4 sgRNA pairs (FIG. 21A) was observed. Analysis of the deletions induced by sgRNA pairs showed that a band was excised with SpCas9 (FIG. 21C), in contrast to the results observed for AsCas12a (FIG. 21D).

Unexpectedly, when the splicing correction of CFTR intron 19 in HEK293 cells stably transfected with the pMG3272-26A>G minigene (HEK293/3272-26A>G) was tested, all of the SpCas9-sgRNA pairs failed to correct the splicing defect, suggesting inefficient cleavage at the chromosomal level (FIG. 21E-F). In contrast, AsCas12a-crRNA+11 resulted in the formation of numerous correct transcripts, >60%, from the pMG3272-26A>G transgene (FIG. 9A-B and FIG. 21G) and efficient DNA editing (approximately 70%; FIG. 9C), clearly indicating the better performance of AsCas12a.

### 6.8.6. Example 6: Comparison of CRISPR-Cas9 and CRISPR-Cas12a Editing of CFTR 3849+10KbC>T Splicing Mutation in Cells and Organoids

The ability of a SpCas9 system, utilizing multiple sgRNAs, with the AsCas12a system, utilizing single gRNAs, to edit the CFTR 3849+10KbC>T mutation in cells and organoids was compared.

6.8.6.1. Materials and Methods

SpCas9 sgRNAs targeting a CFTR gene having a 3849+10KbC>T splicing mutation were designed. Target domains are shown in Table 7.

TABLE 7

| | | SEQ | Target domain and | |
|---|---|---|---|---|
| | | ID | surrounding genomic | SEQ |
| Name* | Target domain | NO: | sequence^{#} | ID NO: |
| −95 | ATTCAATTATAATCACCTT | 154 | aagATTCAATTATAATCACCTTG | 160 |
| | G | | TGGatc | |
| −99 | AACTGAAATTTAGATCCA | 155 | gtcAACTGAAATTTAGATCCACA | 161 |
| | CA | | AGGtga | |
| −143 | CTTGATTTCTGGAGACCA | 156 | catCTTGATTTCTGGAGACCAC | 162 |
| | CA | | AAGGtaa | |
| +34 | GAAAGGAAATGTTCTATT | 157 | cttGAAAGGAAATGTTCTATTCA | 163 |
| | CA | | TGGtac | |
| +119 | CACCTCCTCCCTGAGAAT | 158 | atgCACCTCCTCCCTGAGAATG | 164 |
| | GT | | TTGGatc | |
| +125 | TTGATCCAACATTCTCAG | 159 | atcTTGATCCAACATTCTCAGG | 165 |
| | GG | | GAGGagg | |

*value indicates the distance of the PAM from the mutation;
+ or − indicates the position of the target domain before or after the mutation, respectively;
^{#}PAM is underlined;
lower case font indicates nucleotides around the target site

Sequences encoding sgRNAs were cloned into lentiCRISPR v1 plasmid (Addgene Plasmid 49535). Lentiviral particle production, transduction, cell-based CFTR gene editing studies, and organoid studies were performed as in Examples 3 and 4.

6.8.6.2. Results

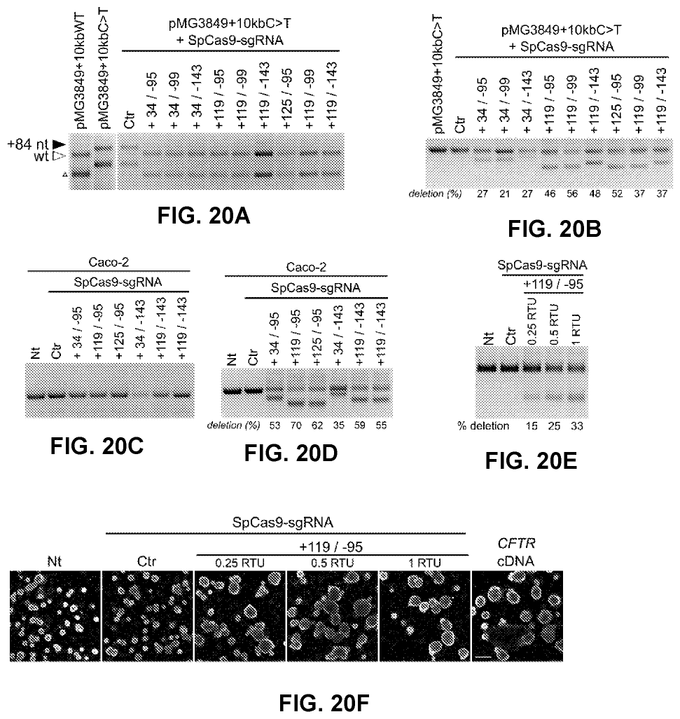

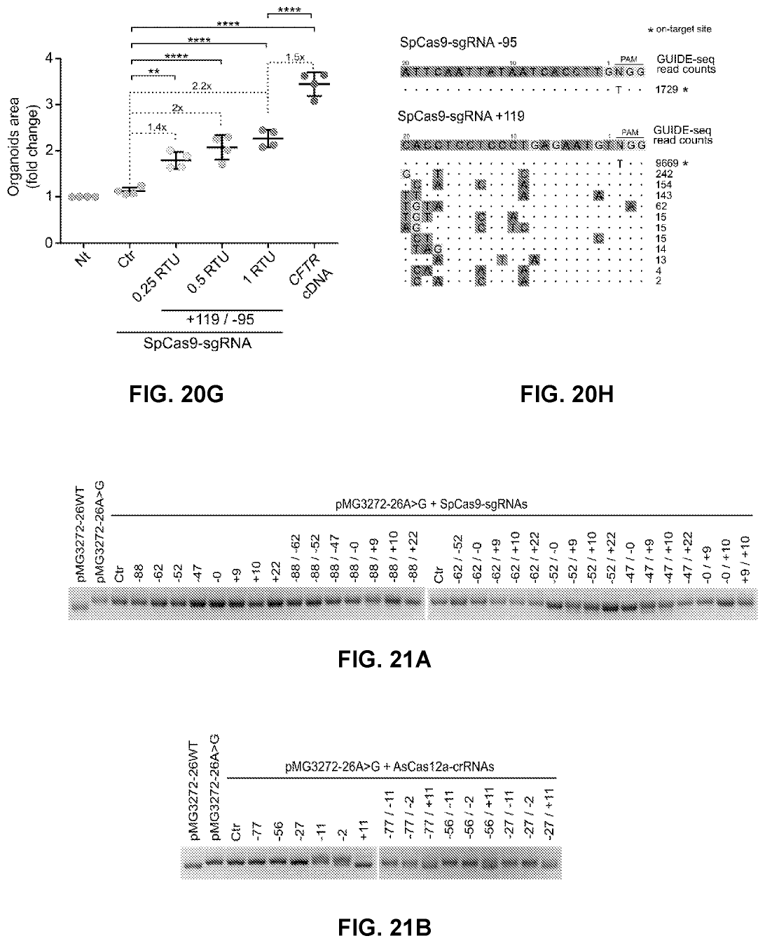

The more conventional strategy to delete the 3849+10kbC>T mutation by SpCas9 with two sgRNAs was carried out in HEK293 and Caco-2 cells (FIG. 20A-D). Select pairs of sgRNAs resulted in a variety of targeted deletions after cleavage with SpCas9-sgRNA pairs with a % deletion ranging from 21% to 56% in HEK293T cells and 35% to 70% in Caco-2 cells.

In patient-derived organoids, the sgRNA-95/+119 appeared to be the best sgRNA pair to obtain efficient intron deletion and splicing correction. Nevertheless, in patient organoids up to 33% of the CFTR 3849+10 kb locus deletion induced an increase of the area of the organoids, which is significantly lower than the area measured after lentiviral delivery of the wild-type CFTR cDNA. (FIG. 20E-G). In addition, while the sgRNA pool was designed in silico to minimize Cas9 off-target activity (Doench, J. G., et al., 2016, Nat. Biotechnol., 34:184-191) the GUIDE-seq assay for sgRNA+119 revealed 11 undesirable off-target sites throughout the genome (FIG. 20H).

In contrast, the correction of the CFTR 3849+10kbC>T splicing defect was efficiently and precisely obtained by using AsCas12a combined with a single allele specific crRNA in patient organoids (Example 4), similarly to the splicing repair of the 3272-26A>G variant (Example 2). The AsCas12a strategy proved superior to the conventional SpCas9 induced genetic deletion obtained in combination with multiple sgRNAs.

### 6.8.7. Example 7: CRISPR-Cas12a Correction of CEP290 IVS26+1655A>G Mutation

6.8.7.1. Materials and Methods

6.8.7.1.1. gRNA Design

The CEP290 IVS26+1655A>G mutation is associated with Leber congenital amaurosis (LCA). A Cas12a gRNA molecule having a targeting sequence corresponding to a target domain in a CEP290 gene having the IVS26+1655A>G mutation is designed (Table 8), with no mismatches between the between the targeting sequence and the complement of the target domain. The loop domain, 5′ to the target domain in the Cas12a gRNA molecule, consists of the sequence UAAUUUCUACUCUUGUAGAU (SEQ ID NO: 25).

TABLE 8

| Gene | | | | |
|---|---|---|---|---|
| (mutation) | | | | |
| (associated | | SEQ ID | PAM and target | SEQ ID |
| disease) | Partial Gene Sequence | NO: | domain | NO: |
| CEP290 | GAGCCACCGCACCTGGCC | 166 | CCCCAGTTGTAAT | 167 |
| (IVS26+1655A>G) | CCAGTTGTAATT/gtga(a>g)ta | | Tgtgagtatctcat | |
| (Leber | tctcatacctatccctattggcagtgtc | | | |
| congenital | | | | |
| amaurosis) | | | | |

PAM sequence is underlined;
exon nucleotides are shown in uppercase;
intron nucleotides are shown in lowercase;
mutation is shown in bold

Using standard golden-gate assembly, a DNA sequence encoding the Cas12a gRNA is cloned into a pY108 lentiAsCas12a plasmid engineered to encode AsCas12a RR to provide a plasmid encoding AsCas12a RR and the Cas12a gRNA. A pY108 lentiAsCas12a plasmid encoding ASCas12a RR and a scramble-truncated gRNA is also prepared for use as a control.

6.8.7.1.2. Minigene Generation

PCR with primers located in CEP290 introns 25 (forward GGGGACAAGTTTGTACAAAAAAGCAGGCTTCGGCCGCTCTTTCTCAAAAGTGGC) (SEQ ID NO: 168) and 27 (reverse GGGGACCACTTTGTACAAGAAAGCTGGGTGCTTGGTGGGGTTAAGTACAGG) (SEQ ID NO: 169) is performed on genomic DNA from a healthy individual and the PCR product is cloned into a pDONR vector using the Gateway system. Via site-directed mutagenesis, the c.2991+1655A>G mutation is introduced using primers mut for (CACCTGGCCCCAGTTGTAATTGTGAGTATCTCATACCTATCCC) (SEQ ID NO: 170) and mut rev (GGGATAGGTATGAGATACTCACAATTACAACTGGGGCCAGGTG) (SEQ ID NO: 171). Both pDONR vectors (mutant and wild-type (WT)) are sequenced and cloned into the destination vector pCi-Neo-Rho-Splicing vector, which allows the cloning of the CEP290 fragment of interest between exons 3 and 5 of RHO under the control of the cytomegalovirus immediate-early promoter as previously described (Shafique, S. et al., 2014, PLoS One, 9:e100146), generating pMG CEP290 WT IVS26+1655A or pMG CEP290 LCA IVS26+1655A>G minigene constructs, as described in Garanto, et al., 2015, Int J Mol Sci, 16(3):5285-5298.

6.8.7.1.3. Cell Culture

HEK293T and HEK293 cells are obtained from American Type Culture Collection (ATCC; www.atcc.org). HEK293T cells and HEK293 cells stably expressing pMG CEP290 WT IVS26+1655A or pMG CEP290 LCA IVS26+1655A>G are cultured in Dulbecco's modified Eagle's medium (DMEM; Life Technologies) supplemented with 10% fetal bovine serum (FBS; Life Technologies), 10 U/ml antibiotics (PenStrep, Life Technologies) and 2 mM L-glutamine at 37° C. in a 5% CO2 humidified atmosphere.

IVS26 patient fibroblasts as described in Burnight, et al., 2014, Gene Ther. 21:662-672 and in Maeder et al., 2019, Nature Medicine, doi: 10.1038/s41591-018-0327-9 are obtained and maintained in Gibco DMEM/F12+glutamax (Thermofisher), supplemented with 1% penicillin/streptomycin, 1% non-essential amino acids and 15% fetal bovine serum.

6.8.7.1.4. Transfection and Transduction

Transfection of HEK293T cells

Transfection is performed in HEK293T cells seeded (150,000 cells/well) in a 24 well plate. Cells are transfected using PEI (polyethylenimine) with 100 ng of minigene plasmids and 700 ng of the plasmid encoding for AsCas12a RR and the Cas12a gRNA.

### Transduction of HEK293 Cells Stably Expressing Minigenes and Patient Fibroblast Cells

Stable minigene cell lines (HEK293/CEP290 WT IVS26+1655A and HEK293/CEP290 LCA IVS26+1655A>G) are produced by transfection with linearized minigene plasmids (pMG CEP290 WT IVS26+1655A or pMG CEP290 LCA IVS26+1655A>G) in HEK293 cells. Cells are selected with 500 μg/ml of G418, 48 h after transfection. Single cell clones are isolated and characterized for the expression of the minigene construct.

Lentiviral particles are produced in HEK293T cells at 80% confluency in 10 cm plates. 10 μg of transfer vector (pY108 lentiAsCas12a RR) plasmid, 3.5 μg of VSV-G and 6.5 μg of Δ8.91 packaging plasmid are transfected using PEI. After over-night incubation, the medium is replaced with complete DMEM. The viral supernatant is collected after 48 h and filtered through a 0.45 μm PES filter. Lentiviral particles are concentrated and purified with a 20% sucrose cushion by ultracentrifugation for 2 hours at 4° C. and 150,000×g. Pellets are resuspended in an appropriate volume of OptiMEM. Aliquots are stored at −80° C. Vector titers are measured as Reverse Transcriptase Units (RTU) by SG-PERT method (see Casini, A., et al., 2015, J. Virol. 89:2966-2971). For transduction studies, HEK293 cells stably expressing the minigene constructs and IVS26 patient fibroblast cells are seeded (300,000 cells/well) in a 12 well plate, and the day after seeding the cells are transduced with 1-5 RTU of lentiviral vectors. Approximately 48 hours later, cells are selected with puromycin (2-10 μg/ml) and collected 10-14 days from transduction.

6.8.7.1.5. RT-PCR and Transcriptional Analysis

RNA is extracted using TRIzol™ Reagent (Invitrogen) and resuspended in DEPC-ddH2O. cDNA is obtained using 500 ng of RNA and RevertAid Reverse Transcriptase (Thermo Scientific), according to the manufacturer's protocol. Target regions are amplified by PCR with Phusion High Fidelity DNA Polymerase (Thermo Fisher) using primers ex26 for (TGCTAAGTACAGGGACATCTTGC (SEQ ID NO: 172)) and ex27rev (AGACTCCACTTGTTCTTTTAAGGAG (SEQ ID NO: 173)) for the CEP290 minigene. PCR products are separated on a 1-2% agarose gel. Fragments representing correctly and aberrantly spliced CEP290 are excised from the gel, purified using Nucleospin Extract II isolation kit (MACHEREY-NAGEL) and sequenced.

6.8.7.2. Results

Minigene transcripts are analyzed two to three days after transfection and exhibit correct and aberrant splicing for the pMG CEP290 WT IVS26+1655A and the plasmids, respectively. Abundant inclusion of the 128 bp cryptic exon is also observed in control cells treated with pY108 lentiAsCas12a RR having a scramble-truncated gRNA, while this aberrant splicing is decreased in transfected cells treated with the CEP290 gRNA. These results are reproduced in HEK293 cells stably transfected with the pMG CEP290 LCA IVS26+1655A>G minigene and transduced with the CEP290 gRNA/AsCas12a lentiviral vector, showing a splicing correction proportional to the gene editing efficiency. CEP290 mRNA transcripts are analyzed 10-14 days after transduction of IVS26+1655A>G and primary patient fibroblasts show that the wild-type transcript is significantly increased and the mutant transcript is decreased relative to the control.

### 6.8.8. Example 8: CRISPR-Cas12a Correction of USH2A c.7595-2144A>G Mutation

6.8.8.1. Materials and Methods

6.8.8.1.1. gRNA Design

The USH2A c.7595-2144A>G mutation is a deep intronic mutation that causes aberrant splicing at a cryptic 5′ splice site and a cryptic 3′ splice site. The mutation is associated with Usher syndrome, Type II (Slijkerman et al., 2016, Mol. Ther. Nucleic Acids, 5(10):e381). Cas12a gRNA molecules having targeting sequences corresponding to the target domains in USH2A shown in Table 9 are designed, with no mismatches between the between the targeting sequence and the complement of the target domain. The Cas12a gRNAs in this example are designed to edit the USH2A gene near the cryptic 5′ splice site (the top four target domains listed in Table 9) or the cryptic 3′ spice site (the bottom four target domains listed in Table 9). The loop domain, 5′ to the target domain in the Cas12a gRNA molecules, consists of the sequence UAAUUUCUACUCUUGUAGAU (SEQ ID NO: 25).

TABLE 9

| Gene | | | | |
|---|---|---|---|---|
| (mutation) | | | | |
| (associated | Partial Gene | SEQ | | SEQ ID |
| disease) | Sequence | ID NO: | PAM and target domain | NO: |
| USH2A | GGCTTTTAAGGGG | 174 | tttc-ttaaagatgatctcttacCTTGG | 176 |
| (c.7595-2144A>G) | GAAACAAATCATG | | TTTC- | 177 |
| (Usher | AAATTGAAATTGA | | CCAAGgtaagagatcatctttaa | |
| syndrome | ACACCTCTCCTTT | | ATTG- | 178 |
| Type II) | CCCAAG/(a>g)gtaa | | AAATTGAACACCTCTCCTTT | |
| | gagatcatctttaagaaaa | | CCC | |
| | ggctgtgtattgtgggggt | | ctta- | 179 |
| | (includes 5′ cryptic | | aagatgatctcttacCTTGGGAA | |
| | splice site) | | | |
| | ttttaacacttccctagcca | 175 | atta- | 180 |
| | aaggagctaattaagctgc | | agctgctttcagCTTCCTCTCCAG | |
| | tttcag/CTTCCTCTC | | ATTC- | 181 |
| | CAGAATCACACAA | | TGGAGAGGAAGctgaaagcagct | |
| | GTTAAAGGACCCT | | CTTG- | 182 |
| | TCTGCAAC | | TGTGATTCTGGAGAGGAAG | |
| | (includes 3′ cryptic | | ctga | |
| | splice site) | | TTTA- | 183 |
| | | | ACTTGTGTGATTCTGGAGA | |
| | | | GGAA | |

PAM sequences are underlined;
exon nucleotides are shown in uppercase;
intron nucleotides are shown in lowercase;
mutations are shown in bold

Using standard golden-gate assembly, DNA sequences encoding the Cas12a gRNAs are cloned into pY108 lentiAsCas12a plasmids engineered to encode AsCas12a RR, AsCas12a RVR, or other Cas12a proteins recognizing the PAM sequences upstream of the target domains to provide plasmids encoding a Cas12a protein and a single Cas12a gRNA. pY108 lentiAsCas12a plasmids encoding a Cas12a protein and a scramble-truncated gRNA are also prepared for use as controls.

6.8.8.1.2. Minigene Generation

A plasmid containing the genomic region of RHO encompassing exons 3-5 cloned into the EcoRI/SalI sites in the pCI-NEO vector (Gamundi, et al., 2008, Hum Mutat 29:869-878) is adapted to the Gateway cloning system, as previously described (Yariz, et al., 2012, Am J Hum Genet, 91:872-882). Gateway cloning technology is used to insert the 152 bp human USH2A pseudoexon 40 (PE40, wild-type and mutant) together with 722 bp of 5′-flanking and 636 bp of 3′-flanking intronic sequences to obtain pMG USH2A-PE40 wt and pMG USH2A-PE40A>G as described in Slijkerman et al., 2016, Mol. Ther. Nucleic Acids, 5(10):e381.

6.8.8.1.3. Cell Culture

HEK293T and HEK293 cells are obtained from American Type Culture Collection (ATCC; www.atcc.org). HEK293T cells and HEK293 cells stably expressing pMG USH2A-PE40 wt or pMG USH2A-PE40A>G are cultured in Dulbecco's modified Eagle's medium (DMEM; Life Technologies) supplemented with 10% fetal bovine serum (FBS; Life Technologies), 10 U/ml antibiotics (PenStrep, Life Technologies) and 2 mM L-glutamine at 37° C. in a 5% CO2 humidified atmosphere.

Primary fibroblasts of an USH2 patient with compound heterozygous USH2A mutations are cultured in DMEM (Sigma-Aldrich D0819) supplemented with 20% fetal bovine serum (Sigma-Aldrich F7524), 1% sodium pyruvate (Sigma-Aldrich S8636) and 1% penicillin-streptomycin (Sigma-Aldrich P4333).

6.8.8.1.4. Transfection and Transduction

### Transfection of HEK293T Cells

Transfection is performed in HEK293T cells seeded (150,000 cells/well) in a 24 well plate. Cells are transfected using PEI (polyethylenimine) with 100 ng of minigene plasmids and 700 ng of the plasmids encoding for the Cas12a proteins and the Cas12a gRNAs.

### Transduction of HEK293 Cells Stably Expressing Minigenes and Patient Fibroblast Cells

Stable minigene cell lines (HEK293/pMG USH2A-PE40 wt and HEK293/pMG USH2A-PE40A>G) are produced by transfection of linearized minigene plasmids (pMG USH2A-PE40 wt or pMG USH2A-PE40A>G) in HEK293 cells. Cells are selected with 500 μg/ml of G418 48 h after transfection. Single cell clones are isolated and characterized for the expression of the minigene constructs.

Lentiviral particles are produced in HEK293T cells at 80% confluency in 10 cm plates. Ten μg of transfer vector plasmid (pY108 lentiAsCas12a plasmids encoding the Cas12a proteins and the Cas12a gRNAs), 3.5 μg of VSV-G and 6.5 μg of Δ8.91 packaging plasmid are transfected into HEK293T cells using PEI. After over-night incubation the medium is replaced with complete DMEM. The viral supernatants are collected after 48 h and filtered through a 0.45 μm PES filter. Lentiviral particles are concentrated and purified with a 20% sucrose cushion by ultracentrifugation for 2 hours at 4° C. and 150,000×g. Pellets are resuspended in an appropriate volume of OptiMEM. Aliquots are stored at −80° C. Vector titers are measured as Reverse Transcriptase Units (RTU) by SG-PERT method (see Casini, A., et al., 2015, J. Virol. 89:2966-2971). For transduction studies, HEK293 cells stably expressing the minigene constructs and USH2 patient fibroblast cells are seeded (300,000 cells/well) in a 12 well plate, and the day after seeding the cells are transduced with 1-5 RTU of the lentiviral vectors. Approximately 48 hours later, cells are selected with puromycin (2-10 μg/ml) and collected 10-14 days from transduction.

6.8.8.1.5. RT-PCR and Transcriptional Analysis

RNA is extracted using TRIzol™ Reagent (Invitrogen) and resuspended in DEPC-ddH2O. cDNA is obtained using 500 ng of RNA and RevertAid Reverse Transcriptase (Thermo Scientific), according to the manufacturer's protocol. Target regions are amplified by PCR with Phusion High Fidelity DNA Polymerase (Thermo Fisher) using primers minigene-USH2A forward (CGGAGGTCAACAACGAGTCT) (SEQ ID NO: 184) and reverse (AGGTGTAGGGGATGGGAGAC (SEQ ID NO: 185)). For the splicing correction experiments in fibroblasts, part of the USH2A cDNA is amplified under standard PCR conditions using Q5 polymerase and primers 5′-GCTCTCCCAGATACCAACTCC-3′ (SEQ ID NO: 186) and 5′-GATTCACATGCCTGACCCTC-3′ (SEQ ID NO: 187) designed for exons 39 and 42, respectively. PCR products are separated on a 1-2% agarose gel. Fragments representing correctly and aberrantly spliced USH2A are excised from the gel, purified using Nucleospin Extract II isolation kit (MACHEREY-NAGEL) and sequenced.

6.8.8.2. Results

Minigene transcripts are analyzed two to three days after transfection and exhibit correct and aberrant splicing for the pMG USH2A-PE40 wt or pMG USH2A-PE40A>G plasmids, respectively. Abundant inclusion of the 152 bp PE40 cryptic exon is also observed in control cells treated with a Cas12a protein and a scramble-truncated gRNA, while this aberrant splicing is decreased in cells treated with at least some of the USH2A PE40 targeting gRNAs. Results are confirmed in HEK293T cells stably transfected with the pMG USH2A-PE40A>G minigene and transduced with USH2A PE40 targeting gRNA/AsCas12a protein lentiviral vectors, showing a splicing correction proportional to the gene editing efficiency. USH2A mRNA transcripts are analyzed 10-14 days after transduction of USH2 patient fibroblast cells and show that the wild-type transcript is significantly increased and the mutant transcript is decreased relative to the control.

### 6.8.9. Example 9: CRISPR-Cas12a Mediated Exon Skipping of Exon 51 of DMD

6.8.9.1. Materials and Methods

6.8.9.1.1. gRNA Design

Mutations in exon 50 of the DMD gene can cause premature truncation of the dystrophin protein. Exon skipping of exon 51 can restore the reading frame and restore expression of a functional dystrophin protein (see, Amoasii et al., 2017, Science Translational Medicine, 9(418):eaan8081). Cas12a gRNA molecules having targeting sequences corresponding to the target domains in DMD shown in Table 10 are designed, with no mismatches between the between the targeting sequence and the complement of the target domain. The loop domain, 5′ to the target domain in the Cas12a gRNA molecules, consists of the sequence UAAUUUCUACUCUUGUAGAU (SEQ ID NO: 25).

TABLE 10

| Gene | | | | |
|---|---|---|---|---|
| (mutation) | | | | |
| (associated | | SEQ ID | | SEQ ID |
| disease) | Partial Gene Sequence | NO: | PAM and target domain | NO: |
| DMD | tttttctttttcttcttttttcctttttgca | 188 | tttq- | 189 |
| (mutation | aaaacccaaaatattttagCT | | caaaaacccaaaatattttagCT | |
| in exon 50) | CCTACTCAGACTGTTA | | tttc- | 190 |
| (Duchenne | CTCTGGTGACACAAC | | ctttttgcaaaaacccaaaatat | |
| muscular | CTGTGGTTACTAAGG | | ttcc- | 191 |
| dystrophy)) | AAA | | tttttgcaaaaacccaaaatatt | |
| | | | GTTG- | 192 |
| | | | TGTCACCAGAGTAACA | |
| | | | GTCTGAG | |
| | | | ttta- | 193 |
| | | | gCTCCTACTCAGACTG | |
| | | | TTACTCT | |

PAM sequencesare underlined;
exon nucleotides are shown in uppercase;
intron nucleotides are shown in lowercase

Using standard golden-gate assembly, DNA sequences encoding the Cas12a gRNAs are cloned into pY108 lentiAsCas12a plasmids engineered to encode AsCas12a RR, AsCas12a RVR, or other Cas12a proteins recognizing the PAM sequences upstream of the target domains to provide plasmids encoding a Cas12a protein and a single Cas12a gRNA. pY108 lentiAsCas12a plasmids encoding a Cas12a protein and a scramble-truncated gRNA are also prepared for use as controls.

6.8.9.1.2. Minigene Generation

Plasmid pCI (Alanis et al., 2012, Hum. Mol. Genet. 21:2389-2398) is used to clone a minigene of DMD Δex50. The minigene is obtained by PCR amplification and cloning of target exons 49 to 52 of DMD from muscle cells or HEK293 cells, excluding exon 50 and including about 200 bp of introns 49, 50 and 51 flanking exons 49, 51, 52 included from the DMD gene. Primers pairs useful for PCR amplification of the genetic regions required for the final minigene assembly (excluding sequences for standard cloning sites used for golden gate assembly) are: 1) exon 49 for GAAACTGAAATAGCAGTTCAAGCTAAACAACC (SEQ ID NO: 194) and intron 49 rev GCCTTAAGATCACAATATATAAATAGGATATGCTG (SEQ ID NO: 195); 2) intron 50 for TGAATCTTTTCATTTTCTACCATGTATTGCT (SEQ ID NO: 196) and intron 51 rev CTTTTTAATGTATGGCTACTTTTGTTATTTGCA (SEQ ID NO: 197); 3) intron 51 for TGAAATATTTTTGATATCTAAGAATGAAACATATTTCCTGT (SEQ ID NO: 198) and exon 52 rev TTCGATCCGTAATGATTGTTCTAGCCTCT (SEQ ID NO: 199).

6.8.9.1.3. Cell Culture

HEK293T and HEK293 cells are obtained from American Type Culture Collection (ATCC; www.atcc.org). HEK293T cells are cultured in Dulbecco's modified Eagle's medium (DMEM; Life Technologies) supplemented with 10% fetal bovine serum (FBS; Life Technologies), 10 U/ml antibiotics (PenStrep, Life Technologies) and 2 mM L-glutamine at 37° C. in a 5% CO2 humidified atmosphere.

6.8.9.1.4. Transfection

Transfection is performed in HEK293T cells seeded (150,000 cells/well) in a 24 well plate. Cells are transfected using PEI (polyethylenimine) with 100 ng of minigene plasmids and 700 ng of the plasmids encoding for Cas12a and the Cas12a gRNAs.

6.8.9.2. Results

Minigene transcripts analyzed two to three days after transfection show the expected splicing pattern including exon 51 in control cells. Decreased exon 51 inclusion is observed in cells transfected with plasmids encoding gRNAs having a targeting sequence corresponding to a target domain in close proximity to or including the intron50-exon51 junction.

### 6.8.10. Example 10: Correction of Various Genetic Defects

6.8.10.1. Materials and Methods

Cas12a gRNA molecules having targeting sequences corresponding to the target domains shown in Table 11 are designed, with no mismatches between the between the targeting sequence and the complement of the target domain. The loop domain, 5′ to the target domain in the Cas12a gRNA molecules, consists of the sequence UAAUUUCUACUCUUGUAGAU (SEQ ID NO: 25). The mutations shown in Table 11 are associated with various genetic diseases (see Section 6.3.4).

TABLE 11

| Gene | | | | |
|---|---|---|---|---|
| (mutation) | | | | |
| (associated | | SEQ | PAM and target | SEQ ID |
| disease) | Partial Gene Sequence | ID NO: | domain | NO: |
| DMD | CCACCAGTTACCTTTGTG | 200 | TTTGTGACCTTTGgt | 222 |
| (IVS9+46806C>T) | ACCTTTG/g(c>t)aagtcatcta | | aagtcatctaat | |
| (muscular | atttttctatttccattt | | GTTACCTTTGTGAC | 223 |
| dystrophy) | | | CTTTGgtaagtca | |
| DMD | GTCCTGATAGTCGATTAT | 201 | ATTATTGATCACAT | 224 |
| (IVS62+62296A>G) | TGATCACATAACAAG/gtca | | AACAAGgtcagtt | |
| (muscular | (a>g)tttatcataactgaagtgcgat | | ATTGATCACATAAC | 225 |
| dystrophy) | cgatt | | AAGgtcagtttat | |
| | | | cttcagttatgataaactgac | 226 |
| | | | CTTGTT | |
| | | | gttatgataaactgacCTT | 227 |
| | | | GTTATGTG | |
| DMD | tattataaaattactctttctcttccttgg | 202 | tttctcttccttggttttgcagC | 228 |
| (IVS1+36846G>A) | ttttgc(g>a)g/CTTCTCG | | TTCT | |
| (muscular | AGTTCATAGG | | ttccttggttttgcagCTTC | 229 |
| dystrophy) | AGACTTTCAGTTTCC | | TCGAGTT | |
| | | | attactctttctcttccttggtttt | 230 |
| | | | gc | |
| DMD | acctagtttgtaataagccatatttcctt | 203 | tttccttgtttctctacatagG | 231 |
| (IVS2+5591T>A) | gtttctctacat(t>a)g/( )GTTGA | | TTGAA | |
| (muscular | ATCTGTTCCTGCAGCAAC | | | |
| dystrophy) | TAGTAAC | | | |
| DMD | tttccccctcctctctatccactccccc | 204 | cccctcctctctatccacctc | 232 |
| (IVS8−15A>G) | a(a>g)/acccttctctgcagATCA | | ccccag | |
| | CGGTCAGTCTAGCACAG | | tttccccctcctctctatccact | 233 |
| | GGATA | | cccc | |
| | | | tccacctcccccagaccctt | 234 |
| | | | ctctgca | |
| | | | tccccctcctctctatccacct | 235 |
| | | | ccccc | |
| DMD | tttttctttttcttcttttttcctttttgcaaaa | 188 | tttg- | 189 |
| (mutation in | acccaaaatattttagCTCCTAC | | caaaaacccaaaatatttta | |
| exon 50) | TCAGACTGTTACTCTGGT | | gCT | |
| (Duchenne | GACACAACCTGTGGTTA | | tttc- | 190 |
| muscular | CTAAGGAAA | | ctttttgcaaaaacccaaaa | |
| dystrophy) | | | tat | |
| | | | ttcc- | 191 |
| | | | tttttgcaaaaacccaaaat | |
| | | | att | |
| | | | GTTG- | 192 |
| | | | TGTCACCAGAGTAA | |
| | | | CAGTCTGAG | |
| | | | ttta- | 193 |
| | | | gCTCCTACTCAGAC | |
| | | | TGTTACTCT | |
| FGB | AATTACTGTGGCCTACCA | 205 | tttattttgcatacctgttcgtta | 236 |
| (IVS6+13C>T) | Ggtaacgaacaggtatgcaaaata | | cCT | |
| (afibrinogenemia) | aaatcattctatttgaaatggg | | tttcaaatagaatgattttatttt | 237 |
| | | | gca | |
| SOD1 | ctttttttccaaag/CAATTAAAA | 206 | tccatggtaagttacactaa | 238 |
| IVS4+792C>G | AAACTGCCAAAGTAAGA | | cCTTAGT | |
| (amyotrophic | GTGACTGCGGAACTAAG/ | | | |
| lateral | gtta(c>g)tgtaacttaccatggagg | | | |
| sclerosis) | attaagggtagcgt | | | |
| HBB | ttctttcagGGCAATAATGATA | 207 | TTTCTGGGTTAAGgt | 239 |
| (IVS2+645C>T) | CAATGTATCATGCCTCTT | | aatagcaatatc | |
| (beta- | TGCACCATTCTAAAGAAT | | tttatatgcagagatattgcta | 240 |
| thalassemia) | AACAGTGATAATTTCTGG | | ttacC | |
| | GTTAAGgtaatagcaatatctctg | | attgctattacCTTAACC | 241 |
| | catataaatatttctgcatataaattgt | | CAGAAATTA | |
| | aactg | | tatgcagagatattgctatta | 242 |
| | | | cCTTAA | |
| HBB | ttccctaatctctttctttcag/GGCA | 208 | TTTCTGCATATAAA | 243 |
| (IVS2+705T>G) | ATAATGATACAATGTATC | | TTGTAACTGAGgt | |
| (beta- | ATGCCTCTTTGCACCATT | | TATAAATTGTAACT | 244 |
| thalassemia) | CTAAAGAATAACAGTGAT | | GAGgtaagaggtt | |
| | AATTTCTGGGTTAAGGCA | | tatgaaacctcttacCTCA | 245 |
| | ATAGCAATATCTCTGCAT | | GTTACAAT | |
| | ATAAATATTTCTGCATATA | | attagcaatatgaaacctctt | 246 |
| | AATTGTAACTGA(T>G)/gta | | acCTCA | |
| | agaggtttcatattgctaatagcagct | | | |
| | acaatc | | | |
| HBB | tcag/( )GGCAATAATGATAC | 209 | ATTGCTAATAGCAG | 247 |
| (IVS2+745C>G) | AATGTATCATGCCTCTTT | | CTACAATCCAGgt | |
| (beta- | GCACCATTCTAAAGAATA | | | |
| thalassemia) | ACAGTGATAATTTCTGGG | | | |
| | TTAAGGCAATAGCAATAT | | | |
| | CTCTGCATATAAATATTT | | | |
| | CTGCATATAAATTGTAAC | | | |
| | TGATGTAAGAGGTTTCAT | | | |
| | ATTGCTAATAGCAGCTAC | | | |
| | AATCCAG/(c>g)taccattctgct | | | |
| | tttattttatggttgggataag | | | |
| CFTR | acag/AAGTACCAACAATT | 210 | TATGTACTTGAGAT | 248 |
| (IVS11+194A>G) | ACATGTATAAACAGAGAA | | gtaagtaaggtta | |
| (cystic fibrosis) | TCCTATGTACTTGAGAT/ | | attgatagtaaccttacttac | 249 |
| | (a>g)taagtaaggttactatcaatca | | ATCTCA | |
| | cacctgaaaaatttaaat | | | |
| CFTR | GCTTGATCAATGGCATG | 211 | gttaaaattccatcttacCA | 250 |
| (IVS19+11505C>G) | GGAAAACAGGCAATACA | | ATTCTAA | |
| (cystic fibrosis) | GTTAGAATTGgtaagatggaa | | attgaacgttaaaattccatc | 251 |
| | ttttaacgttcaattaaggatctatctct | | ttacCA | |
| | a | | | |
| QDPR | GTTTTGTCATCTGTAAAA | | | |
| (IVS3+2552A>G) | TAAG/(a>g)taaaatagtgtctcct | 212 | TTTGTCATCTGTAA | 252 |
| (Dihydropteridine | ttatatatatggtggttgtaccttgt | | AATAAGagtaaaa | |
| reductase | | | | |
| deficiency | | | | |
| GLA | ttctcagAGCTCCACACTATT | 213 | GTTACCATGTCTCC | 253 |
| (IVS4+919G>A) | TGGAAGTATTTGTTGACT | | CCACTAAAGTgta | |
| (Fabry | TGTTACCATGTCTCCCCA | | | |
| disease) | CTA(G>A)AGT/gtaagtttcatg | | | |
| | agggcagggaccttgtctg | | | |
| LDLR | GGGCAACCGGAAGACCA | 214 | TTTGAGgtgtggcttagg | 254 |
| (IVS12+11C>G) | TCTTGGAGGATGAAAAG | | tacgagatg | |
| (familial | AGGCTGGCCCACCCCTT | | | |
| hypercholester | CTCCTTGGCCGTCTTTGA | | | |
| ol-emia) | Ggtgtggctta(c>g)/gtacgagat | | | |
| | gcaagcacttaggtggcggataga | | | |
| | c | | | |
| BRIP1 | GTAGATGAAGGCTGAGA | 215 | gttataaaattcttacatacC | 255 |
| (IVS11+2767A>T) | CTCAGGTTTCAAAG/g(a>t) | | TTTGAA | |
| (Fanconi | atgtaagaattttataacttgttgctaa | | | |
| anemia) | tactttaaaaactt | | | |
| F9 | AAGTCCTGTGAACCAGC | 216 | tttaaaaaatcttactcagatt | 256 |
| (IVS5+13A>G+12) | AGgtcataatctga/(a>g)taagat | | atgac | |
| (hemophilia B) | tttttaaagaaaatctgtatctgaaac | | tttctttaaaaaatcttactca | 257 |
| | | | gatta | |
| CEP290 | GAGCCACCGCACCTGGC | 166 | CCCCAGTTGTAATT | 167 |
| (IVS26+1655A>G) | CCCAGTTGTAATT/gtga(a>g) | | gtgagtatctcat | |
| (Leber | tatctcatacctatccctattggcag | | | |
| congenital | tgtc | | | |
| amaurosis) | | | | |
| COL2A1 | tttctccatccacaccgc(g>a)g/gg | 217 | tttctccatccacaccgcag | 258 |
| (IVS23+135A-234) | agagggagtctgatcctgatttgtgc | | ggagag | |
| (Stickler | cgc | | | |
| syndrome) | | | | |
| USH2A | gaggtgggacatttccaagaggtct | 218 | tttctggatttattttagtttaca | 259 |
| (IVS40−8C>G) | gactttctggatttatttta(c>g)/tttac | | gAA | |
| (Usher | agAACCTGGACCTGTAGT | | tttccaagaggtctgactttct | 260 |
| syndrome, type | TCCTCCGATTCTTCTGGA | | ggatt | |
| II) | TGTGAAGT | | tccaagaggtctgactttctg | 261 |
| | | | gattta | |
| | | | TCCAGGTTctgtaaact | 262 |
| | | | aaaataaatc | |
| | | | tttattttagtttacagAACC | 263 |
| | | | TGGACC | |
| USH2A | GGCAGAAGGA | 219 | ttca- | 264 |
| (IVS66+39C>T) | TGAAGAAACT | | tatgtctgtacacatacCTT | |
| (Usher | AACAAG/g(c>t)atgtgta | | GTT | |
| syndrome) | cagacatatgaactcatggt | | gttc- | 265 |
| | atagcctact | | atatgtctgtacacatacCT | |
| | | | TGT | |
| USH2A | GGCTTTTAAGGGGGAAA | 174 | tttc- | 176 |
| (c.7595−2144A>G) | CAAATCATGAAATTGAAA | | ttaaagatgatctcttacCT | |
| (Usher | TTGAACACCTCTCCTTTC | | TGG | |
| syndrome Type | CCAAG/(a>g)gtaagagatcatc | | TTTC- | 177 |
| II) | tttaagaaaaggctgtgtattgtgggg | | CCAAGgtaagagatcat | |
| | gt | | ctttaa | |
| | | | ATTG- | 178 |
| | | | AAATTGAACACCTC | |
| | | | TCCTTTCCC | |
| | | | ctta- | 179 |
| | | | aagatgatctcttacCTTG | |
| | | | GGAA | |
| | ttttaacacttccctagccaaaggag | 175 | atta- | 180 |
| | ctaattaagctgctttcag/CTTCC | | agctgctttcagCTTCCT | |
| | TCTCCAGAATCACACAAG | | CTCCAG | |
| | TTAAAGGACCCTTCTGCA | | ATTC- | 181 |
| | AC | | TGGAGAGGAAGctg | |
| | | | aaagcagct | |
| | | | CTTG- | 182 |
| | | | TGTGATTCTGGAGA | |
| | | | GGAAGctga | |
| | | | TTTA- | 183 |
| | | | ACTTGTGTGATTCT | |
| | | | GGAGAGGAA | |
| GAA | agtgccgcccctcccg | 220 | tccc- | 266 |
| (IVS1−13T>G) | cctccctgct | | tgctgagcccgcttgcttctc | |
| (Glycogen | gagcccgctt/(t>g)cttct | | cc | |
| storage | cccgcagGCC | | tccc- | 267 |
| disease type II | TGTAGGAGCT | | gcctccctgctgagcccgct | |
| | GTCCAGGCCA | | tgc | |
| | | | cccc- | 268 |
| | | | tcccgcctccctgctgagcc | |
| | | | cgc | |
| GAA | gcccccgccccaaggctccctcct | 221 | tccc- | 269 |
| (IVS6−22T>G) | ccctccctca(t>g)/gaag | | tcctccctccctcaggaagt | |
| (Glycogen | tcggcgttggcctgcagGAT | | cgg | |
| storage | ACCCGTTCAT | | cccc- | 270 |
| disease type II) | | | aaggctccctcctccctccct | |
| | | | ca | |
| | | | tccc- | 271 |
| | | | tccctcaggaagtcggcgtt | |
| | | | ggc | |

PAM sequences are underlined;
exon nucleotides are shown in uppercase;
intron nucleotides are shown in lowercase;
mutations are shown in bold

Lentivirus particles encoding single Cas12a gRNAs and Cas12a proteins are produced according to methods similar to those described in Example 1. Stable minigene cell lines expressing the wild-type and mutant mini-genes corresponding to the genes listed in Table 11 are produced in a manner similar to Example 1, and transduced with the lentivirus particles. Approximately 10 days after transduction, cells are collected and DNA and RNA are extracted from the cells. DNA is analyzed for Cas12a induced genome editing, and RNA is analyzed for corrected splicing, similar to Example 1.

Organoids from subjects having the mutations described in Table 11 are transduced with the lentivirus particles using procedures similar to the procedure described in Example 2. Fourteen days after transduction organoids are analyzed for reversion of disease phenotype.

6.8.10.2. Results

Cas12a proteins in combination with single Cas12a gRNAs correct splicing defects caused by the mutations identified in Table 11 in minigene models and restores dystrophin expression in a minigene model of a deleterious mutation in exon 50 of DMD. In the organoids, Cas12a proteins in combination with single Cas12a gRNAs reverse the disease phenotypes.

### 6.8.11. Example 11: CRISPR-Cas12a Correction of USH2A c.7595-2144A>G Mutation

6.8.11.1. Materials and Methods

6.8.11.1.1. gRNA Design

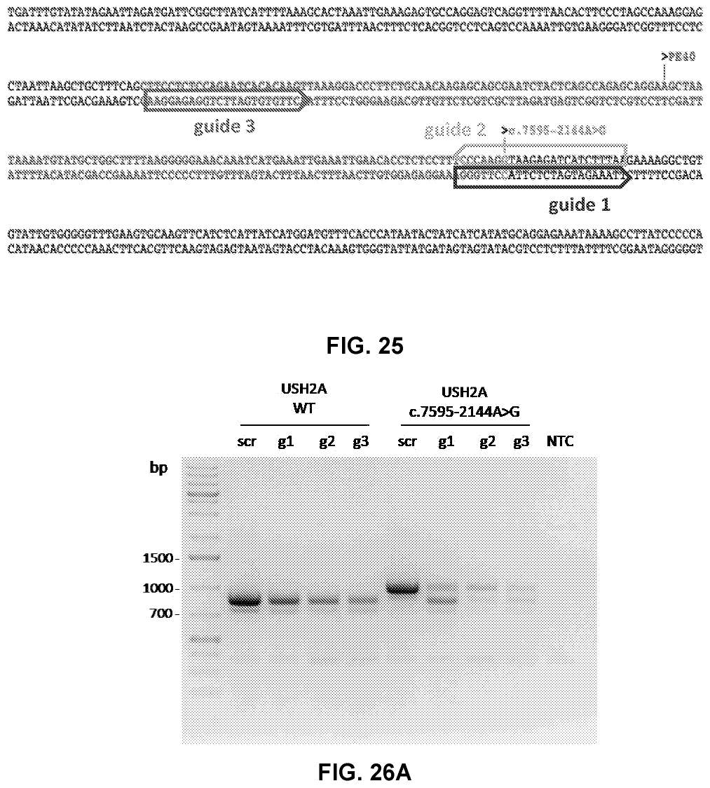

Cas12a gRNA molecules having targeting sequences corresponding to the target domains in USH2A shown in Table 12 were designed, with no mismatches between the targeting sequence and the complement of the target domain. The Cas12a gRNAs in this example were designed to edit the USH2A gene near the cryptic 5′ splice site (the top two target domains listed in Table 12) or the cryptic 3′ spice site (the bottom target domain listed in Table 12). The loop domain, 5′ to the target domain in the Cas12a gRNA molecules, consists of the sequence UAAUUUCUACUCUUGUAGAU (SEQ ID NO: 25) (AsCas12a) or UAAUUUCUACUAAGUGUAGAU (SEQ ID NO: 31) (LbCas12a). A schematic representation of the positions of the selected target domains is reported in FIG. 25.

TABLE 12
Spacers and target sequences for the USH2A c.7595-2144A>G mutation

| | SEQ | | SEQ | Guide |
|---|---|---|---|---|
| Partial Gene Sequence | ID NO: | PAM and target domain | ID NO: | name |
| GGCTTTTAAGGGGGAAA | 174 | tttc-ttaaagatgatctcttacCTTGG | 176 | guide |
| CAAATCATGAAATTGAAA | | | | 1 |
| TTGAACACCTCTCCTTTC | | TTTC-CCAAGgtaagagatcatctttaa | 177 | guide |
| CCAAG/(a>g)gtaagagatcatc | | | | 2 |
| tttaagaaaaggctgtgtattgtgggggt | | | | |
| (includes 5′ cryptic splice site) | | | | |
| ttttaacacttccctagccaaaggag | 175 | TTTA- | 183 | guide |
| ctaattaagctgctttcag/CTTCC | | ACTTGTGTGATTCTGGAGAGGAA | | 3 |
| TCTCCAGAATCACACAAG | | | | |
| TTAAAGGACCCTTCTGCAAC | | | | |
| (includes 3′ cryptic splice site) | | | | |

PAM sequences are underlined.
Intronic sequences are reported lowercase, exonic sequences are uppercase.
The mutated base is reported in bold.
A dash indicates the intron-exon junction.

Using standard golden-gate assembly, DNA sequences encoding the Cas12a gRNAs were cloned into the pY108 (Addgene plasmid number 84739, encoding AsCas12a) or pY109 (Addgene plasmid number 84740, encoding LbCas12a) lentiviral vectors. These vectors were engineered to encode Cas12a proteins together with their respective gRNAs in order to recognize the PAM sequences upstream of the selected target domains. pY108 and pY109 plasmids encoding the AsCas12a and LbCas12a proteins, respectively, together with a scramble-truncated gRNA were also prepared for use as controls. The oligonucleotides used to generate the above described vectors are reported in Table 13.

TABLE 13
Oligonucleotides used to clone gRNAs

| Guide | | SEQ | | SEQ |
|---|---|---|---|---|
| name | Oligo 1 (5′ to 3′) | ID NO: | Oligo 2 (5′ to 3′) | ID NO: |
| guide 1 | agatTTAAAGATGATCTCTTACCT | 272 | aaaaCCAAGGTAAGAGATCATCT | 275 |
| | TGG | | TTAA | |
| guide 2 | agatCCAAGGTAAGAGATCATCT | 273 | aaaaTTAAAGATGATCTCTTACC | 276 |
| | TTAA | | TTGG | |
| guide 3 | agatACTTGTGTGATTCTGGAGA | 274 | aaaaTTCCTCTCCAGAATCACAC | 277 |
| | GGAA | | AAGT | |

Cloning overhangs are reported in lowercase.
Spacers are reported uppercase.

6.8.11.1.1. Minigenes Generation

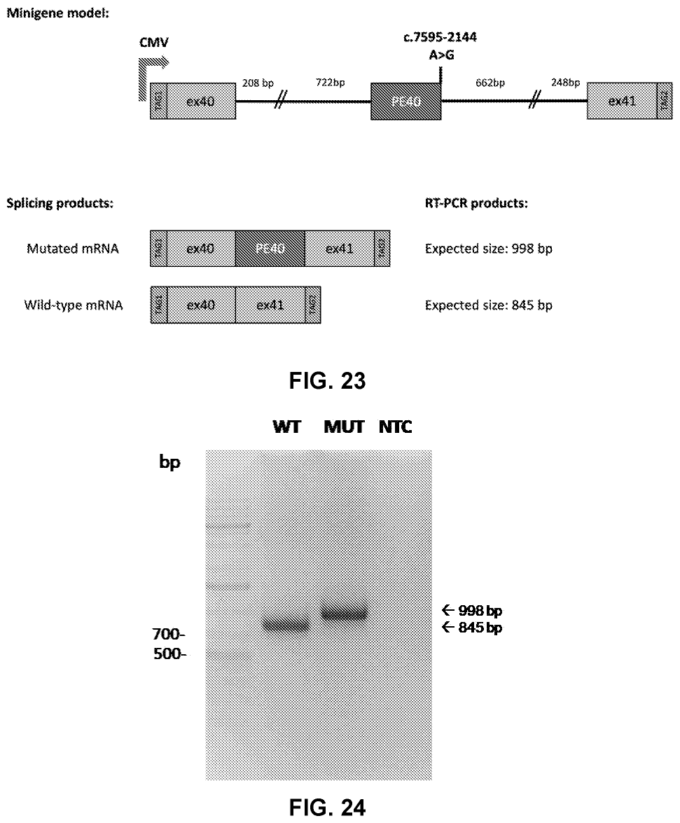

Minigene models were generated to mimic the splicing pattern of the wild-type USH2A gene and its mutated counterpart. USH2A exon 40 and exon 41, together with the genomic region corresponding to PE40 were amplified from genomic DNA extracted from HEK293T cells using the primers listed in Table 14. The amplicon corresponding to exon 40 includes additional 208 bp of the 5′-end of intron 40; the amplicon corresponding to exon 41 further includes 248 bp of the 3′-end of intron 40; the amplicon corresponding to PE40 further includes portions of intron 40 up to 722 bp upstream and 622 bp downstream of the pseudoexon itself. These fragments were then assembled using golden-gate assembly and cloned into the KpnI and BglII sites of a previously published pcDNA3 vector (Cesaratto et al., 2015, J. Biotechnol. 212:159-166) to allow expression under the control of a CMV promoter. The construct also included two protein tags, a V5-tag and a roTag (Petris et al., 2014, PLoS One, 9(5):e96700) respectively, at the 5′- and 3′-end of the minigene to aid its expression. The minigene containing the USH2A c.7595-2144A>G mutation was obtained from the wild-type minigene through standard procedures of site-directed mutagenesis using the primers reported in Table 14 (oligonucleotides USH2A_mutA2144G_F and USH2A_mutA2144G_R). A schematic representation of the minigene construct is reported in FIG. 23.

TABLE 14
Oligonucleotides used to generate USH2A minigenes

| Oligo Name | Sequence (5′ to 3′) | SEQ ID NO: |
|---|---|---|
| Kpn-USH2A_ex40-F | ttaggtaccgaGTTATTTTCCAATCCTTCTGCATC | 278 |
| BsmBI-USH2A_ex40-R | ttccgtctcataatGCCTAACCCTCCAACCCTCC | 279 |
| BsmBI-USH2A_PE40-F | gaacgtctctATTACTCTATTTTAGGCTGGGGC | 280 |
| BsmBI-USH2A_PE40-R | tctcgtctcatcaaTGTATCCTATTTGAAGAGAAATCC | 281 |
| BsmBI-USH2A_ex41-F | agacgtctcaTTGATAAAGCTGTCTTAGAGAGGA | 282 |
| BgIII-USH2A_ex41-R | taatagatctCTGGACTGCATCGGGTTCC | 283 |
| USH2A_mutA2144G_F | CTCTCCTTTCCCAAGgTAAGAGATCATCTTTAAG | 284 |
| USH2A_mutA2144G_R | CTTAAAGATGATCTCTTAcCTTGGGAAAGGAGAG | 285 |

6.8.11.1.1. Cell Culture

HEK293T and HEK293 cells were obtained from the American Type Culture Collection (ATCC; www.atcc.org). Cells were cultured in Dulbecco's modified Eagle's medium (DMEM; Life Technologies) supplemented with 10% fetal bovine serum (FBS; Life Technologies), 10 U/ml antibiotics (PenStrep, Life Technologies) and 2 mM L-glutamine at 37° C. in a 5% CO2 humidified atmosphere.

HEK293 cells stably expressing USH2A wild-type and mutated minigenes were generated by stable transfection of linearized minigene plasmids. Cells were selected with 600 μg/ml of G418 (Invivogen) starting from 48 h after transfection. Single cell clones were isolated and characterized for the minigenes copy number and the expression of the minigene constructs. Stable clones were maintained in culture as indicated above with the additional supplementation of 500 μg/ml of G418.

6.8.11.1.1. Determination of Minigene Copy Number in HEK293 Stable Clones

Determination of minigene copy number was performed by qPCR analysis on genomic DNA extracted using the NucleoSpin Tissue kit (Macherey-Nagel). Genomic DNA was diluted to 86.2 ng/μl and qPCR was performed using primers reported in Table 15. GAPDH was used as control to determine the relative copy number. Standard curves for both the minigene and GAPDH were obtained with serial dilutions of the minigene plasmids or pcDNA3-GAPDH-fragment, respectively. The pcDNA3-GAPDH-fragment construct was obtained by blunt-end cloning of a GAPDH fragment amplified using the GAPDH_CN_For and GAPDH_CN_Rev primers reported in Table 15, which were the same primers used for GAPDH qPCR amplification.

TABLE 15
Oligonucleotides used for copy number
determination

| | | SEQ |
|---|---|---|
| | | ID |
| Oligo name | Sequence (5′ to 3′) | NO: |
| USH2A_minigene_CN_For | GGCAAACCAATCCCAAACCC | 286 |
| USH2A_minigene_CN_Rev | ATTGGAGGCAACCAACCGAA | 287 |
| GAPDH_CN_For | CACAGTCCAGTCCTGGGAAC | 288 |
| GAPDH_CN_Rev | TAGTAGCCGGGCCCTACTTT | 289 |

6.8.11.1.1. Transfection and Transduction

### Transfection of HEK293 Cells

Transfections were performed in HEK293 cells seeded (100,000 cells/well) in a 24 well plate. 24 hours after seeding, cells were transfected with 100 ng of minigene plasmids and 700 ng of the plasmids encoding for the Cas12a proteins and the Cas12a gRNAs targeting USH2A using TransIT-LT1 (Mirus Bio) according to manufacturer's instructions. Cells were split at confluence and collected 6 days post-transfection. Pellets were subsequently divided into two for DNA and RNA extraction to compare editing efficiency and splicing correction within the same samples.

### Transduction of HEK293 Cells Stably Expressing Minigenes

Lentiviral particles were produced in HEK293T cells at 80% confluency in 10 cm plates. Briefly, 10 μg of transfer vector plasmid (pY108 or pY109 plasmids, encoding the Cas12a proteins and the Cas12a gRNAs), 3.5 μg of a VSV-G expressing plasmid (pMD2.G, Addgene plasmid number 12259) and 6.5 μg of a lentiviral packaging plasmid (pCMV-dR8.91) were transfected into HEK293T cells using the polyethyleneimine method (PEI) (see Casini A et al., 2015, J. Virol. 89: 2966-2971). After over-night incubation the medium was replaced with complete DMEM. The viral supernatants were collected after 48 h and filtered through a 0.45 μm PES filter. Aliquots were stored at −80° C. until use. Vector titers were measured as Reverse Transcriptase Units (RTU) by the SG-PERT method (see Casini, A., et al., 2015, J. Virol. 89:2966-2971). For transduction studies, HEK293 cells stably expressing the minigene constructs were seeded (100,000 cells/well) in a 24 well plate, and the day after seeding the cells were transduced with 1 RTU of the lentiviral vectors by centrifuging vector-containing medium on the cells for 2 hours at 1600×g 25° C. Approximately 48 hours later, cells were selected with puromycin (1 μg/ml) and collected at 10 days from transduction.

6.8.11.1.1. RT-PCR and Transcriptional Analysis

RNA was extracted using NucleoZOL Reagent (Macherey-Nagel) and resuspended in RNase free-ddH2O. cDNA was obtained from 1 μg of RNA using the RevertAid RT Reverse Transcription kit (Thermo Scientific), according to the manufacturer's protocol. Target regions were amplified by PCR with the HOT FIREPol MultiPlex Mix (Solis Biodyne) using primers V5tag_For and TEVsite_Rev (reported in Table 13). PCR products were run on 1.5% agarose gel and images were obtained with the UVIdoc HD5 system (Uvitec Cambridge). Bands quantification was performed using the Uvitec Alliance Software (Uvitec Cambridge).

6.8.11.1.1. Evaluation of Indel Formation

Genomic DNA was extracted from cell pellets using the QuickExtract solution (Lucigen) according to manufacturer's instructions. The HOT FIREPol MultiPlex Mix (Solis Biodyne) was used to amplify the integrated USH2A minigene using primers TIDE-USH2A-PE40-F (reported in Table 16) and TEVsite_Rev (reported in Table 16), specifically detecting the integrated USH2A minigene. The amplicon pools were Sanger sequenced (Mix2seq kits, Eurofins Genomics) and the indel levels were evaluated using the TIDE webtool (tide.deskgen.com/) or the Synthego ICE webtool (ice.synthego.com/).

TABLE 16
Oligonucleotides used for RT-PCR and TIDE analyses

| Oligo name | Sequence (5′ to 3′) | SEQ ID NO: |
|---|---|---|
| V5tag_For | CAAACCAATCCCAAACCCACT | 290 |
| TEVsite_Rev | CGCCCTGGAAGTATAAATTCTC | 291 |
| TIDE-USH2A-PE40-F | AGTTGCAGGCCAGTTGATTT | 292 |

6.8.11.2.

6.8.11.3. Results

6.8.11.3.1. Design of Minigenes to Recapitulate USH2A c.7595-2144A>G Splicing

A minigene to recapitulate the aberrant USH2A c.7595-2144A>G splicing was generated by cloning the human genomic regions coding for USH2A exon 40 and exon 41, as well as portions of USH2A intron 40 corresponding to the pseudoexon 40 (PE40), into a CMV-driven mammalian expression vector based on pcDNA3 (Cesaratto et al., J. Biotechnol. 212, 159-166, 2015). In addition, to preserve important splicing regulatory sequences, the minigene included also parts of USH2A intron 40 immediately downstream and upstream of exons 40 and 41, respectively. A schematic representation of minigene design is reported in FIG. 1A. In addition, both a wild-type minigene and a minigene containing the c.7595-2144A>G mutation were constructed in order to evaluate the effect of the designed genome editing strategy on the splicing of the wild-type and the mutated USH2A sequence. The splicing patterns of both the wild-type and the mutated minigenes were first evaluated by RT-PCR after transient transfection of the two constructs in HEK293 cells. As expected, the splicing product deriving from the mutated minigene showed an increase of 153 bp in length, corresponding to the inclusion of PE40 in the expressed mRNA. Inclusion of PE40 was further confirmed by Sanger sequencing of the PCR products.

6.8.11.3.1. Correction of USH2A c.7595-2144A>G Splicing Using Cas12a-Mediated Genome Editing

Cas12a guide RNAs targeting the 5′ and 3′-cryptic splice sites promoting the inclusion of PE40 in the USH2A transcript were designed for both AsCas12a and LbCas12a. While guide 1 and guide 2 span the 3′ cryptic splice site and the c.7595-2144A>G mutation, guide 3 is positioned at the level of the 5′ cryptic splice site, at the beginning of the sequence corresponding to PE40 (FIG. 25).

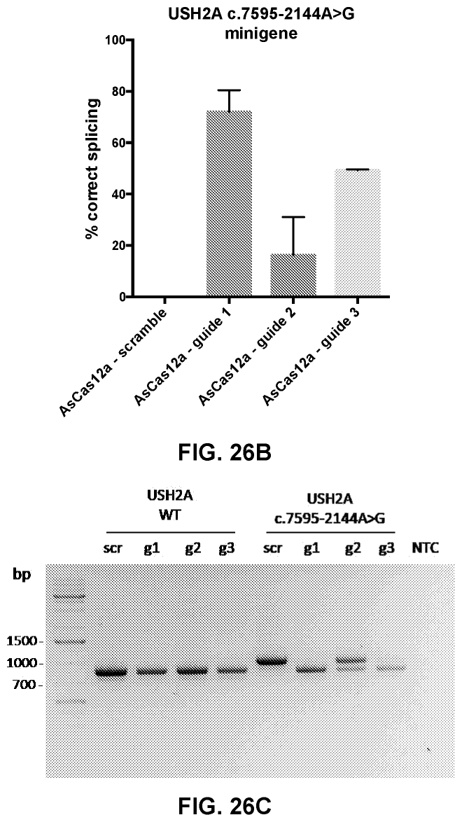

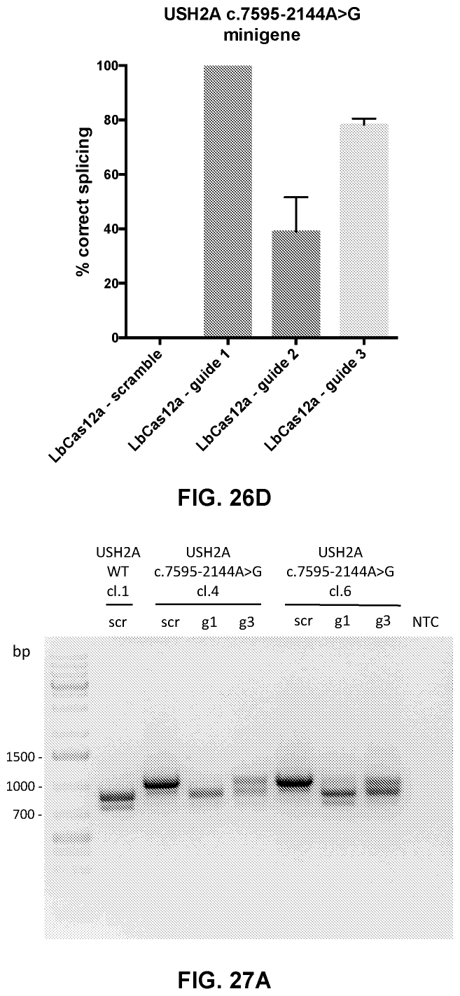

The levels of splicing correction promoted by AsCas12a and LbCas12a in combination with the 3 designed gRNAs were first tested by transient transfection of HEK293 cells with each nuclease-gRNA pair together with the USH2A minigene bearing the c.7595-2144A>G mutation. A scramble non-targeting gRNA (scr) was included in the studies as a control. Cells were collected at 6 days post transfection and USH2A splicing pattern was analyzed by RT-PCR on total extracted mRNA. As shown in FIG. 26A and FIG. 26C both AsCas12a and LbCas12a were able to revert PE40 inclusion in the mature transcript. Guide 1 was the most efficient gRNA (approx. 70-100% splicing correction, see FIG. 26B and FIG. 26D), followed by Guide 3 (approx. 50-80% splicing correction, see FIG. 26B and FIG. 26D). Guide 2 was able to promote only lower levels of splicing restoration (approx. 15-40% or correct products, see FIG. 26B and FIG. 26D). In addition, surprisingly, LbCas12a was much more efficient in promoting splicing correction than AsCas12a, with almost a 2-fold improvement in the percentage of transcripts not including PE40 (compare FIGS. 26A-B and FIGS. 26C-D). To verify the absence of detrimental effects of the genome editing strategy on the wild-type USH2A transcript, similar transient transfection studies were performed using the wild-type USH2A minigene. As shown in FIG. 26A and FIG. 26C (left sides of the panels), both AsCas12a and LbCas12a in combination with all the tested gRNAs were not perturbing the splicing of wild-type USH2A minigene transcript (compare lanes scr, scramble, with lanes g1-g3).

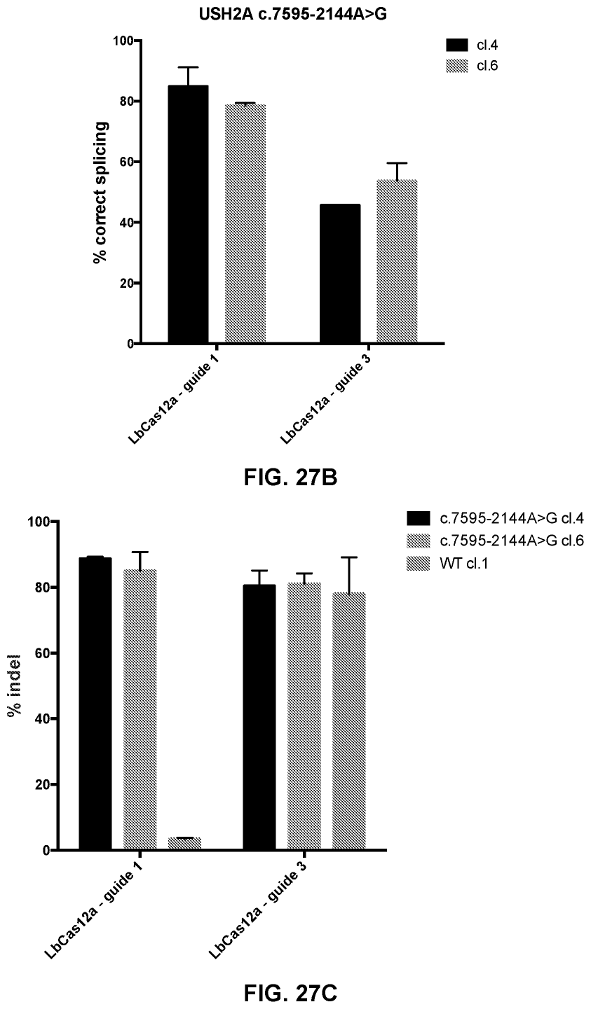

To further confirm the efficiency of the correction strategy, HEK293 clones stably expressing the c.7595-2144A>G USH2A mutated minigene and its wild-type counterpart were generated and characterized for copy number using a qPCR assay. Three clones were selected for subsequent studies: two clones expressing the mutated minigene (clone 4, bearing 2 copies of the mutated minigene; clone 6, bearing 1 copy of the mutated minigene) and a single clone (clone 1) characterized by 5 copies of the wild-type minigene. In addition, only LbCas12a in combination with guide 1 and guide 3 was further tested since those resulted to be the best performing combinations in transient transfection studies. Lentiviral vectors encoding LbCas12a and either guide 1, guide 3 or a scramble non-targeting gRNA were produced. HEK293 clones bearing the mutated minigenes were transduced with each of the three lentiviral vectors and kept for 10 days under puromycin selection to isolate transduced cells. The levels of USH2A splicing correction were then assessed by RT-PCR on total extracted RNA, revealing the restoration of the corrected transcript with both gRNAs (FIGS. 27A-B) with guide 1 showing higher efficiency than guide 3 (approx. 80% vs 40-50%, respectively, see FIG. 27B), in accordance with previous data obtained in transient transfection studies. Notably, splicing correction was consistent in both tested clones (FIGS. 27A-B), further confirming the efficacy of the approach.

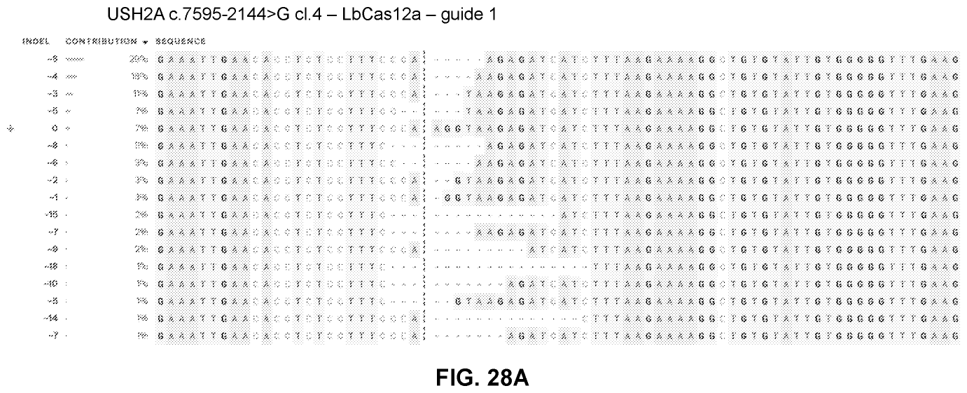

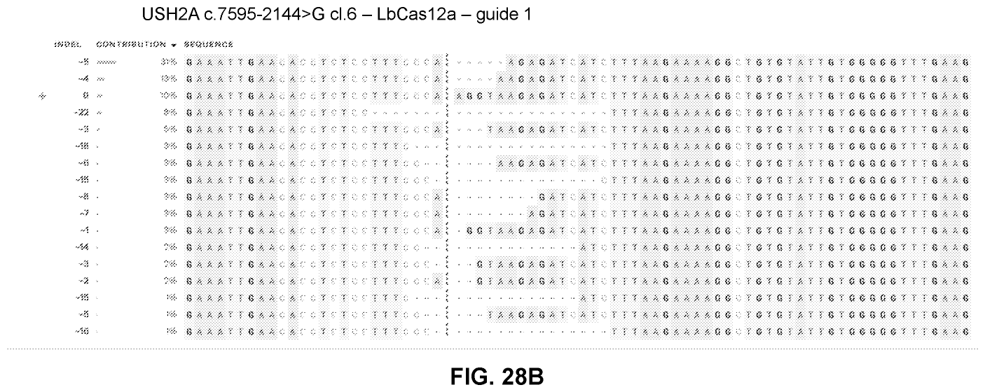

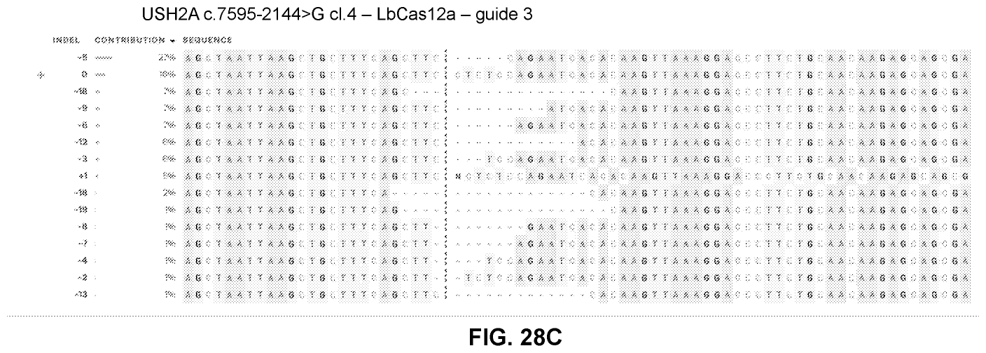

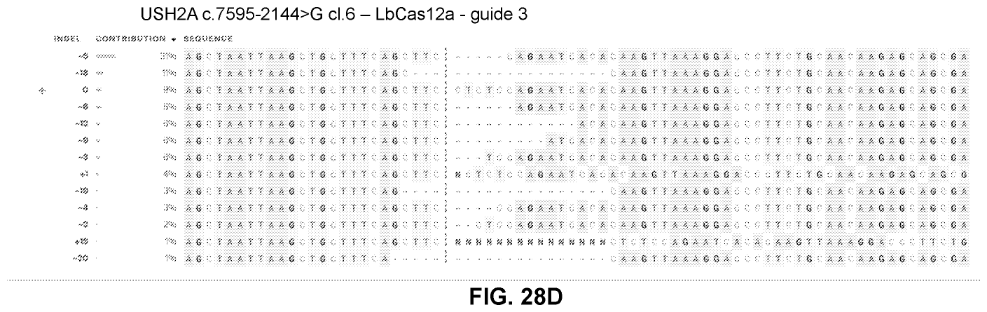

The levels of indel formation on both the wild-type and mutated minigenes generated by the different LbCas12a-gRNA combinations were also evaluated in order to assess their allele-specificity. Genomic DNA extracted from the same samples employed for transcript evaluation was PCR amplified, Sanger sequenced and analyzed using the TIDE web tool (FIG. 27C). For all tested gRNAs appreciable indel formation on the mutated USH2A minigene was measured (approx. 80%, see FIG. 27C), showing good consistency among the two different tested clones (clone 4 and clone 6). Indel formation was then evaluated after transduction of the HEK293 clone 1, stably expressing the wild-type USH2A minigene. As expected, Guide 3 did not show any allelic specificity since the target domain of this gRNA is not positioned on the c.7595-2144A>G mutation and therefore its target is present both in wild-type and mutated minigenes (FIG. 27C). On the other hand, Guide 1, which is targeting the c.7595-2144A>G mutation, was indeed able to produce indels on the mutated minigene in clones 4 and 6, while background levels of editing were detected in clone 1 expressing the wild-type USH2A construct (FIG. 27C). Furthermore, the indel profiles generated by guide 1 and guide 3 in c.7595-2144A>G USH2A clones 4 and 6 were analyzed using the Synthego ICE webtool, revealing a wide range of deletions ranging from −1 nt up to −22nt (FIGS. 28A-28D). Interestingly, guide 3 contrary to guide 1 is also producing insertions, despite at low frequency. In addition, there was good consistency among the two clones with respect to the detected indels, even though their relative frequency was not always conserved among the two cell lines.

### 7. SPECIFIC EMBODIMENTS

The present disclosure is exemplified by the specific embodiments below.
1. A Cas12a guide RNA (gRNA) molecule comprising:
   (a) a protospacer domain containing a targeting sequence; and
   (b) a loop domain;
wherein
   (i) the targeting sequence corresponds to a target domain in a genomic DNA sequence;
   (ii) the target domain is adjacent to a protospacer-adjacent motif (PAM) of a Cas12a protein; and
   (iii) upon introduction of the gRNA and the Cas12a protein into a cell containing the genomic sequence, the Cas12a cleaves the genomic DNA up to 15 nucleotides from a splice site encoded by the genomic DNA.
2. The Cas12a gRNA of embodiment 1, wherein the cell is a eukaryotic cell.
3. The Cas12a gRNA of embodiment 2, wherein the cell is a mammalian cell.
4. The Cas12a gRNA of embodiment 3, wherein the cell is a human cell.
5. A Cas12a guide RNA (gRNA) molecule comprising:
   (a) a protospacer domain containing a targeting sequence; and
   (b) a loop domain;
wherein
   (i) the targeting sequence corresponds to a target domain in a genomic DNA sequence;
   (ii) the target domain is adjacent to a protospacer-adjacent motif (PAM) of a Cas12a protein; and
   (iii) the PAM is within 40 nucleotides of a splice site encoded by the genomic DNA.
6. The Cas12a gRNA of embodiment 5, wherein the PAM is within 4 to 38 nucleotides of the splice site.
7. The Cas12a gRNA of embodiment 5 or embodiment 6, wherein upon introduction of the gRNA and the Cas12a protein into a cell containing the genomic sequence, the Cas12a cleaves the genomic DNA up to 15 nucleotides from the splice site.
8. The Cas12a gRNA of embodiment 7, wherein the cell is a eukaryotic cell.
9. The Cas12a gRNA of embodiment 8, wherein the cell is a mammalian cell.
10. The Cas12a gRNA of embodiment 9, wherein the cell is a human cell.
11. The Cas12a gRNA molecule of any one of embodiments 1 to 10, wherein upon introduction of the gRNA and the Cas12a protein into a cell containing the genomic sequence, the Cas12a cleaves the genomic DNA up to 10 nucleotides from the splice site.
12. The Cas12a gRNA molecule of any one of embodiments 1 to 10, wherein upon introduction of the gRNA and the Cas12a protein into a cell containing the genomic sequence, the Cas12a cleaves the genomic DNA 10-15 nucleotides from the splice site.
13. The Cas12a gRNA molecule of any one of embodiments 1 to 12, wherein the splice site is a cryptic splice site.
14. The Cas12a gRNA molecule of embodiment 13, wherein the cryptic splice site is created by a mutation in the genomic DNA sequence.
15. The Cas12a gRNA molecule of embodiment 13, wherein the cryptic splice site is activated by a mutation in the genomic DNA sequence.
16. The Cas12a gRNA of embodiment 14 or embodiment 15, wherein the mutation is located 1 to 23 nucleotides 3′ of the PAM sequence.
17. The Cas12a gRNA of any one of embodiments 14 to 16, wherein the mutation is a single nucleotide polymorphism.
18. The Cas12a gRNA of any one of embodiments 14 to 16, wherein the mutation is a deletion.
19. The Cas12a gRNA of embodiment 18, wherein the deletion is a deletion of 1 to 10⁶ nucleotides.
20. The Cas12a gRNA of embodiment 18, wherein the deletion is a deletion of 1 to 10⁵ nucleotides.
21. The Cas12a gRNA of embodiment 18, wherein the deletion is a deletion of 1 to 10⁴ nucleotides.
22. The Cas12a gRNA of embodiment 18, wherein the deletion is a deletion of 1 to 10³ nucleotides.
23. The Cas12a gRNA of embodiment 18, wherein the deletion is a deletion of 1 to 100 nucleotides.
24. The Cas12a gRNA of embodiment 18, wherein the deletion is a deletion of 1 to 10 nucleotides.
25. The Cas12a gRNA of any one of embodiments 14 to 16, wherein the mutation is an insertion.
26. The Cas 12 gRNA of embodiment 25, wherein the insertion is an insertion of 1 to 10⁶ nucleotides.
27. The Cas 12 gRNA of embodiment 25, wherein the insertion is an insertion of 1 to 10⁶ nucleotides.
28. The Cas 12 gRNA of embodiment 25, wherein the insertion is an insertion of 1 to 10⁴ nucleotides.
29. The Cas 12 gRNA of embodiment 25, wherein the insertion is an insertion of 1 to 10³ nucleotides.
30. The Cas 12 gRNA of embodiment 25, wherein the insertion is an insertion of 1 to 100 nucleotides.
31. The Cas 12 gRNA of embodiment 25, wherein the insertion is an insertion of 1 to 10 nucleotides.
32. The Cas12a gRNA of any one of embodiments 14 to 31, wherein upon introduction of the gRNA and the Cas12a protein into population of cells containing the genomic sequence in vitro, cleavage of the genomic DNA by the Cas12a protein deletes the mutation in 10% to 50% of the resulting indels.
33. The Cas12a gRNA of any one of embodiments 14 to 31, wherein upon introduction of the gRNA and the Cas12a protein into a population cells containing the genomic sequence in vitro, cleavage of the genomic DNA by the Cas12a protein deletes the mutation in 10% to 40% of the resulting indels.
34. The Cas12a gRNA of any one of embodiments 14 to 31, wherein upon introduction of the gRNA and the Cas12a protein into a population of cells containing the genomic sequence in vitro, cleavage of the genomic DNA by the Cas12a protein deletes the mutation in 10% to 30% of the resulting indels.
35. The Cas12a gRNA of any one of embodiments 14 to 31, wherein upon introduction of the gRNA and the Cas12a protein into a population of cells containing the genomic sequence in vitro, cleavage of the genomic DNA by the Cas12a protein deletes the mutation in 10% to 20% of the resulting indels.
36. The Cas12a gRNA of any one of embodiments 13 to 35, wherein splicing at the cryptic splice site results in a disease phenotype.
37. The Cas12a gRNA of any one of embodiments 13 to 36, which when introduced with the Cas12a protein into a cell having the genomic DNA sequence, aberrant splicing caused by the cryptic splice site is corrected.
38. The Cas12a gRNA of any one of embodiments 13 to 36, which when introduced with the Cas12a protein into a population of cells having the genomic DNA sequence in vitro, normal splicing is restored in at least 10% of the cells.
39. The Cas12a gRNA of embodiment 38, which when introduced with the Cas12a protein into a population of cells having the genomic DNA sequence in vitro, normal splicing is restored in 10% to 20% of the cells.
40. The Cas12a gRNA of any one of embodiments 13 to 36, which when introduced with the Cas12a protein into a population of cells having the genomic DNA sequence in vitro, normal splicing is restored in at least 20% of the cells.
41. The Cas12a gRNA of embodiment 40, which when introduced with the Cas12a protein into a population of cells having the genomic DNA sequence in vitro, normal splicing is restored in 20% to 30% of the cells.
42. The Cas12a gRNA of any one of embodiments 13 to 36, which when introduced with the Cas12a protein into a population of cells having the genomic DNA sequence in vitro, normal splicing is restored in at least 30% of the cells.
43. The Cas12a gRNA of embodiment 42, which when introduced with the Cas12a protein into a population of cells having the genomic DNA sequence in vitro, normal splicing is restored in 30% to 40% of the cells.
44. The Cas12a gRNA of any one of embodiments 13 to 36, which when introduced with the Cas12a protein into a population of cells having the genomic DNA sequence in vitro, normal splicing is restored in at least 40% of the cells.
45. The Cas12a gRNA of embodiment 44, which when introduced with the Cas12a protein into a population of cells having the genomic DNA sequence in vitro, normal splicing is restored in 40% to 50% of the cells.
46. The Cas12a gRNA of any one of embodiments 13 to 36, which when introduced with the Cas12a protein into a population of cells having the genomic DNA sequence in vitro, normal splicing is restored in at least 50% of the cells.
47. The Cas12a gRNA of embodiment 46, which when introduced with the Cas12a protein into a population of cells having the genomic DNA sequence in vitro, normal splicing is restored in 50% to 60% of the cells.
48. The Cas12a gRNA of any one of embodiments 13 to 36, which when introduced with the Cas12a protein into a population of cells having the genomic DNA sequence in vitro, normal splicing is restored in at least 60% of the cells.
49. The Cas12a gRNA of embodiment 48, which when introduced with the Cas12a protein into a population of cells having the genomic DNA sequence in vitro, normal splicing is restored in 60% to 70% of the cells.
50. The Cas12a gRNA of any one of embodiments 13 to 36, which when introduced with the Cas12a protein into a population of cells having the genomic DNA sequence in vitro, normal splicing is restored in at least 70% of the cells.
51. The Cas12a gRNA of embodiment 50, which when introduced with the Cas12a protein into a population of cells having the genomic DNA sequence in vitro, normal splicing is restored in 70% to 80% of the cells.
52. The Cas12a gRNA of embodiment 50, which when introduced with the Cas12a protein into a population of cells having the genomic DNA sequence in vitro, normal splicing is restored in 70% to 90% of the cells.
53. The Cas12a gRNA molecule of any one of embodiments 13 to 52, wherein the cryptic splice site is a cryptic 3′ splice site.
54. The Cas12a gRNA molecule of embodiment 53, wherein upon introduction of the gRNA and the Cas12a protein into a cell containing the genomic sequence, activity of the cryptic 3′ splice site is reduced.
55. The Cas12a gRNA molecule of embodiment 54, wherein upon introduction of the gRNA and the Cas12a protein into a cell containing the genomic sequence, the branch site of the cryptic 3′ splice site is disrupted.
56. The Cas12a gRNA molecule of embodiment 54, wherein upon introduction of the gRNA and the Cas12a protein into a cell containing the genomic sequence, the polypyrimidine tract of the cryptic 3′ splice site is disrupted.
57. The Cas12a gRNA molecule of embodiment 54, wherein upon introduction of the gRNA and the Cas12a protein into a cell containing the genomic sequence, the intron/exon junction of the cryptic 3′ splice site is disrupted.
58. The Cas12a gRNA of any one of embodiments 53 to 57, wherein the cryptic 3′ splice site is upstream of a 3′ canonical splice site.
59. The Cas12a gRNA of any one of embodiments embodiment 53 to 58, wherein the cryptic 3′ splice site is upstream of a 5′ cryptic splice site.
60. The Cas12a gRNA molecule of any one of embodiments 13 to 52, wherein the cryptic splice site is a cryptic 5′ splice site.
61. The Cas12a gRNA molecule of embodiment 60, wherein upon introduction of the gRNA and the Cas12a protein into a cell containing the genomic sequence, activity of the cryptic 5′ splice site is reduced.
62. The Cas12a gRNA molecule of embodiment 61, wherein upon introduction of the gRNA and the Cas12a protein into a cell containing the genomic sequence, the intron/exon junction of the cryptic 5′ splice site is disrupted.
63. The Cas12a gRNA of any one of embodiments 60 to 62, wherein the cryptic 5′ splice site is downstream of a 3′ cryptic splice site.
64. The Cas12a gRNA of any one of embodiments 60 to 62, wherein the cryptic 5′ splice site is downstream of a 5′ canonical splice site.
65. The Cas12a gRNA of any one of embodiments 1 to 12, wherein the splice site is a canonical splice site.
66. The Cas12a gRNA of embodiment 65, wherein the canonical splice site is a canonical 3′ splice site.
67. The Cas12a gRNA molecule of embodiment 66, wherein upon introduction of the gRNA and the Cas12a protein into a cell containing the genomic sequence, activity of the canonical 3′ splice site is disrupted.
68. The Cas12a gRNA molecule of embodiment 67, wherein upon introduction of the gRNA and the Cas12a protein into a cell containing the genomic sequence, the branch site of the canonical 3′ splice site is disrupted.
69. The Cas12a gRNA molecule of embodiment 67, wherein upon introduction of the gRNA and the Cas12a protein into a cell containing the genomic sequence, the polypyrimidine tract of the canonical 3′ splice site is disrupted.
70. The Cas12a gRNA molecule of embodiment 67, wherein upon introduction of the gRNA and the Cas12a protein into a cell containing the genomic sequence, the intron/exon junction of the canonical 3′ splice site is disrupted.
71. The Cas12a gRNA of embodiment 65, wherein the canonical splice site is a canonical 5′ splice site.
72. The Cas12a gRNA molecule of embodiment 71, wherein upon introduction of the gRNA and the Cas12a protein into a cell containing the genomic sequence, activity of the canonical 5′ splice site is reduced.
73. The Cas12a gRNA molecule of embodiment 71, wherein upon introduction of the gRNA and the Cas12a protein into a cell containing the genomic sequence, the intron/exon junction of the canonical 5′ splice site is disrupted.
74. The Cas12a gRNA of any one of embodiments 1 to 73, which is 40-44 nucleotides long.
75. The Cas12a gRNA of any one of embodiments 1 to 74, wherein the targeting sequence is 20-24 nucleotides in length.
76. The Cas12a gRNA of any one of embodiments 1 to 75, wherein the protospacer domain is 17-26 nucleotides in length.
77. The Cas12a gRNA of any one of embodiments 1 to 75, wherein the protospacer domain is 20-24 nucleotides in length.
78. The Cas12a gRNA of any one of embodiments 1 to 77, wherein the targeting sequence is 23 nucleotides in length.
79. The Cas12a gRNA of any one of embodiments 1 to 78, wherein there are no mismatches between the targeting sequence and the complement of the target domain.
80. The Cas12a gRNA of any one of embodiments 1 to 79, wherein the protospacer domain is 23 nucleotides in length.
81. The Cas12a gRNA of any one of embodiments 1 to 80, wherein the PAM sequence is TTTV, where V is A, C, or G.
82. The Cas12a gRNA of any one of embodiments 1 to 80, wherein the PAM sequence is TYCV, where Y is C or T and V is A, C, or G.
83. The Cas12a gRNA of any one of embodiments 1 to 80, wherein the PAM sequence is CCCC.
84. The Cas12a gRNA of any one of embodiments 1 to 80, wherein the PAM sequence is ACCC.
85. The Cas12a gRNA of any one of embodiments 1 to 80, wherein the PAM sequence is TATV, where V is A, C, or G.
86. The Cas12a gRNA of any one of embodiments 1 to 80, wherein the PAM sequence is RATR.
87. The Cas12a gRNA of any one of embodiments 1 to 80, wherein the PAM sequence is NTTN, where N is any nucleotide.
88. The Cas12a gRNA of any one of embodiments 1 to 80, wherein the PAM sequence is TCTN, where N is any nucleotide.
89. The Cas12a gRNA of any one of embodiments 1 to 80, wherein the PAM sequence is TTTN, where N is any nucleotide.
90. The Cas12a gRNA of any one of embodiments 1 to 80, wherein the PAM sequence is TTN, where N is any nucleotide.
91. The Cas12a gRNA of any one of embodiments 1 to 80, wherein the PAM sequence is YYN, where Y is C or T and N is any nucleotide.
92. The Cas12a gRNA of any one of embodiments 1 to 80, wherein the PAM sequence is YTN, wherein Y is C or T and N is any nucleotide.
93. The Cas12a gRNA of any one of embodiments 1 to 80, wherein the PAM sequence is TYYN, where Y is C or T and N is any nucleotide.
94. The Cas12a gRNA of any one of embodiments 1 to 93, wherein the genomic sequence is a eukaryotic genomic sequence.
95. The Cas12a gRNA of embodiment 94, wherein the eukaryotic genomic sequence is a mammalian genomic sequence.
96. The Cas12a gRNA of embodiment 95, wherein the mammalian genomic sequence is a human genomic sequence.
97. The Cas12a gRNA of embodiment 96, wherein the target domain is in a human genomic sequence which is a CFTR gene, a DMD gene, a HBB gene, a FGB gene, a SOD1 gene, a QDPR gene, a GLA gene, a LDLR gene, a BRIP1 gene, a F9 gene, a CEP290 gene, a COL2A1 gene, a USH2A gene, or a GAA gene.
98. The Cas12a gRNA of embodiment 96, wherein the target domain is in a human genomic sequence which is a CFTR gene, a DMD gene, a FGB gene, a SOD1 gene, a QDPR gene, a GLA gene, a LDLR gene, a BRIP1 gene, a F9 gene, a CEP290 gene, a COL2A1 gene, a USH2A gene, or a GAA gene.
99. The Cas12a gRNA of any one of embodiments 1 to 96, wherein the target domain is not in a human HBB gene.
100. The Cas12a gRNA of any one of embodiments 1 to 99, wherein the target domain comprises or consists of a nucleotide sequence other than GGTAATAGCAATATTTCTGCATA (SEQ ID NO: 293).
101. The Cas12a gRNA of any one of embodiments 1 to 10, wherein the target domain is in a human genomic sequence which is a CFTR gene, a DMD gene, a HBB gene, a FGB gene, a SOD1 gene, a QDPR gene, a GLA gene, a LDLR gene, a BRIP1 gene, a F9 gene, a CEP290 gene, a COL2A1 gene, a USH2A gene, or a GAA gene.
102. The Cas12a gRNA of 101, wherein the target domain is in a CFTR gene.
103. The Cas12a gRNA of embodiment 102, wherein the CFTR gene has a mutation which is a 3272-26A>G mutation, a 3849+10kbC>T mutation, a IVS11+194A>G mutation, or a IVS19+11505C>G mutation.
104. The Cas12a gRNA of embodiment 103, wherein the mutation is a 3272-26A>G mutation.
105. The Cas12a gRNA of embodiment 104, wherein the target domain has the nucleotide sequence CATAGAAAACACTGCAAATAACA (SEQ ID NO: 38).
106. A Cas12a guide RNA (gRNA) molecule comprising a protospacer domain containing a targeting sequence and a loop domain, wherein the targeting sequence corresponds to a target domain having the nucleotide sequence CATAGAAAACACTGCAAATAACA (SEQ ID NO: 38).
107. The Cas12a gRNA of embodiment 103, wherein the mutation is a 3849+10kbC>T mutation.
108. The Cas12a gRNA of embodiment 107, wherein the target domain has the nucleotide sequence AGGGTGTCTTACTCACCATTTTA (SEQ ID NO: 39).
109. A Cas12a guide RNA (gRNA) molecule comprising a protospacer domain containing a targeting sequence and a loop domain, wherein the targeting sequence corresponds to a target domain having the nucleotide sequence AGGGTGTCTTACTCACCATTTTA (SEQ ID NO: 39).
110. The Cas12a gRNA of embodiment 103, wherein the mutation is a IVS11+194A>G mutation.
111. The Cas12a gRNA of embodiment 110, wherein the target domain has the nucleotide sequence TACTTGAGATGTAAGTAAGGTTA (SEQ ID NO: 40).
112. A Cas12a guide RNA (gRNA) molecule comprising a protospacer domain containing a targeting sequence and a loop domain, wherein the targeting sequence corresponds to a target domain having the nucleotide sequence TACTTGAGATGTAAGTAAGGTTA (SEQ ID NO: 40).
113. The Cas12a gRNA of embodiment 110, wherein the target domain has the nucleotide sequence ATAGTAACCTTACTTACATCTCA (SEQ ID NO: 41).
114. A Cas12a guide RNA (gRNA) molecule comprising a protospacer domain containing a targeting sequence and a loop domain, wherein the targeting sequence corresponds to a target domain having the nucleotide sequence ATAGTAACCTTACTTACATCTCA (SEQ ID NO: 41).
115. The Cas12a gRNA of embodiment 103, wherein the mutation is a IVS19+11505C>G mutation.
116. The Cas12a gRNA of embodiment 115, wherein the target domain has the nucleotide sequence AAATTCCATCTTACCAATTCTAA (SEQ ID NO: 42).
117. A Cas12a guide RNA (gRNA) molecule comprising a protospacer domain containing a targeting sequence and a loop domain, wherein the targeting sequence corresponds to a target domain having the nucleotide sequence AAATTCCATCTTACCAATTCTAA (SEQ ID NO: 42).
118. The Cas12a gRNA of embodiment 115, wherein the target domain has the nucleotide sequence AACGTTAAAATTCCATCTTACCA (SEQ ID NO: 43).
119. A Cas12a guide RNA (gRNA) molecule comprising a protospacer domain containing a targeting sequence and a loop domain, wherein the targeting sequence corresponds to a target domain having the nucleotide sequence AACGTTAAAATTCCATCTTACCA (SEQ ID NO: 43).
120. The Cas12a gRNA of embodiment 101, wherein the target domain is in a DMD gene.
121. The Cas12a gRNA of embodiment 120, wherein the DMD gene has a mutation which is a IVS9+46806C>T mutation, a IVS62+62296A>G mutation, a IVS1+36947G>A mutation, a IVS1+36846G>A mutation, a IVS1+36846G>A mutation, a IVS2+5591T>A mutation or a IVS8-15A>G mutation.
122. The Cas12a gRNA of embodiment 121, wherein the mutation is a IVS9+46806C>T mutation.
123. The Cas12a gRNA of embodiment 122, wherein the target domain has the nucleotide sequence TGACCTTTGGTAAGTCATCTAAT (SEQ ID NO: 44).
124. A Cas12a guide RNA (gRNA) molecule comprising a protospacer domain containing a targeting sequence and a loop domain, wherein the targeting sequence corresponds to a target domain having the nucleotide sequence TGACCTTTGGTAAGTCATCTAAT (SEQ ID NO: 44).
125. The Cas12a gRNA of embodiment 122, wherein the target domain has the nucleotide sequence CCTTTGTGACCTTTGGTAAGTCA (SEQ ID NO: 45).
126. A Cas12a guide RNA (gRNA) molecule comprising a protospacer domain containing a targeting sequence and a loop domain, wherein the targeting sequence corresponds to a target domain having the nucleotide sequence CCTTTGTGACCTTTGGTAAGTCA (SEQ ID NO: 45).
127. The Cas12a gRNA of embodiment 121, wherein the mutation is a IVS62+62296A>G mutation.
128. The Cas12a gRNA of embodiment 127, wherein the target domain has the nucleotide sequence TTGATCACATAACAAGGTCAGTT (SEQ ID NO: 46).
129. A Cas12a guide RNA (gRNA) molecule comprising a protospacer domain containing a targeting sequence and a loop domain, wherein the targeting sequence corresponds to a target domain having the nucleotide sequence TTGATCACATAACAAGGTCAGTT (SEQ ID NO: 46).
130. The Cas12a gRNA of embodiment 127, wherein the target domain has the nucleotide sequence ATCACATAACAAGGTCAGTTTAT (SEQ ID NO: 47).
131. A Cas12a guide RNA (gRNA) molecule comprising a protospacer domain containing a targeting sequence and a loop domain, wherein the targeting sequence corresponds to a target domain having the nucleotide sequence ATCACATAACAAGGTCAGTTTAT (SEQ ID NO: 47).
132. The Cas12a gRNA of embodiment 127, which has the nucleotide sequence AGTTATGATAAACTGACCTTGTT (SEQ ID NO: 48).
133. A Cas12a guide RNA (gRNA) molecule comprising a protospacer domain containing a targeting sequence and a loop domain, wherein the targeting sequence corresponds to a target domain having the nucleotide sequence AGTTATGATAAACTGACCTTGTT (SEQ ID NO: 48).
134. The Cas12a gRNA of embodiment 127, which has the nucleotide sequence TGATAAACTGACCTTGTTATGTG (SEQ ID NO: 49).
135. A Cas12a guide RNA (gRNA) molecule comprising a protospacer domain containing a targeting sequence and a loop domain, wherein the targeting sequence corresponds to a target domain having the nucleotide sequence TGATAAACTGACCTTGTTATGTG (SEQ ID NO: 49).
136. The Cas12a gRNA of embodiment 121, wherein the mutation is a IVS1+36947G>A mutation.
137. The Cas12a gRNA of embodiment 136, wherein the target domain has the nucleotide sequence TCTTCCTTGGTTTTGCAGCTTCT (SEQ ID NO: 50).
138. A Cas12a guide RNA (gRNA) molecule comprising a protospacer domain containing a targeting sequence and a loop domain, wherein the targeting sequence corresponds to a target domain having the nucleotide sequence TCTTCCTTGGTTTTGCAGCTTCT (SEQ ID NO: 50).
139. The Cas12a gRNA of embodiment 136, wherein the target domain has the nucleotide sequence TTGGTTTTGCAGCTTCTCGAGTT (SEQ ID NO: 51).
140. A Cas12a guide RNA (gRNA) molecule comprising a protospacer domain containing a targeting sequence and a loop domain, wherein the targeting sequence corresponds to a target domain having the nucleotide sequence TTGGTTTTGCAGCTTCTCGAGTT (SEQ ID NO: 51).
141. The Cas12a gRNA of embodiment 136, wherein the target domain has the nucleotide sequence CTCTTTCTCTTCCTTGGTTTTGC (SEQ ID NO: 52).
142. A Cas12a guide RNA (gRNA) molecule comprising a protospacer domain containing a targeting sequence and a loop domain, wherein the targeting sequence corresponds to a target domain having the nucleotide sequence CTCTTTCTCTTCCTTGGTTTTGC (SEQ ID NO: 52).
143. The Cas12a gRNA of embodiment 121, wherein the mutation is a IVS2+5591T>A mutation.
144. The Cas12a gRNA of embodiment 143, wherein the target domain has the nucleotide sequence CTTGTTTCTCTACATAGGTTGAA (SEQ ID NO: 53).
145. A Cas12a guide RNA (gRNA) molecule comprising a protospacer domain containing a targeting sequence and a loop domain, wherein the targeting sequence corresponds to a target domain having the nucleotide sequence CTTGTTTCTCTACATAGGTTGAA (SEQ ID NO: 53).
146. The Cas12a gRNA of embodiment 121, wherein the mutation is a IVS8-15A>G mutation.
147. The Cas12a gRNA of embodiment 146, wherein the target domain has the nucleotide sequence TCCTCTCTATCCACCTCCCCCAG (SEQ ID NO: 54).
148. A Cas12a guide RNA (gRNA) molecule comprising a protospacer domain containing a targeting sequence and a loop domain, wherein the targeting sequence corresponds to a target domain having the nucleotide sequence TCCTCTCTATCCACCTCCCCCAG (SEQ ID NO: 54).
149. The Cas12a gRNA of embodiment 146, wherein the target domain has the nucleotide sequence CCTCCCCCAGACCCTTCTCTGCA (SEQ ID NO: 55).
150. A Cas12a guide RNA (gRNA) molecule comprising a protospacer domain containing a targeting sequence and a loop domain, wherein the targeting sequence corresponds to a target domain having the nucleotide sequence CCTCCCCCAGACCCTTCTCTGCA (SEQ ID NO: 55).
151. The Cas12a gRNA of embodiment 146, wherein the target domain has the nucleotide sequence CCCCTCCTCTCTATCCACTCCCC (SEQ ID NO: 56).
152. A Cas12a guide RNA (gRNA) molecule comprising a protospacer domain containing a targeting sequence and a loop domain, wherein the targeting sequence corresponds to a target domain having the nucleotide sequence CCCCTCCTCTCTATCCACTCCCC (SEQ ID NO: 56).
153. The Cas12a gRNA of embodiment 146, wherein the target domain has the nucleotide sequence CCTCCTCTCTATCCACCTCCCCC (SEQ ID NO: 57).
154. A Cas12a guide RNA (gRNA) molecule comprising a protospacer domain containing a targeting sequence and a loop domain, wherein the targeting sequence corresponds to a target domain having the nucleotide sequence CCTCCTCTCTATCCACCTCCCCC (SEQ ID NO: 57).
155. The Cas12a gRNA of embodiment 120, wherein the target domain is in intron 50 and/or exon 51 of DMD.
156. The Cas12a gRNA of embodiment 155, wherein the target domain has the nucleotide sequence CAAAAACCCAAAATATTTTAGCT (SEQ ID NO: 58).
157. A Cas12a guide RNA (gRNA) molecule comprising a protospacer domain containing a targeting sequence and a loop domain, wherein the targeting sequence corresponds to a target domain having the nucleotide sequence CAAAAACCCAAAATATTTTAGCT (SEQ ID NO: 58).
158. The Cas12a gRNA of embodiment 155, wherein the target domain has the nucleotide sequence CTTTTTGCAAAAACCCAAAATAT (SEQ ID NO: 59).
159. A Cas12a guide RNA (gRNA) molecule comprising a protospacer domain containing a targeting sequence and a loop domain, wherein the targeting sequence corresponds to a target domain having the nucleotide sequence CTTTTTGCAAAAACCCAAAATAT (SEQ ID NO: 59).
160. The Cas12a gRNA of embodiment 155, wherein the target domain has the nucleotide sequence TTTTTGCAAAAACCCAAAATATT (SEQ ID NO: 60).
161. A Cas12a guide RNA (gRNA) molecule comprising a protospacer domain containing a targeting sequence and a loop domain, wherein the targeting sequence corresponds to a target domain having the nucleotide sequence TTTTTGCAAAAACCCAAAATATT (SEQ ID NO: 60).
162. The Cas12a gRNA of embodiment 155, wherein the target domain has the nucleotide sequence TGTCACCAGAGTAACAGTCTGAG (SEQ ID NO: 61).
163. A Cas12a guide RNA (gRNA) molecule comprising a protospacer domain containing a targeting sequence and a loop domain, wherein the targeting sequence corresponds to a target domain having the nucleotide sequence TGTCACCAGAGTAACAGTCTGAG (SEQ ID NO: 61).
164. The Cas12a gRNA of embodiment 155, wherein the target domain has the nucleotide sequence GCTCCTACTCAGACTGTTACTCT (SEQ ID NO: 62).
165. A Cas12a guide RNA (gRNA) molecule comprising a protospacer domain containing a targeting sequence and a loop domain, wherein the targeting sequence corresponds to a target domain having the nucleotide sequence GCTCCTACTCAGACTGTTACTCT (SEQ ID NO: 62).
166. The Cas12a gRNA of embodiment 101, wherein the target domain is in a HBB gene.
167. The Cas12a gRNA of embodiment 166, wherein the HBB gene has a mutation which is a IVS2+645C>T, IVS2+705T>G, or IVS2+745C>G mutation.
168. The Cas12a gRNA of embodiment 167, wherein the mutation is a IVS2+645C>T mutation.
169. The Cas12a gRNA of any one of embodiments 166 to 168, wherein the target domain comprises or consists of a nucleotide sequence other than GGTAATAGCAATATTTCTGCATA (SEQ ID NO: 293).
170. The Cas12a gRNA of embodiment 168, wherein the target domain has the nucleotide sequence TGGGTTAAGGTAATAGCAATATC (SEQ ID NO: 63).
171. A Cas12a guide RNA (gRNA) molecule comprising a protospacer domain containing a targeting sequence and a loop domain, wherein the targeting sequence corresponds to a target domain having the nucleotide sequence TGGGTTAAGGTAATAGCAATATC (SEQ ID NO: 63).
172. The Cas12a gRNA of embodiment 168, wherein the target domain has the nucleotide sequence TATGCAGAGATATTGCTATTACC (SEQ ID NO: 64).
173. A Cas12a guide RNA (gRNA) molecule comprising a protospacer domain containing a targeting sequence and a loop domain, wherein the targeting sequence corresponds to a target domain having the nucleotide sequence TATGCAGAGATATTGCTATTACC (SEQ ID NO: 64).
174. The Cas12a gRNA of embodiment 168, wherein the target domain has the nucleotide sequence CTATTACCTTAACCCAGAAATTA (SEQ ID NO: 65).
175. A Cas12a guide RNA (gRNA) molecule comprising a protospacer domain containing a targeting sequence and a loop domain, wherein the targeting sequence corresponds to a target domain having the nucleotide sequence CTATTACCTTAACCCAGAAATTA (SEQ ID NO: 65).
176. The Cas12a gRNA of embodiment 168, wherein the target domain has the nucleotide sequence CAGAGATATTGCTATTACCTTAA (SEQ ID NO: 66).
177. A Cas12a guide RNA (gRNA) molecule comprising a protospacer domain containing a targeting sequence and a loop domain, wherein the targeting sequence corresponds to a target domain having the nucleotide sequence CAGAGATATTGCTATTACCTTAA (SEQ ID NO: 66).
178. The Cas12a gRNA of embodiment 167, wherein the mutation is a IVS2+705T>G mutation.
179. The Cas12a gRNA of embodiment 178, wherein the target domain has the nucleotide sequence TGCATATAAATTGTAACTGAGGT (SEQ ID NO: 67).
180. A Cas12a guide RNA (gRNA) molecule comprising a protospacer domain containing a targeting sequence and a loop domain, wherein the targeting sequence corresponds to a target domain having the nucleotide sequence TGCATATAAATTGTAACTGAGGT (SEQ ID NO: 67).
181. The Cas12a gRNA of embodiment 178, wherein the target domain has the nucleotide sequence AATTGTAACTGAGGTAAGAGGTT (SEQ ID NO: 68).
182. A Cas12a guide RNA (gRNA) molecule comprising a protospacer domain containing a targeting sequence and a loop domain, wherein the targeting sequence corresponds to a target domain having the nucleotide sequence AATTGTAACTGAGGTAAGAGGTT (SEQ ID NO: 68).
183. The Cas12a gRNA of embodiment 178, wherein the target domain has the nucleotide sequence AAACCTCTTACCTCAGTTACAAT (SEQ ID NO: 69).
184. A Cas12a guide RNA (gRNA) molecule comprising a protospacer domain containing a targeting sequence and a loop domain, wherein the targeting sequence corresponds to a target domain having the nucleotide sequence AAACCTCTTACCTCAGTTACAAT (SEQ ID NO: 69).
185. The Cas12a gRNA of embodiment 178, wherein the target domain has the nucleotide sequence GCAATATGAAACCTCTTACCTCA (SEQ ID NO: 70).
186. A Cas12a guide RNA (gRNA) molecule comprising a protospacer domain containing a targeting sequence and a loop domain, wherein the targeting sequence corresponds to a target domain having the nucleotide sequence GCAATATGAAACCTCTTACCTCA (SEQ ID NO: 70).
187. The Cas12a gRNA of embodiment 167, wherein the mutation is a IVS2+745C>G mutation.
188. The Cas12a gRNA of embodiment 187, wherein the target domain has the nucleotide sequence CTAATAGCAGCTACAATCCAGGT (SEQ ID NO: 71).
189. A Cas12a guide RNA (gRNA) molecule comprising a protospacer domain containing a targeting sequence and a loop domain, wherein the targeting sequence corresponds to a target domain having the nucleotide sequence CTAATAGCAGCTACAATCCAGGT (SEQ ID NO: 71).
190. The Cas12a gRNA of embodiment 101, wherein the target domain is in a FGB gene.
191. The Cas12a gRNA of embodiment 190, wherein the FGB gene has a IVS6+13C>T mutation.
192. The Cas12a gRNA of embodiment 191, wherein the target domain has the nucleotide sequence TTTTGCATACCTGTTCGTTACCT (SEQ ID NO: 72).
193. A Cas12a guide RNA (gRNA) molecule comprising a protospacer domain containing a targeting sequence and a loop domain, wherein the targeting sequence corresponds to a target domain having the nucleotide sequence TTTTGCATACCTGTTCGTTACCT (SEQ ID NO: 72).
194. The Cas12a gRNA of embodiment 191, wherein the target domain has the nucleotide sequence AAATAGAATGATTTTATTTTGCA (SEQ ID NO: 73).
195. A Cas12a guide RNA (gRNA) molecule comprising a protospacer domain containing a targeting sequence and a loop domain, wherein the targeting sequence corresponds to a target domain having the nucleotide sequence AAATAGAATGATTTTATTTTGCA (SEQ ID NO: 73).
196. The Cas12a gRNA of embodiment 101, wherein the target domain is in a SOD1 gene.
197. The Cas12a gRNA of embodiment 196, wherein the SOD1 gene has a IVS4+792C>G mutation.
198. The Cas12a gRNA of embodiment 197, wherein the target domain has the nucleotide sequence TGGTAAGTTACACTAACCTTAGT (SEQ ID NO: 74).
199. A Cas12a guide RNA (gRNA) molecule comprising a protospacer domain containing a targeting sequence and a loop domain, wherein the targeting sequence corresponds to a target domain having the nucleotide sequence TGGTAAGTTACACTAACCTTAGT (SEQ ID NO: 74).
200. The Cas12a gRNA of embodiment 101, wherein the target domain is in a QDPR gene.
201. The Cas12a gRNA of embodiment 200, wherein the mutation is a IVS3+2552A>G mutation.
202. The Cas12a gRNA of embodiment 201, wherein the target domain has the nucleotide sequence TCATCTGTAAAATAAGAGTAAAA (SEQ ID NO: 75).
203. A Cas12a guide RNA (gRNA) molecule comprising a protospacer domain containing a targeting sequence and a loop domain, wherein the targeting sequence corresponds to a target having the nucleotide sequence TCATCTGTAAAATAAGAGTAAAA (SEQ ID NO: 75).
204. The Cas12a gRNA of embodiment 101, wherein the target domain is in a GLA gene.
205. The Cas12a gRNA of embodiment 204, wherein the GLA gene has a IVS4+919G>A mutation.
206. The Cas12a gRNA of embodiment 205, which has the nucleotide sequence CCATGTCTCCCCACTAAAGTGTA (SEQ ID NO: 76).
207. A Cas12a guide RNA (gRNA) molecule comprising a protospacer domain containing a targeting sequence and a loop domain, wherein the targeting sequence corresponds to a target domain having the nucleotide sequence CCATGTCTCCCCACTAAAGTGTA (SEQ ID NO: 76).
208. The Cas12a gRNA of embodiment 101, wherein the target domain is in a LDLR gene.
209. The Cas12a gRNA of embodiment 208, wherein the LDLR gene has a IVS12+11C>G mutation.
210. The Cas12a gRNA of embodiment 209, wherein the target domain has the nucleotide sequence AGGTGTGGCTTAGGTACGAGATG (SEQ ID NO: 77).
211. A Cas12a guide RNA (gRNA) molecule comprising a protospacer domain containing a targeting sequence and a loop domain, wherein the targeting sequence corresponds to a target domain having the nucleotide sequence AGGTGTGGCTTAGGTACGAGATG (SEQ ID NO: 77).
212. The Cas12a gRNA of embodiment 101, wherein the target domain is in a BRIP1 gene.
213. The Cas12a gRNA of embodiment 212, wherein the BRIP1 gene has a IVS11+2767A>T mutation.
214. The Cas12a gRNA of embodiment 213, wherein the target domain has the nucleotide sequence TAAAATTCTTACATACCTTTGAA (SEQ ID NO: 78).
215. A Cas12a guide RNA (gRNA) molecule comprising a protospacer domain containing a targeting sequence and a loop domain, wherein the targeting sequence corresponds to a target having the nucleotide sequence TAAAATTCTTACATACCTTTGAA (SEQ ID NO: 78).
216. The Cas12a gRNA of embodiment 101, wherein the target domain is in a F9 gene.
217. The Cas12a gRNA of embodiment 216, wherein the F9 gene has a IVS5+13A>G mutation.
218. The Cas12a gRNA of embodiment 217, wherein the target domain has the nucleotide sequence AAAAATCTTACTCAGATTATGAC (SEQ ID NO: 79).
219. A Cas12a guide RNA (gRNA) molecule comprising a protospacer domain containing a targeting sequence and a loop domain, wherein the targeting sequence corresponds to a target domain having the nucleotide sequence AAAAATCTTACTCAGATTATGAC (SEQ ID NO: 79).
220. The Cas12a gRNA of embodiment 217, wherein the target domain has the nucleotide sequence TTTAAAAAATCTTACTCAGATTA (SEQ ID NO: 80).
221. A Cas12a guide RNA (gRNA) molecule comprising a protospacer domain containing a targeting sequence and a loop domain, wherein the targeting sequence corresponds to a target domain having the nucleotide sequence TTTAAAAAATCTTACTCAGATTA (SEQ ID NO: 80).
222. The Cas12a gRNA of embodiment 101, wherein the target domain is in a CEP290 gene.
223. The Cas12a gRNA of embodiment 222, wherein the CEP290 gene has a IVS26+1655A>G mutation.
224. The Cas12a gRNA of embodiment 223, wherein the target domain has the nucleotide sequence AGTTGTAATTGTGAGTATCTCAT (SEQ ID NO: 81).
225. A Cas12a guide RNA (gRNA) molecule comprising a protospacer domain containing a targeting sequence and a loop domain, wherein the targeting sequence corresponds to a target domain having the nucleotide sequence AGTTGTAATTGTGAGTATCTCAT (SEQ ID NO: 81).
226. The Cas12a gRNA of 101, wherein the target domain is in a COL2A1 gene.
227. The Cas12a gRNA of embodiment 226, wherein the COL2A1 gene has a IVS23+135G>A mutation.
228. The Cas12a gRNA of embodiment 227, wherein the target domain has the nucleotide sequence TCCATCCACACCGCAGGGAGAG (SEQ ID NO: 82).
229. A Cas12a guide RNA (gRNA) molecule comprising a protospacer domain containing a targeting sequence and a loop domain, wherein the targeting sequence corresponds to a target domain having the nucleotide sequence TCCATCCACACCGCAGGGAGAG (SEQ ID NO: 82).
230. The Cas12a gRNA of embodiment 101, wherein the target domain is in a USH2A gene.
231. The Cas12a gRNA of embodiment 230, wherein the USH2A gene has a IVS40-8C>G mutation.
232. The Cas12a gRNA of embodiment 231, wherein the target domain has the nucleotide sequence TGGATTTATTTTAGTTTACAGAA (SEQ ID NO: 83).
233. A Cas12a guide RNA (gRNA) molecule comprising a protospacer domain containing a targeting sequence and a loop domain, wherein the targeting sequence corresponds to a target domain having the nucleotide sequence TGGATTTATTTTAGTTTACAGAA (SEQ ID NO: 83).
234. The Cas12a gRNA of embodiment 231, wherein the target domain has the nucleotide sequence TTTTAGTTTACAGAACCTGGACC (SEQ ID NO: 84).
235. A Cas12a guide RNA (gRNA) molecule comprising a protospacer domain containing a targeting sequence and a loop domain, wherein the targeting sequence corresponds to a target domain having the nucleotide sequence TTTTAGTTTACAGAACCTGGACC (SEQ ID NO: 84).
236. The Cas12a gRNA of embodiment 231, wherein the target domain has the nucleotide sequence CAAGAGGTCTGACTTTCTGGATT (SEQ ID NO: 85).
237. A Cas12a guide RNA (gRNA) molecule comprising a protospacer domain containing a targeting sequence and a loop domain, wherein the targeting sequence corresponds to a target domain having the nucleotide sequence CAAGAGGTCTGACTTTCTGGATT (SEQ ID NO: 85).
238. The Cas12a gRNA of embodiment 231, wherein the target domain has the nucleotide sequence AGAGGTCTGACTTTCTGGATTTA (SEQ ID NO: 86).
239. A Cas12a guide RNA (gRNA) molecule comprising a protospacer domain containing a targeting sequence and a loop domain, wherein the targeting sequence corresponds to a target domain having the nucleotide sequence AGAGGTCTGACTTTCTGGATTTA (SEQ ID NO: 86).
240. The Cas12a gRNA of embodiment 231, wherein the target domain has the nucleotide sequence GGTTCTGTAAACTAAAATAAATC (SEQ ID NO: 87).
241. A Cas12a guide RNA (gRNA) molecule comprising a protospacer domain containing a targeting sequence and a loop domain, wherein the targeting sequence corresponds to a target domain having the nucleotide sequence GGTTCTGTAAACTAAAATAAATC (SEQ ID NO: 87).
242. The Cas12a gRNA of embodiment 230, wherein the USH2A gene has a IVS66+39C>T mutation.
243. The Cas12a gRNA of embodiment 242, wherein the target domain has the nucleotide sequence TATGTCTGTACACATACCTTGTT (SEQ ID NO: 88).
244. A Cas12a guide RNA (gRNA) molecule comprising a protospacer domain containing a targeting sequence and a loop domain, wherein the targeting sequence corresponds to a target domain having the nucleotide sequence TATGTCTGTACACATACCTTGTT (SEQ ID NO: 88).
245. The Cas12a gRNA of embodiment 242, wherein the target domain has the nucleotide sequence ATATGTCTGTACACATACCTTGT (SEQ ID NO: 89).
246. A Cas12a guide RNA (gRNA) molecule comprising a protospacer domain containing a targeting sequence and a loop domain, wherein the targeting sequence corresponds to a target domain having the nucleotide sequence ATATGTCTGTACACATACCTTGT (SEQ ID NO: 89).
247. The Cas12a gRNA of embodiment 230, wherein the USH2A gene has a c.7595-2144A>G mutation.
248. The Cas12a gRNA of embodiment 247, wherein the target domain has the nucleotide sequence TTAAAGATGATCTCTTACCTTGG (SEQ ID NO: 90).
249. A Cas12a guide RNA (gRNA) molecule comprising a protospacer domain containing a targeting sequence and a loop domain, wherein the targeting sequence corresponds to a target domain having the nucleotide sequence TTAAAGATGATCTCTTACCTTGG (SEQ ID NO: 90).
250. The Cas12a gRNA of embodiment 247, wherein the target domain has the nucleotide sequence CCAAGGTAAGAGATCATCTTTAA (SEQ ID NO: 91).
251. A Cas12a guide RNA (gRNA) molecule comprising a protospacer domain containing a targeting sequence and a loop domain, wherein the targeting sequence corresponds to a target domain having the nucleotide sequence CCAAGGTAAGAGATCATCTTTAA (SEQ ID NO: 91).
252. The Cas12a gRNA of embodiment 247, wherein the target domain has the nucleotide sequence AAATTGAACACCTCTCCTTTCCC (SEQ ID NO: 92).
253. A Cas12a guide RNA (gRNA) molecule comprising a protospacer domain containing a targeting sequence and a loop domain, wherein the targeting sequence corresponds to a target domain having the nucleotide sequence AAATTGAACACCTCTCCTTTCCC (SEQ ID NO: 92).
254. The Cas12a gRNA of embodiment 247, wherein the target domain has the nucleotide sequence AAGATGATCTCTTACCTTGGGAA (SEQ ID NO: 93).
255. A Cas12a guide RNA (gRNA) molecule comprising a protospacer domain containing a targeting sequence and a loop domain, wherein the targeting sequence corresponds to a target domain having the nucleotide sequence AAGATGATCTCTTACCTTGGGAA (SEQ ID NO: 93).
256. The Cas12a gRNA of embodiment 247, wherein the target domain has the nucleotide sequence AGCTGCTTTCAGCTTCCTCTCCAG (SEQ ID NO: 94).
257. A Cas12a guide RNA (gRNA) molecule comprising a protospacer domain containing a targeting sequence and a loop domain, wherein the targeting sequence corresponds to a target domain having the nucleotide sequence AGCTGCTTTCAGCTTCCTCTCCAG (SEQ ID NO: 94).
258. The Cas12a gRNA of embodiment 247, wherein the target domain has the nucleotide sequence TGGAGAGGAAGCTGAAAGCAGCT (SEQ ID NO: 95).
259. A Cas12a guide RNA (gRNA) molecule comprising a protospacer domain containing a targeting sequence and a loop domain, wherein the targeting sequence corresponds to a target domain having the nucleotide sequence TGGAGAGGAAGCTGAAAGCAGCT (SEQ ID NO: 95).
260. The Cas12a gRNA of embodiment 247, wherein the target domain has the nucleotide sequence TGTGATTCTGGAGAGGAAGCTGA (SEQ ID NO: 96).
261. A Cas12a guide RNA (gRNA) molecule comprising a protospacer domain containing a targeting sequence and a loop domain, wherein the targeting sequence corresponds to a target domain having the nucleotide sequence TGTGATTCTGGAGAGGAAGCTGA (SEQ ID NO: 96).
262. The Cas12a gRNA of embodiment 247, wherein the target domain has the nucleotide sequence ACTTGTGTGATTCTGGAGAGGAA (SEQ ID NO: 97).
263. A Cas12a guide RNA (gRNA) molecule comprising a protospacer domain containing a targeting sequence and a loop domain, wherein the targeting sequence corresponds to a target domain having the nucleotide sequence ACTTGTGTGATTCTGGAGAGGAA (SEQ ID NO: 97).
264. The Cas12a gRNA of 101, wherein the target domain is in a GAA gene.
265. The Cas12a gRNA of embodiment 264, wherein the GAA gene has a IVS1-13T>G mutation.
266. The Cas12a gRNA of embodiment 265, wherein the target domain has the nucleotide sequence TGCTGAGCCCGCTTGCTTCTCCC (SEQ ID NO: 98).
267. A Cas12a guide RNA (gRNA) molecule comprising a protospacer domain containing a targeting sequence and a loop domain, wherein the targeting sequence corresponds to a target domain having the nucleotide sequence TGCTGAGCCCGCTTGCTTCTCCC (SEQ ID NO: 98).
268. The Cas12a gRNA of embodiment 265, wherein the target domain has the nucleotide sequence GCCTCCCTGCTGAGCCCGCTTGC (SEQ ID NO: 99).
269. A Cas12a guide RNA (gRNA) molecule comprising a protospacer domain containing a targeting sequence and a loop domain, wherein the targeting sequence corresponds to a target domain having the nucleotide sequence GCCTCCCTGCTGAGCCCGCTTGC (SEQ ID NO: 99).
270. The Cas12a gRNA of embodiment 265, wherein the target domain has the nucleotide sequence TCCCGCCTCCCTGCTGAGCCCGC (SEQ ID NO: 100).
271. A Cas12a guide RNA (gRNA) molecule comprising a protospacer domain containing a targeting sequence and a loop domain, wherein the targeting sequence corresponds to a target domain having the nucleotide sequence TCCCGCCTCCCTGCTGAGCCCGC (SEQ ID NO: 100).
272. The Cas12a gRNA of embodiment 264, wherein the GAA gene has a IVS6-22T>G mutation.
273. The Cas12a gRNA of embodiment 272, wherein the target domain has the nucleotide sequence TCCTCCCTCCCTCAGGAAGTCGG (SEQ ID NO: 101).
274. A Cas12a guide RNA (gRNA) molecule comprising a protospacer domain containing a targeting sequence and a loop domain, wherein the targeting sequence corresponds to a target domain having the nucleotide sequence TCCTCCCTCCCTCAGGAAGTCGG (SEQ ID NO: 101).
275. The Cas12a gRNA of embodiment 272, wherein the target domain has the nucleotide sequence AAGGCTCCCTCCTCCCTCCCTCA (SEQ ID NO: 102).
276. A Cas12a guide RNA (gRNA) molecule comprising a protospacer domain containing a targeting sequence and a loop domain, wherein the targeting sequence corresponds to a target domain having the nucleotide sequence AAGGCTCCCTCCTCCCTCCCTCA (SEQ ID NO: 102).
277. The Cas12a gRNA of embodiment 272, wherein the target domain has the nucleotide sequence TCCCTCAGGAAGTCGGCGTTGGC (SEQ ID NO: 103).
278. A Cas12a guide RNA (gRNA) molecule comprising a protospacer domain containing a targeting sequence and a loop domain, wherein the targeting sequence corresponds to a target domain having the nucleotide sequence TCCCTCAGGAAGTCGGCGTTGGC (SEQ ID NO: 103).
279. The Cas12a gRNA of any one of embodiments 1 to 278, wherein the loop domain is 5′ to the protospacer domain.
280. The Cas12a gRNA of any one of embodiments 1 to 279, wherein the loop domain comprises a nucleotide sequence selected from UCUACUGUUGUAGA (SEQ ID NO: 1), UCUACUGUUGUAGAU (SEQ ID NO: 2), UCUGCUGUUGCAGA (SEQ ID NO: 3), UCUGCUGUUGCAGAU (SEQ ID NO: 4), UCCACUGUUGUGGA (SEQ ID NO: 5), UCCACUGUUGUGGAU (SEQ ID NO: 6), CCUACUGUUGUAGG (SEQ ID NO: 7), CCUACUGUUGUAGGU (SEQ ID NO: 8), UCUACUAUUGUAGA (SEQ ID NO: 9), UCUACUAUUGUAGAU (SEQ ID NO: 10), UCUACUGCUGUAGAU (SEQ ID NO: 11), UCUACUGCUGUAGAUU (SEQ ID NO: 12), UCUACUUUCUAGAU (SEQ ID NO: 13), UCUACUUUCUAGAUU (SEQ ID NO: 14), UCUACUUUGUAGA (SEQ ID NO: 15), UCUACUUUGUAGAU (SEQ ID NO: 16), UCUACUUGUAGA (SEQ ID NO: 17), UCUACUUGUAGAU (SEQ ID NO: 18), UAAUUUCUACUGUUGUAGAU (SEQ ID NO: 19), AGAAAUGCAUGGUUCUCAUGC (SEQ ID NO: 20), AAAAUUACCUAGUAAUUAGGU (SEQ ID NO: 21), GGAUUUCUACUUUUGUAGAU (SEQ ID NO: 22), AAAUUUCUACUUUUGUAGAU (SEQ ID NO: 23), CGCGCCCACGCGGGGCGCGAC (SEQ ID NO: 24), UAAUUUCUACUCUUGUAGAU (SEQ ID NO: 25), GAAUUUCUACUAUUGUAGAU (SEQ ID NO: 26), GAAUCUCUACUCUUUGUAGAU (SEQ ID NO: 27), UAAUUUCUACUUUGUAGAU (SEQ ID NO: 28), AAAUUUCUACUGUUUGUAGAU (SEQ ID NO: 29), GAAUUUCUACUUUUGUAGAU (SEQ ID NO: 30), UAAUUUCUACUAAGUGUAGAU (SEQ ID NO: 31), UAAUUUCUACUAUUGUAGAU (SEQ ID NO: 32), UAAUUUCUACUUCGGUAGAU (SEQ ID NO: 33), UAAUUUCUACUAUUGUAGAU (SEQ ID NO: 32), AUUUCUACUAGUGUAGAU (SEQ ID NO: 34), AUUUCUACUGUGUGUAGA (SEQ ID NO: 35), AUUUCUACUAUUGUAGAU (SEQ ID NO: 36), and AUUUCUACUUUGGUAGAU (SEQ ID NO: 37).
281. The Cas12a gRNA of any one of embodiments 1 to 279, wherein the loop domain has the nucleotide sequence UAAUUUCUACUCUUGUAGAU (SEQ ID NO: 25).
282. The Cas12a gRNA of any one of embodiments 1 to 279, wherein the loop domain has the nucleotide sequence UAAUUUCUACUAAGUGUAGAU (SEQ ID NO: 31).
283. The Cas12a gRNA of any one of embodiments 1 to 281, wherein the loop domain is 20 nucleotides in length.
284. A nucleic acid encoding the Cas12a gRNA of any one of embodiments 1 to 283.
285. The nucleic acid of embodiment 284, which further encodes a Cas12a protein.
286. The nucleic acid of embodiment 284 or embodiment 285, which is a plasmid.
287. The nucleic acid of embodiment 284 or embodiment 285, which is a virus.
288. A particle comprising the Cas12a gRNA of any one of embodiments 1 to 283.
289. The particle of embodiment 288, further comprising a Cas12a protein.
290. The particle of embodiment 288 or embodiment 289, wherein the particle is a liposome, a vesicle, or a gold nanoparticle.
291. The particle of embodiment 290, which is a liposome.
292. The particle of embodiment 290, which is a vesicle.
293. The particle of embodiment 290, which is a gold nanoparticle.
294. The particle of any one of embodiments 288 to 293, wherein:
   (a) when the PAM sequence is TTTV, the Cas12a is wild-type AsCas12a or wild-type LbCas12a;
   (b) when the PAM sequence is TYCV, CCCC, or ACCC, the Cas12a protein is AsCas12 RR; and
   (c) when the PAM sequence is TATV or RATR, the Cas12a protein is AsCas12RVR.
295. The particle of any one of embodiments 288 to 294, wherein the particle contains only a single species of Cas12a gRNA.
296. A system comprising a Cas12a protein and a gRNA molecule of any one of embodiments 1 to 283.
297. The system of embodiment 296, which comprises a single gRNA molecule.
298. The system of embodiment 296 or embodiment 297, wherein:
   (a) when the PAM sequence is TTTV, then the Cas12a is wild-type AsCas12a or wild-type LbCas12a;
   (b) when the PAM sequence is TYCV, CCCC, or ACCC, the Cas12a protein is AsCas12 RR; and
   (c) when the PAM sequence is TATV or RATR, the Cas12a protein is AsCas12 RVR.
299. The system of embodiment 298, wherein when the PAM sequence is TTTV, the Cas12a is wild-type AsCas12a.
300. The system of embodiment 298, wherein when the PAM sequence is TTTV, the Cas12a is wild-type LbCas12a.
301. The system of any one of embodiments 296 to 300, further comprising the genomic DNA.
302. A cell comprising a nucleic acid according to any one of embodiments 284 to 287.
303. A cell comprising a particle according to any one of embodiments 288 to 295.
304. A cell comprising a system of any one of embodiments 296 to 301.
305. A population of cells according to any one of embodiments 302 to 304.
306. A method of altering a cell, comprising contacting the cell with the particle of any one of embodiments 289 to 295 or the system of any one of embodiments 296 to 300.
307. The method of embodiment 306, which comprises contacting the cell with the particle of any one of embodiments 289 to 295.
308. The method of embodiment 306, which comprises contacting the cell with the system of any one of embodiments 296 to 300.
309. The method of embodiment 308, wherein the contacting comprises delivering the system to the cell via a particle or a vector.
310. The method of embodiment 308, wherein the contacting comprises delivering the system to the cell via a particle.
311. The method of embodiment 310, wherein the particle is a liposome, a vesicle, or a gold nanoparticle.
312. The method of embodiment 311, wherein the particle is a liposome.
313. The method of embodiment 311, wherein the particle is a vesicle.
314. The method of embodiment 311, wherein the particle is a gold nanoparticle.
315. The method of embodiment 309, wherein the contacting comprises delivering the system to the cell via a vector.
316. The method of embodiment 315, wherein the vector is a viral vector.
317. The method of embodiment 316, wherein the viral vector is a lentivirus, an adenovirus, or an adeno-associated virus.
318. The method of embodiment 317, wherein the viral vector is a lentivirus.
319. The method of embodiment 317, wherein the viral vector is an adenovirus.
320. The method of embodiment 317, wherein the viral vector is an adeno-associated virus.
321. The method of any one of embodiments 306 to 320, wherein the cell is a stem cell.
322. The method of any one of embodiments 306 to 321, wherein the cell is an iPS cell.
323. The method of any one of embodiments 306 to 322, wherein the contacting reduces the activity of a splice site that causes a disease phenotype.
324. The method of any one of embodiments 306 to 323, wherein the contacting restores normal splicing in the cell.
325. The method of any one of embodiments 306 to 324, wherein the cell is from a subject having a genetic disease or is derived from a cell from a subject having a genetic disease.
326. The method of embodiment 325, wherein the contacting is performed ex vivo.
327. The method of embodiment 326, further comprising returning the contacted cell to the subject's body.
328. The method of embodiment 325, wherein the contacting is performed in vivo.
329. A method of treating a subject having a CFTR gene with a 3272-26A>G mutation, comprising contacting a cell of the subject, or a cell derived from a cell of the subject with a system comprising the Cas12a gRNA of any one of embodiments 104 to 106 and a Cas12a protein.
330. A method of treating a subject having a CFTR gene with a 3849+10kbC>T mutation, comprising contacting a cell of the subject, or a cell derived from a cell of the subject with a system comprising the Cas12a gRNA of any one of embodiments 107 to 109 and a Cas12a protein.
331. A method of treating a subject having a CFTR gene with a IVS11+194A>G mutation, comprising contacting a cell of the subject, or a cell derived from a cell of the subject with a system comprising the Cas12a gRNA of any one of embodiments 110 to 114 and a Cas12a protein.
332. A method of treating a subject having a CFTR gene with a IVS19+11505C>G mutation, comprising contacting a cell of the subject, or a cell derived from a cell of the subject with a system comprising the Cas12a gRNA of any one of embodiments 115 to 119 and a Cas12a protein.
333. A method of treating a subject having a DMD gene with a IVS9+46806C>T mutation, comprising contacting a cell of the subject, or a cell derived from a cell of the subject with a system comprising the Cas12a gRNA of any one of embodiments 122 to 126 and a Cas12a protein.
334. A method of treating a subject having a DMD gene with a IVS62+62296A>G mutation, comprising contacting a cell of the subject, or a cell derived from a cell of the subject with a system comprising the Cas12a gRNA of any one of embodiments 127 to 135 and a Cas12a protein.
335. A method of treating a subject having a DMD gene with a IVS1+36947G>A mutation, comprising contacting a cell of the subject, or a cell derived from a cell of the subject with a system comprising the Cas12a gRNA of any one of embodiments 136 to 142 and a Cas12a protein.
336. A method of treating a subject having a DMD gene with a IVS2+5591T>A mutation, comprising contacting a cell of the subject, or a cell derived from a cell of the subject with a system comprising the Cas12a gRNA of any one of embodiments 143 to 145 and a Cas12a protein.
337. A method of treating a subject having a DMD gene with a IVS8-15A>G mutation, comprising contacting a cell of the subject, or a cell derived from a cell of the subject with a system comprising the Cas12a gRNA of any one of embodiments 146 to 154 and a Cas12a protein.
338. A method of treating a subject having a DMD gene a mutation in exon 50, comprising contacting a cell of the subject, or a cell derived from a cell of the subject with a system comprising the Cas12a gRNA of any one of embodiments 155 to 165 and a Cas12a protein.
339. A method of treating a subject having a HBB gene with a IVS2+645C>T mutation, comprising contacting a cell of the subject, or a cell derived from a cell of the subject with a system comprising the Cas12a gRNA of any one of embodiments 168 to 177 and a Cas12a protein.
340. A method of treating a subject having a HBB gene with a IVS2+705T>G mutation, comprising contacting a cell of the subject, or a cell derived from a cell of the subject with a system comprising the Cas12a gRNA of any one of embodiments 178 to 186 and a Cas12a protein.
341. A method of treating a subject having a HBB gene with a IVS2+745C>G mutation, comprising contacting a cell of the subject, or a cell derived from a cell of the subject with a system comprising the Cas12a gRNA of any one of embodiments 187 to 189 and a Cas12a protein.
342. A method of treating a subject having a FGB gene with a IVS6+13C>T mutation, comprising contacting a cell of the subject, or a cell derived from a cell of the subject with a system comprising the Cas12a gRNA of any one of embodiments 191 to 195 and a Cas12a protein.
343. A method of treating a subject having a SOD1 gene with a IVS4+792C>G mutation, comprising contacting a cell of the subject, or a cell derived from a cell of the subject with a system comprising the Cas12a gRNA of any one of embodiments 197 to 199 and a Cas12a protein.
344. A method of treating a subject having a QDPR gene with a IVS3+2552A>G mutation, comprising contacting a cell of the subject, or a cell derived from a cell of the subject with a system comprising the Cas12a gRNA of any one of embodiments 201 to 203 and a Cas12a protein.
345. A method of treating a subject having a GLA gene with a IVS4+919G>A mutation, comprising contacting a cell of the subject, or a cell derived from a cell of the subject with a system comprising the Cas12a gRNA of any one of embodiments 205 to 207 and a Cas12a protein.
346. A method of treating a subject having a LDLR gene with a IVS12+11C>G mutation, comprising contacting a cell of the subject, or a cell derived from a cell of the subject with a system comprising the Cas12a gRNA of any one of embodiments 209 to 211 and a Cas12a protein.
347. A method of treating a subject having a BRIP1 gene with a IVS11+2767A>T mutation, comprising contacting a cell of the subject, or a cell derived from a cell of the subject with a system comprising the Cas12a gRNA of any one of embodiments 213 to 215 and a Cas12a protein.
348. A method of treating a subject having a F9 gene with a IVS5+13A>G mutation, comprising contacting a cell of the subject, or a cell derived from a cell of the subject with a system comprising the Cas12a gRNA of any one of embodiments 217 to 221 and a Cas12a protein.
349. A method of treating a subject having a CEP290 gene with a IVS26+1655A>G mutation, comprising contacting a cell of the subject, or a cell derived from a cell of the subject with a system comprising the Cas12a gRNA of any one of embodiments 223 to 225 and a Cas12a protein.
350. A method of treating a subject having a COL2A1 gene with a IVS23+135G>A mutation, comprising contacting a cell of the subject, or a cell derived from a cell of the subject with a system comprising the Cas12a gRNA of any one of embodiments 227 to 229 and a Cas12a protein.
351. A method of treating a subject having a USH2A gene with a IVS40-8C>G mutation, comprising contacting a cell of the subject, or a cell derived from a cell of the subject with a system comprising the Cas12a gRNA of any one of embodiments 231 to 241 and a Cas12a protein.
352. A method of treating a subject having a USH2A gene with a IVS66+39C>T mutation, comprising contacting a cell of the subject, or a cell derived from a cell of the subject with a system comprising the Cas12a gRNA of any one of embodiments 242 to 246 and a Cas12a protein.
353. A method of treating a subject having a USH2A gene with a c.7595-2144A>G mutation, comprising contacting a cell of the subject, or a cell derived from a cell of the subject with a system comprising the Cas12a gRNA of any one of embodiments 247 to 263 and a Cas12a protein.
354. A method of treating a subject having a GAA gene with a IVS1-13T>G mutation, comprising contacting a cell of the subject, or a cell derived from a cell of the subject with a system comprising the Cas12a gRNA of any one of embodiments 265 to 271 and a Cas12a protein.
355. A method of treating a subject having a GAA gene with a IVS6-22T>G mutation, comprising contacting a cell of the subject, or a cell derived from a cell of the subject with a system comprising the Cas12a gRNA of any one of embodiments 272 to 278 and a Cas12a protein.
356. The method of any one of embodiments 329 to 355, which comprises contacting a cell of the subject with the system.
357. The method of embodiment 356, wherein the cell is a stem cell.
358. The method of embodiment 356 or embodiment 357, wherein the contacting is performed ex vivo, and the method further comprises returning the cell to the subject's body after contacting the cell with the system.
359. The method of embodiment 356 or embodiment 357, wherein the contacting is performed in vivo.
360. The method of any one of embodiments 329 to 355, which comprises contacting a cell derived from a cell of the subject with the system ex vivo, and the method further comprises returning the cell to the subject's body after contacting the cell with the system.
361. The method of embodiment 360, wherein the cell contacted with the system is an iPS cell.
362. The method of any one embodiments 329 to 361, wherein:
   (a) when the PAM sequence is TTTV, then the Cas12a protein is wild-type AsCas12a or wild-type LbCas12a;
   (b) when the PAM sequence is TYCV, CCCC, or ACCC, the Cas12a protein is AsCas12 RR; and
   (c) when the PAM sequence is TATV or RATR, the Cas12a protein is AsCas12RVR.
363. The method of embodiment 362, wherein when the PAM sequence is TTTV, the Cas12a protein is wild-type AsCas12a.
364. The method of embodiment 362, wherein when the PAM sequence is TTTV, the Cas12a protein is wild-type LbCas12a.
365. The method of any one of embodiments 329 to 364, wherein the contacting the cell with the system comprises delivering the system to the cell via a particle or a vector.
366. The method of embodiment 365, wherein the contacting comprises delivering the system to the cell via a particle.
367. The method of embodiment 366, wherein the particle is a liposome, a vesicle, or a gold nanoparticle.
368. The method of embodiment 367, wherein the particle is a liposome.
369. The method of embodiment 367, wherein the particle is a vesicle.
370. The method of embodiment 367, wherein the particle is a gold nanoparticle.
371. The method of embodiment 365, wherein the contacting comprises delivering the system to the cell via a vector.
372. The method of embodiment 371, wherein the vector is a viral vector.
373. The method of embodiment 372, wherein the viral vector is a lentivirus, an adenovirus, or an adeno-associated virus.
374. The method of embodiment 373, wherein the viral vector is a lentivirus.
375. The method of embodiment 373, wherein the viral vector is an adenovirus.
376. The method of embodiment 373, wherein the viral vector is an adeno-associated virus.

While various specific embodiments have been illustrated and described, it will be appreciated that various changes can be made without departing from the spirit and scope of the disclosure(s).

### 8. CITATION OF REFERENCES

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

What is claimed is:

1. A Cas12a guide RNA (gRNA) molecule for editing a human USH2A gene or a human CFTR gene comprising:
(a) a protospacer domain containing a targeting sequence; and
(b) a loop domain;
wherein
(i) the targeting sequence corresponds to a target domain in a human USH2A or human CFTR genomic DNA sequence;
(ii) the target domain is adjacent to a protospacer-adjacent motif (PAM) of a Cas12a protein; and
(iii) (a) upon introduction of the gRNA and the Cas12a protein into a human cell containing the genomic sequence, the Cas12a cleaves the genomic DNA up to 15 nucleotides from a splice site encoded by the genomic DNA and/or (b) the PAM is within 40 nucleotides of a splice site encoded by the genomic DNA.

2. The Cas12a gRNA molecule of claim 1, wherein the splice site is a cryptic splice site, optionally wherein the cryptic splice site is created by a mutation in the genomic DNA sequence or activated by a mutation in the genomic DNA sequence.

3. The Cas12a gRNA of claim 2, wherein the cryptic splice site is created by a mutation in the genomic DNA sequence or activated by a mutation in the genomic DNA sequence, and wherein the mutation is located 1 to 23 nucleotides 3′ of the PAM sequence.

4. The Cas12a gRNA of claim 2 or claim 3, wherein the mutation is a single nucleotide polymorphism.

5. The Cas12a gRNA of any one of claims 2 to 4, wherein splicing at the cryptic splice site results in a disease phenotype.

6. The Cas12a gRNA molecule of any one of claims 2 to 5, wherein the cryptic splice site is a cryptic 3′ splice site.

7. The Cas12a gRNA molecule of any one of claims 2 to 5, wherein the cryptic splice site is a cryptic 5′ splice site.

8. The Cas12a gRNA of any one of claims 1 to 7, which is 40-44 nucleotides long.

9. The Cas12a gRNA of any one of claims 1 to 8, wherein the targeting sequence is 20-24 nucleotides in length.

10. The Cas12a gRNA of any one of claims 1 to 9, wherein the protospacer domain is 17-26 nucleotides in length.

11. The Cas12a gRNA of any one of claims 1 to 10, wherein there are no mismatches between the targeting sequence and the complement of the target domain.

12. The Cas12a gRNA of claim 1, wherein the target domain is in a human USH2A gene.

13. The Cas12a gRNA of claim 12, wherein the USH2A gene has a c.7595-2144A>G mutation, a IVS40-8C>G mutation, or a IVS66+39C>T mutation.

14. The Cas12a gRNA of claim 13, wherein the USH2A gene has a c.7595-2144A>G mutation.

15. The Cas12a gRNA of claim 14, wherein the target domain has the nucleotide sequence TTAAAGATGATCTCTTACCTTGG (SEQ ID NO: 90), ACTTGTGTGATTCTGGAGAGGAA (SEQ ID NO: 97), CCAAGGTAAGAGATCATCTTTAA (SEQ ID NO: 91), AAATTGAACACCTCTCCTTTCCC (SEQ ID NO: 92), AAGATGATCTCTTACCTTGGGAA (SEQ ID NO: 93), AGCTGCTTTCAGCTTCCTCTCCAG (SEQ ID NO: 94), TGGAGAGGAAGCTGAAAGCAGCT (SEQ ID NO: 95), or TGTGATTCTGGAGAGGAAGCTGA (SEQ ID NO: 96).

16. A Cas12a guide RNA (gRNA) molecule comprising a protospacer domain containing a targeting sequence and a loop domain, wherein the targeting sequence corresponds to a target domain having the nucleotide sequence TTAAAGATGATCTCTTACCTTGG (SEQ ID NO: 90), ACTTGTGTGATTCTGGAGAGGAA (SEQ ID NO: 97), CCAAGGTAAGAGATCATCTTTAA (SEQ ID NO: 91), AAGATGATCTCTTACCTTGGGAA (SEQ ID NO: 93), AGCTGCTTTCAGCTTCCTCTCCAG (SEQ ID NO: 94), TGGAGAGGAAGCTGAAAGCAGCT (SEQ ID NO: 95), or TGTGATTCTGGAGAGGAAGCTGA (SEQ ID NO: 96).

17. The Cas12a gRNA of claim 13, wherein the USH2A gene has a IVS40-8C>G mutation.

18. The Cas12a gRNA of claim 17, wherein the target domain has the nucleotide sequence TGGATTTATTTTAGTTTACAGAA (SEQ ID NO: 83), TTTTAGTTTACAGAACCTGGACC (SEQ ID NO: 84), CAAGAGGTCTGACTTTCTGGATT (SEQ ID NO: 85), AGAGGTCTGACTTTCTGGATTTA (SEQ ID NO: 86), or GGTTCTGTAAACTAAAATAAATC (SEQ ID NO: 87).

19. A Cas12a guide RNA (gRNA) molecule comprising a protospacer domain containing a targeting sequence and a loop domain, wherein the targeting sequence corresponds to a target domain having the nucleotide sequence TGGATTTATTTTAGTTTACAGAA (SEQ ID NO: 83), TTTTAGTTTACAGAACCTGGACC (SEQ ID NO: 84), CAAGAGGTCTGACTTTCTGGATT (SEQ ID NO: 85), AGAGGTCTGACTTTCTGGATTTA (SEQ ID NO: 86), or GGTTCTGTAAACTAAAATAAATC (SEQ ID NO: 87).

20. The Cas12a gRNA of claim 13, wherein the USH2A gene has a IVS66+39C>T mutation.

21. The Cas12a gRNA of claim 20, wherein the target domain has the nucleotide sequence TATGTCTGTACACATACCTTGTT (SEQ ID NO: 88) or ATATGTCTGTACACATACCTTGT (SEQ ID NO: 89).

22. A Cas12a guide RNA (gRNA) molecule comprising a protospacer domain containing a targeting sequence and a loop domain, wherein the targeting sequence corresponds to a target domain having the nucleotide sequence TATGTCTGTACACATACCTTGTT (SEQ ID NO: 88) or ATATGTCTGTACACATACCTTGT (SEQ ID NO: 89).

23. The Cas12a gRNA of claim 1, wherein the target domain is in a human CFTR gene.

24. The Cas12a gRNA of claim 23, wherein the CFTR gene has a has a mutation which is a 3272-26A>G mutation, a 3849+10kbC>T mutation, a IVS11+194A>G mutation, or a IVS19+11505C>G mutation.

25. The Cas12a gRNA of claim 24, wherein the mutation is a 3272-26A>G mutation.

26. The Cas12a gRNA of claim 25, wherein the target domain has the nucleotide sequence CATAGAAAACACTGCAAATAACA (SEQ ID NO: 38).

27. A Cas12a guide RNA (gRNA) molecule comprising a protospacer domain containing a targeting sequence and a loop domain, wherein the targeting sequence corresponds to a target domain having the nucleotide sequence CATAGAAAACACTGCAAATAACA (SEQ ID NO: 38).

28. The Cas12a gRNA of claim 24, wherein the mutation is a 3849+10kbC>T mutation.

29. The Cas12a gRNA of claim 28, wherein the target domain has the nucleotide sequence AGGGTGTCTTACTCACCATTTTA (SEQ ID NO: 39).

30. A Cas12a guide RNA (gRNA) molecule comprising a protospacer domain containing a targeting sequence and a loop domain, wherein the targeting sequence corresponds to a target domain having the nucleotide sequence AGGGTGTCTTACTCACCATTTTA (SEQ ID NO: 39).

31. The Cas12a gRNA of claim 24, wherein the mutation is a IVS11+194A>G mutation.

32. The Cas12a gRNA of claim 31, wherein the target domain has the nucleotide sequence TACTTGAGATGTAAGTAAGGTTA (SEQ ID NO: 40) or ATAGTAACCTTACTTACATCTCA (SEQ ID NO: 41).

33. A Cas12a guide RNA (gRNA) molecule comprising a protospacer domain containing a targeting sequence and a loop domain, wherein the targeting sequence corresponds to a target domain having the nucleotide sequence TACTTGAGATGTAAGTAAGGTTA (SEQ ID NO: 40) or ATAGTAACCTTACTTACATCTCA (SEQ ID NO: 41).

34. The Cas12a gRNA of claim 24, wherein the mutation is a IVS19+11505C>G mutation.

35. The Cas12a gRNA of claim 34, wherein the target domain has the nucleotide sequence AAATTCCATCTTACCAATTCTAA (SEQ ID NO: 42) or AACGTTAAAATTCCATCTTACCA (SEQ ID NO: 43).

36. A Cas12a guide RNA (gRNA) molecule comprising a protospacer domain containing a targeting sequence and a loop domain, wherein the targeting sequence corresponds to a target domain having the nucleotide sequence AAATTCCATCTTACCAATTCTAA (SEQ ID NO: 42) or AACGTTAAAATTCCATCTTACCA (SEQ ID NO: 43).

37. A Cas12a guide RNA (gRNA) molecule comprising a protospacer domain containing a targeting sequence and a loop domain, wherein the targeting sequence corresponds to a target domain having the nucleotide sequence:
(SEQ ID NO: 44)
TGACCTTTGGTAAGTCATCTAAT,
(SEQ ID NO: 45)
CCTTTGTGACCTTTGGTAAGTCA,
(SEQ ID NO: 46)
TTGATCACATAACAAGGTCAGTT,
(SEQ ID NO: 47)
ATCACATAACAAGGTCAGTTTAT,
(SEQ ID NO: 48)
AGTTATGATAAACTGACCTTGTT,
(SEQ ID NO: 49)
TGATAAACTGACCTTGTTATGTG,
(SEQ ID NO: 50)
TCTTCCTTGGTTTTGCAGCTTCT,
(SEQ ID NO: 51)
TTGGTTTTGCAGCTTCTCGAGTT,
(SEQ ID NO: 51)
TTGGTTTTGCAGCTTCTCGAGTT,
(SEQ ID NO: 52)
CTCTTTCTCTTCCTTGGTTTTGC,
(SEQ ID NO: 53)
CTTGTTTCTCTACATAGGTTGAA,
(SEQ ID NO: 54)
TCCTCTCTATCCACCTCCCCCAG,
(SEQ ID NO: 55)
CCTCCCCCAGACCCTTCTCTGCA,
(SEQ ID NO: 56)
CCCCTCCTCTCTATCCACTCCCC,
(SEQ ID NO: 57)
CCTCCTCTCTATCCACCTCCCCC,
(SEQ ID NO: 58)
CAAAAACCCAAAATATTTTAGCT,
(SEQ ID NO: 59)
CTTTTTGCAAAAACCCAAAATAT,
(SEQ ID NO: 60)
TTTTTGCAAAAACCCAAAATATT,
(SEQ ID NO: 61)
TGTCACCAGAGTAACAGTCTGAG,
(SEQ ID NO: 62)
GCTCCTACTCAGACTGTTACTCT,
(SEQ ID NO: 63)
TGGGTTAAGGTAATAGCAATATC,
(SEQ ID NO: 64)
TATGCAGAGATATTGCTATTACC,
(SEQ ID NO: 65)
CTATTACCTTAACCCAGAAATTA,
(SEQ ID NO: 66)
CAGAGATATTGCTATTACCTTAA,
(SEQ ID NO: 67)
TGCATATAAATTGTAACTGAGGT,
(SEQ ID NO: 68)
AATTGTAACTGAGGTAAGAGGTT,
(SEQ ID NO: 69)
AAACCTCTTACCTCAGTTACAAT,
(SEQ ID NO: 70)
GCAATATGAAACCTCTTACCTCA,
(SEQ ID NO: 71)
CTAATAGCAGCTACAATCCAGGT,
(SEQ ID NO: 72)
TTTTGCATACCTGTTCGTTACCT,
(SEQ ID NO: 73)
AAATAGAATGATTTTATTTTGCA,
(SEQ ID NO: 74)
TGGTAAGTTACACTAACCTTAGT,
(SEQ ID NO: 75)
TCATCTGTAAAATAAGAGTAAAA,
(SEQ ID NO: 76)
CCATGTCTCCCCACTAAAGTGTA,
(SEQ ID NO: 77)
AGGTGTGGCTTAGGTACGAGATG,
(SEQ ID NO: 78)
TAAAATTCTTACATACCTTTGAA,
(SEQ ID NO: 79)
AAAAATCTTACTCAGATTATGAC,
(SEQ ID NO: 80)
TTTAAAAAATCTTACTCAGATTA,
(SEQ ID NO: 81)
AGTTGTAATTGTGAGTATCTCAT,
(SEQ ID NO: 82)
TCCATCCACACCGCAGGGAGAG,
(SEQ ID NO: 98)
TGCTGAGCCCGCTTGCTTCTCCC,
(SEQ ID NO: 99)
GCCTCCCTGCTGAGCCCGCTTGC,
(SEQ ID NO: 100)
TCCCGCCTCCCTGCTGAGCCCGC,
(SEQ ID NO: 101)
TCCTCCCTCCCTCAGGAAGTCGG,
(SEQ ID NO: 102)
AAGGCTCCCTCCTCCCTCCCTCA,
or
(SEQ ID NO: 103)
TCCCTCAGGAAGTCGGCGTTGGC.

38. The Cas12a gRNA of any one of claims 1 to 37, wherein the loop domain is 5′ to the protospacer domain.

39. The Cas12a gRNA of any one of claims 1 to 38, wherein the loop domain has the nucleotide sequence UAAUUUCUACUAAGUGUAGAU (SEQ ID NO: 31) or UAAUUUCUACUCUUGUAGAU (SEQ ID NO: 25).

40. A nucleic acid encoding the Cas12a gRNA of any one of claims 1 to 39.

41. The nucleic acid of claim 40, which further encodes a Cas12a protein.

42. A particle comprising the Cas12a gRNA of any one of claims 1 to 39 and a Cas12a protein.

43. A system comprising a Cas12a protein and a gRNA molecule of any one of claims 1 to 39.

44. A cell comprising a nucleic acid according to claim 40 or claim 41, a particle according to claim 42, or a system according to claim 43.

45. A method of altering a cell, comprising contacting the cell with the particle of claim 42 or the system of claim 43.

46. The method of claim 45, wherein the contacting reduces the activity of a splice site that causes a disease phenotype and/or restores normal splicing in the cell.

47. The method of claim 45 or claim 46, wherein the cell is from a subject having a genetic disease or is derived from a cell from a subject having a genetic disease.

48. The method of claim 47, wherein the contacting is performed ex vivo, and optionally wherein the method further comprises returning the contacted cell to the subject's body.

49. The method of claim 47, wherein the contacting is performed in vivo.

50. A method of treating a subject having a USH2A gene with a c.7595-2144A>G mutation, comprising contacting a cell of the subject, or a cell derived from a cell of the subject with a system comprising the Cas12a gRNA of any one of claims 14 to 16 and a Cas12a protein.

51. A method of treating a subject having a USH2A gene with a IVS40-8C>G mutation, comprising contacting a cell of the subject, or a cell derived from a cell of the subject with a system comprising the Cas12a gRNA of any one of claims 17 to 19 and a Cas12a protein.

52. A method of treating a subject having a USH2A gene with a IVS66+39C>T mutation, comprising contacting a cell of the subject, or a cell derived from a cell of the subject with a system comprising the Cas12a gRNA of any one of claims 20 to 22 and a Cas12a protein.

53. A method of treating a subject having a CFTR gene with a 3272-26A>G mutation, comprising contacting a cell of the subject, or a cell derived from a cell of the subject with a system comprising the Cas12a gRNA of any one of claims 25 to 27 and a Cas12a protein.

54. A method of treating a subject having a CFTR gene with a 3849+10kbC>T mutation, comprising contacting a cell of the subject, or a cell derived from a cell of the subject with a system comprising the Cas12a gRNA of any one of claims 28 to 30 and a Cas12a protein.

55. A method of treating a subject having a CFTR gene with a IVS11+194A>G mutation, comprising contacting a cell of the subject, or a cell derived from a cell of the subject with a system comprising the Cas12a gRNA of any one of claims 31 to 33 and a Cas12a protein.

56. A method of treating a subject having a CFTR gene with a IVS19+11505C>G mutation, comprising contacting a cell of the subject, or a cell derived from a cell of the subject with a system comprising the Cas12a gRNA of any one of claims 34 to 36 and a Cas12a protein.

57. The method of any one of claims 50 to 56, which comprises contacting a cell of the subject with the system ex vivo, and wherein the method further comprises returning the cell to the subject's body after contacting the cell with the system.

58. The method of any one of claims 50 to 56, which comprises contacting a cell of the subject with the system in vivo.

59. The method of any one of claims 50 to 56, which comprises contacting a cell derived from a cell of the subject with the system ex vivo, and wherein the method further comprises returning the cell to the subject's body after contacting the cell with the system.
